(19) United States
(12) Patent Application Publication
(10) Pub. No.: US 2022/0226298 A1
(43) Pub. Date: Jul. 21, 2022

(54) GPR40 AGONISTS

(71) Applicant: Kallyope, Inc., New York, NY (US)

(72) Inventors: Iyassu SEBHAT, Jersey City, NJ (US); Shuwen HE, Fanwood, NJ (US)

(21) Appl. No.: 17/614,100

(22) PCT Filed: May 22, 2020
(86) PCT No.: PCT/US2020/034226

§ 371 Date: Nov. 24, 2021

Related U.S. Application Data
(60) Provisional application No. 62/854,249, filed on May 29, 2019.

(51) Int. Cl. A61K 31/4418; A61K 45/06; C07C 69/94; C07D 261/18; C07D 207/16; C07D 213/647; C07D 295/205; C07D 211/62; C07D 213/75; C07D 401/10; C07D 405/12; C07F 9/38; C07F 9/30; C07F 9/58; A61K 31/235; A61K 31/415; A61K 31/40; A61K 31/495; A61K 31/445; A61K 31/44; A61K 31/454; A61K 31/397; A61K 31/194; A61K 31/443; A61K 31/675

(52) U.S. Cl. CPC A61K 31/4418; A61K 31/675; C07C 69/94; C07D 261/18; C07D 207/16; C07D 213/647; C07D 295/205; C07D 211/62; C07D 213/75; C07D 401/10; C07D 405/12; C07F 9/3808; C07F 9/306; C07F 9/58; A61K 31/235; A61K 31/415; A61K 31/40; A61K 31/495; A61K 31/445; A61K 31/44; A61K 31/454; A61K 31/397; A61K 31/194; A61K 31/443; A61K 45/06

(57) ABSTRACT

This disclosure is directed, at least in part, to GPR40 agonists useful for the treatment of conditions or disorders involving the gut-brain axis. In some embodiments, the GPR40 agonists are gut-restricted compounds. In some embodiments, the GPR40 agonists are full agonists or partial agonists. In some embodiments, the condition or disorder is a metabolic disorder, such as diabetes, obesity, nonalcoholic steatohepatitis (NASH), or a nutritional disorder such as short bowel syndrome.

### CROSS-REFERENCE TO RELATED APPLICATIONS

This application claims the benefit of U.S. Provisional Application No. 62/854,249 filed on May 29, 2019, which is incorporated herein by reference in its entirety.

### BRIEF SUMMARY OF THE INVENTION

Disclosed herein, in certain embodiments, are free fatty acid receptor 1 (GPR40) agonists useful for the treatment of conditions or disorders involving the gut-brain axis. In some embodiments, the GPR40 agonists are gut-restricted or selectively modulate GPR40 located in the gut. In some embodiments, the GPR40 agonists are soft drugs, as described herein. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

Disclosed herein, in certain embodiments, is a compound of Formula (I):

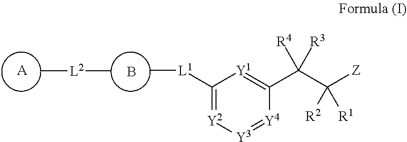

or a pharmaceutically acceptable salt, solvate, stereoisomer, or prodrug thereof, wherein:
Z is —C(O)OH, —C(O)OR⁵, —C(O)NR⁶, —C(O)NHS(O)₂R⁵, —S(O)₂NHC(O)R⁵, —P(O)(R⁵)OR⁶, —P(O)(OR⁶)₂, or —S(O)₂OR⁶;
or Z is a 4- or 5-membered carbocycle or heterocycle which is unsubstituted or substituted with 1, 2, 3, or 4 substituents selected from C₁-C₆ alkyl, —O—(C₁-C₆ alkyl), —OH, and ═O;
R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —P(O)(OH)₂, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

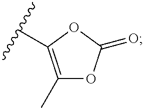 [SMILES: *c1oc(=O)oc1C]

R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
R¹, R², R³, and R⁴ are each independently hydrogen, halogen, —OH, —O—(C₁-C₆ alkyl), C₁-C₆ alkyl, C₃-C₆ cycloalkyl, or 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
Y¹, Y², Y³, and Y⁴ are each independently N, CH, or C—R^{Y};
each R^{Y} is independently halogen, —CN, —OH, —O—(C₁-C₆ alkyl), —NH₂, —NH—(C₁-C₆ alkyl), —N(C₁-C₆ alkyl)₂, C₁-C₆ alkyl, C₃-C₆ cycloalkyl, and 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
L¹ is *—O—C(O)—, or *—C(O)—O—; wherein * represents the connection to Ring B;
Ring B is arylene, heteroarylene, C₃-C₁₀ cycloalkylene, or 3- to 10-membered heterocycloalkylene; wherein the arylene, heteroarylene, cycloalkylene, or heterocycloalkylene is unsubstituted or substituted with 1, 2, 3, or 4 R^{B} substituents; Ring A is aryl, heteroaryl, C₃-C₁₀ cycloalkyl, or 3- to 10-membered heterocycloalkyl; wherein the aryl, heteroaryl, cycloalkyl, or heterocycloalkyl is unsubstituted or substituted with 1, 2, 3, 4, or 5 R^{A} substituents;
L² is a bond, C₁-C₆ alkylene, or —(C₁-C₆ alkylene)-O—; wherein the alkylene is unsubstituted or substituted with 1, 2, or 3 substituents selected from the group consisting of halogen, —CN, —OH, C₁-C₆ alkyl, and —O—(C₁-C₆ alkyl);
each R^{A} is independently halogen, C₁-C₁₀ alkyl, C₂-C₁₀ alkenyl, C₂-C₁₀ alkynyl, C₁-C₁₀ fluoroalkyl, -L^{A}-CN, -L^{A}-OH, -L^{A}-OR¹⁰, -L^{A}-NR¹¹R¹¹, -L^{A}-C(═O)R¹⁰, -L^{A}C(═O)OR¹¹, -L^{A}-OC(═O)R¹¹, -L^{A}-C(═O)NR¹¹R¹¹, -L^{A}-NR¹¹C(═O)R¹¹, -L^{A}-NR¹¹C(═O)NR¹¹R¹¹, -L^{A}-OC(═O)NR¹¹R¹¹, -L^{A}-NR¹¹C(═O)OR¹⁰, -L^{A}-OC(═O)OR¹⁰, -L^{A}-aryl, -L^{A}-heteroaryl, -L^{A}-(C₃-C₁₀ cycloalkyl), or -L^{A}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each R^{B} is independently halogen, C₁-C₁₀ alkyl, C₂-C₁₀ alkenyl, C₂-C₁₀ alkynyl, C₁-C₁₀ fluoroalkyl, -L^{B}-CN, -L^{B}-OH, -L^{B}-OR¹⁰, -L^{B}-NR¹¹R¹¹, -L^{B}-C(═O)R¹⁰, -L^{B}-C(═O)OR¹¹, -L^{B}-OC(═O)R¹¹, -L^{B}-C(═O)NR¹¹R¹¹, -L^{B}-NR¹¹C(═O)R¹¹, -L^{B}-NR¹¹C(═O)NR¹¹R¹¹, -L^{B}-OC(═O)NR¹¹R¹¹, -L^{B}-NR¹¹C(═O)OR¹⁰, -L^{B}-OC(═O)OR¹⁰, -L^{B}-aryl, -L^{B}-heteroaryl, -L^{B}-(C₃-C₁₀ cycloalkyl), or -L^{B}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each L^{A} and L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl;
each R¹⁰ is independently C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl); and
each R¹¹ is independently hydrogen, C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
or two R¹¹ on the same nitrogen atom are taken together with the nitrogen to which they are attached to form a 3- to 10-membered N-heterocycloalkyl; wherein the heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl).

Any combination of the groups described above or below for the various variables is contemplated herein. Throughout the specification, groups and substituents thereof are chosen by one skilled in the field to provide stable moieties and compounds.

In some embodiments of a compound of Formula (I), or a pharmaceutically acceptable salt, solvate, stereoisomer, or prodrug thereof, Y¹, Y², Y³, and Y⁴ are each independently N, CH, or C—R^{Y}; and each R^{Y} is independently F, Cl, Br, —CN, —OH, —O—(C₁-C₆ alkyl), or C₁-C₆ alkyl. In some embodiments, Y¹, Y², Y³, and Y⁴ are each independently N or CH.

In some embodiments of a compound of Formula (I), or a pharmaceutically acceptable salt, solvate, stereoisomer, or prodrug thereof, Z is —C(O)OH, —C(O)OR⁵, —C(O)NHR⁶, —C(O)NHS(O)₂R⁵, —S(O)₂NHC(O)R⁵, —P(O)(R⁵)OR⁶, —P(O)(OR⁶)₂, or —S(O)₂OR⁶; R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —P(O)(OH)₂, —O—(C₁-C₆ alkyl), C₁-C₆ alkyl, C₁-C₆ hydroxyalkyl, and

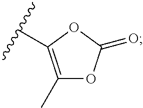 [SMILES: *c1oc(=O)oc1C]

and R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —O—(C₁-C₆ alkyl), C₁-C₆ alkyl, and C₁-C₆ hydroxyalkyl. In some embodiments, Z is —C(O)OH.

In some embodiments, the compound of Formula (I), or a pharmaceutically acceptable salt, solvate, stereoisomer, or prodrug thereof, is a compound of Formula (II):

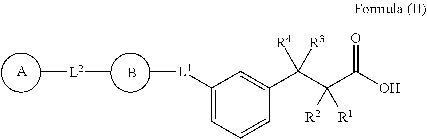

or a pharmaceutically acceptable salt, solvate, stereoisomer, or prodrug thereof.

In some embodiments of a compound of Formula (I) or (II), or a pharmaceutically acceptable salt, solvate, stereoisomer, or prodrug thereof, R¹, R², R³, and R⁴ are each independently hydrogen, halogen, C₁-C₆ alkyl, C₃-C₆ cycloalkyl. In some embodiments, R¹, R², and R³ are each independently hydrogen, halogen, or C₁-C₆ alkyl; and R⁴ is C₃-C₆ cycloalkyl.

In some embodiments, the compound of Formula (I) or (II), or a pharmaceutically acceptable salt, solvate, stereoisomer, or prodrug thereof, is a compound of Formula (III):

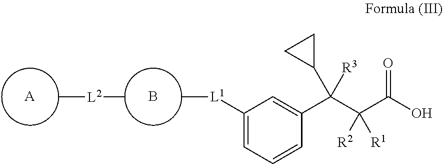

or a pharmaceutically acceptable salt, solvate, stereoisomer, or prodrug thereof, wherein: R¹, R², and R³ are each independently hydrogen, —F, or methyl.

In some embodiments, the compound of Formula (I), (II), or (III), or a pharmaceutically acceptable salt, solvate, stereoisomer, or prodrug thereof, is a compound of Formula (IV):

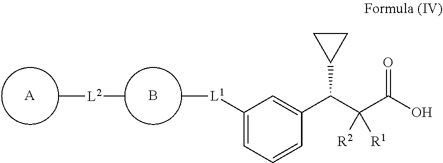

or a pharmaceutically acceptable salt, solvate, stereoisomer, or prodrug thereof, wherein: R¹ and R² are each independently hydrogen, —F, or methyl.

In some embodiments, the compound of Formula (I), (II), (III), or (IV), or a pharmaceutically acceptable salt, solvate, stereoisomer, or prodrug thereof, is a compound of Formula (Va) or Formula (Vb):

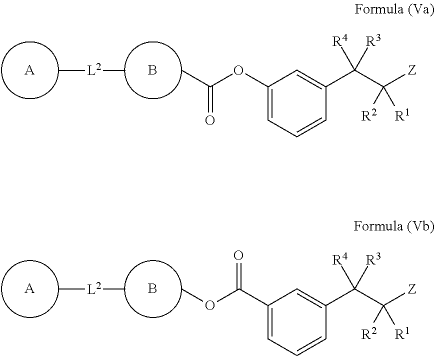

or a pharmaceutically acceptable salt, solvate, stereoisomer, or prodrug thereof.

In some embodiments of a compound of Formula (I), (II), (III), (IV), (Va), or (Vb), or a pharmaceutically acceptable salt, solvate, stereoisomer, or prodrug thereof, Ring B is 3- to 6-membered heterocycloalkylene; wherein the heterocycloalkylene is unsubstituted or substituted with 1, 2, 3, or 4 R^{B} substituents; each R^{B} is independently unsubstituted C₁-C₁₀ alkyl; L² is C₁-C₆ alkylene; wherein the alkylene is unsubstituted or substituted with 1, 2, or 3 substituents selected from the group consisting of —OH, C₁-C₆ alkyl, and —O—(C₁-C₆ alkyl); and Ring A is aryl or heteroaryl; wherein the aryl or heteroaryl is unsubstituted or substituted with 1, 2, or 3 R^{A} substituents.

In some embodiments, the compound of Formula (I), (II), (III), (IV), (Va), or (Vb), or a pharmaceutically acceptable salt, solvate, stereoisomer, or prodrug thereof, is a compound of Formula (VIa) or Formula (VIb):

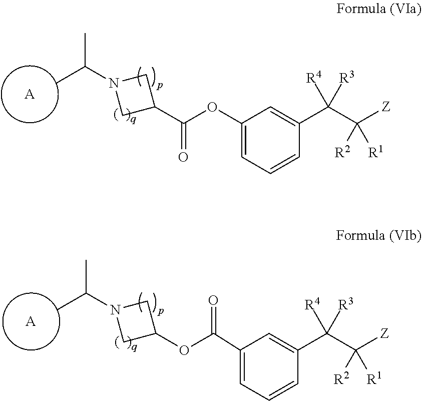

or a pharmaceutically acceptable salt, solvate, stereoisomer, or prodrug thereof; wherein: p and q are each independently 1 or 2.

In some embodiments of a compound of Formula (I), (II), (III), (IV), (Va), (Vb), (VIa), or (VIb), or a pharmaceutically acceptable salt, solvate, stereoisomer, or prodrug thereof, Ring A is phenyl or 5- or 6-membered monocyclic heteroaryl; wherein the phenyl or heteroaryl is unsubstituted or is substituted with 1, 2, or 3 R^{A} substituents; each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹; wherein the alkyl is unsubstituted or substituted with 1, 2, or 3 substituents selected from the group consisting of halogen, —OH, C₁-C₆ fluoroalkyl, —O—(C₁-C₆ alkyl), and —O—(C₁-C₆ fluoroalkyl); and each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, each R^{A} is independently halogen, C₁-C₆ alkyl, C₁-C₆ fluoroalkyl, -L^{A}-OH, -L^{A}-OR¹⁰; wherein the alkyl is unsubstituted or substituted with 1, 2, or 3 substituents selected from the group consisting of halogen, —OH, and C₁-C₆ fluoroalkyl; and each L^{A} is independently a bond or unsubstituted C₁-C₆ alkylene.

In some embodiments, the compound of Formula (I), (II), (III), (IV), (Va), or (Vb), or a pharmaceutically acceptable salt, solvate, stereoisomer, or prodrug thereof, is a compound of Formula (VIIa) or Formula (VIIb):

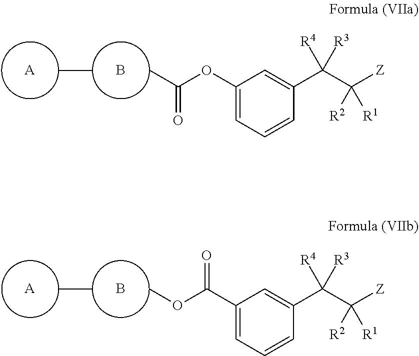

or a pharmaceutically acceptable salt, solvate, stereoisomer, or prodrug thereof; wherein: Ring B is arylene or heteroarylene; wherein the arylene or heteroarylene is unsubstituted or substituted with 1, 2, 3, or 4 R^{B} substituents; and Ring A is aryl or heteroaryl; wherein the aryl or heteroaryl is unsubstituted or substituted with 1, 2, 3, 4, or 5 R^{A} substituents.

In some embodiments of a compound of Formula (I), (II), (III), (IV), (Va), (Vb), (VIIa), or (VIIb), or a pharmaceutically acceptable salt, solvate, stereoisomer, or prodrug thereof, Ring B is phenylene or 5- or 6-membered monocyclic heteroarylene; wherein the phenylene or heteroarylene is unsubstituted or is substituted with 1, 2, or 3 R^{B} substituents; each R^{B} is independently halogen, C₁-C₆ alkyl, C₁-C₆ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-C(═O)NR¹¹R¹¹, or -L^{B}-(3- to 10-membered heterocycloalkyl); wherein each alkyl and heterocycloalkyl is unsubstituted or substituted with 1, 2, or 3 substituents selected from the group consisting of halogen, —CN, —OH, C₁-C₆ alkyl, C₁-C₆ fluoroalkyl, —O—(C₁-C₆ alkyl), and —O—(C₁-C₆ fluoroalkyl); each L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl; Ring A is phenyl or 5- or 6-membered monocyclic heteroaryl; wherein the phenyl or heteroaryl is unsubstituted or is substituted with 1, 2, or 3 R^{A} substituents; each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹; wherein the alkyl is unsubstituted or substituted with 1, 2, or 3 substituents selected from the group consisting of —OH, C₁-C₆ alkyl, and —O—(C₁-C₆ alkyl); and each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, Ring B is phenylene or 5- or 6-membered monocyclic heteroarylene; wherein the phenylene or heteroarylene is unsubstituted or is substituted with 1, 2, or 3 R^{B} substituents; each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of C₁-C₆ alkyl; each L^{B} is independently a bond or unsubstituted C₁-C₆ alkylene; Ring A is phenyl or 6-membered monocyclic heteroaryl; wherein the phenyl or heteroaryl is unsubstituted or is substituted with 1, 2, or 3 R^{A} substituents; each R^{A} is independently fluoro, chloro, C₁-C₆ alkyl, C₁-C₆ fluoroalkyl, -L^{A}-OH, -L^{A}-OR¹⁰, -L^{A}-NR¹¹R¹¹, or -L^{A}-C(═O)NR¹¹R¹¹; and each L^{A} is independently a bond or unsubstituted C₁-C₆ alkylene.

In some embodiments, the compound of Formula (I), (II), (III), (IV), (Va), (Vb), (VIIa), or (VIIb), or a pharmaceutically acceptable salt, solvate, stereoisomer, or prodrug thereof, is a compound of Formula (VIIIa) or Formula (VIIIb):

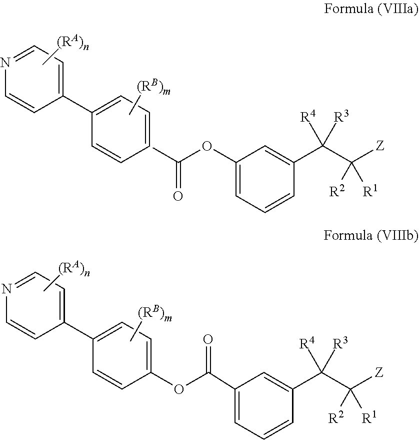

or a pharmaceutically acceptable salt, solvate, stereoisomer, or prodrug thereof; wherein: n and m are each independently 0, 1, 2, or 3.

In some embodiments, the compound of Formula (I), (II), (III), (IV), (Va), (Vb), (VIIa), (VIIb), (VIIIa), or (VIIIb), or a pharmaceutically acceptable salt, solvate, stereoisomer, or prodrug thereof, each R¹⁰ is independently C₁-C₆ alkyl; wherein each alkyl is unsubstituted or substituted with 1, 2, 3, 4, or 5 substituents selected from the group consisting of halogen, —OH, C₁-C₆ alkyl and C₁-C₆ hydroxyalkyl; and each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of halogen, —OH, C₁-C₆ alkyl and C₁-C₆ hydroxyalkyl; or two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

Disclosed herein, in certain embodiments, are pharmaceutical compositions comprising a compound disclosed herein, or a pharmaceutically acceptable salt, solvate, stereoisomer, or prodrug thereof, and at least one pharmaceutically acceptable excipient.

Disclosed herein, in certain embodiments, are methods of treating a condition or disorder involving the gut-brain axis in a subject in need thereof, the method comprising administering to the subject a therapeutically effective amount of a compound disclosed herein, or a pharmaceutically acceptable salt, solvate, stereoisomer, or prodrug thereof. In some embodiments, the condition or disorder is associated with GPR40 activity. In some embodiments, the condition or disorder is a metabolic disorder. In some embodiments, the condition or disorder is type 2 diabetes, hyperglycemia, metabolic syndrome, obesity, hypercholesterolemia, nonalcoholic steatohepatitis, or hypertension. In some embodiments, the condition or disorder is a nutritional disorder. In some embodiments, the condition or disorder is short bowel syndrome, intestinal failure, or intestinal insufficiency. In some embodiments, the compound disclosed herein is gut-restricted. In some embodiments, the compound disclosed herein is a soft drug. In some embodiments, the compound disclosed herein has low systemic exposure.

In some embodiments, the methods disclosed herein further comprise administering one or more additional therapeutic agents to the subject. In some embodiments, the one or more additional therapeutic agents are selected from a TGR5 agonist, a GPR119 agonist, an SSTR5 antagonist, an SSTR5 inverse agonist, a CCK1 agonist, a PDE4 inhibitor, a DPP-4 inhibitor, or a combination thereof. In some embodiments, the TGR5 agonist, GPR119 agonist, SSTR5 antagonist, SSTR5 inverse agonist or CCK1 agonist is gut-restricted.

Also disclosed herein, in certain embodiments, is the use of a compound disclosed herein, or a pharmaceutically acceptable salt, solvate, stereoisomer, or prodrug thereof, for the preparation of a medicament for the treatment of a condition or disorder involving the gut-brain axis in a subject in need thereof.

Also disclosed herein, in certain embodiments, are methods of treating a condition or disorder involving the gut-brain axis in a subject in need thereof, the method comprising administering to the subject a therapeutically effective amount of a gut-restricted GPR40 modulator.

Also disclosed herein, in certain embodiments, is the use of a gut-restricted GPR40 modulator for the preparation of a medicament for the treatment of a condition or disorder involving the gut-brain axis in a subject in need thereof.

### DETAILED DESCRIPTION OF THE INVENTION

This disclosure is directed, at least in part, to GPR40 agonists useful for the treatment of conditions or disorders involving the gut-brain axis. In some embodiments, the GPR40 agonists are gut-restricted compounds. In some embodiments, the GPR40 agonists are full agonists or partial agonists.

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulas, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

“Alkyl” refers to an optionally substituted straight-chain, or optionally substituted branched-chain saturated hydrocarbon monoradical having from one to about ten carbon atoms, or more preferably, from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₅ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{a}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon or an sp³-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl (—C(CH₃)═CH₂), butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. Unless stated otherwise specifically in the specification, an alkenyl group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms, wherein an sp-hybridized carbon or an sp³-hybridized carbon of the alkynyl residue is attached to the rest of the molecule by a single bond. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. Unless stated otherwise specifically in the specification, an alkylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. Unless stated otherwise specifically in the specification, an alkenylene group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. Unless stated otherwise specifically in the specification, an alkynylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkoxy” or “alkoxyl” refers to a radical bonded through an oxygen atom of the formula —O-alkyl, where alkyl is an alkyl chain as defined above.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from 6 to 18 carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some embodiments, the aryl is a C₆-C₁₀ aryl. In some embodiments, the aryl is a phenyl. Unless stated otherwise specifically in the specification, the term “aryl” or the prefix “ar-” (such as in “aralkyl”) is meant to include aryl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N+(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

An “arylene” refers to a divalent radical derived from an “aryl” group as described above linking the rest of the molecule to a radical group. The arylene is attached to the rest of the molecule through a single bond and to the radical group through a single bond. In some embodiments, the arylene is a phenylene. Unless stated otherwise specifically in the specification, an arylene group is optionally substituted as described above for an aryl group.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, 7,7-dimethyl-bicyclo[2.2.1]heptanyl, and the like. Unless otherwise stated specifically in the specification, the term “cycloalkyl” is meant to include cycloalkyl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N+(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “cycloalkylene” refers to a divalent radical derived from a “cycloalkyl” group as described above linking the rest of the molecule to a radical group. The cycloalkylene is attached to the rest of the molecule through a single bond and to the radical group through a single bond. Unless stated otherwise specifically in the specification, a cycloalkylene group is optionally substituted as described above for a cycloalkyl group.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like.

“Haloalkoxy” or “haloalkoxyl” refers to an alkoxyl radical, as defined above, that is substituted by one or more halo radicals, as defined above.

“Fluoroalkoxy” or “fluoroalkoxyl” refers to an alkoxy radical, as defined above, that is substituted by one or more fluoro radicals, as defined above, for example, trifluoromethoxy, difluoromethoxy, fluoromethoxy, and the like.

“Hydroxyalkyl” refers to an alkyl radical, as defined above, that is substituted by one or more hydroxy radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 8-membered heterocycloalkyl. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. More preferably, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N+(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals.

“C-heterocycloalkyl” refers to a heterocycloalkyl radical as defined above and where the point of attachment of the heterocycloalkyl radical to the rest of the molecule is through a carbon atom in the heterocycloalkyl radical. A C-heterocycloalkyl radical is optionally substituted as described above for heterocycloalkyl radicals.

A “heterocycloalkylene” refers to a divalent radical derived from a “heterocycloalkyl” group as described above linking the rest of the molecule to a radical group. The heterocycloalkylene is attached to the rest of the molecule through a single bond and to the radical group through a single bond. Unless stated otherwise specifically in the specification, a heterocycloalkylene group is optionally substituted as described above for a heterocycloalkyl group.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) 7r-electron system in accordance with the Hückel theory. In some embodiments, the heteroaryl is a 5- to 10-membered heteroaryl. In some embodiments, the heteroaryl is a monocyclic heteroaryl, or a monocyclic 5- or 6-membered heteroaryl. In some embodiments, the heteroaryl is a 6,5-fused bicyclic heteroaryl. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N+(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “heteroarylene” refers to a divalent radical derived from a “heteroaryl” group as described above linking the rest of the molecule to a radical group. The heteroarylene is attached to the rest of the molecule through a single bond and to the radical group through a single bond. Unless stated otherwise specifically in the specification, a heteroarylene group is optionally substituted as described above for a heteroaryl group.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “modulate” or “modulating” or “modulation” refers to an increase or decrease in the amount, quality, or effect of a particular activity, function or molecule. By way of illustration and not limitation, agonists, partial agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or target enzyme and activates the receptor or enzyme to produce a biological response. By way of example, “GPR40 agonist” can be used to refer to a compound that exhibits an EC₅₀ with respect to GPR40 activity of no more than about 100 μM, as measured in the as measured in the inositol phosphate accumulation assay. In some embodiments, the term “agonist” includes full agonists or partial agonists.

The term “full agonist” refers to a modulator that binds to and activates a receptor or target enzyme with the maximum response that an agonist can elicit at the receptor or enzyme.

The term “partial agonist” refers to a modulator that binds to and activates a receptor or target enzyme, but has partial efficacy, that is, less than the maximal response, at the receptor or enzyme relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or target enzyme by a modulator, or antagonist. Antagonism of a receptor, for example, is when a molecule binds to the receptor or target enzyme and does not allow activity to occur.

The term “antagonist” or “neutral antagonist” as used herein refers to a modulator that binds to a receptor or target enzyme and blocks a biological response. By way of example, “SSTR5 antagonist” can be used to refer to a compound that exhibits an IC₅₀ with respect to SSTR5 activity of no more than about 100 μM, as measured in the as measured in the inositol phosphate accumulation assay. An antagonist has no activity in the absence of an agonist or inverse agonist but can block the activity of either, causing no change in the biological response.

The term “inverse agonist” refers to a modulator that binds to the same receptor or target enzyme as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of an agonist (e.g., a GPR40 agonist) that is required for 50% activation of a receptor or target enzyme (e.g., GPR40).

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an SSTR5 antagonist) that is required for 50% inhibition of a receptor or a target enzyme (e.g., SSTR5).

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., a GPR40 agonist, that is predominantly active in the gastrointestinal system. In some embodiments, the biological activity of the gut-restricted compound, e.g., a gut-restricted GPR40 agonist, is restricted to the gastrointestinal system. In some embodiments, gastrointestinal concentration of a gut-restricted modulator, e.g., a gut-restricted GPR40 agonist, is higher than the IC₅₀ value or the EC₅₀ value of the gut-restricted modulator against its receptor or target enzyme, e.g., GPR40, while the plasma levels of said gut-restricted modulator, e.g., gut-restricted GPR40 agonist, are lower than the IC₅₀ value or the EC₅₀ value of the gut-restricted modulator against its receptor or target enzyme, e.g., GPR40. In some embodiments, the gut-restricted compound, e.g., a gut-restricted GPR40 agonist, is non-systemic. In some embodiments, the gut-restricted compound, e.g., a gut-restricted GPR40 agonist, is a non-absorbed compound. In other embodiments, the gut-restricted compound, e.g., a gut-restricted GPR40 agonist, is absorbed, but is rapidly metabolized to metabolites that are significantly less active than the modulator itself toward the target receptor or enzyme, i.e., a “soft drug.” In other embodiments, the gut-restricted compound, e.g., a gut-restricted GPR40 agonist, is minimally absorbed and rapidly metabolized to metabolites that are significantly less active than the modulator itself toward the target receptor or enzyme.

In some embodiments, the gut-restricted modulator, e.g., a gut-restricted GPR40 agonist, is non-systemic but is instead localized to the gastrointestinal system. For example, the modulator, e.g., a gut-restricted GPR40 agonist, may be present in high levels in the gut, but low levels in serum. In some embodiments, the systemic exposure of a gut-restricted modulator, e.g., a gut-restricted GPR40 agonist, is, for example, less than 100, less than 50, less than 20, less than 10, or less than 5 nM, bound or unbound, in blood serum. In some embodiments, the intestinal exposure of a gut-restricted modulator, e.g., a gut-restricted GPR40 agonist, is, for example, greater than 1000, 5000, 10000, 50000, 100000, or 500000 nM. In some embodiments, a modulator, e.g., a GPR40 agonist, is gut-restricted due to poor absorption of the modulator itself, or because of absorption of the modulator which is rapidly metabolized in serum resulting in low systemic circulation, or due to both poor absorption and rapid metabolism in the serum. In some embodiments, a modulator, e.g., a GPR40 agonist, is covalently bonded to a kinetophore, optionally through a linker, which changes the pharmacokinetic profile of the modulator.

In particular embodiments, the gut-restricted GPR40 agonist is a soft drug. The term “soft drug” as used herein refers to a compound that is biologically active but is rapidly metabolized to metabolites that are significantly less active than the compound itself toward the target receptor. In some embodiments, the gut-restricted GPR40 agonist is a soft drug that is rapidly metabolized in the blood to significantly less active metabolites. In some embodiments, the gut-restricted GPR40 agonist is a soft drug that is rapidly metabolized in the liver to significantly less active metabolites. In some embodiments, the gut-restricted GPR40 agonist is a soft drug that is rapidly metabolized in the blood and the liver to significantly less active metabolites. In some embodiments, the gut-restricted GPR40 agonist is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug gut-restricted GPR40 agonist.

The term “kinetophore” as used herein refers to a structural unit tethered to a small molecule modulator, e.g., a GPR40 agonist, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator, e.g., a GPR40 agonist, and has minimal changes to the binding to or association with a receptor or target enzyme. The defining feature of a kinetophore is not its interaction with the target, for example a receptor, but rather its effect on specific physiochemical characteristics of the modulator to which it is attached, e.g., a GPR40 agonist. In some instances, kinetophores are used to restrict a modulator, e.g., a GPR40 agonist, to the gut.

The term “linked” as used herein refers to a covalent linkage between a modulator, e.g., a GPR40 agonist, and a kinetophore. The linkage can be through a covalent bond, or through a “linker.” As used herein, “linker” refers to one or more bifunctional molecules which can be used to covalently bond to the modulator, e.g., a GPR40 agonist, and kinetophore. In some embodiments, the linker is attached to any part of the modulator, e.g., a GPR40 agonist, so long as the point of attachment does not interfere with the binding of the modulator to its receptor or target enzyme. In some embodiments, the linker is non-cleavable. In some embodiments, the linker is cleavable. In some embodiments, the linker is cleavable in the gut. In some embodiments, cleaving the linker releases the biologically active modulator, e.g., a GPR40 agonist, in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

### Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need. Diseases and conditions affected by the gut-brain axis, include central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

### GPR40 in the Gut-Brain Axis

Free fatty acid receptor 1 (FFA1, FFAR1), also known as GPR40, is a class A G-protein coupled receptor. This membrane protein binds free fatty acids, acting as a nutrient sensor for regulating energy homeostasis. In some instances, GPR40 is expressed in enteroendocrine cells and pancreatic islet β cells. In some instances, GPR40 is expressed in enteroendocrine cells. Several naturally-occurring medium to long-chain fatty acids act as ligands for GPR40. GPR40 agonists or partial agonists may be useful in the treatment of metabolic diseases such as obesity, diabetes, and NASH, and other diseases involving the gut-brain axis.

In some instances, modulators of GPR40, for example, GPR40 agonists or partial agonists, induce insulin secretion. In some instances, modulators of GPR40, for example, GPR40 agonists or partial agonists, induce an increase in cytosolic Ca²⁺. In some instances, modulators of GPR40, for example, GPR40 agonists or partial agonists, induce higher levels of intracellular cAMP. In some instances, GPR40 modulation is in enteroendocrine cells. In some instances, modulators of GPR40, for example, GPR40 agonists or partial agonists, induce the secretion of GLP-1, GLP-2, GIP, PYY, CCK, or other hormones. In some instances, modulators of GPR40, for example, GPR40 agonists, induce the secretion of GLP-1, GIP, CCK or PYY. In some instances, modulators of GPR40, for example, GPR40 agonists, induce the secretion of GLP-1.

Described herein is a method of treating a condition or disorder involving the gut-brain axis in an individual in need thereof, the method comprising administering to the individual a GPR40 receptor modulator. In some embodiments, the GPR40 receptor modulator is a GPR40 agonist or partial agonist. In some embodiments, the GPR40 receptor modulator is a GPR40 agonist. In some embodiments, the GPR40 receptor modulator is a GPR40 partial agonist. In some embodiments, the GPR40 receptor modulator is a GPR40 positive allosteric modulator. In some embodiments, the GPR40 modulator is a gut-restricted GPR40 modulator. In some embodiments, the GPR40 modulator is a soft drug.

In some embodiments, the condition or disorder involving the gut-brain axis is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is type 2 diabetes, hyperglycemia, metabolic syndrome, obesity, hypercholesterolemia, nonalcoholic steatohepatitis, or hypertension. In some embodiments, the metabolic disorder is diabetes. In other embodiments, the metabolic disorder is obesity. In other embodiments, the metabolic disorder is nonalcoholic steatohepatitis. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome. In some embodiments, the condition involving the gut-brain axis is enteritis. In some embodiments, the condition involving the gut-brain axis is chemotherapy-induced enteritis or radiation-induced enteritis. In some embodiments, the condition involving the gut-brain axis is weight loss or preventing weight gain or weight regain. In some embodiments, the condition involving the gut-brain axis is weight loss or preventing weight gain or weight regain post-bariatric surgery. In some embodiments, the condition involving the gut-brain axis is weight loss or preventing weight gain or weight regain, wherein the subject has had bariatric surgery.

### Gut-Restricted Modulators

In some instances, differentiation of systemic effects of a GPR40 agonist from beneficial, gut-driven effects would be critical for the development of a GPR40 agonist for the treatment of disease.

In some instances, activation of GPR40 by a GPR40 agonist recapitulates the lipotoxicity of free fatty acids on pancreatic beta-cells. In some instances, activation of GPR40 by a GPR40 agonist leads to beta-cell degeneration, islet insulin depletion, glucose intolerance and hyperglycemia. In some instances, the detrimental effects on beta-cells by a GPR40 agonist may be mediated through ER stress and NF-kB signaling pathways. In some instances, differentiation of deleterious systemic effects of a GPR40 agonist on beta-cell function and viability from beneficial, gut-driven effects would be critical for the development of a GPR40 agonist for the treatment of disease.

In some embodiments, the GPR40 agonist is gut-restricted. In some embodiments, the GPR40 agonist is designed to be substantially non-permeable or substantially non-bioavailable in the blood stream. In some embodiments, the GPR40 agonist is designed to activate GPR40 activity in the gut and is substantially non-systemic. In some embodiments, the GPR40 agonist has low systemic exposure.

In some embodiments, a gut-restricted GPR40 agonist has low oral bioavailability. In some embodiments, a gut-restricted GPR40 agonist has <40% oral bioavailability, <30% oral bioavailability, <20% oral bioavailability, <10% oral bioavailability, <8% oral bioavailability, <5% oral bioavailability, <3% oral bioavailability, or <2% oral bioavailability.

In some embodiments, the unbound plasma levels of a gut-restricted GPR40 agonist are lower than the EC₅₀ value of the GPR40 agonist against GPR40. In some embodiments, the unbound plasma levels of a gut-restricted GPR40 agonist are significantly lower than the EC₅₀ value of the gut-restricted GPR40 agonist against GPR40. In some embodiments, the unbound plasma levels of the GPR40 agonist are 2-fold, 10-fold, 20-fold, 30-fold, 40-fold, 50-fold, or 100-fold lower than the EC₅₀ value of the gut-restricted GPR40 agonist against GPR40.

In some embodiments, a gut-restricted GPR40 agonist has low systemic exposure. In some embodiments, the systemic exposure of a gut-restricted GPR40 agonist is, for example, less than 500, less than 200, less than 100, less than 50, less than 20, less than 10, or less than 5 nM, bound or unbound, in blood serum. In some embodiments, the systemic exposure of a gut-restricted GPR40 agonist is, for example, less than 500, less than 200, less than 100, less than 50, less than 20, less than 10, or less than 5 ng/mL, bound or unbound, in blood serum.

In some embodiments, a gut-restricted GPR40 agonist has low pancreatic exposure. In some embodiments, the pancreatic exposure of a gut-restricted GPR40 agonist is, for example, less than 500, less than 200, less than 100, less than 50, less than 20, less than 10, or less than 5 nM in the pancreas. In some embodiments, the pancreatic exposure of a gut-restricted GPR40 agonist is, for example, less than 500, less than 200, less than 100, less than 50, less than 20, less than 10, or less than 5 ng/mL in the pancreas.

In some embodiments, a gut-restricted GPR40 agonist has low permeability. In some embodiments, a gut-restricted GPR40 agonist has low intestinal permeability. In some embodiments, the permeability of a gut-restricted GPR40 agonist is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted GPR40 agonist has low absorption. In some embodiments, the absorption of a gut-restricted GPR40 agonist is less than less than 40%, less than 30%, less than 20%, or less than 10%, less than 5%, or less than 1%.

In some embodiments, a gut-restricted GPR40 agonist has high plasma clearance. In some embodiments, a gut-restricted GPR40 agonist is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments, a gut-restricted GPR40 agonist is rapidly metabolized upon administration. In some embodiments, the internal ester of the compounds described herein is rapidly cleaved upon administration. In some embodiments, a gut-restricted GPR40 agonist has a short half-life. In some embodiments, the half-life of a gut-restricted GPR40 agonist is less than less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted GPR40 agonist have rapid clearance. In some embodiments, the metabolites of a gut-restricted GPR40 agonist are undetectable in less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted GPR40 agonist have low bioactivity. In some embodiments, the EC₅₀ value of the metabolites of a gut-restricted GPR40 agonist is 10-fold, 20-fold, 30-fold, 40-fold, 50-fold, 100-fold, 500-fold, or 1000-fold higher than the EC₅₀ value of the gut-restricted GPR40 agonist against GPR40. In some embodiments, the metabolites of a gut-restricted GPR40 agonist have rapid clearance and low bioactivity.

In some embodiments of the methods described herein, the GPR40 modulator is gut-restricted. In some embodiments, the GPR40 modulator is a gut-restricted GPR40 agonist. In some embodiments, the GPR40 agonist is a gut-restricted GPR40 full agonist. In some embodiments, the GPR40 agonist is a gut-restricted GPR40 partial agonist. In some embodiments, the GPR40 agonist is covalently bonded to a kinetophore. In some embodiments, the GPR40 agonist is covalently bonded to a kinetophore through a linker.

### Compounds

Disclosed herein, in certain embodiments, is a compound of Formula (I):

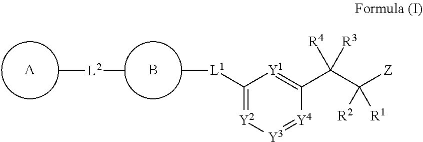

or a pharmaceutically acceptable salt, solvate, stereoisomer, or prodrug thereof, wherein:
Z is —C(O)OH, —C(O)OR⁵, —C(O)NR⁶, —C(O)NHS(O)₂R⁵, —S(O)₂NHC(O)R⁵, —P(O)(R⁵)OR⁶, —P(O)(OR⁶)₂, —S(O)₂OR⁶;
or Z is a 4- or 5-membered heterocycle which is unsubstituted or substituted with 1, 2, 3, or 4 substituents selected from C₁-C₆ alkyl, —O—(C₁-C₆ alkyl), —OH, and ═O;
R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —P(O)(OH)₂, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

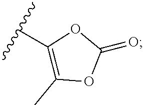 [SMILES: *c1oc(=O)oc1C]

R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
R¹, R², R³, and R⁴ are each independently hydrogen, halogen, —OH, —O—(C₁-C₆ alkyl), C₁-C₆ alkyl, C₃-C₆ cycloalkyl, or 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
Y¹, Y², Y³, and Y⁴ are each independently N, CH, or C—R^{Y};
each R^{Y} is independently halogen, —CN, —OH, —O—(C₁-C₆ alkyl), —NH₂, —NH—(C₁-C₆ alkyl), —N(C₁-C₆ alkyl)₂, C₁-C₆ alkyl, C₃-C₆ cycloalkyl, and 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
L¹ is *—O—C(O)—, or *—C(O)—O—; wherein * represents the connection to Ring B;
Ring B is arylene, heteroarylene, C₃-C₁₀ cycloalkylene, or 3- to 10-membered heterocycloalkylene; wherein the arylene, heteroarylene, cycloalkylene, or heterocycloalkylene is unsubstituted or substituted with 1, 2, 3, or 4 R^{B} substituents;
Ring A is aryl, heteroaryl, C₃-C₁₀ cycloalkyl, or 3- to 10-membered heterocycloalkyl; wherein the aryl, heteroaryl, cycloalkyl, or heterocycloalkyl is unsubstituted or substituted with 1, 2, 3, 4, or 5 R^{A} substituents;
L² is a bond, C₁-C₆ alkylene, or —(C₁-C₆ alkylene)-O—; wherein the alkylene is unsubstituted or substituted with 1, 2, or 3 substituents selected from the group consisting of halogen, —CN, —OH, C₁-C₆ alkyl, and —O—(C₁-C₆ alkyl);
each R^{A} is independently halogen, C₁-C₁₀ alkyl, C₂-C₁₀ alkenyl, C₂-C₁₀ alkynyl, C₁-C₁₀ fluoroalkyl, -L^{A}-CN, -L^{A}-OH, -L^{A}-OR¹⁰, -L^{A}-NR¹¹R¹¹, -L^{A}-C(═O)R¹⁰, -L^{A}C(═O)OR¹¹, -L^{A}-OC(═O)R¹¹, -L^{A}-C(═O)NR¹¹R¹¹, -L^{A}-NR¹¹C(═O)R¹¹, -L^{A}-NR¹C(═O)NR¹¹R¹¹, -L^{A}-OC(═O)NR¹¹R¹¹, -L^{A}-NR¹¹C(═O)OR¹⁰, -L^{A}-OC(═O)OR¹⁰, -L^{A}-aryl, -L^{A}-heteroaryl, -L^{A}-(C₃-C₁₀ cycloalkyl), or -L^{A}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each R^{B} is independently halogen, C₁-C₁₀ alkyl, C₂-C₁₀ alkenyl, C₂-C₁₀ alkynyl, C₁-C₁₀ fluoroalkyl, -L^{B}-CN, -L^{B}-OH, -L^{B}-OR¹⁰, -L^{B}-NR¹¹R¹¹, -L^{B}-C(═O)R¹⁰, -L^{B}-C(═O)OR¹¹, -L^{B}-OC(═O)R¹¹, -L^{B}-C(═O)NR¹¹R¹¹, -L^{B}-NR¹¹C(═O)R¹¹, -L^{B}-NR¹¹C(═O)NR¹¹R¹¹, -L^{B}-OC(═O)NR¹¹R¹¹, -L^{B}-NR¹¹C(═O)OR¹⁰, -L^{B}-OC(═O)OR¹⁰, -L^{B}-aryl, -L^{B}-heteroaryl, -L^{B}-(C₃-C₁₀ cycloalkyl), or -L^{B}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each L^{A} and L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl;
each R¹⁰ is independently C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl); and
each R¹¹ is independently hydrogen, C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
or two R¹¹ on the same nitrogen atom are taken together with the nitrogen to which they are attached to form a 3- to 10-membered N-heterocycloalkyl; wherein the heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl).

Any combination of the groups described above or below for the various variables is contemplated herein. Throughout the specification, groups and substituents thereof are chosen by one skilled in the field to provide stable moieties and compounds.

In some embodiments of a compound of Formula (I) or a pharmaceutically acceptable salt, solvate, stereoisomer, or prodrug thereof, L¹ is *—C(O)—O—; wherein * represents the connection to Ring B. In some embodiments, the compound of Formula (I), or a pharmaceutically acceptable salt, solvate, stereoisomer, or prodrug thereof, is a compound of Formula (Ia):

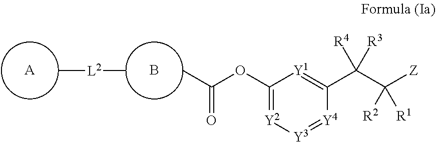

or a pharmaceutically acceptable salt, solvate, stereoisomer, or prodrug thereof.

In some embodiments of a compound of Formula (I) or a pharmaceutically acceptable salt, solvate, stereoisomer, or prodrug thereof, L¹ is *—O—C(O)—, wherein * represents the connection to Ring B. In some embodiments, the compound of Formula (I), or a pharmaceutically acceptable salt, solvate, stereoisomer, or prodrug thereof, is a compound of Formula (Ib):

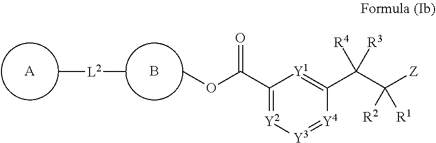

or a pharmaceutically acceptable salt, solvate, stereoisomer, or prodrug thereof.

In some embodiments of a compound of Formula (I), (Ia), or (Ib), or a pharmaceutically acceptable salt, solvate, stereoisomer, or prodrug thereof, Y¹, Y², Y³, and Y⁴ are each independently N, CH, or C—R^{Y}; and each R^{Y} is independently halogen, —CN, —OH, —O—(C₁-C₆ alkyl), C₁-C₆ alkyl, or C₃-C₆ cycloalkyl; wherein each alkyl, and cycloalkyl is unsubstituted or substituted with 1, 2, or 3 substituents selected from halogen, —CN, —OH, —O—(C₁-C₆ alkyl), and C₁-C₆ alkyl. In some embodiments, each R^{Y} is independently halogen, —CN, —OH, —O—(C₁-C₆ alkyl), C₁-C₆ alkyl. In some embodiments, each R^{Y} is independently halogen, —CN, —OH, —O-(unsubstituted C₁-C₆ alkyl), or unsubstituted C₁-C₆ alkyl. In some embodiments, each R^{Y} is independently F, Cl, Br, —CN, —OH, —O—(C₁-C₆ alkyl), or C₁-C₆ alkyl. In some embodiments, each R^{Y} is independently F, Cl, Br, —CN, —OH, —O-(unsubstituted C₁-C₆ alkyl), or unsubstituted C₁-C₆ alkyl. In some embodiments, Y¹, Y², Y³, and Y⁴ are each independently N or CH. In some embodiments, Y¹, Y², Y³, and Y⁴ are each CH.

In some embodiments of a compound of Formula (I), (Ia), or (Ib), or a pharmaceutically acceptable salt, solvate, stereoisomer, or prodrug thereof, Z is —C(O)OH, —C(O)OR⁵, —C(O)NHR⁶, —C(O)NHS(O)₂R⁵, —S(O)₂NHC(O)R⁵, —P(O)(R⁵)OR⁶, —P(O)(OR⁶)₂, or —S(O)₂OR⁶. In some embodiments, R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —P(O)(OH)₂, —O—(C₁-C₆ alkyl), C₁-C₆ alkyl, C₁-C₆ hydroxyalkyl, and

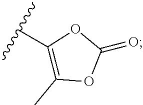 [SMILES: *c1oc(=O)oc1C]

and R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —O—(C₁-C₆ alkyl), C₁-C₆ alkyl, and C₁-C₆ hydroxyalkyl. In some embodiments, R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one —P(O)(OH)₂ or

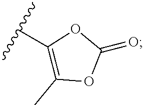 [SMILES: *c1oc(=O)oc1C]

and R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted. In some embodiments, R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted; and R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted. In some embodiments, Z is —C(O)OH, —C(O)OMe, —C(O)OEt, —C(O)O-iPr, —C(O)O-tBu, —C(O)NH₂, —C(O)NHS(O)₂Me, —S(O)₂NHC(O)Me, —P(O)(Me)OH, —P(O)(Me)OMe, —P(O)(OH)₂, —P(O)(OMe)₂, or —S(O)₂OH. In some embodiments, Z is —C(O)OH, —C(O)O-tBu, —P(O)(Me)OH, —P(O)(OH)₂, or —S(O)₂OH. In some embodiments, Z is —C(O)OH.

In some embodiments of a compound of Formula (I), (Ia), or (Ib), or a pharmaceutically acceptable salt, solvate, stereoisomer, or prodrug thereof, Z is a 4- or 5-membered carbocycle or heterocycle which is unsubstituted or substituted with 1, 2, 3, or 4 substituents selected from C₁-C₆ alkyl, —O—(C₁-C₆ alkyl), —OH, and ═O. In some embodiments, Z is a 4- or 5-membered carbocycle or heterocycle selected from:

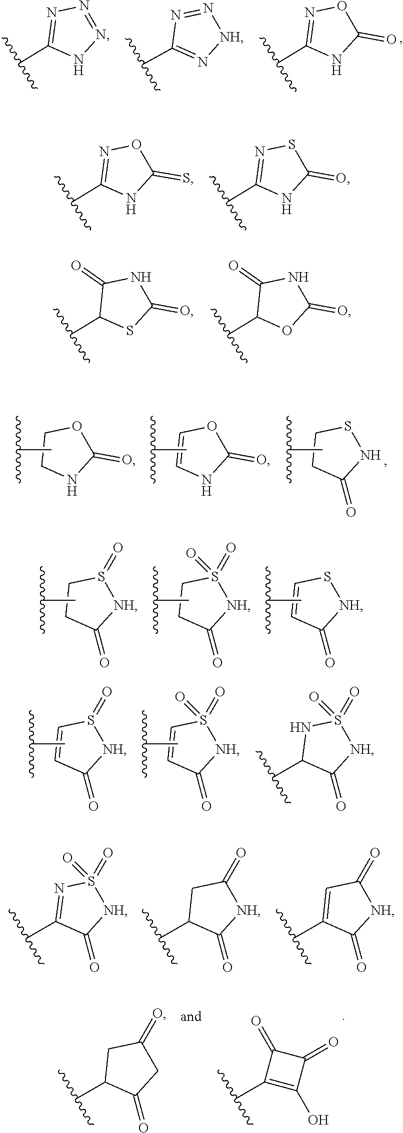

In some embodiments, the compound of Formula (I), (I), (Ia), or (Ib), or a pharmaceutically acceptable salt, solvate, stereoisomer, or prodrug thereof, is a compound of Formula (II), (IIa), or (IIb):

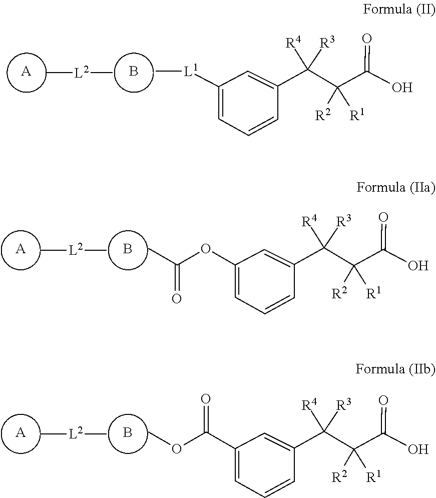

or a pharmaceutically acceptable salt, solvate, stereoisomer, or prodrug thereof.

In some embodiments of a compound of Formula (I), (Ia), (Ib), (II), (IIa), or (IIb), or a pharmaceutically acceptable salt, solvate, stereoisomer, or prodrug thereof, R¹, R², R³, and R⁴ are each independently hydrogen, halogen, C₁-C₆ alkyl, C₃-C₆ cycloalkyl. In some embodiments, R¹, R², R³, and R⁴ are each independently hydrogen, halogen, C₁-C₆ alkyl, C₃-C₆ cycloalkyl; wherein each alkyl and cycloalkyl is unsubstituted or substituted with 1, 2, or 3 substituents selected from halogen, —CN, —OH, —O—(C₁-C₆ alkyl), and C₁-C₆ alkyl. In some embodiments, R¹, R², R³, and R⁴ are each independently hydrogen, halogen, unsubstituted C₁-C₆ alkyl, or unsubstituted C₃-C₆ cycloalkyl. In some embodiments, R¹, R², R³, and R⁴ are each independently hydrogen, —F, methyl, or unsubstituted C₃-C₆ cycloalkyl.

In some embodiments of a compound of Formula (I), (Ia), (Ib), (II), (IIa), or (IIb), or a pharmaceutically acceptable salt, solvate, stereoisomer, or prodrug thereof, R⁴ is C₃-C₆ cycloalkyl. In some embodiments, R⁴ in unsubstituted C₃-C₆ cycloalkyl. In some embodiments, R⁴ is cyclopropyl, cyclobutyl, cyclopentyl, or cyclohexyl. In some embodiments, R⁴ is cyclopropyl. In some embodiments, R⁴ is unsubstituted cyclopropyl.

In some embodiments of a compound of Formula (I), (Ia), (Ib), (II), (IIa), or (IIb), or a pharmaceutically acceptable salt, solvate, stereoisomer, or prodrug thereof, R¹, R², R³, and R⁴ are each independently hydrogen, halogen, C₁-C₆ alkyl, C₃-C₆ cycloalkyl. In some embodiments, R¹, R², and R³ are each independently hydrogen, halogen, or C₁-C₆ alkyl; and R⁴ is C₃-C₆ cycloalkyl. In some embodiments, R¹, R², and R³ are each independently hydrogen, —F, or methyl; and R⁴ is unsubstituted C₃-C₆ cycloalkyl. In some embodiments, R¹, R², and R³ are each independently hydrogen, —F, or methyl; and R⁴ is unsubstituted cyclopropyl. In some embodiments, R⁴ is unsubstituted cyclopropyl; R³ is hydrogen; and R¹ and R² are independently hydrogen, —F, or methyl. In some embodiments, R⁴ is unsubstituted cyclopropyl; R³ is hydrogen; and R¹ and R² are independently —F or methyl.

In some embodiments, the compound of Formula (I), (Ia), (Ib), (II), (IIa), or (IIb), or a pharmaceutically acceptable salt, solvate, stereoisomer, or prodrug thereof, is a compound of Formula (III), (IIIa), or (IIIb):

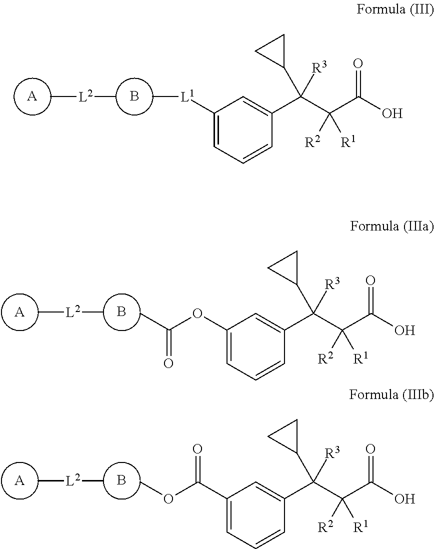

or a pharmaceutically acceptable salt, solvate, stereoisomer, or prodrug thereof.

In some embodiments of a compound of Formula (I), (Ia), (Ib), (II), (IIa), (IIb), (III), (IIIa), or (IIIb), or a pharmaceutically acceptable salt, solvate, stereoisomer, or prodrug thereof, R¹, R², and R³ are each independently hydrogen, —F, or methyl.

In some embodiments, the compound of Formula (I), (Ia), (Ib), (II), (IIa), (IIb), (III), (IIIa), or (IIIb), or a pharmaceutically acceptable salt, solvate, stereoisomer, or prodrug thereof, is a compound of Formula (IV), (IVa), or (IVb):

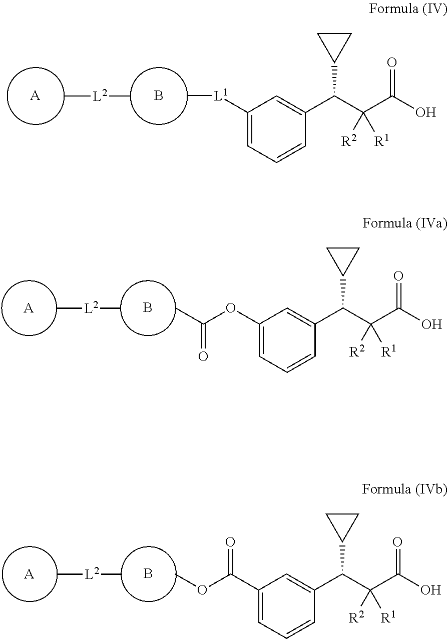

or a pharmaceutically acceptable salt, solvate, stereoisomer, or prodrug thereof.

In some embodiments of a compound of Formula (I), (Ia), (Ib), (II), (IIa), (IIb), (III), (IIIa), (IIIb), (IV), (IVa), or (IVb), or a pharmaceutically acceptable salt, solvate, stereoisomer, or prodrug thereof, R¹ and R² are each independently hydrogen, —F, or methyl.

In some embodiments, the compound of Formula (I), (Ia), (Ib), (II), (IIa), (IIb), (III), (IIIa), (IIIb), (IV), (IVa), or (IVb), or a pharmaceutically acceptable salt, solvate, stereoisomer, or prodrug thereof, is a compound of Formula (V), (Va), or (Vb):

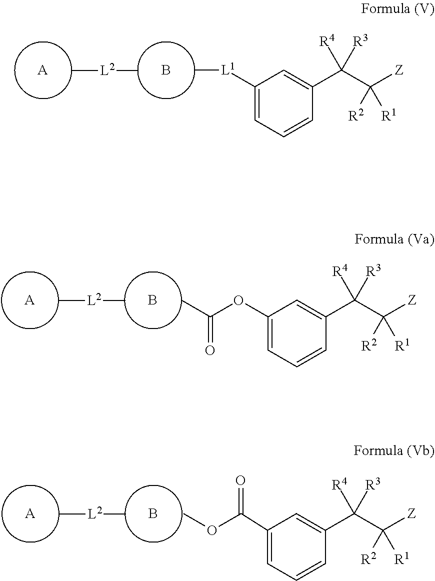

or a pharmaceutically acceptable salt, solvate, stereoisomer, or prodrug thereof.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring B is phenylene, monocyclic heteroarylene, C₃-C₆ cycloalkylene, or 3- to 6-membered heterocycloalkylene; wherein the phenylene, heteroarylene, cycloalkylene, or heterocycloalkylene is unsubstituted or substituted with 1, 2, 3, or 4 R^{B} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring B is C₃-C₁₀ cycloalkylene or 3- to 10-membered heterocycloalkylene; wherein the cycloalkylene or heterocycloalkylene is unsubstituted or substituted with 1, 2, 3, or 4 R^{B} substituents. In some embodiments, Ring B is C₃-C₆ cycloalkylene or 3- to 6-membered heterocycloalkylene wherein the cycloalkylene or heterocycloalkylene is unsubstituted or substituted with 1, 2, 3, or 4 R^{B} substituents. In some embodiments, Ring B is C₃-C₆ cycloalkylene which is unsubstituted or substituted with 1, 2, 3, or 4 R^{B} substituents. In some embodiments, Ring B is unsubstituted C₃-C₆ cycloalkylene. In some embodiments, Ring B is cyclohexylene which is unsubstituted or substituted with 1, 2, 3, or 4 R^{B} substituents. In some embodiments, Ring B is cyclohexylene. In some embodiments, Ring B is 3- to 6-membered heterocycloalkylene which is unsubstituted or substituted with 1, 2, 3, or 4 R^{B} substituents. In some embodiments, Ring B is 3- to 6-membered nitrogen containing heterocycloalkylene which is unsubstituted or substituted with 1, 2, 3, or 4 R^{B} substituents. In some embodiments, Ring B is unsubstituted 3- to 6-membered heterocycloalkylene. In some embodiments, Ring B is 5- or 6-membered heterocycloalkylene which is substituted with 1 R^{B} substituent. In some embodiments, Ring B is

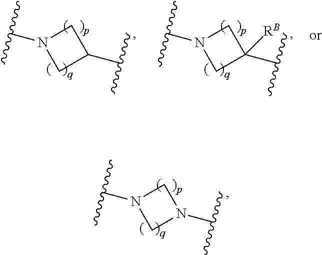

wherein p and q are each independently 1 or 2.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring B is arylene or heteroarylene; wherein the arylene or heteroarylene is unsubstituted or substituted with 1, 2, 3, or 4 R^{B} substituents. In some embodiments, Ring B is phenylene or 5- or 6-membered monocyclic heteroarylene; wherein the phenylene or heteroarylene is unsubstituted or is substituted with 1, 2, 3, or 4 R^{B} substituents. In some embodiments, Ring B is phenylene or 5- or 6-membered monocyclic heteroarylene; wherein the 5- or 6-membered monocyclic heteroarylene is a furanylene, thienylene, pyrrolylene, imidazolylene, pyrazolylene, triazolylene, oxazolylene, isoxazolylene, thiazolylene, isothiazolylene, oxadiazolylene, thiadiazolylene, pyridinylene, pyrimidinylene, pyridazinylene, pyrazinylene, or triazinylene; and wherein the phenylene or heteroarylene is unsubstituted or is substituted with 1, 2, 3, or 4 R^{B} substituents. In some embodiments, Ring B is phenylene or a 5- or 6-membered monocyclic heteroarylene; wherein the 5- or 6-membered monocyclic heteroarylene is an isoxazolylene, pyridinylene, or pyrazinylene; and wherein the phenylene or heteroarylene is unsubstituted or is substituted with 1, 2, 3, or 4 R^{B} substituents. In some embodiments, Ring B is phenylene which is unsubstituted or is substituted with 1, 2, 3, or 4 R^{B} substituents. In some embodiments, Ring B is phenylene which is unsubstituted or is substituted with 1 or 2 R^{B} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring B is unsubstituted. In some embodiments, Ring B is substituted with 1, 2, 3, or 4 R^{B} substituents. In some embodiments, Ring B is substituted with 1 or 2 R^{B} substituents. In some embodiments, Ring B is substituted with 1 R^{B} substituent. In some embodiments, Ring B is substituted with 2 R^{B} substituents. In some embodiments, Ring B is substituted with 3 R^{B} substituents. In some embodiments, Ring B is substituted with 4 R^{B} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each R^{B} is independently halogen, C₁-C₁₀ alkyl, C₁-C₁₀ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-OC(═O)R¹¹, -L^{B}-C(═O)NR¹¹R¹¹, -L^{B}-NR¹¹C(═O)R¹¹, -L^{B}-(C₃-C₁₀ cycloalkyl) or -L^{B}-(3- to 10-membered heterocycloalkyl). In some embodiments, each R^{B} is independently halogen, C₁-C₁₀ alkyl, C₁-C₁₀ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-OC(═O)R¹¹, -L^{B}-C(═O)NR¹¹R¹¹, -L^{B}-NR¹¹C(═O)R¹¹, -L^{B}-(C₃-C₁₀ cycloalkyl) or -L^{B}-(3- to 10-membered heterocycloalkyl); wherein each alkyl and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R^{B} is independently halogen, C₁-C₆ alkyl, C₁-C₆ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-C(═O)NR¹¹R¹¹, or -L^{B}-(3- to 10-membered heterocycloalkyl). In some embodiments, each R^{B} is independently halogen, C₁-C₆ alkyl, C₁-C₆ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-C(═O)NR¹¹R¹¹, or -L^{B}-(3- to 10-membered heterocycloalkyl); wherein each alkyl and heterocycloalkyl is unsubstituted or substituted with 1, 2, or 3 substituents selected from the group consisting of halogen, —CN, —OH, C₁-C₆ alkyl, C₁-C₆ fluoroalkyl, —O—(C₁-C₆ alkyl), and —O—(C₁-C₆ fluoroalkyl). In some embodiments, each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl). In some embodiments, each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of halogen, —OH, C₁-C₆ alkyl, and C₁-C₆ fluoroalkyl. In some embodiments, each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of C₁-C₆ alkyl.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each L^{B} is a bond. In some embodiments, each L^{B} is independently C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, each L^{B} is independently unsubstituted C₁-C₆ alkylene. In some embodiments, each L^{B} is independently unsubstituted C₁-C₂ alkylene. In some embodiments, each L^{B} is —CH₂—.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, one R^{B} is -L^{B}-NR¹¹R¹¹; wherein -L^{B}- is —CH₂—; and each R¹¹ is independently hydrogen or C₁-C₁₀ alkyl. In some embodiments, one R^{B} is -L^{B}-NR¹¹R¹¹; wherein -L^{B}- is —CH₂—; and each R¹¹ is independently hydrogen or C₁-C₁₀ alkyl which is unsubstituted or substituted with 1, 2, 3, 4, or 5 —OH substituents. In some embodiments, one R^{B} is -L^{B}-NR¹¹R¹¹; wherein -L^{B}- is —CH₂—; and the two R¹¹ are taken together with the nitrogen to which they are attached to form a 3- to 10-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl. In some embodiments, one R^{B} is -L^{B}-NR¹¹R¹¹; wherein -L^{B}- is —CH₂—; and the two R¹¹ are taken together with the nitrogen to which they are attached to form a 3- to 10-membered N-heterocycloalkyl; wherein the heterocycloalkyl is unsubstituted or substituted with 1, 2, 3, 4, or 5 C₁-C₆ alkyl substituents. In some embodiments, one R^{B} is -L^{B}-(3- to 10-membered heterocycloalkyl); wherein -L^{B}- is —CH₂—; and heterocycloalkyl is unsubstituted or substituted with 1, 2, 3, 4, or 5 C₁-C₆ alkyl substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, L² is a bond.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, L² is C₁-C₆ alkylene, or —(C₁-C₆ alkylene)-O—; wherein the alkylene is unsubstituted or substituted with 1, 2, or 3 substituents selected from the group consisting of halogen, —CN, —OH, C₁-C₆ alkyl, and —O—(C₁-C₆ alkyl). In some embodiments, L² is C₁-C₆ alkylene; wherein the alkylene is unsubstituted or substituted with 1, 2, or 3 substituents selected from the group consisting of —OH, C₁-C₆ alkyl, and —O—(C₁-C₆ alkyl). In some embodiments, L² is C₁-C₆ alkylene; wherein the alkylene is unsubstituted or substituted with C₁-C₆ alkyl. In some embodiments, L² is C₁-C₂ alkylene; wherein the alkylene is unsubstituted or substituted with C₁-C₆ alkyl. In some embodiments, L² is —CH(CH₃)—.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring A is C₃-C₁₀ cycloalkylene or 3- to 10-membered heterocycloalkylene; wherein the cycloalkylene or heterocycloalkylene is unsubstituted or substituted with 1, 2, 3, 4, or 5 R^{A} substituents. In some embodiments, Ring A is C₃-C₆ cycloalkylene or 3- to 6-membered heterocycloalkylene wherein the cycloalkylene or heterocycloalkylene is unsubstituted or substituted with 1, 2, 3, 4, or 5 R^{A} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring A is aryl or heteroaryl; wherein the aryl or heteroaryl is unsubstituted or substituted with 1, 2, 3, 4, or 5 R^{A} substituents. In some embodiments, Ring A is phenyl or 5- or 6-membered monocyclic heteroaryl; wherein the phenyl or heteroaryl is unsubstituted or is substituted with 1, 2, 3, 4, or 5 R^{A} substituents. In some embodiments, Ring A is phenyl or 5- or 6-membered monocyclic heteroaryl; wherein the phenyl or heteroaryl is unsubstituted or is substituted with 1, 2, or 3 R^{A} substituents. In some embodiments, Ring A is phenyl. In some embodiments, Ring B is 5- or 6-membered heteroaryl. In some embodiments, Ring A is 5-membered heteroaryl. In some embodiments, Ring A is furanyl, thienyl, pyrrolyl, imidazolyl, pyrazolyl, triazolyl, tetrazolyl, oxazolyl, isoxazolyl, thiazolyl, or isothiazolyl. In some embodiments, Ring B is 6-membered heteroaryl. In some embodiments, Ring A is pyridinyl, pyrimidinyl, pyridazinyl, pyrazinyl, or triazinyl. In some embodiments, Ring A is pyridinyl. In some embodiments, Ring A is phenyl or 6-membered monocyclic heteroaryl; wherein the phenyl or heteroaryl is unsubstituted or is substituted with 1, 2, or 3 R^{A} substituents. In some embodiments, Ring A is phenyl or pyridinyl; wherein the phenyl or pyridinyl is unsubstituted or is substituted with 1, 2, or 3 R^{A} substituents. In some embodiments, Ring A is phenyl which is unsubstituted or is substituted with 1, 2, or 3 R^{A} substituents. In some embodiments, Ring A is pyridinyl which is unsubstituted or is substituted with 1, 2, or 3 R^{A} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring A is unsubstituted. In some embodiments, Ring A is substituted with 1, 2, 3, 4, or 5 R^{A} substituents. In some embodiments, Ring A is substituted with 1, 2, or 3 R^{A} substituents. In some embodiments, Ring A is substituted with 1 R^{A} substituent. In some embodiments, Ring A is substituted with 2 R^{A} substituents. In some embodiments, Ring A is substituted with 3 R^{A} substituents. In some embodiments, Ring A is substituted with 4 R^{A} substituents. In some embodiments, Ring A is substituted with 5 R^{A} substituents.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each R^{A} is independently halogen, C₁-C₁₀ alkyl, C₁-C₁₀ fluoroalkyl, -L^{A}-CN, -L^{A}-OH, -L^{A}-OR¹⁰, -L^{A}-NR¹¹R¹¹, -L^{A}-C(═O)OR¹¹, -L^{A}-OC(═O)R¹¹, -L^{A}-C(═O)NR¹¹R¹¹, -L^{A}-NR₁₁C(═O)R¹¹, -L^{A}-(C₃-C₁₀ cycloalkyl) or -L^{A}-(3- to 10-membered heterocycloalkyl). In some embodiments, each R^{A} is independently halogen, C₁-C₁₀ alkyl, C₁-C₁₀ fluoroalkyl, -L^{A}-CN, -L^{A}-OH, -L^{A}-OR¹⁰, -L^{A}-NR¹¹R¹¹, -L^{A}-C(═O)OR¹¹, -L^{A}-OC(═O)R¹¹, -L^{A}-C(═O)NR¹¹R¹¹, -L^{A}-NR₁₁C(═O)R¹¹, -L^{A}-(C₃-C₁₀ cycloalkyl) or -L^{A}-(3- to 10-membered heterocycloalkyl); wherein each alkyl and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)OR¹¹, -L^{A}-C(═O)NR¹¹R¹¹, or -L^{A}-(3- to 10-membered heterocycloalkyl). In some embodiments, each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)OR¹¹, -L^{A}-C(═O)NR¹¹R¹¹, or -L^{A}-(3- to 10-membered heterocycloalkyl); wherein each alkyl and heterocycloalkyl is unsubstituted or substituted with 1, 2, or 3 substituents selected from the group consisting of halogen, —CN, —OH, C₁-C₆ alkyl, C₁-C₆ fluoroalkyl, —O—(C₁-C₆ alkyl), and —O—(C₁-C₆ fluoroalkyl). In some embodiments, each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹; wherein the alkyl is unsubstituted or substituted with 1, 2, or 3 substituents selected from the group consisting of halogen, —OH, C₁-C₆ fluoroalkyl, —O—(C₁-C₆ alkyl), and —O—(C₁-C₆ fluoroalkyl). In some embodiments, each R^{A} is independently halogen, C₁-C₆ alkyl, C₁-C₆ fluoroalkyl, -L^{A}-OH, or -L^{A}-OR¹⁰; wherein the alkyl is unsubstituted or substituted with 1, 2, or 3 substituents selected from the group consisting of halogen, —OH, and C₁-C₆ fluoroalkyl. In some embodiments, each R^{A} is independently fluoro, chloro, C₁-C₆ alkyl, C₁-C₆ fluoroalkyl, -L^{A}-OH, -L^{A}-OR¹⁰, -L^{A}-NR¹¹R¹¹, or -L^{A}-C(═O)NR¹¹R¹¹.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each L^{A} is a bond. In some embodiments, each L^{A} is independently C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, each L^{A} is independently unsubstituted C₁-C₆ alkylene. In some embodiments, each L^{A} is independently unsubstituted C₁-C₂ alkylene. In some embodiments, each L^{A} is —CH₂—.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each R¹⁰ is independently C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl. In some embodiments, each R¹⁰ is independently C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted. In some embodiments, each R¹⁰ is independently C₁-C₁₀ alkyl, C₃-C₁₀ cycloalkyl, 3- to 10-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹⁰ is independently C₁-C₁₀ alkyl, C₃-C₁₀ cycloalkyl, 3- to 10-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹⁰ is independently C₁-C₆ alkyl, C₃-C₆ cycloalkyl, 3- to 6-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹⁰ is independently C₁-C₆ alkyl, C₃-C₆ cycloalkyl, 3- to 6-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹⁰ is independently C₁-C₆ alkyl, C₃-C₆ cycloalkyl, or 3- to 6-membered heterocycloalkyl; wherein each alkyl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹⁰ is independently C₁-C₆ alkyl; wherein each alkyl is unsubstituted or substituted with 1, 2, 3, 4, or 5 substituents selected from the group consisting of —F, —Cl, —Br, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹⁰ is independently C₁-C₆ alkyl; wherein each alkyl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹⁰ is independently C₁-C₆ alkyl; wherein each alkyl is unsubstituted or substituted with 1, 2, 3, 4, or 5 substituents selected from the group consisting of —OH, C₁-C₆ alkyl and C₁-C₆ hydroxyalkyl.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each R¹¹ is independently hydrogen, C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl. In some embodiments, each R¹¹ is independently hydrogen, C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted. In some embodiments, each R¹¹ is independently hydrogen, C₁-C₁₀ alkyl, C₃-C₁₀ cycloalkyl, 3- to 10-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹¹ is independently hydrogen, C₁-C₁₀ alkyl, C₃-C₁₀ cycloalkyl, 3- to 10-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹¹ is independently hydrogen, C₁-C₆ alkyl, C₃-C₆ cycloalkyl, 3- to 6-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹¹ is independently hydrogen, C₁-C₆ alkyl, C₃-C₆ cycloalkyl, 3- to 6-membered heterocycloalkyl, phenyl, or monocyclic heteroaryl; wherein each alkyl, phenyl, heteroaryl, cycloalkyl, and heterocycloalkyl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of —F, —Cl, —Br, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of halogen, —OH, C₁-C₆ alkyl and C₁-C₆ hydroxyalkyl. In some embodiments, each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of —OH, C₁-C₆ alkyl and C₁-C₆ hydroxyalkyl.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, two R¹¹ on the same nitrogen atom are taken together with the nitrogen to which they are attached to form a 3- to 10-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl. In some embodiments, two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl). In some embodiments, two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl. In some embodiments, two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of —OH, C₁-C₆ alkyl, and C₁-C₆ hydroxyalkyl.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, each R¹⁰ is independently C₁-C₆ alkyl; wherein each alkyl is unsubstituted or substituted with 1, 2, 3, 4, or 5 substituents selected from the group consisting of halogen, —OH, C₁-C₆ alkyl and C₁-C₆ hydroxyalkyl; and each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of halogen, —OH, C₁-C₆ alkyl and C₁-C₆ hydroxyalkyl; or two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

In some embodiments of a compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, Ring B is 3- to 6-membered heterocycloalkylene; wherein the heterocycloalkylene is unsubstituted or substituted with 1, 2, 3, or 4 R^{B} substituents; each R^{B} is independently unsubstituted C₁-C₁₀ alkyl; L² is C₁-C₆ alkylene; wherein the alkylene is unsubstituted or substituted with 1, 2, or 3 substituents selected from the group consisting of —OH, C₁-C₆ alkyl, and —O—(C₁-C₆ alkyl); and Ring A is aryl or heteroaryl; wherein the aryl or heteroaryl is unsubstituted or substituted with 1, 2, or 3 R^{A} substituents.

In some embodiments, the compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, is a compound of Formula (VI), (VIa), (VIb), (VIc), (VId), (VIe), or (VIf):

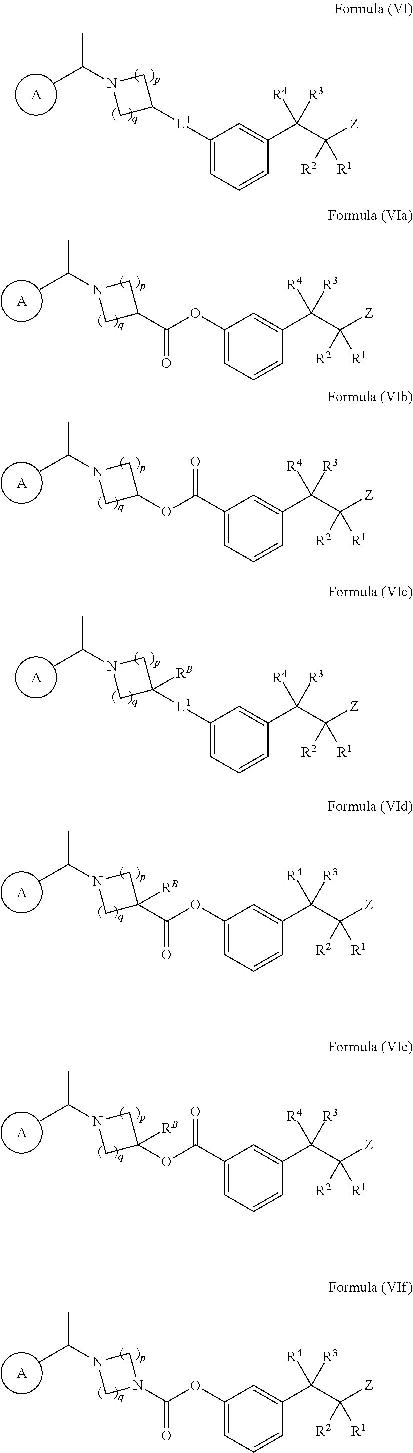

or a pharmaceutically acceptable salt, solvate, stereoisomer, or prodrug thereof; wherein: p and q are each independently 1 or 2; and each R^{B} is independently unsubstituted C₁-C₁₀ alkyl.

In some embodiments of a compound of Formula (VI), (VIa), (VIb), (VIc), (VId), (VIe), or (VIf), or a pharmaceutically acceptable salt, solvate, stereoisomer, or prodrug thereof, p is 1 or 2; and q is 1 or 2. In some embodiments, p is 1. In some embodiments, p is 2. In some embodiments, q is 1. In some embodiments, q is 2. In some embodiments, p is 1 or 2; and q is 2. In some embodiments, p and q are each 1. In some embodiments, p and q are each 2.

In some embodiments of a compound of Formula (I), (Ia), (Ib), (II), (IIa), (IIb), (III), (IIIa), (IIIb), (IV), (IVa), (IVb), (V), (Va), (Vb), (VI), (VIa), (VIb), (VIc), (VId), (VIe), or (VIf), or a pharmaceutically acceptable salt, solvate, stereoisomer, or prodrug thereof, Ring A is phenyl or 5- or 6-membered monocyclic heteroaryl; wherein the phenyl or heteroaryl is unsubstituted or is substituted with 1, 2, or 3 R^{A} substituents; each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹; wherein the alkyl is unsubstituted or substituted with 1, 2, or 3 substituents selected from the group consisting of halogen, —OH, C₁-C₆ fluoroalkyl, —O—(C₁-C₆ alkyl), and —O—(C₁-C₆ fluoroalkyl); and each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, each R^{A} is independently halogen, C₁-C₆ alkyl, C₁-C₆ fluoroalkyl, -L^{A}-OH, -L^{A}-OR¹⁰; wherein the alkyl is unsubstituted or substituted with 1, 2, or 3 substituents selected from the group consisting of halogen, —OH, and C₁-C₆ fluoroalkyl; and each L^{A} is independently a bond or unsubstituted C₁-C₆ alkylene. In some embodiments, Ring A is phenyl that is substituted by 2 —CF₃ substituents.

In some embodiments, the compound of Formula (I), (Ia), (Ib), (II), (IIa), (IIb), (III), (IIIa), (IIIb), (IV), (IVa), (IVb), (V), (Va), or (Vb), or a pharmaceutically acceptable salt, solvate, stereoisomer, or prodrug thereof, is a compound of Formula (VII), (VIIa) or Formula (VIIb):

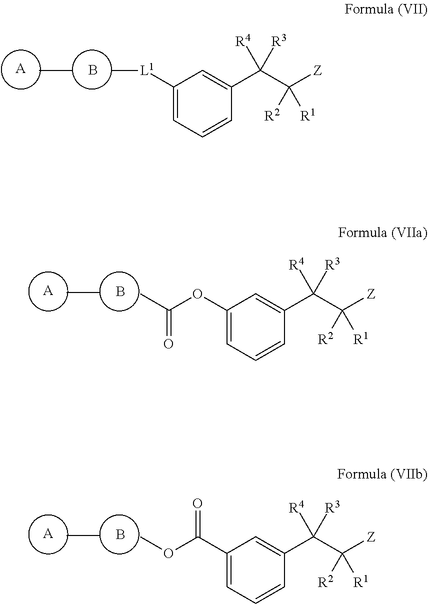

or a pharmaceutically acceptable salt, solvate, stereoisomer, or prodrug thereof; wherein: Ring B is arylene or heteroarylene; wherein the arylene or heteroarylene is unsubstituted or substituted with 1, 2, 3, or 4 R^{B} substituents; and Ring A is aryl or heteroaryl; wherein the aryl or heteroaryl is unsubstituted or substituted with 1, 2, 3, 4, or 5 R^{A} substituents.

In some embodiments of a compound of Formula (I), (Ia), (Ib), (II), (IIa), (IIb), (III), (IIIa), (IIIb), (IV), (IVa), (IVb), (V), (Va), (Vb), (VII), (VIIa), or (VIIb), or a pharmaceutically acceptable salt, solvate, stereoisomer, or prodrug thereof, Ring B is phenylene or 5- or 6-membered monocyclic heteroarylene; wherein the phenylene or heteroarylene is unsubstituted or is substituted with 1, 2, or 3 R^{B} substituents; and Ring A is phenyl or 5- or 6-membered monocyclic heteroaryl; wherein the phenyl or heteroaryl is unsubstituted or is substituted with 1, 2, or 3 R^{A} substituents. In some embodiments, Ring B is phenylene or 5- or 6-membered monocyclic heteroarylene; wherein the phenylene or heteroarylene is unsubstituted or is substituted with 1, 2, or 3 R^{B} substituents; each R^{B} is independently halogen, C₁-C₆ alkyl, C₁-C₆ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-C(═O)NR¹¹R¹¹, or -L^{B}-(3- to 10-membered heterocycloalkyl); wherein each alkyl and heterocycloalkyl is unsubstituted or substituted with 1, 2, or 3 substituents selected from the group consisting of halogen, —CN, —OH, C₁-C₆ alkyl, C₁-C₆ fluoroalkyl, —O—(C₁-C₆ alkyl), and —O—(C₁-C₆ fluoroalkyl); each L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl; Ring A is phenyl or 5- or 6-membered monocyclic heteroaryl; wherein the phenyl or heteroaryl is unsubstituted or is substituted with 1, 2, or 3 R^{A} substituents; each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)R¹¹, -L^{A}-C(═O)NR¹¹R¹¹; wherein the alkyl is unsubstituted or substituted with 1, 2, or 3 substituents selected from the group consisting of —OH, C₁-C₆ alkyl, and —O—(C₁-C₆ alkyl); and each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl. In some embodiments, Ring B is phenylene or 5- or 6-membered monocyclic heteroarylene; wherein the phenylene or heteroarylene is unsubstituted or is substituted with 1, 2, or 3 R^{B} substituents; each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of C₁-C₆ alkyl; each L^{B} is independently a bond or unsubstituted C₁-C₆ alkylene; Ring A is phenyl or 6-membered monocyclic heteroaryl; wherein the phenyl or heteroaryl is unsubstituted or is substituted with 1, 2, or 3 R^{A} substituents; each R^{A} is independently fluoro, chloro, C₁-C₆ alkyl, C₁-C₆ fluoroalkyl, -L^{A}-OR¹⁰, -L^{A}-NR¹¹R¹¹, or -L^{A}-C(═O)NR¹¹R¹¹; and each L^{A} is independently a bond or unsubstituted C₁-C₆ alkylene. In some embodiments, Ring B is phenylene or 5- or 6-membered monocyclic heteroarylene; wherein the phenylene or heteroarylene is unsubstituted or is substituted with 1, 2, or 3 R^{B} substituents; each R^{B} is independently fluoro, C₁-C₆ alkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of C₁-C₆ alkyl; each L^{B} is independently unsubstituted C₁-C₆ alkylene; Ring A is phenyl or 6-membered monocyclic heteroaryl; wherein the phenyl or heteroaryl is unsubstituted or is substituted with 1, 2, or 3 R^{A} substituents; each R^{A} is independently fluoro, C₁-C₆ alkyl, C₁-C₆ fluoroalkyl, -L^{A}-OR¹⁰, -L^{A}-NR¹¹R¹¹, or -L^{A}-C(═O)NR¹¹R¹¹; and each L^{A} is independently a bond. In some embodiments, Ring B is phenylene that is substituted with 1 substituent that is -L^{B}-NR¹¹R¹¹ or -L^{B}-(3- to 10-membered heterocycloalkyl) and L^{B} is unsubstituted C₁-C₆ alkylene; Ring A is phenyl or 6-membered monocyclic heteroaryl; wherein the phenyl or heteroaryl is unsubstituted or is substituted with 1 or 2 R^{A} substituents; each R^{A} is independently fluoro, C₁-C₆ alkyl, C₁-C₆ fluoroalkyl, or —OR¹⁰. In some embodiments, Ring B is phenylene that is substituted with —CH₂—NR¹¹R¹¹; and Ring A is pyridinyl that is substituted with 2 R^{A} substituents independently selected from fluoro and —OR¹⁰.

In some embodiments, the compound of Formula (I), (Ia), (Ib), (II), (IIa), (IIb), (III), (IIIa), (IIIb), (IV), (IVa), (IVb), (V), (Va), (Vb), (VII), (VIIa), or (VIIb), or a pharmaceutically acceptable salt, solvate, stereoisomer, or prodrug thereof, is a compound of Formula (VIII), (VIIIa), or (VIIIb):

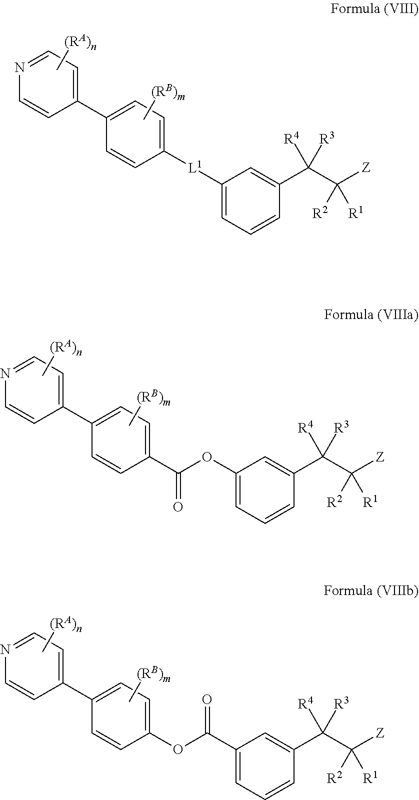

or a pharmaceutically acceptable salt, solvate, stereoisomer, or prodrug thereof; wherein: n and m are each independently 0, 1, 2, or 3.

In some embodiments of a compound of Formula (VIII), (VIIIa), or (VIIIb), or a pharmaceutically acceptable salt, solvate, stereoisomer, or prodrug thereof, n is 0, 1, 2, or 3; and m is 0, 1, 2, or 3. In some embodiments, n is 0. In some embodiments, n is 1. In some embodiments, n is 2. In some embodiments, n is 3. In some embodiments, m is 0. In some embodiments, m is 1. In some embodiments, m is 2. In some embodiments, m is 3. In some embodiments, n is 0, 1, or 2; and m is 0, 1, or 2. In some embodiments, n 1 or 2; and m is 1 or 2. In some embodiments, n 2; and m is 1.

In some embodiments, disclosed herein is a compound, or a pharmaceutically acceptable salt, solvate, stereoisomer, or prodrug thereof, selected from:

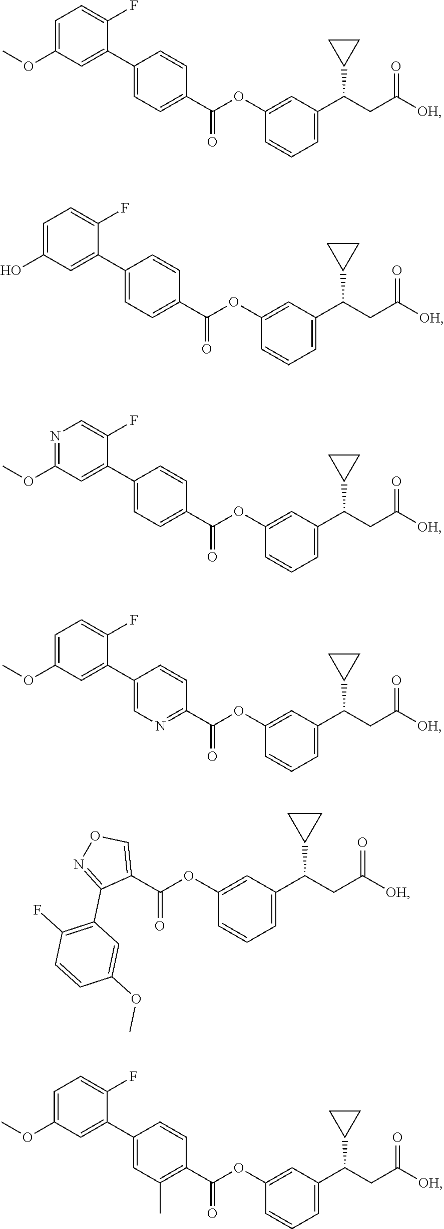

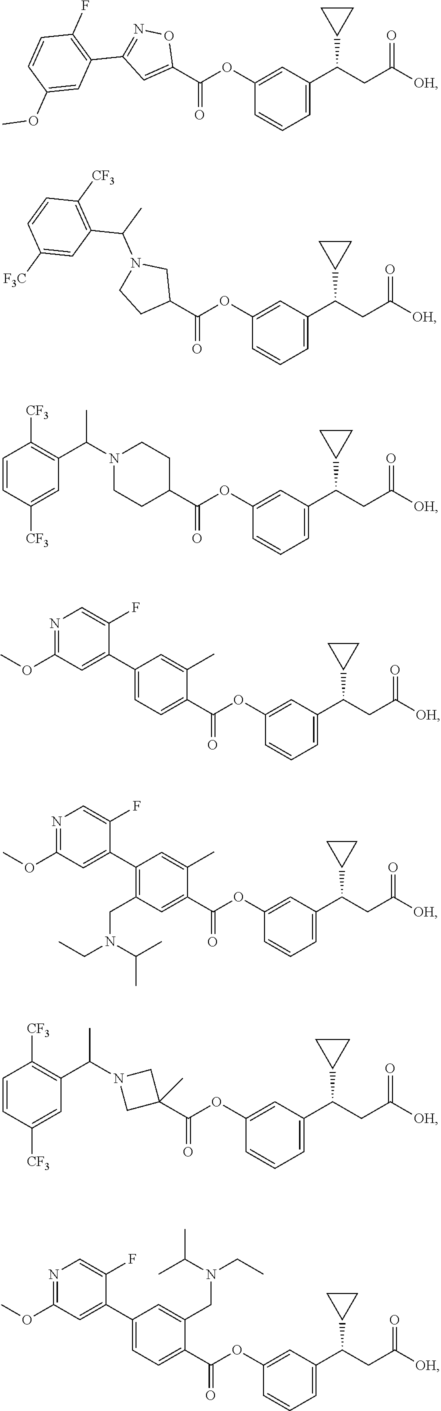

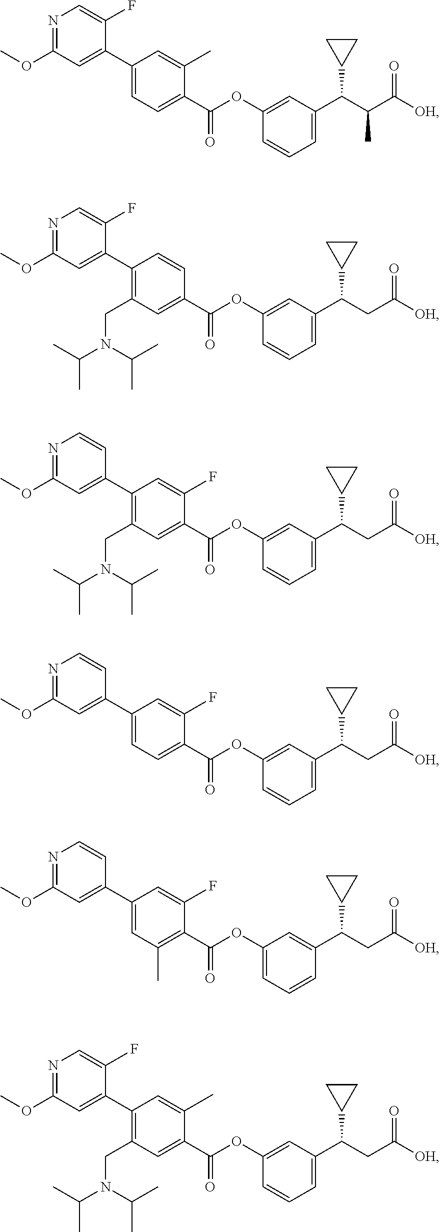

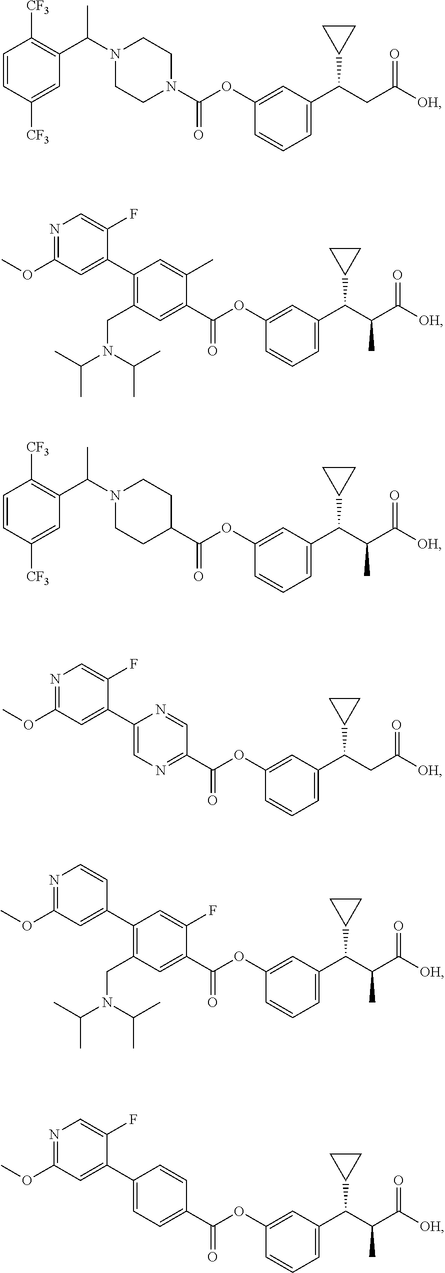 [SMILES: CC(c1cc(C(F)(F)F)ccc1C(F)(F)F)N1CCN(C(=O)Oc2cccc([C@@H](CC(=O)O)C3CC3)c2)CC1 ; COc1cc(-c2cc(C)c(C(=O)Oc3cccc([C@H](C4CC4)[C@H](C)C(=O)O)c3)cc2CN(C(C)C)C(C)C)c(F)cn1 ; CC(c1cc(C(F)(F)F)ccc1C(F)(F)F)N1CCC(C(=O)Oc2cccc([C@H](C3CC3)[C@H](C)C(=O)O)c2)CC1 ; COc1cc(-c2cnc(C(=O)Oc3cccc([C@@H](CC(=O)O)C4CC4)c3)cn2)c(F)cn1 ; COc1cc(-c2cc(F)c(C(=O)Oc3cccc([C@H](C4CC4)[C@H](C)C(=O)O)c3)cc2CN(C(C)C)C(C)C)ccn1 ; COc1cc(-c2ccc(C(=O)Oc3cccc([C@H](C4CC4)[C@H](C)C(=O)O)c3)cc2)c(F)cn1]

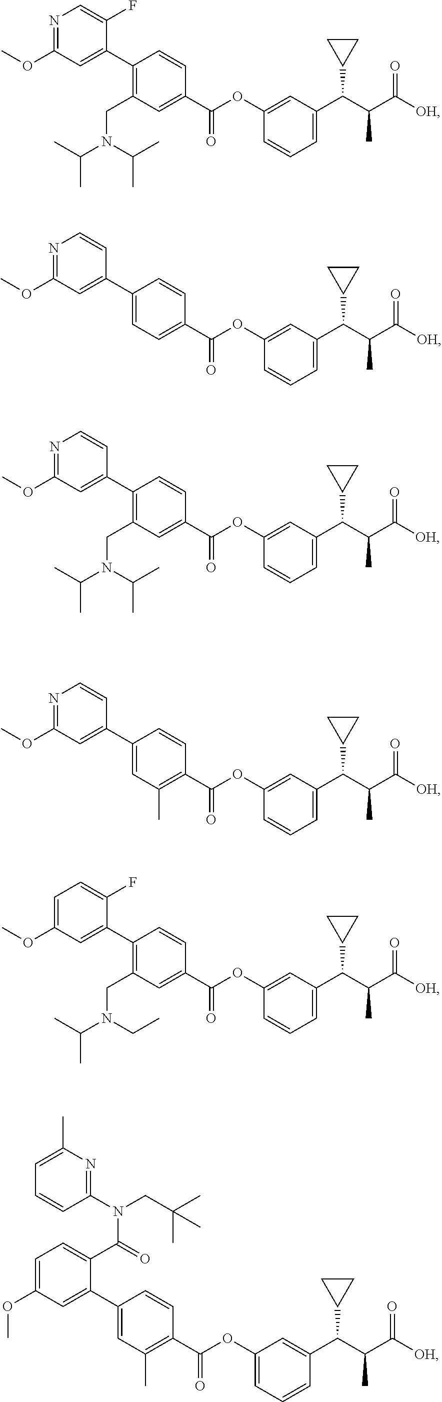 [SMILES: COc1cc(-c2ccc(C(=O)Oc3cccc([C@H](C4CC4)[C@H](C)C(=O)O)c3)cc2CN(C(C)C)C(C)C)c(F)cn1 ; COc1cc(-c2ccc(C(=O)Oc3cccc([C@H](C4CC4)[C@H](C)C(=O)O)c3)cc2)ccn1 ; COc1cc(-c2ccc(C(=O)Oc3cccc([C@H](C4CC4)[C@H](C)C(=O)O)c3)cc2CN(C(C)C)C(C)C)ccn1 ; COc1cc(-c2ccc(C(=O)Oc3cccc([C@H](C4CC4)[C@H](C)C(=O)O)c3)c(C)c2)ccn1 ; CCN(Cc1cc(C(=O)Oc2cccc([C@H](C3CC3)[C@H](C)C(=O)O)c2)ccc1-c1cc(OC)ccc1F)C(C)C ; COc1ccc(C(=O)N(CC(C)(C)C)c2cccc(C)n2)c(-c2ccc(C(=O)Oc3cccc([C@H](C4CC4)[C@H](C)C(=O)O)c3)c(C)c2)c1]

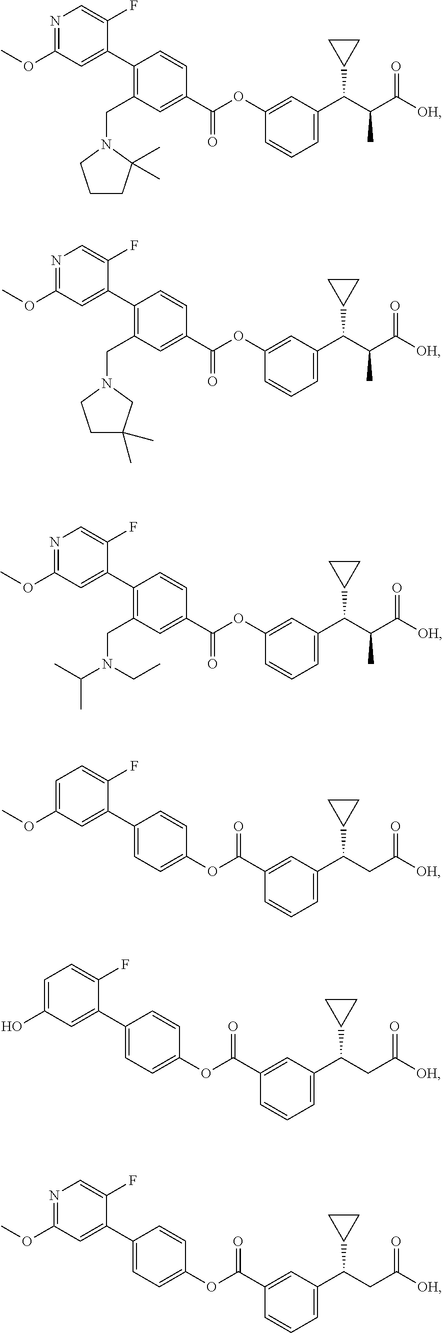 [SMILES: COc1cc(-c2ccc(C(=O)Oc3cccc([C@H](C4CC4)[C@H](C)C(=O)O)c3)cc2CN2CCCC2(C)C)c(F)cn1 ; COc1cc(-c2ccc(C(=O)Oc3cccc([C@H](C4CC4)[C@H](C)C(=O)O)c3)cc2CN2CCC(C)(C)C2)c(F)cn1 ; CCN(Cc1cc(C(=O)Oc2cccc([C@H](C3CC3)[C@H](C)C(=O)O)c2)ccc1-c1cc(OC)ncc1F)C(C)C ; COc1ccc(F)c(-c2ccc(OC(=O)c3cccc([C@@H](CC(=O)O)C4CC4)c3)cc2)c1 ; O=C(O)C[C@H](c1cccc(C(=O)Oc2ccc(-c3cc(O)ccc3F)cc2)c1)C1CC1 ; COc1cc(-c2ccc(OC(=O)c3cccc([C@@H](CC(=O)O)C4CC4)c3)cc2)c(F)cn1]

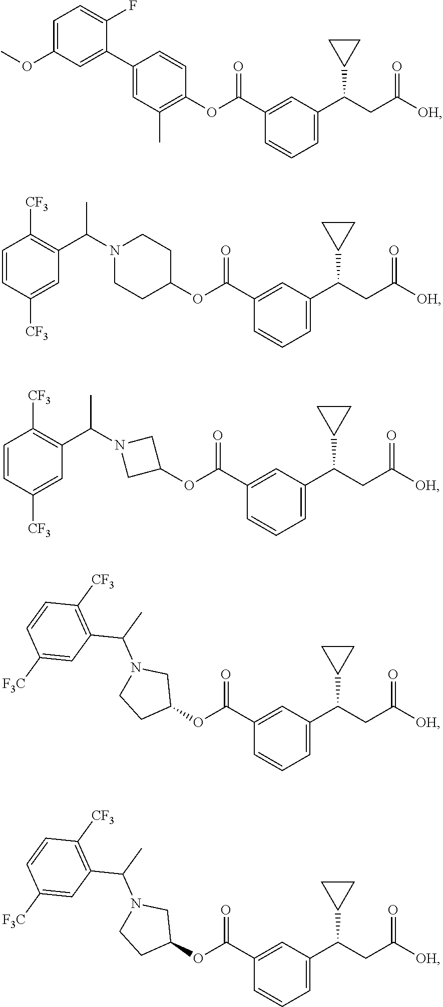 [SMILES: COc1ccc(F)c(-c2ccc(OC(=O)c3cccc([C@@H](CC(=O)O)C4CC4)c3)c(C)c2)c1 ; CC(c1cc(C(F)(F)F)ccc1C(F)(F)F)N1CCC(OC(=O)c2cccc([C@@H](CC(=O)O)C3CC3)c2)CC1 ; CC(c1cc(C(F)(F)F)ccc1C(F)(F)F)N1CC(OC(=O)c2cccc([C@@H](CC(=O)O)C3CC3)c2)C1 ; CC(c1cc(C(F)(F)F)ccc1C(F)(F)F)N1CC[C@@H](OC(=O)c2cccc([C@@H](CC(=O)O)C3CC3)c2)C1 ; CC(c1cc(C(F)(F)F)ccc1C(F)(F)F)N1CC[C@H](OC(=O)c2cccc([C@@H](CC(=O)O)C3CC3)c2)C1]

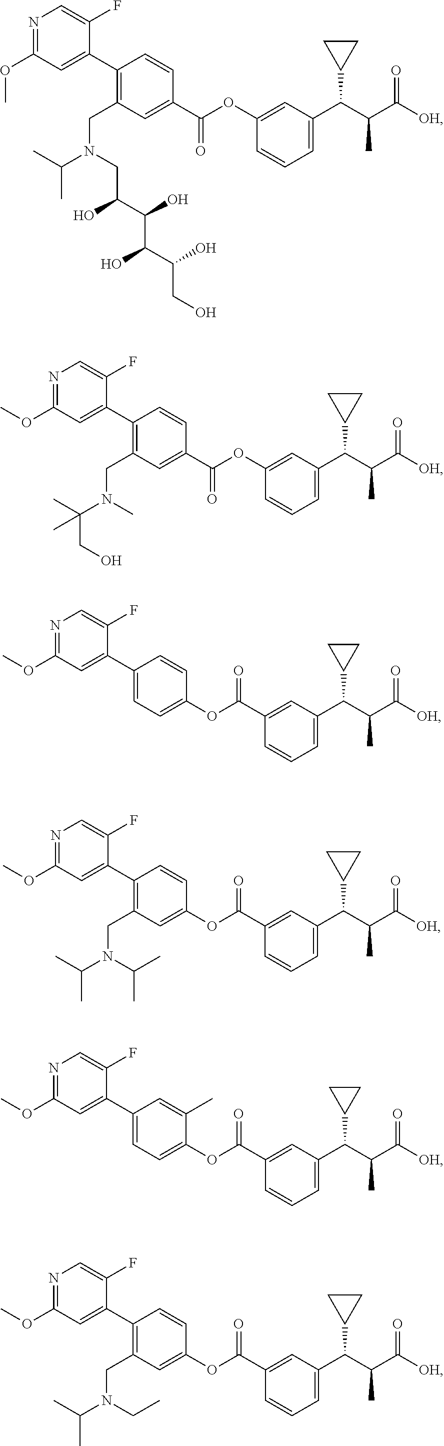

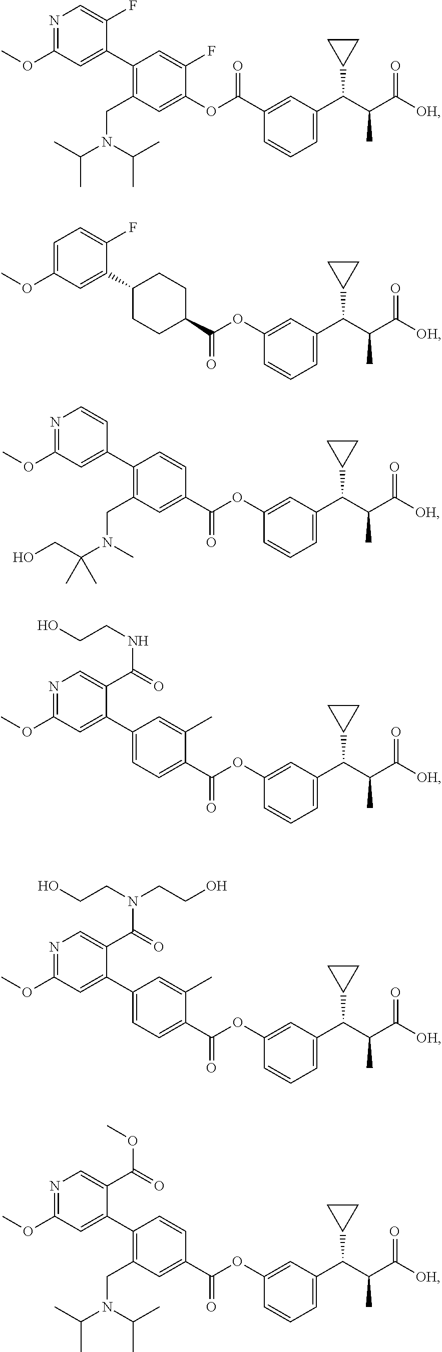

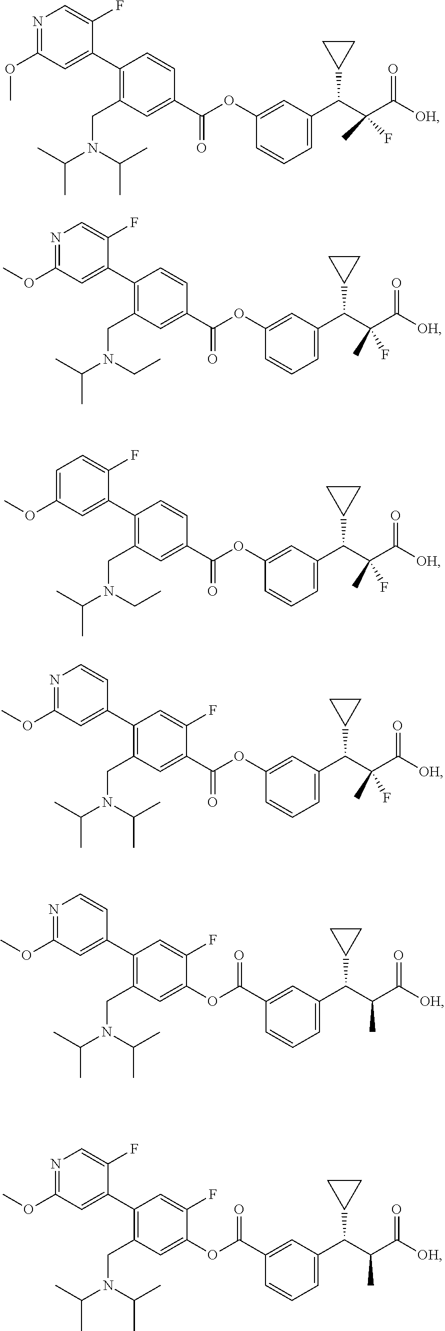

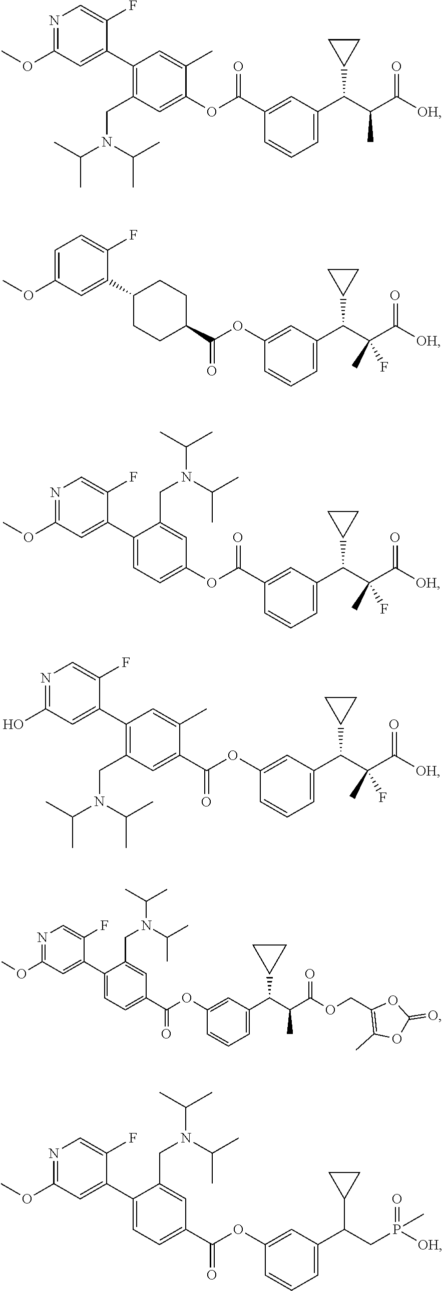

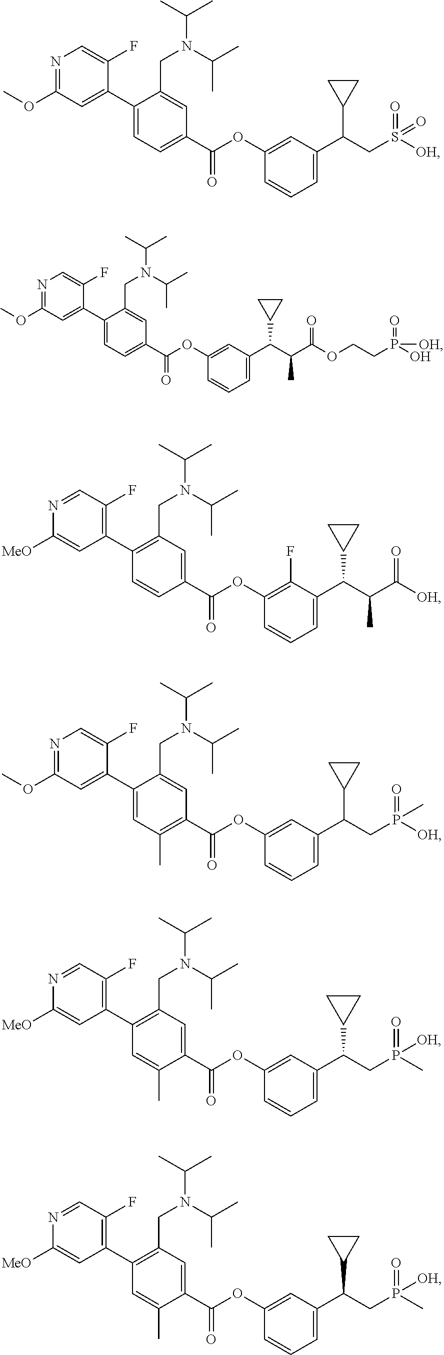

or a pharmaceutically acceptable salt, solvate, stereoisomer, or prodrug thereof.

In some embodiments, disclosed herein is a compound, or a pharmaceutically acceptable salt, solvate, stereoisomer, or prodrug thereof, selected from:

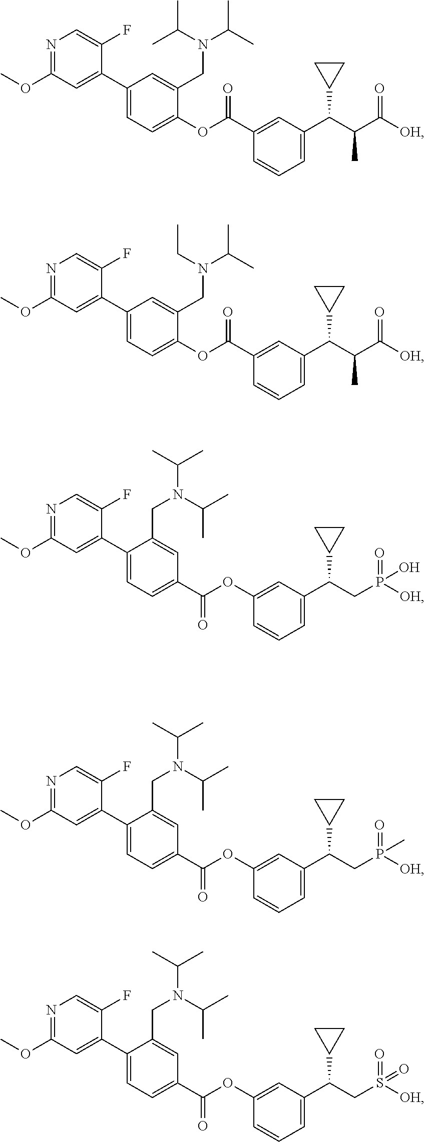 [SMILES: COc1cc(-c2ccc(OC(=O)c3cccc([C@H](C4CC4)[C@H](C)C(=O)O)c3)c(CN(C(C)C)C(C)C)c2)c(F)cn1 ; CCN(Cc1cc(-c2cc(OC)ncc2F)ccc1OC(=O)c1cccc([C@H](C2CC2)[C@H](C)C(=O)O)c1)C(C)C ; COc1cc(-c2ccc(C(=O)Oc3cccc([C@@H](CP(=O)(O)O)C4CC4)c3)cc2CN(C(C)C)C(C)C)c(F)cn1 ; COc1cc(-c2ccc(C(=O)Oc3cccc([C@@H](CP(C)(=O)O)C4CC4)c3)cc2CN(C(C)C)C(C)C)c(F)cn1 ; COc1cc(-c2ccc(C(=O)Oc3cccc([C@@H](CS(=O)(=O)O)C4CC4)c3)cc2CN(C(C)C)C(C)C)c(F)cn1]

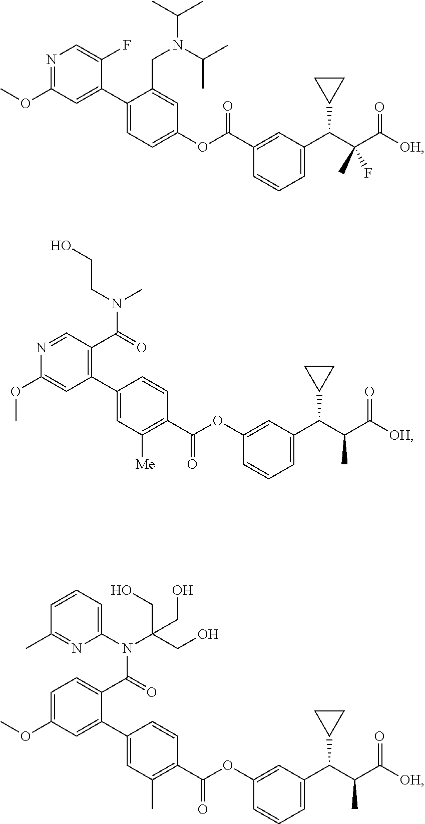 [SMILES: COc1cc(-c2ccc(OC(=O)c3cccc([C@H](C4CC4)[C@@](C)(F)C(=O)O)c3)cc2CN(C(C)C)C(C)C)c(F)cn1 ; COc1cc(-c2ccc(C(=O)Oc3cccc([C@H](C4CC4)[C@H](C)C(=O)O)c3)c(C)c2)c(C(=O)N(C)CCO)cn1 ; COc1ccc(C(=O)N(c2cccc(C)n2)C(CO)(CO)CO)c(-c2ccc(C(=O)Oc3cccc([C@H](C4CC4)[C@H](C)C(=O)O)c3)c(C)c2)c1]

or a pharmaceutically acceptable salt, solvate, stereoisomer, or prodrug thereof

### Further Forms of Compounds

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

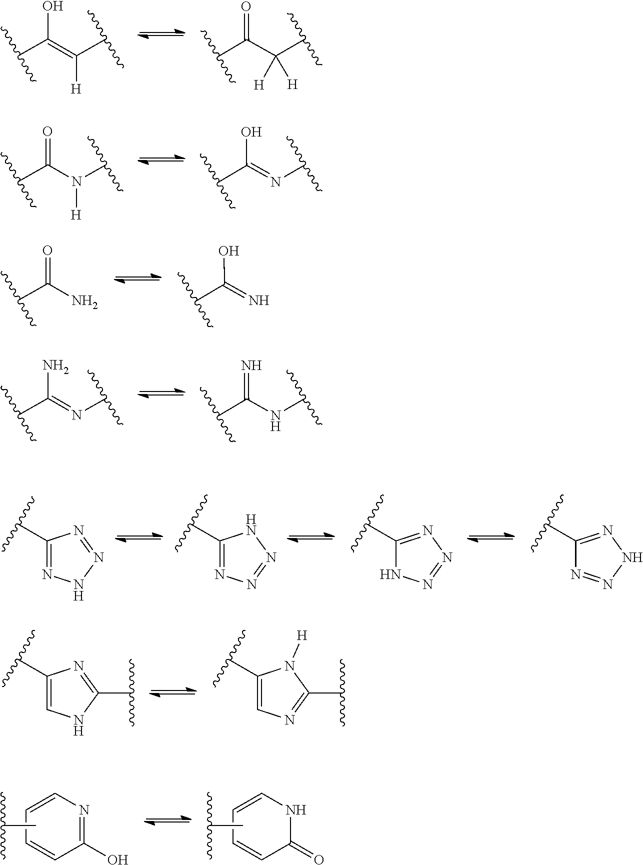

In some situations, the compounds described herein possess one or more chiral centers and each center exists in the (R)-configuration or (S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

The term “positional isomer” refers to structural isomers around a central ring, such as ortho-, meta-, and para-isomers around a benzene ring.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that is, in some embodiments, converted under physiological conditions or by solvolysis to an active compound described herein. Thus, the term prodrug refers to a precursor of an active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, carboxy, or mercapto group is bonded to any group that, when the prodrug of the active compound is administered to a mammalian subject, cleaves to form a free hydroxy, free amino, free carboxy, or free mercapto group, respectively. Examples of prodrugs include, but are not limited to, acetate, formate and benzoate derivatives of alcohol or amine functional groups in the active compounds and the like.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. “Hydrates” are formed when the solvent is water, or “alcoholates” are formed when the solvent is alcohol. Solvates of compounds described herein are conveniently prepared or formed during the processes described herein. The compounds provided herein optionally exist in either unsolvated as well as solvated forms.

The compounds disclosed herein, in some embodiments, are used in different enriched isotopic forms, e.g., enriched in the content of ²H, ³H, ¹¹C, ¹³C and/or ¹⁴C. In some embodiments, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or 14C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (³H), iodine-125 (¹²⁵I) or carbon-14 (¹⁴C). Isotopic substitution with ²H, ³H, ¹¹C, ¹³C, ¹⁴C, ¹⁵C, ¹²N, ¹³N ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art. In some embodiments deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments, the compounds described herein are labeled by other means, including, but not limited to, the use of chromophores or fluorescent moieties, bioluminescent labels, or chemiluminescent labels.

In certain embodiments, the compounds described herein, or a pharmaceutically acceptable salt, solvate, stereoisomer, or prodrug thereof, as described herein are substantially pure, in that it contains less than about 5%, or less than about 1%, or less than about 0.1%, of other organic small molecules, such as contaminating intermediates or by-products that are created, for example, in one or more of the steps of a synthesis method.

### Preparation of the Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described as outlined in the Examples.

### Pharmaceutical Compositions

In some embodiments, disclosed herein is a pharmaceutical composition comprising a GPR40 agonist described herein, or a pharmaceutically acceptable salt, solvate, stereoisomer, or prodrug thereof, and a pharmaceutically acceptable excipient. In some embodiments, the GPR40 agonist is combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt or solvate thereof, together with a pharmaceutically acceptable excipient.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

### Combination Therapies

In certain embodiments, it is appropriate to administer at least one compound described herein, or a pharmaceutically acceptable salt, solvate, stereoisomer, or prodrug thereof, in combination with one or more other therapeutic agents. In some embodiments, a compound described herein, or a pharmaceutically acceptable salt, solvate, stereoisomer, or prodrug thereof, is administered in combination with a TGR5 agonist, a GPR119 agonist, an SSTR5 antagonist, an SSTR5 inverse agonist, a CCK1 agonist, a PDE4 inhibitor, a DPP-4 inhibitor, a GLP-1 receptor agonist, a ghrelin O-acyltransferase (GOAT) inhibitor, metformin, or combinations thereof. In some embodiments, a compound described herein, or a pharmaceutically acceptable salt, solvate, stereoisomer, or prodrug thereof, is administered in combination with a TGR5 agonist, a GPR119 agonist, an SSTR5 antagonist, an SSTR5 inverse agonist, a CCK1 agonist, a PDE4 inhibitor, a DPP-4 inhibitor, or combinations thereof. In certain embodiments, the pharmaceutical composition further comprises one or more anti-diabetic agents. In certain embodiments, the pharmaceutical composition further comprises one or more anti-obesity agents. In certain embodiments, the pharmaceutical composition further comprises one or more agents to treat nutritional disorders.

Examples of a TGR5 agonist to be used in combination with a compound described herein, or a pharmaceutically acceptable salt, solvate, stereoisomer, or prodrug thereof, include: INT-777, XL-475, SRX-1374, RDX-8940, RDX-98940, SB-756050, and those disclosed in WO-2008091540, WO-2010059853, WO-2011071565, WO-2018005801, WO-2010014739, WO-2018005794, WO-2016054208, WO-2015160772, WO-2013096771, WO-2008067222, WO-2008067219, WO-2009026241, WO-2010016846, WO-2012082947, WO-2012149236, WO-2008097976, WO-2016205475, WO-2015183794, WO-2013054338, WO-2010059859, WO-2010014836, WO-2016086115, WO-2017147159, WO-2017147174, WO-2017106818, WO-2016161003, WO-2014100025, WO-2014100021, WO-2016073767, WO-2016130809, WO-2018226724, WO-2018237350, WO-2010093845, WO-2017147137, WO-2015181275, WO-2017027396, WO-2018222701, WO-2018064441, WO-2017053826, WO-2014066819, WO-2017079062, WO-2014200349, WO-2017180577, WO-2014085474.

Examples of a GPR119 agonist to be used in combination with a compound described herein, or a pharmaceutically acceptable salt, solvate, stereoisomer, or prodrug thereof, include: DS-8500a, HD-2355, LC34AD3, PSN-491, HM-47000, PSN-821, MBX-2982, GSK-1292263, APD597, DA-1241, and those described in WO-2009141238, WO-2010008739, WO-2011008663, WO-2010013849, WO-2012046792, WO-2012117996, WO-2010128414, WO-2011025006, WO-2012046249, WO-2009106565, WO-2011147951, WO-2011127106, WO-2012025811, WO-2011138427, WO-2011140161, WO-2011061679, WO-2017175066, WO-2017175068, WO-2015080446, WO-2013173198, US-20120053180, WO-2011044001, WO-2010009183, WO-2012037393, WO-2009105715, WO-2013074388, WO-2013066869, WO-2009117421, WO-201008851, WO-2012077655, WO-2009106561, WO-2008109702, WO-2011140160, WO-2009126535, WO-2009105717, WO-2013122821, WO-2010006191, WO-2009012275, WO-2010048149, WO-2009105722, WO-2012103806, WO-2008025798, WO-2008097428, WO-2011146335, WO-2012080476, WO-2017106112, WO-2012145361, WO-2012098217, WO-2008137435, WO-2008137436, WO-2009143049, WO-2014074668, WO-2014052619, WO-2013055910, WO-2012170702, WO-2012145604, WO-2012145603, WO-2011030139, WO-2018153849, WO-2017222713, WO-2015150565, WO-2015150563, WO-2015150564, WO-2014056938, WO-2007120689, WO-2016068453, WO-2007120702, WO-2013167514, WO-2011113947, WO-2007003962, WO-2011153435, WO-2018026890, WO-2011163090, WO-2011041154, WO-2008083238, WO-2008070692, WO-2011150067, and WO-2009123992.

Examples of a SSTR5 antagonist or inverse agonist to be used in combination with a compound described herein, or a pharmaceutically acceptable salt, solvate, stereoisomer, or prodrug thereof, include those described in: WO-03104816, WO-2009050309, WO-2015052910, WO-2011146324, WO-2006128803, WO-2010056717, WO-2012024183, and WO-2016205032.

Examples of a CCK1 agonist to be used in combination with a compound described herein, or a pharmaceutically acceptable salt, solvate, stereoisomer, or prodrug thereof, include: A-70874, A-71378, A-71623, A-74498, CE-326597, GI-248573, GSKI-181771X, NN-9056, PD-149164, PD-134308, PD-135158, PD-170292, PF-04756956, SR-146131, SSR-125180, and those described in EP-00697403, US-20060177438, WO-2000068209, WO-2000177108, WO-2000234743, WO-2000244150, WO-2009119733, WO-2009314066, WO-2009316982, WO-2009424151, WO-2009528391, WO-2009528399, WO-2009528419, WO-2009611691, WO-2009611940, WO-2009851686, WO-2009915525, WO-2005035793, WO-2005116034, WO-2007120655, WO-2007120688, WO-2008091631, WO-2010067233, WO-2012070554, and WO-2017005765.

Examples of a PDE4 inhibitor to be used in combination with a compound described herein, or a pharmaceutically acceptable salt, solvate, stereoisomer, or prodrug thereof, include: apremilast, cilomilast, crisaborole, diazepam, luteolin, piclamilast, and roflumilast.

Examples of a DPP-4 inhibitor to be used in combination with a compound described herein, or a pharmaceutically acceptable salt, solvate, stereoisomer, or prodrug thereof, include: sitagliptin, vildagliptin, saxagliptin, linagliptin, gemigliptin, teneligliptin, alogliptin, trelagliptin, omarigliptin, evogliptin, gosogliptin, and dutogliptin.

Examples of a GLP-1 receptor agonist to be used in combination with a compound described herein, or a pharmaceutically acceptable salt, solvate, stereoisomer, or prodrug thereof, include: albiglutide, dulaglutide, exenatide, extended-release exenatide, liraglutide, lixisenatide, and semaglutide.

Examples of a GOAT inhibitors to be used in combination with a compound described herein, or a pharmaceutically acceptable salt, solvate, stereoisomer, or prodrug thereof, include: T-3525770 (RM-852), GLWL-01, BOS-704, and those described in U.S. Ser. No. 08/013,015, U.S. Ser. No. 09/340,578, WO-2019149959, US-20170056373, WO-2018035079, WO-2016044467, WO-2010039461, WO-2018024653, WO-2019149660, WO-2019149659, WO-2015073281, WO-2019149658, WO-2016168225, WO-2016168222, WO-2019149657, WO-2013125732, and WO-2019152889.

Examples of anti-diabetic agents to be used in combination with a compound described herein, or a pharmaceutically acceptable salt, solvate, stereoisomer, or prodrug thereof, include: GLP-1 receptor agonists such as exenatide, liraglutide, taspoglutide, lixisenatide, albiglutide, dulaglutide, semaglutide, OWL833 and ORMD 0901; SGLT2 inhibitors such as dapagliflozin, canagliflozin, empagliflozin, ertugliflozin, ipragliflozin, luseogliflozin, remogliflozin, sergliflozin, sotagliflozin, and tofogliflozin; biguinides such as metformin; insulin and insulin analogs.

Examples of anti-obesity agents to be used in combination with a compound described herein, or a pharmaceutically acceptable salt, solvate, stereoisomer, or prodrug thereof, include: GLP-1 receptor agonists such as liraglutide, semaglutide; SGLT1/2 inhibitors such as LIK066, pramlintide and other amylin analogs such as AM-833, AC2307, and BI 473494; PYY analogs such as NN-9747, NN-9748, AC-162352, AC-163954, GT-001, GT-002, GT-003, and RHS-08; GIP receptor agonists such as APD-668 and APD-597; GLP-1/GIP co-agonists such as tirzepatide (LY329176), BHM-089, LBT-6030, CT-868, SCO-094, NNC-0090-2746, RG-7685, NN-9709, and SAR-438335; GLP-1/glucagon co-agonist such as cotadutide (MED10382), BI 456906, TT-401, G-49, H&D-001A, ZP-2929, and HM-12525A; GLP-1/GIP/glucagon triple agonist such as SAR-441255, HM-15211, and NN-9423; GLP-1/secretin co-agonists such as GUB06-046; leptin analogs such as metreleptin; GDF15 modulators such as those described in WO2012138919, WO2015017710, WO2015198199, WO-2017147742 and WO-2018071493; FGF21 receptor modulators such as NN9499, NGM386, NGM313, BFKB8488A (RG7992), AKR-001, LLF-580, CVX-343, LY-2405319, BI089-100, and BMS-986036; MC4 agonists such as setmelanotide; MetAP2 inhibitors such as ZGN-1061; ghrelin receptor modulators such as HM04 and AZP-531; ghrelin O-acyltransferase inhibitors such as T-3525770 (RM-852) and GLWL-01; and oxytocin analogs such as carbetocin.

Examples of agents for nutritional disorders to be used in combination with a compound described herein, or a pharmaceutically acceptable salt, solvate, stereoisomer, or prodrug thereof, include: GLP-2 receptor agonists such as tedaglutide, glepaglutide (ZP1848), elsiglutide (ZP1846), apraglutide (FE 203799), HM-15912, NB-1002, GX-G8, PE-0503, SAN-134, and those described in WO-2011050174, WO-2012028602, WO-2013164484, WO-2019040399, WO-2018142363, WO-2019090209, WO-2006117565, WO-2019086559, WO-2017002786, WO-2010042145, WO-2008056155, WO-2007067828, WO-2018229252, WO-2013040093, WO-2002066511, WO-2005067368, WO-2009739031, WO-2009632414, and WO2008028117; and GLP-1/GLP-2 receptor co-agonists such as ZP-GG-72 and those described in WO-2018104561, WO-2018104558, WO-2018103868, WO-2018104560, WO-2018104559, WO-2018009778, WO-2016066818, and WO-2014096440.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt, solvate, stereoisomer, or prodrug thereof, is co-administered with one or more additional therapeutic agents, wherein the compound described herein, or a pharmaceutically acceptable salt, solvate, stereoisomer, or prodrug thereof, and the additional therapeutic agent(s) modulate different aspects of the disease, disorder or condition being treated, thereby providing a greater overall benefit than administration of either therapeutic agent alone. In some embodiments, the additional therapeutic agent(s) is a TGR5 agonist, a GPR119 agonist, an SSTR5 antagonist, an SSTR5 inverse agonist, a CCK1 agonist, a PDE4 inhibitor, a DPP-4 inhibitor, a GOAT inhibitor, a GLP-1 receptor agonist, metformin, or combinations thereof. In some embodiments, the additional therapeutic agent(s) is a TGR5 agonist, a GPR119 agonist, an SSTR5 antagonist, an SSTR5 inverse agonist, a CCK1 agonist, a PDE4 inhibitor, a DPP-4 inhibitor, or combinations thereof. In some embodiments, the additional therapeutic agent(s) is a GPR119 agonist, an SSTR5 antagonist, an SSTR5 inverse agonist, or combinations thereof. In some embodiments, the additional therapeutic agent(s) is a GPR119 agonist, an SSTR5 antagonist, or combinations thereof. In some embodiments, the additional therapeutic agents is a GPR119 agonist. In some embodiments, the additional therapeutic agents is an SSTR5 antagonist. In some embodiments, the additional therapeutic agent(s) is a combination of a GPR119 agonist and an SSTR5 antagonist. In some embodiments, the additional therapeutic agent is an anti-diabetic agent. In some embodiments, the additional therapeutic agent is an anti-obesity agent. In some embodiments, the additional therapeutic agent is an agent to treat nutritional disorders.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or pharmaceutically acceptable salts, solvates, stereoisomers, or prodrugs thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease.

In some embodiments, a compound described herein, or a pharmaceutically acceptable salt thereof, is administered in combination with anti-inflammatory agent, anti-cancer agent, immunosuppressive agent, steroid, non-steroidal anti-inflammatory agent, antihistamine, analgesic, hormone blocking therapy, radiation therapy, monoclonal antibodies, or combinations thereof.

### EXAMPLES

### List of Abbreviations

As used above, and throughout the description of the invention, the following abbreviations, unless otherwise indicated, shall be understood to have the following meanings:
ACN or MeCN acetonitrile
AcOH acetic acid
AIBN azobisisobutyronitrile
BPO benzoyl peroxide
Boc or BOC tert-butyloxycarbonyl
Bn benzyl
BnBr benzyl bromide
DCC N,N′-dicyclohexylcarbodiimide
DCE 1,1-dichloroethane
DCM dichloromethane (CH₂Cl₂)
DIAD diisopropyl azodicarboxylate
DIPEA or DIEA diisopropylethylamine
DMAP 4-dimethylaminopyridine
DMF dimethylformamide
DMP Dess-Martin periodinane
DMSO dimethylsulfoxide
EDCI 1-ethyl-3-(3-dimethylaminopropyl)carbodiimide
eq equivalent(s)
Et ethyl
EtOH ethanol
EtOAc ethyl acetate
FA formic acid
h, hr(s) hour(s)
HATU 1-[bis(dimethylamino)methylene]-1H-1,2,3-triazolo[4,5-b]pyridinium 3-oxid hexafluorophosphate
HPLC high performance liquid chromatography
IPA or i-PrOH isopropanol
LCMS liquid chromatography-mass spectrometry
Me methyl
MeOH methanol
MS mass spectroscopy
Ms methanesulfonyl (mesyl)
MsCl methanesulfonyl chloride (mesyl chloride)
MTBE methyl tert-butyl ether
NBS N-bromosuccinimide
NMR nuclear magnetic resonance
Rt or RT room temperature
SFC supercritical fluid chromatography
TEA triethylamine
Tf trifluoromethylsulfonyl (triflyl)
TFA trifluoroacetic acid
THE tetrahydrofuran
TLC thin layer chromatography
TMSBr trimethylsilyl bromide
Tol or tol toluene
tR retention time
Ts 4-toluenesulfonyl (tosyl)

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: Preparation of (S)-3-cyclopropyl-3-(3-((2′-fluoro-5′-methoxy-[1,1′-biphenyl]-4-carbonyl)oxy)phenyl) propanoic acid (Compound 1)

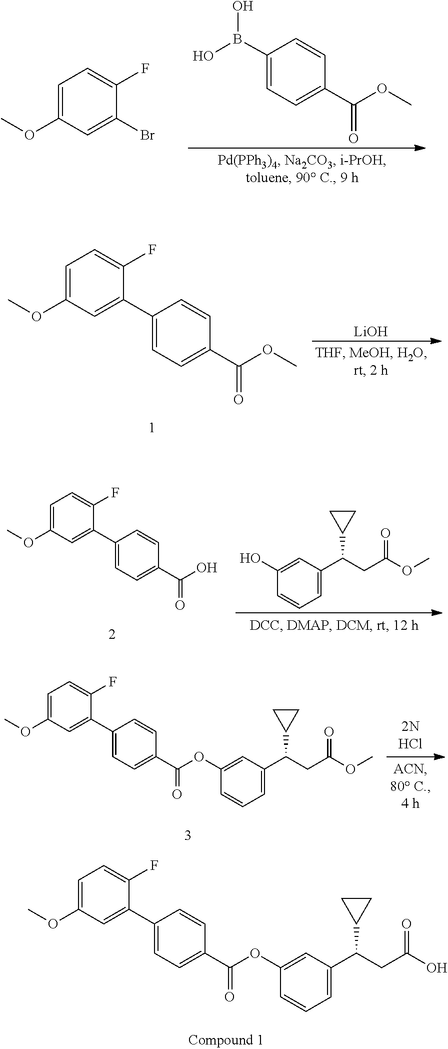

Step 1: methyl 2′-fluoro-5′-methoxy-[1,1′-biphenyl]-4-carboxylate (1): To a solution of 2-bromo-1-fluoro-4-methoxy-benzene (1.0 g, 4.9 mmol, 1 eq) and (4-(methoxycarbonyl)phenyl)boronic acid (0.97 g, 5.4 mmol, 1.1 eq) in i-PrOH (10 mL) and toluene (10 mL) was added Pd(PPh₃)₄ (0.28 g, 0.24 mmol, 0.05 eq) and Na₂CO₃ (2 M, 12 mL, 5 eq) under N₂. The mixture was stirred at 90° C. for 9 hours. The mixture was poured into water (20 mL), and then extracted with ethyl acetate (100 mL×2). The combine organic layers were washed with saturated brine (30 mL×2), concentrated in vacuo to give 1 (1.5 g, crude) as a yellow solid. LCMS: tR=0.896 min., (ES⁺) m/z (M+H)⁺=261.1.

Step 2: 2′-fluoro-5′-methoxy-[1,1′-biphenyl]-4-carboxylic acid (2): To a solution of 1 (1.5 g, 5.8 mmol, 1 eq) in THE (15 mL), MeOH (15 mL) and H₂O (15 mL) was added LiOH H₂O (0.48 mg, 12 mmol, 2 eq) under N₂. The mixture was stirred at room temperature for 2 hours. The mixture was concentrated in vacuo to give a residue. The residue was added water (50 mL), washed with ethyl acetate (10 mL×2). The water phase was adjusted pH to 5.0 with 2 N HCl, and then filtered. The filter residue was dried in vacuo to give 2 (0.90 g, 62% yield) as a white solid. LCMS: tR=0.836 min., (ES+) m/z (M+H)⁺=247.1. ¹H-NMR (CDCl₃, 400 MHz): δ 8.21 (d, J=8.4 Hz, 2H), 7.68 (d, J=7.2 Hz, 2H), 7.12 (t, J=9.2 Hz, 1H), 6.99-6.97 (m, 1H), 6.92-6.88 (m, 1H), 3.85 (s, 3H).

Step 3: (S)-3-(1-cyclopropyl-3-methoxy-3-oxopropyl)phenyl 2′-fluoro-5′-methoxy-[1,1′-biphenyl]-4-carboxylate (3): 2 (0.10 g, 0.41 mmol, 1 eq) was dissolved in dry DCM (5 mL) under N₂ atmosphere and (S)-methyl 3-cyclopropyl-3-(3-hydroxyphenyl)propanoate (89 mg, 0.41 mmol, 1 eq), DCC (0.13 g, 0.61 mmol, 1.5 eq), DMAP (25 mg, 0.20 mmol, 0.5 eq) was slowly added and stirred at room temperature for 12 hours. The mixture was poured into water (5 mL), and then extracted with dichloromethane (20 mL×2). The combine organic layers were washed with saturated brine (5 mL×2), concentrated in vacuo to give crude. The residue was purified by prep-TLC (Petroleum ether:Ethyl acetate=5:1) to give 3 (0.15 g, 82% yield) as a colorless oil. LCMS: tR=1.069 min., (ES+) m/z (M+H)⁺=449.2.

Step 4: (S)-3-cyclopropyl-3-(3-((2′-fluoro-5′-methoxy-[1,1′-biphenyl]-4-carbonyl)oxy)phenyl) propanoic acid (Compound 1): To a solution of 3 (0.14 g, 0.31 mmol, 1 eq) in ACN (2.8 mL) was added HCl (2 M, 2.8 mL, 18 eq) under N₂. The mixture was stirred at 80° C. for 4 hours. The mixture was poured into water (5 mL), and then extracted with ethyl acetate (20 mL×2). The combine organic layers were washed with saturated brine (5 mL×2), concentrated in vacuo to give crude. The residue was purified by prep-TLC (Petroleum ether:Ethyl acetate=1:1) to give Compound 1 (94 mg, 65% yield) as a colorless oil. LCMS: tR=1.002 min., (ES+) m/z (M+Na)+=457.0. ¹H-NMR (CDCl₃, 400 MHz): δ 8.28 (d, J=8.4 Hz, 2H), 7.70 (d, J=8.0 Hz, 2H), 7.39 (t, J=8.0 Hz, 1H), 7.19-7.10 (m, 4H), 7.01-6.98 (m, 1H), 6.92-6.88 (m, 1H), 3.86 (s, 3H), 2.89-2.78 (m, 2H), 2.49-2.43 (m, 1H), 1.10-1.04 (m, 1H), 0.64-0.60 (m, 1H), 0.49-0.48 (m, 1H), 0.35-0.32 (m, 1H), 0.23-0.19 (m, 1H).

### Example 2: Preparation of (3S)-3-cyclopropyl-3-[3-[3-(2-fluoro-5-methoxy-phenyl)isoxazole-4-carbonyl]oxyphenyl]propanoic acid (Compound 5)

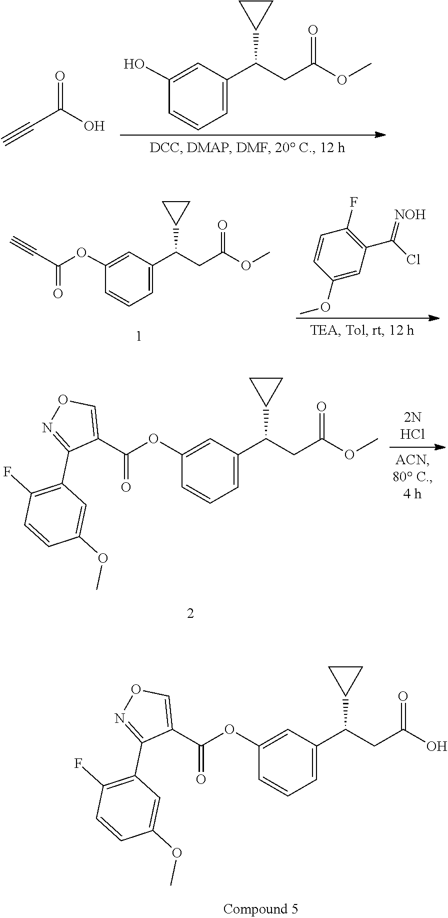

Step 1: methyl (3S)-3-cyclopropyl-3-(3-prop-2-ynoyloxyphenyl)propanoate (1): To a solution of prop-2-ynoic acid (0.10 g, 1.4 mmol, 1 eq) in DCM (2 mL) was added methyl (3S)-3-cyclopropyl-3-(3-hydroxyphenyl)propanoate (0.35 g, 1.6 mmol, 1.1 eq); DCC (0.44 g, 2.1 mmol, 1.5 eq); DMAP (87 mg, 0.71 mmol, 0.5 eq). The reaction was stirred at 20° C. for 12 hours. To this reaction was added H₂O (50.0 mL) and extracted with DCM (50.0 mL×2). The combined organic phase was washed with saturated brine (50.0 mL), dried with anhydrous Na₂SO₄, filtered and concentrated in vacuo. The residue was purified by prep-TLC (SiO₂, PE:EA=3:1) to give 1 (0.34 g, 86% yield) as a yellow oil.

Step 2: [3-[(1S)-1-cyclopropyl-3-methoxy-3-oxo-propyl]phenyl] 3-(2-fluoro-5-methoxy-phenyl)isoxazole-4-carboxylate (2): To a solution of 2-fluoro-N-hydroxy-5-methoxybenzimidoyl chloride (0.12 g, 0.59 mmol, 1 eq) in toluene (7 mL) was added 1 (160.49 mg, 589.40 μmol, 1 eq), Et₃N (77 mg, 0.76 mmol, 1.30 eq) at 0° C. over 1 hour. The reaction was stirred at 20° C. for 12 hours. To this reaction was added H₂O (50 mL) and extracted with ethyl acetate (50 mL×2). The combined organic phase was washed with saturated brine (50 mL), dried with anhydrous Na₂SO₄, filtered and concentrated in vacuo. The residue was purified by column chromatography (SiO₂, Petroleum ether:Ethyl acetate=10:1 to 5:1) to give 2 (92 mg, 25% yield) as a yellow oil. LCMS: tR=0.956 min, (ES⁺) m/z (M+H)⁺=440.0.

Step 3: (3S)-3-cyclopropyl-3-[3-[3-(2-fluoro-5-methoxy-phenyl)isoxazole-4-carbonyl]oxyphenyl]propanoic acid (Compound 5): To a solution of 2 (80 mg, 0.18 mmol, 1 eq) in ACN (1.00 mL) was added HCl (2 M, 1 mL, 11 eq) at 20° C. The reaction was stirred at 80° C. for 4 hours. The reaction was concentrated in vacuo. The residue was purified by prep-HPLC (column: Phenomenex Synergi C18 150×25 mm×10 μm; mobile phase: [water (0.1% TFA) ACN]; B %: 50%-80%, 11 min) to give Compound 5 (42 mg, 54% yield) as a white solid. LCMS: tR=0.864 min, (ES+) m/z (M+H)⁺=426.2. ¹H NMR (CDCl₃, 400 MHz) δ=9.19 (s, 1H), 7.34-7.28 (m, 1H), 7.15-7.07 (m, 3H), 7.03-6.97 (m, 3H), 3.82 (s, 3H), 2.84-2.68 (m, 2H), 2.43-2.35 (m, 1H), 1.07-0.95 (m, 1H), 0.65-0.56 (m, 1H), 0.49-0.40 (m, 1H), 0.30 (m, 1H), 0.15 (m, 1H).

### Example 3: Preparation of (S)-3-cyclopropyl-3-(3-((3-(2-fluoro-5-methoxyphenyl)isoxazole-5-carbonyl)oxy) phenyl)propanoic acid (Compound 7)

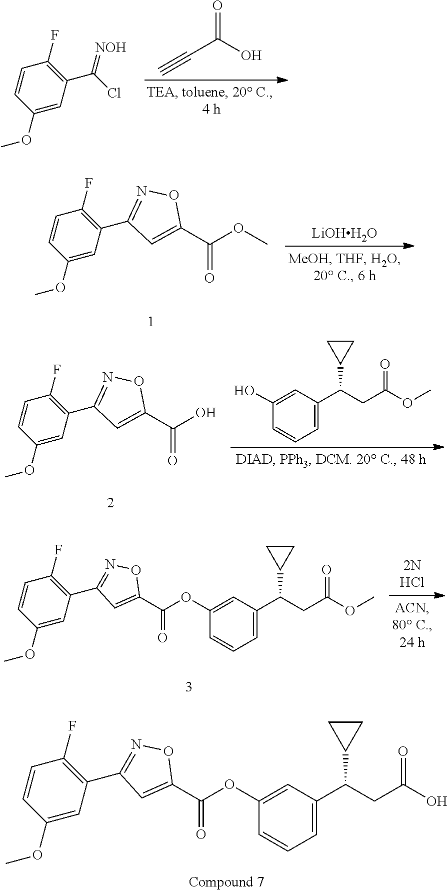

Step 1: methyl 3-(2-fluoro-5-methoxy-phenyl)isoxazole-5-carboxylate (1): To a solution of 2-fluoro-N-hydroxy-5-methoxy-benzimidoyl chloride (0.48 g, 2.4 mmol, 1 eq) in toluene (7 mL) was added methyl prop-2-ynoate (0.23. g, 2.8 mmol, 1.18 eq), Et₃N (0.31 g, 3.1 mmol, 1.3 eq) at 0° C. over 1 hour. The reaction was stirred at 20° C. for 4 hours. To this reaction was added H₂O (50 mL) and extracted with ethyl acetate (50 mL×2). The combined organic phase was washed with saturated brine (50 mL), dried with anhydrous Na₂SO₄, filtered and concentrated in vacuo. The residue was purified by column chromatography (SiO₂, Petroleum ether:Ethyl acetate=50:1 to 20:1) to give 1 (0.29 g, 49% yield) as a yellow solid. ¹H NMR (CD₃OD, 400 MHz) δ=7.46 (m, 1H), 7.43 (d, J=3.2 Hz, 1H), 7.25-7.18 (m, 1H), 7.12-7.06 (m, 1H), 3.98 (s, 3H), 3.84 (s, 3H).

Step 2: 3-(2-fluoro-5-methoxy-phenyl)isoxazole-5-carboxylic acid (2): To a solution of 1 (0.15 g, 0.60 mmol, 1 eq) in a mixture of MeOH (1 mL), THF (1 mL) and H₂O (1 mL) was added LiOH.H₂O (63 mg, 1.5 mmol, 2.5 eq). The reaction was stirred at 20° C. for 6 hours. The reaction was adjusted to pH 3 with 1 N HCl, and concentrated in vacuo to give 2 (0.13 g, crude) as a white solid. LCMS: tR=0.723 min, (ES+) m/z (M+H)⁺=238.0.

Step 3: (S)-3-(1-cyclopropyl-3-methoxy-3-oxopropyl)phenyl 3-(2-fluoro-5-methoxyphenyl) isoxazole-5-carboxylate (3): To a solution of 2 (0.10 g, 0.42 mmol, 1 eq) in DCM (1 mL) was added methyl (3S)-3-cyclopropyl-3-(3-hydroxyphenyl)propanoate (0.11 g, 0.51 mmol, 1.2 eq), DIAD (0.13 g, 0.63 mmol, 1.5 eq), PPh₃ (0.17 g, 0.63 mmol, 1.5 eq) at 20° C. The reaction was stirred at 20° C. for 48 hours. The reaction was concentrated in vacuo. The residue was purified by prep-TLC (SiO₂, PE:EA=3:1) to give 3 (50 mg, 27% yield) as a colorless oil. ¹H NMR (CDCl₃, 400 MHz) δ=7.61-7.54 (m, 2H), 7.44-7.37 (m, 1H), 7.23-7.11 (m, 4H), 7.03 (m, 1H), 3.88 (s, 3H), 3.63 (s, 3H), 2.85-2.71 (m, 2H), 2.48-2.40 (m, 1H), 1.10-1.00 (m, 1H), 0.67-0.57 (m, 1H), 0.53-0.44 (m, 1H), 0.30 (m, 1H), 0.18 (m, 1H).

Step 4: (S)-3-cyclopropyl-3-(3-((3-(2-fluoro-5-methoxyphenyl)isoxazole-5-carbonyl)oxy) phenyl)propanoic acid (Compound 7): To a solution of 3 (50 mg, 0.11 mmol, 1 eq) in ACN (1 mL) was added HCl (2 M, 1 mL). The reaction was stirred at 80° C. for 24 hours. The residue was purified by prep-HPLC (column: Phenomenex Synergi C18 150×30 mm×4 μm; mobile phase: [water (0.225% FA )-ACN]; B %: 50%-80%, 10 min) to give Compound 7 (5.0 mg, 10% yield) as a yellow solid. LCMS: tR=0.914 min, (ES+) m/z (M+H)⁺=426.2. ¹H NMR (CDCl₃, 400 MHz) δ=7.59-7.53 (m, 2H), 7.42-7.35 (m, 1H), 7.20 (br d, J=7.2 Hz, 1H), 7.17-7.10 (m, 3H), 7.02 (m, 1H), 3.87 (s, 3H), 2.81 (br s, 2H), 2.43 (m, 1H), 1.04 (m, 1H), 0.61 (m, 1H), 0.52-0.43 (m, 1H), 0.38-0.27 (m, 1H), 0.19 (m, 1H).

### Example 4: Preparation of (3S)-3-(3-((1-(1-(2,5-bis(trifluoromethyl)phenyl)ethyl)pyrrolidine-3-carbonyl)oxy)phenyl)-3-cyclopropylpropanoic acid (Compound 8)

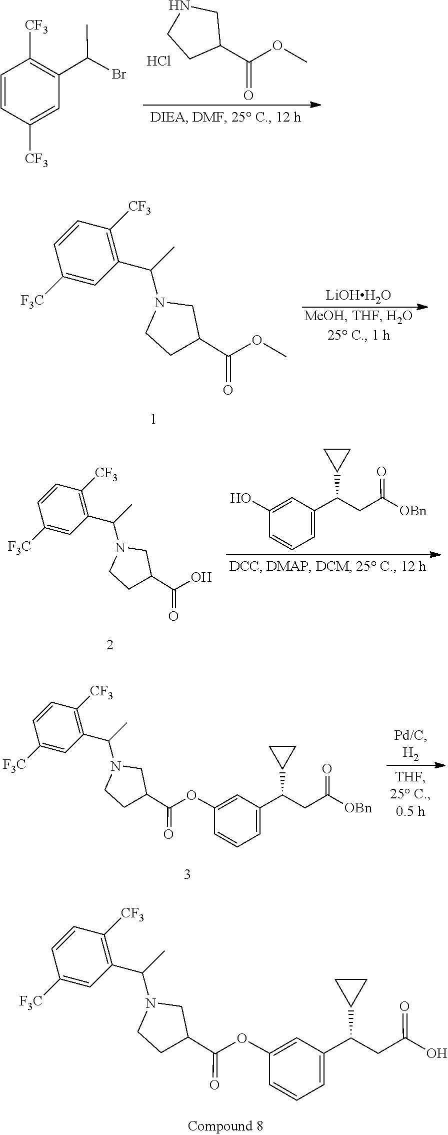

Step 1: methyl 1-(1-(2,5-bis(trifluoromethyl)phenyl)ethyl)pyrrolidine-3-carboxylate (1): To a solution of 2-(1-bromoethyl)-1,4-bis(trifluoromethyl)benzene (0.20 g, 0.62 mmol) in DMF (2 mL) was added DIEA (0.40 g, 3.1 mmol) and methyl pyrrolidine-3-carboxylate hydrochloride (0.31 g, 1.9 mmol). The mixture was stirred at 25° C. for 12 h. The residue was diluted by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by prep-TLC (SiO₂, PE:EA=10:1) to give 1 (50 mg, 21% yield) as a colorless oil. ¹H NMR (CDCl₃, 400 MHz) δ=8.19 (d, J=7.2 Hz, 1H), 7.73 (d, J=8.0 Hz, 1H), 7.58 (d, J=8.0 Hz, 1H), 3.72 (s, 1H), 3.68 (d, J=1.6 Hz, 3H), 3.07-2.94 (m, 1H), 2.89-2.78 (m, 1H), 2.75-2.61 (m, 2H), 2.49-2.33 (m, 1H), 2.20-2.07 (m, 2H), 1.36 (d, J=6.4 Hz, 3H).

Step 2: 1-(1-(2,5-bis(trifluoromethyl)phenyl)ethyl)pyrrolidine-3-carboxylic acid (2): To a solution of 1 (50 mg, 0.14 mmol) in MeOH (0.5 mL), THE (0.5 mL) and H₂O (0.5 mL) was added LiOH.H₂O (11 mg, 0.27 mmol). The mixture was stirred at 25° C. for 1 h. The reaction mixture was concentrated under reduced pressure to give a residue. The residue was diluted by H₂O (10 mL) and adjusted to pH about 7 by 1 N HCl. The solution was extracted with ethyl acetate (20 mL×3). The combined organic layers were dried over anhydrous Na₂SO₄, filtered and concentrated under reduced pressure to give 2 (40 mg, crude) as a colorless oil. LCMS: tR=0.743 min., (ES⁺) m/z (M+H)⁺=356.1.

Step 3: 3-((S)-3-(benzyloxy)-1-cyclopropyl-3-oxopropyl)phenyl 1-(1-(2,5-bis(trifluoromethyl)phenyl)ethyl)azetidine-3-carboxylate (3): To a solution of 2 (40 mg, 0.11 mmol) in DCM (1 mL) was added DCC (35 mg, 0.17 mmol), DMAP (6.9 mg, 56 μmol) and benzyl (3S)-3-cyclopropyl-3-(3-hydroxyphenyl)propanoate (33 mg, 0.11 mmol). The mixture was stirred at 25° C. for 12 h. The residue was diluted by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by prep-TLC (SiO₂, PE:EA=5:1) to give 3 (40 mg, 56% yield) as a yellow oil. ¹H NMR (CDCl₃, 400 MHz) δ=8.23 (d, J=7.2 Hz, 1H), 7.75 (d, J=8.0 Hz, 1H), 7.60 (s, 1H), 7.36-7.28 (m, 4H), 7.26-7.22 (m, 2H), 7.08 (d, J=7.6 Hz, 1H), 6.96-6.90 (m, 2H), 5.10-4.98 (m, 2H), 3.79 (s, 1H), 3.33-3.19 (m, 1H), 2.93-2.68 (m, 4H), 2.57-2.36 (m, 2H), 2.33-2.11 (m, 2H), 1.78-1.67 (m, 1H), 1.39 (dd, J₁=2.4 Hz, J₂=2.4 Hz, 3H), 1.06-0.95 (m, 1H), 0.59-0.50 (m, 1H), 0.47-0.38 (m, 1H), 0.29-0.20 (m, 1H), 0.18-0.10 (m, 1H).

Step 4: (3S)-3-(3-((1-(1-(2,5-bis(trifluoromethyl)phenyl)ethyl)pyrrolidine-3-carbonyl)oxy)phenyl)-3-cyclopropylpropanoic acid (Compound 8): To a solution of 3 (40 mg, 63 μmol) in THE (1 mL) was added 5% Pd/C (4.0 mg, 63 μmol) under H₂. The mixture was stirred at 25° C. for 0.5 h under 15 psi H₂. The reaction mixture was filtered and concentrated under reduced pressure to give a residue. The residue was purified by prep-HPLC (neutral condition; column: Phenomenex Gemini 150×25 mm×10 μm; mobile phase: [water (0.04% NH₃H₂O+10 mM NH₄HCO₃)-ACN]; B %: 40%-70%, min) to give Compound 8 (11 mg, 31% yield) as a yellow oil. LCMS: tR=0.853 min., (ES⁺) m/z (M+H)⁺=544.2. ¹H NMR (DMSO-D₆, 400 MHz) δ=8.17 (d, J=12.4 Hz, 1H), 7.95 (d, J=8.0 Hz, 1H), 7.85 (d, J=7.6 Hz, 1H), 7.30 (t, J=1.6 Hz, 1H), 7.15 (d, J=7.6 Hz, 1H), 7.03-6.96 (m, 1H), 6.91 (dd, J₁=1.6 Hz, J₂=1.6 Hz, 1H), 3.69 (dd, J₁=6.4 Hz, J₂=6.4 Hz 1H), 3.32 (d, J=7.2 Hz, 1H), 2.86-2.76 (m, 1H), 2.75-2.67 (m, 2H), 2.66-2.56 (m, 2H), 2.47-2.24 (m, 2H), 2.21-2.07 (m, 2H), 1.34 (d, J=6.4 Hz, 3H), 1.04-0.93 (m, 1H), 0.49 (dd, J₁=2.4 Hz, J₂=2.4 Hz 1H), 0.35-0.20 (m, 2H), 0.11 (d, J=4.8 Hz, 1H).

### Example 5: Preparation of (S)-3-cyclopropyl-3-(3-((4-(5-fluoro-2-methoxypyridin-4-yl)-2-methylbenzoyl)oxy) phenyl)propanoic acid (Compound 10)

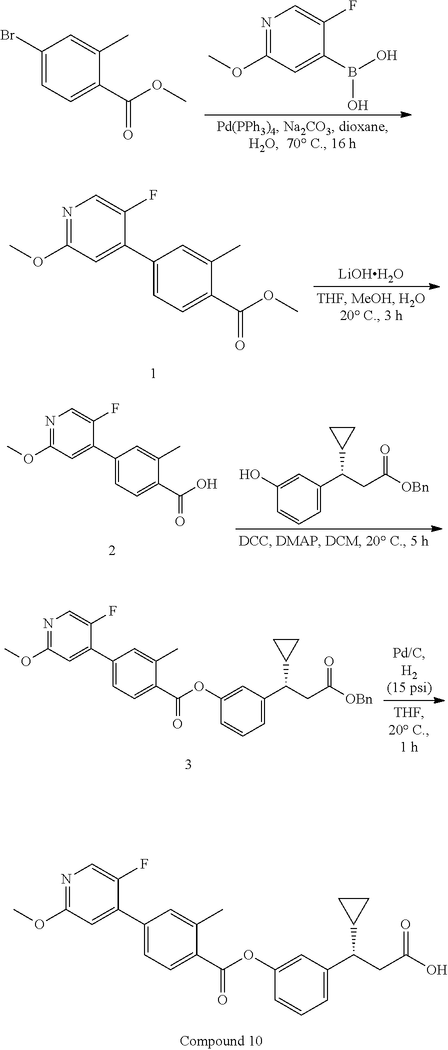

Step 1: methyl 4-(5-fluoro-2-methoxypyridin-4-yl)-2-methylbenzoate (1): To a solution of methyl 4-bromo-2-methylbenzoate (1.0 g, 4.4 mmol), (5-fluoro-2-methoxypyridin-4-yl)boronic acid (1.1 g, 6.6 mmol) in dioxane (10 mL) and H₂O (2 mL) was added Na₂CO₃ (0.93 g, 8.7 mmol) and Pd(PPh₃)₂Cl₂ (0.15 g, 0.22 mmol). The mixture was stirred at 70° C. for 16 hrs. The reaction mixture was quenched by addition water (20 mL), and then diluted with ethyl acetate (20 mL), extracted with ethyl acetate (20 mL×3). The combined organic layers were washed with saturated brine (20 mL), dried over Na₂SO₄, filtered and concentrated under reduced pressure to give a residue. The residue was purified by column chromatography (SiO₂, Petroleum ether:Ethyl acetate=1:0 to 100:1) to give 1 (1.0 g, 83% yield) as a yellow solid. ¹H NMR (400 MHz, DMSO-d₆) δ 8.00-7.98 (d, J=2.4 Hz, 1H), 7.94-7.89 (d, J=8.4 Hz, 1H), 7.40-7.33 (d, J=6.4 Hz, 2H), 6.75-6.70 (d, J=5.2 Hz, 1H), 3.86 (s, 3H), 3.84 (s, 3H), 2.58 (s, 3H).

Step 2: 4-(5-fluoro-2-methoxypyridin-4-yl)-2-methylbenzoic acid (2): To a solution of 1 (0.15 g, 0.54 mmol) in THE (1 mL), H₂O (1 mL) and MeOH (1 mL) was added LiOH.H₂O (46 mg, 1.1 mmol). The mixture was stirred at 25° C. for 3 hrs. The reaction mixture was quenched by addition saturated NH₄Cl solution (3 mL), and then diluted with ethyl acetate (5 mL) and extracted with ethyl acetate (5 mL×3). The combined organic layers were washed with saturated brine (5 mL), dried over Na₂SO₄, filtered and concentrated under reduced pressure to give a residue. The residue was purified by prep-TLC (SiO₂, PE:EA=0:1) to give 2 (69 mg, 48% yield) as a white solid. LCMS: tR=0.823 min, (ES⁺) m/z (M+H)⁺=262. ¹H-NMR (CDCl₃, 400 MHz): δ 8.26-8.28 (d, J=2.4 Hz, 1H), 7.90-7.95 (d, J=8 Hz, 1H), 7.52-7.59 (t, J₁=8.8 Hz, J₂=8 Hz, 2H), 7.03-7.06 (d, J=5.2 Hz, 1H), 3.86-3.91 (s, 3H), 2.57-2.60 (s, 3H).

Step 3: (S)-3-(3-(benzyloxy)-1-cyclopropyl-3-oxopropyl)phenyl 4-(5-fluoro-2-methoxypyridin-4-yl)-2-methylbenzoate (3): To a solution of 2 (69 mg, 0.26 mmol) and (S)-benzyl 3-cyclopropyl-3-(3-hydroxyphenyl)propanoate (78 mg, 0.26 mmol) in DCM (2 mL) was added DCC (82 mg, 0.40 mmol) and DMAP (32 mg, 0.26 mmol). The mixture was stirred at 20° C. for 5 hrs. The mixture was filtered and concentrated under reduced pressure to give a residue. The residue was purified by prep-TLC (SiO₂, PE:EA=20:1) to give 3 (42 mg, 29% yield) as a white solid. LCMS: tR=1.104 min, (ES⁺) m/z (M+H)⁺=540.2.

Step 4: (S)-3-cyclopropyl-3-(3-((4-(5-fluoro-2-methoxypyridin-4-yl)-2-methylbenzoyl)oxy) phenyl)propanoic acid (Compound 10): To a solution of 3 (42 mg, 78 μmol) in THE (1 mL) was added 10% Pd/C (5 mg). The mixture was stirred at 20° C. for 1 hr under 15 psi. The reaction mixture was filtered and concentrated under reduced pressure to give a residue. The residue was purified by prep-HPLC (column: Phenomenex Synergi C18 150×25 mm×10 μm; mobile phase: [water (0.05% HCl)-ACN]; B %: 55%-75%, 9 min) to give Compound 10 (9.0 mg, 26% yield, HCl salt) as a white solid. LCMS: tR=0.982 min, (ES⁺) m/z (M+H)⁺=450.2. ¹H-NMR (CDCl₃; 400 MHz): δ 8.26 (d, J=8.0 Hz, 1H), 8.11 (d, J=2.0 Hz, 1H), 7.55-7.53 (m, 2H), 7.39 (t, J=7.6 Hz, 1H), 7.19 (d, J=7.6 Hz, 1H), 7.12-7.11 (m, 2H), 6.85 (d, J=4.2 Hz, 1H), 3.97 (s, 3H), 2.85-2.77 (m, 2H), 2.75 (s, 3H), 2.49-2.43 (m, 1H), 1.09-1.07 (m, 1H), 0.64-0.61 (m, 1H), 0.50-0.48 (m, 1H), 0.36-0.33 (m, 1H), 0.23-0.21 (m, 1H).

### Example 6: (S)-3-cyclopropyl-3-(3-((5-((ethyl(isopropyl)amino)methyl)-4-(5-fluoro-2-methoxypyridin-4-yl)-2-methylbenzoyl)oxy)phenyl)propanoic acid (Compound 11)

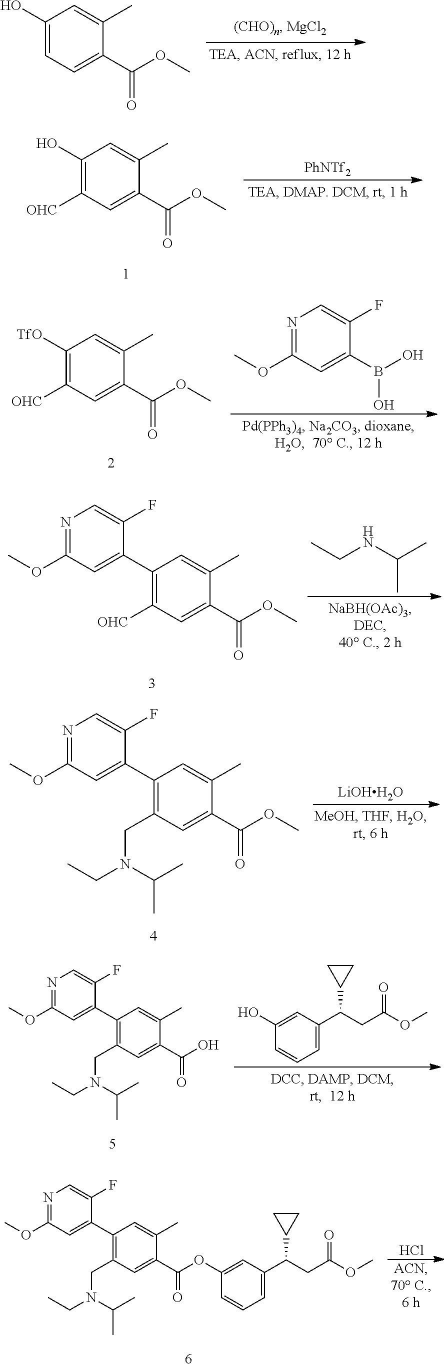

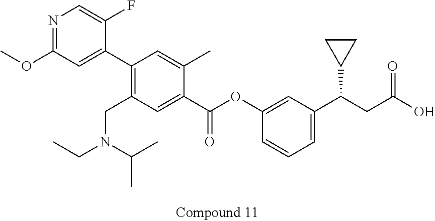 [SMILES: CCN(Cc1cc(C(=O)Oc2cccc([C@@H](CC(=O)O)C3CC3)c2)c(C)cc1-c1cc(OC)ncc1F)C(C)C]

Step 1: methyl 5-formyl-4-hydroxy-2-methylbenzoate (1): To a solution of methyl 4-hydroxy-2-methylbenzoate (2.0 g, 12 mmol, 1 eq), paraformaldehyde (1.8 g, 60 mmol, 5 eq) and MgCl₂ (1.7 g, 18 mmol, 1.5 eq) in ACN (200 mL) was added TEA (4.6 g, 46 mmol, 3.8 eq). The mixture was stirred at 80° C. for 12 hr. The yellow solution was poured into 5% HCl (40 mL), and then diluted with water (100 mL) and extracted with ethyl acetate (100 mL). The combined organic layers were washed with saturated brine (50 mL), filtered and concentrated under reduced pressure to give a residue. The residue was purified by column chromatography (SiO₂, Petroleum ether:Ethyl acetate=20:1) to give 1 (1.1 g, 49% yield) as a white solid. LCMS: tR=0.643 min, (ES+) m/z (M+H)⁺=195.1. ¹H NMR (400 MHz, CDCl₃) δ=11.23 (s, 1H), 9.88 (s, 1H), 8.26 (s, 1H), 6.86 (s, 1H), 3.90 (s, 3H), 2.66 (s, 3H).

Step 2: methyl 5-formyl-2-methyl-4-(trifluoromethylsulfonyloxy)benzoate (2): To a solution of 1 (1.1 g, 5.7 mmol, 1 eq) in DCM (15 mL) was added TEA (1.2 g, 11 mmol, 2 eq) and DMAP (69 mg, 0.57 mmol, 0.1 eq). The mixture was stirred at 20° C. for 0.5 hr. Then 1,1,1-trifluoro-N-phenyl-N-(trifluoromethylsulfonyl)methanesulfonamide (2.4 g, 6.8 mmol, 1.2 eq) was added to the mixture. The mixture was stirred at 20° C. for 0.5 hr. The reaction mixture was concentrated under reduced pressure to give a residue. The residue was purified by column chromatography (SiO₂, Petroleum ether:Ethyl acetate=1:0 to 20:1) to give 2 (1.4 g, 75% yield) as a light yellow oil. LCMS: tR=0.887 min, (ES+) m/z (M+H)⁺=327.

Step 3: methyl 4-(5-fluoro-2-methoxy-4-pyridyl)-5-formyl-2-methyl-benzoate (3): To a solution of 2 (1.4 g, 4.2 mmol, 1 eq) and (5-fluoro-2-methoxypyridin-4-yl)boronic acid (1.1 g, 6.3 mmol, 1.5 eq) in dioxane (14 mL) and H₂O (2.8 mL) was added Pd(PPh₃)₂Cl₂ (0.15 g, 0.21 mmol, 0.05 eq) and Na₂CO₃ (0.90 g, 8.5 mmol, 2 eq). The mixture was stirred at 70° C. for 12 hr. The reaction mixture was quenched by addition water (60 mL), and then diluted with ethyl acetate (60 mL), extracted with ethyl acetate (60 mL×3). The combined organic layers were washed with saturated brine (30 mL×2), filtered and concentrated under reduced pressure to give a residue. The residue was purified by column chromatography (SiO₂, Petroleum ether:Ethyl acetate=100:1 to 30:1) to give 3 (0.70 g, 55% yield) as a light yellow solid. LCMS: tR=0.861 min, (ES+) m/z (M+H)⁺=304.1. ¹H NMR (400 MHz, CDCl3) δ=9.93 (s, 1H), 8.58 (s, 1H), 8.11 (s, 1H), 7.28 (s, 1H), 6.73 (s, 1H), 3.98 (s, 6H), 2.74 (s, 3H).

Step 4: methyl 5-((ethyl(isopropyl)amino)methyl)-4-(5-fluoro-2-methoxypyridin-4-yl)-2-methylbenzoate (4): To a solution of 3 (0.30 g, 0.99 mmol, 1 eq) in DCE (1 mL) was added N-ethylpropan-2-amine (0.86 g, 9.9 mmol, 10 eq) and NaBH(OAc)₃ (0.42 mg, 2.0 mmol, 2 eq). The mixture was stirred at 40° C. for 2 hrs under N₂. The reaction mixture was quenched by addition water (5 mL), and then diluted with ethyl acetate (5 mL) and extracted with ethyl acetate (5 mL×3). The combined organic layers were washed with brine (10 mL), dried over Na₂SO₄, filtered, and concentrated under reduced pressure to give a residue. The residue was purified by column chromatography (SiO₂, Petroleum ether:Ethyl acetate=30:1) to give 4 (0.25 g, 62% yield) as a colorless oil. LCMS: tR=0.65 min, (ES+) m/z (M+H)⁺=375.1.

Step 5: 5-((ethyl(isopropyl)amino)methyl)-4-(5-fluoro-2-methoxypyridin-4-yl)-2-methylbenzoic acid (5): To a solution of 4 (0.25 g, 0.61 mmol, 1 eq) in THE (1.2 mL), MeOH (1.2 mL) and H₂O (1.2 mL) was added LiOH.H₂O (0.26 mg, 6.1 mmol, 10 eq). The mixture was stirred at 20° C. for 6 hr. The mixture was concentrated to give a residue. The residue was poured into 5% HCl (4 mL), and diluted with water (4 mL) and extracted with ethyl acetate (4 mL×2). The combined organic layers were washed with saturated brine (4 mL×2), filtered and concentrated under reduced pressure to give 5 (70 mg, crude) as a yellow oil.

Step 6: (S)-3-(1-cyclopropyl-3-methoxy-3-oxopropyl)phenyl 5-((ethyl(isopropyl)amino)methyl)-4-(5-fluoro-2-methoxypyridin-4-yl)-2-methylbenzoate (6): To a solution of 5 (70 mg, 0.19 mmol, 1 eq) and methyl (3S)-3-cyclopropyl-3-(3-hydroxyphenyl)propanoate (43 mg, 0.19 mmol, 1 eq) in DCM (1.5 mL) was added DMAP (24 mg, 0.19 mmol, 1 eq) and DCC (60 mg, 0.29 mmol, 1.5 eq). The mixture solution was stirred at 20° C. for 12 hours. The reaction mixture was diluted with water (2 mL), and extracted with DCM (2 mL×3). The combined organic layers were washed with saturated brine (3 mL×2), filtered. The residue was purified by prep-TLC (Petroleum ether:Ethyl acetate=3:1) to give 6 as a light yellow solid. LCMS: tR=0.834 min, (ES+) m/z (M+H)⁺=563.3.

Step 7: (S)-3-cyclopropyl-3-(3-((5-((ethyl(isopropyl)amino)methyl)-4-(5-fluoro-2-methoxypyridin-4-yl)-2-methylbenzoyl)oxy)phenyl)propanoic acid (Compound 11): To a solution of 6 (70 mg, 0.12 mmol, 1 eq) in ACN (1 mL) was added HCl (2 M, 62 μL, 1 eq). The mixture was stirred at 70° C. for 6 hr. The reaction solution was purified directly without work-up. The residue was purified by prep-HPLC (column: Phenomenex Synergi C18 150×25 mm×10 μm; mobile phase: [water (0.225% FA)-ACN]; B %: 18%-48%, 10 min). The solution was lyophilized to give Compound 11 (26 mg, 35% yield, FA salt) as a white solid. LCMS: tR=0.794 min., (ES⁺) m/z (M+H)⁺=549.3. ¹H NMR (400 MHz, CD₃OD) δ ppm 8.43 (s, 1H), 8.16 (s, 1H), 7.42-7.37 (m, 2H), 7.27 (d, J=7.6 Hz, 1H), 7.23 (s, 1H), 7.11-7.18 (m, 1H), 6.85 (d, J=4.8 Hz, 1H), 3.99-3.96 (m, 5H), 3.24-3.31 (m, 1H), 2.82-2.67 (m, 7H), 2.46-2.42 (m, 1H), 1.12-1.05 (m, 10H), 0.63-0.60 (m, 1H), 0.45-0.43 (m, 1H), 0.37-0.33 (m, 1H), 0.20-0.18 (m, 1H).

### Example 7: (S)-3-cyclopropyl-3-(3-((3-((diisopropylamino)methyl)-4-(5-fluoro-2-methoxypyridin-4-yl)benzoyl)oxy)phenyl)propanoic acid (Compound 15)

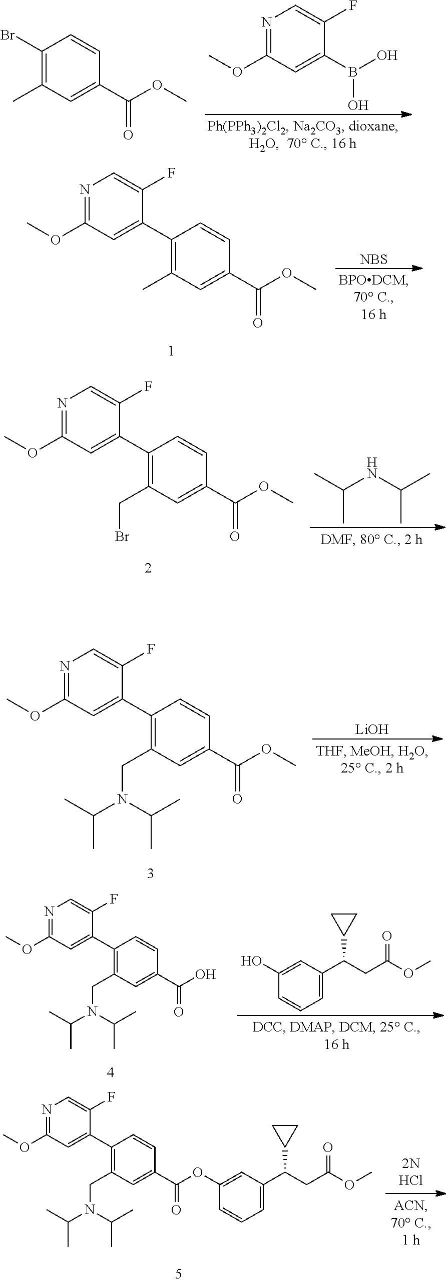

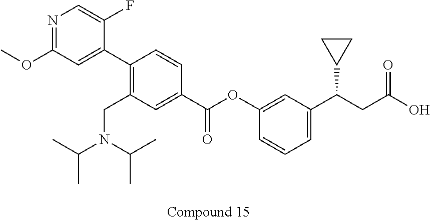 [SMILES: COc1cc(-c2ccc(C(=O)Oc3cccc([C@@H](CC(=O)O)C4CC4)c3)cc2CN(C(C)C)C(C)C)c(F)cn1]

Step 1: methyl 4-(5-fluoro-2-methoxypyridin-4-yl)-3-methylbenzoate (1): To a solution of methyl 4-bromo-3-methyl-benzoate (1.0 g, 4.4 mmol, 1 eq), (5-fluoro-2-methoxy-4-pyridyl)boronic acid (0.90 g, 5.2 mmol, 1.2 eq) in dioxane (10 mL) and H₂O (2 mL) was added Na₂CO₃ (0.83 g, 8.7 mmol, 2 eq) and Pd(PPh₃)₂Cl₂ (0.15 g, 0.22 mmol, 0.05 eq). The mixture was stirred at 70° C. for 16 hrs. The reaction mixture was quenched by addition water (20 mL), and then diluted with ethyl acetate (20 mL), extracted with ethyl acetate (20 mL×3). The combined organic layers were washed with saturated brine (20 mL), dried over Na₂SO₄, filtered and concentrated under reduced pressure to give a residue. The residue was purified by column chromatography (SiO₂, Petroleum ether:Ethyl acetate=1:0 to 100:1) to give 1 (1.0 g, 83% yield) as a white solid. ¹H NMR (400 MHz, CDCl₃) δ=8.06 (s, 1H), 7.97 (s, 1H), 7.91 (d, J=8.2 Hz, 1H), 7.25 (s, 1H), 6.62 (d, J=4.8 Hz, 1H), 3.94 (s, 3H), 3.93 (s, 3H), 2.26 (s, 3H).

Step 2: methyl 3-(bromomethyl)-4-(5-fluoro-2-methoxypyridin-4-yl)benzoate (2): To a solution of 1 (1.0 g, 3.6 mmol, 1 eq) in CCl₄ (20 mL) was added NBS (0.71 g, 4.0 mmol, 1.1 eq) and BPO (44 mg, 0.18 mmol, 0.05 eq). The mixture was stirred at 70° C. for 16 hrs. The mixture was concentrated to give a residue. The residue was purified by column chromatography (SiO₂, Petroleum ether:Ethyl acetate=1:0 to 200:1) to give 2 (0.95 g, 64% yield) as a colorless oil LCMS: tR=0.974 min. (ES⁺) m/z (M+H)⁺=354.0.

Step 3: methyl 3-((diisopropylamino)methyl)-4-(5-fluoro-2-methoxypyridin-4-yl)benzoate (3): A solution of 2 (0.45 g, 1.1 mmol, 1 eq) and N-isopropylpropan-2-amine (0.22 g, 2.2 mmol, 2 eq) in DMF (5 mL) was stirred at 80° C. for 2 hrs. The mixture was concentrated to give a residue. The residue was purified by prep-TLC (SiO₂, PE:EA=5:1) to give 3 (0.42 g, 74% yield) as a colorless oil. LCMS: tR=0.797 min. (ES⁺) m/z (M+H)+*=375.2.

Step 4: 3-((diisopropylamino)methyl)-4-(5-fluoro-2-methoxypyridin-4-yl)benzoic acid (4): A solution of 3 (0.42 g, 0.81 mmol, 1 eq) in THE (2 mL), MeOH (2 mL), H₂O (2 mL) was added LiOH H₂O (68 mg, 1.6 mmol, 2 eq). The mixture was stirred at 25° C. for 2 hrs. The mixture was concentrated to give a residue, the residue was then added 1N HCl (1 mL) and diluted with ethyl acetate (5 mL), extracted with ethyl acetate (5 mL×3). The combined organic layers were washed with saturated brine (10 mL), dried over Na₂SO₄, filtered and concentrated under reduced pressure to give 4 (0.28 g, crude) as a white solid LCMS: tR=0.741 min. (ES⁺) m/z (M+H)⁺=361.2.

Step 5: (S)-3-(1-cyclopropyl-3-methoxy-3-oxopropyl)phenyl 3-((diisopropylamino)methyl)-4-(5-fluoro-2-methoxypyridin-4-yl)benzoate (5): To a solution of 4 (0.28 g, 0.58 mmol, 1 eq) and methyl (3S)-3-cyclopropyl-3-(3-hydroxyphenyl)propanoate (0.13 g, 0.58 mmol, 1 eq) in DCM (3 mL) was added DMAP (36 mg, 0.29 mmol, 0.5 eq) and DCC (0.18 g, 0.87 mmol, 1.5 eq). The mixture was stirred at 25° C. for 16 hrs. The reaction mixture was quenched by addition water (10 mL), and then diluted with ethyl acetate (10 mL), extracted with ethyl acetate (10 mL×3). The combined organic layers were washed with saturated brine (20 mL), dried over Na₂SO₄, filtered and concentrated under reduced pressure to give a residue. The residue was purified by prep-TLC (SiO2, PE:EA=2:1) to give 5 (0.28 g, 84% yield) as a white solid. LCMS: tR=0.906 min. (ES⁺) m/z (M+H)⁺=563.2.

Step 6: (S)-3-cyclopropyl-3-(′3-((3-((diisopropylamino)methyl)-4-(5-fluoro-2-methoxypyridin-4-yl)benzoyl)oxy)phenyl)propanoic acid (Compound 15): To a solution of 5 (0.28 g, 0.50 mmol, 1 eq) in ACN (4 mL) was added HCl (2 M, 5.0 mL, 20 eq). The mixture was stirred at 70° C. for 1 hr. The mixture was concentrated to give a residue. The residue was purified by prep-HPLC (column: Phenomenex Synergi C18 150×25 mm×10 μm; mobile phase: [water (0.225% FA)-ACN]; B %: 23%-53%, 10 min) to give Compound 15 (52 mg, 17% yield, FA salt) as a white solid. LCMS: tR=0.859 min. (ES⁺) m/z (M+H)⁺=549.2. ¹H NMR (400 MHz, CDCl₃) δ=8.61 (s, 1H), 8.16-8.05 (m, 2H), 7.41-7.35 (m, 1H), 7.29 (d, J=8.0 Hz, 1H), 7.20-7.10 (m, 3H), 6.66 (d, J=4.8 Hz, 1H), 3.98 (s, 3H), 3.61 (s, 2H), 3.03-2.96 (m, 2H), 2.87-2.76 (m, 2H), 2.50-2.43 (m, 1H), 1.14-1.02 (m, 1H), 0.95 (d, J=6.4 Hz, 12H), 0.67-0.58 (m, 1H), 0.52-0.44 (m, 1H), 0.34-0.32 (m, 1H), 0.22-0.20 (m, 1H).

### Example 8: (S)-3-cyclopropyl-3-(3-((5-((diisopropylamino)methyl)-2-fluoro-4-(2-methoxypyridin-4-yl)benzoyl)oxy)phenyl)propanoic acid (Compound 16)

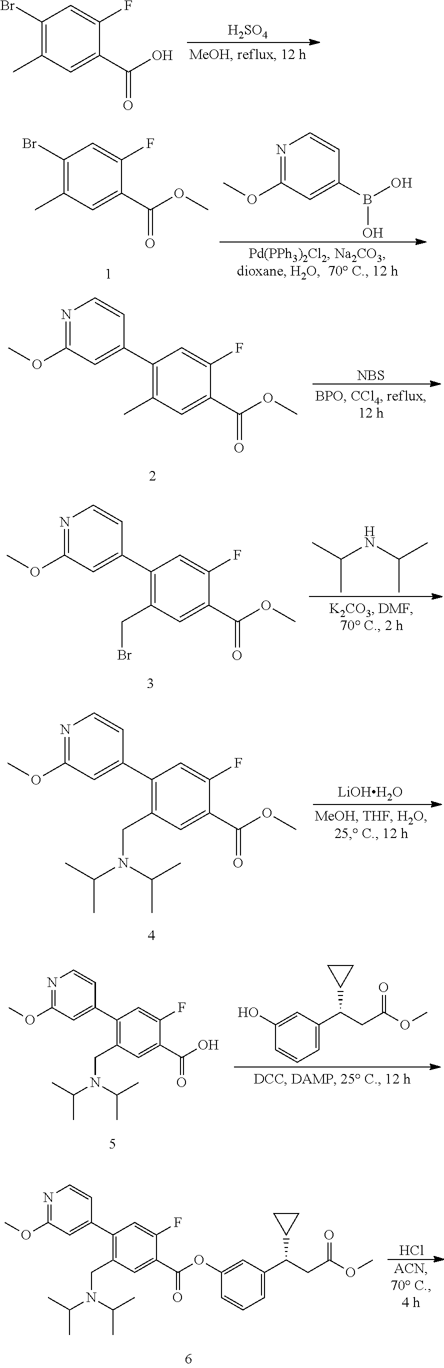

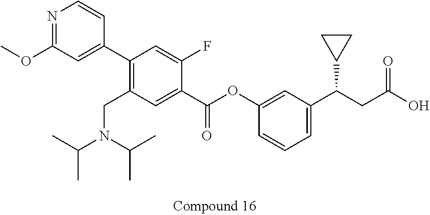 [SMILES: COc1cc(-c2cc(F)c(C(=O)Oc3cccc([C@@H](CC(=O)O)C4CC4)c3)cc2CN(C(C)C)C(C)C)ccn1]

Step 1: methyl 4-bromo-2-fluoro-5-methyl-benzoate (1): To a solution of 4-bromo-2-fluoro-5-methylbenzoic acid (5.0 g, 21 mmol) in MeOH (10 mL) was added H₂SO₄ (3.7 g, 38 mmol, 2 mL). The mixture was stirred at reflux for 12 hr. The reaction mixture was concentrated under reduced pressure to give a residue. The residue was diluted with ethyl acetate (30 mL) and water (20 mL), extracted with ethyl acetate (20 mL×3). The combined organic layers were washed with saturated NaHCO₃ solution (10 mL), dried over Na₂SO₄, filtered and concentrated under reduced pressure to give 1 (4.8 g, 89% yield) as a white solid. LCMS: tR=0.962 min, (ES⁺) m/z (M+H)⁺=249.0.

Step 2: methyl 2-fluoro-4-(2-methoxypyridin-4-yl)-5-methylbenzoate (2): To a solution of 1 (1.0 g, 4.1 mmol) and (2-methoxy-4-pyridyl)boronic acid (0.90 g, 6.1 mmol) in dioxane (12 mL) and H₂O (2 mL) was added Pd(PPh₃)₂Cl₂ (0.14 g, 0.20 mmol) and Na₂CO₃ (0.86 g, 8.1 mmol). The mixture was stirred at 70° C. for 12 hr. The reaction mixture was quenched by addition water (20 mL), then diluted with ethyl acetate (30 mL), extracted with ethyl acetate (20 mL×3). The combined organic layers were washed with saturated brine (20 mL), dried over Na₂SO₄, filtered and concentrated under reduced pressure to give a residue. The residue was purified by column chromatography (SiO₂, Petroleum ether:Ethyl acetate=1:0 to 50:1) to give 2 (1.1 g, 76% yield) as a white solid. LCMS: tR=0.919 min, (ES⁺) m/z (M+H)⁺=276.1. ¹H NMR (CDCl₃, 400 MHz): δ 8.23 (d, J=5.2 Hz, 1H), 7.84 (d, J=7.2 Hz, 1H), 7.01 (d, J=10.8 Hz, 1H), 6.82 (dd, J₁=5.2 Hz, J₂=1.2 Hz, 1H), 6.68 (d, J=1.2 Hz, 1H), 3.99 (s, 3H), 3.96 (s, 3H), 2.26 (s, 3H).

Step 3: methyl 5-(bromomethyl)-2-fluoro-4-(2-methoxypyridin-4-yl)benzoate (3): To a solution of 2 (1.1 g, 3.9 mmol) in CCl₄ (10 mL) was added NBS (0.90 g, 5.1 mmol) and BPO (47 mg, 0.20 mmol). The mixture was stirred at 70° C. for 12 hr. The reaction mixture was quenched by addition water (20 mL), then diluted with ethyl acetate (30 mL), extracted with ethyl acetate (20 mL×3). The combined organic layers were washed with saturated brine (20 mL), dried over Na₂SO₄, filtered and concentrated under reduced pressure to give a residue. The residue was purified by column chromatography (SiO₂, Petroleum ether:Ethyl acetate=1:0 to 10:1) to give 3 (0.51 g, 36% yield) as a white solid. LCMS: tR=0.995 min, (ES⁺) m/z (M+H)⁺=354.0.

Step 4: methyl 5-((diisopropylamino)methyl)-2-fluoro-4-(2-methoxypyridin-4-yl)benzoate (4): To a solution of 3 (0.51 g, 1.4 mmol) and nisopropylpropan-2-amine (0.22 g, 2.2 mmol) in DMF (5 mL) was added K₂CO₃ (0.40 g, 2.9 mmol). The mixture was stirred at 70° C. for 2 hr. The reaction mixture was quenched by addition water (20 mL), then diluted with ethyl acetate (30 mL), extracted with ethyl acetate (20 mL×3). The combined organic layers were washed with saturated brine (20 mL), dried over Na₂SO₄, filtered and concentrated under reduced pressure to give a residue. The residue was purified by column chromatography (SiO₂, Petroleum ether:Ethyl acetate=1:0 to 10:1) to give 4 (0.42 g, 71% yield) as a yellow oil. LCMS: tR=0.785 min, (ES⁺) m/z (M+H)⁺=375.2.

Step 5: methyl 5-((diisopropylamino)methyl)-2-fluoro-4-(2-methoxypyridin-4-yl)benzoic acid (5): To a solution of 4 (0.42 g, 1.1 mmol) in THE (4 mL), H₂O (4 mL) and MeOH (4 mL) was added LiOH.H₂O (95 mg, 2.3 mmol). The mixture was stirred at 25° C. for 3 hr. The reaction mixture was quenched by addition 1 N HCl (20 mL), then diluted with ethyl acetate (30 mL), extracted with ethyl acetate (20 mL×6). The combined organic layers were washed with saturated brine (20 mL), dried over Na₂SO₄, filtered and concentrated under reduced pressure to give 5 (0.18 g, 36% yield) as a yellow solid. LCMS: tR=0.754 min, (ES⁺) m/z (M+H)⁺=361.2.

Step 6: (S)-3-(1-cyclopropyl-3-methoxy-3-oxopropyl)phenyl 5-((diisopropylamino)methyl)-2-fluoro-4-(2-methoxypyridin-4-yl)benzoate (6): To a solution of 5 (0.18 g, 0.50 mmol) and (S)-methyl 3-cyclopropyl-3-(3-hydroxyphenyl)propanoate (0.17 g, 0.75 mmol) in DCM (5 mL) was added DCC (0.15 g, 0.75 mmol) and DMAP (61 mg, 0.50 mmol). The mixture was stirred at 25° C. for 12 hr. The reaction mixture was concentrated under reduced pressure to give a residue. The residue was purified by prep-TLC (SiO₂, PE:EA=5:1) to give 6 (0.14 g, 47% yield) as a white solid. LCMS: tR=0.868 min, (ES⁺) m/z (M+H)⁺=563.3.

Step 7: (S)-3-cyclopropyl-3-(3-((5-((diisopropylamino)methyl)-2-fluoro-4-(2-methoxypyridin-4-yl)benzoyl)oxy)phenyl)propanoic acid (Compound 16): To a solution of 6 (0.14 g, 0.25 mmol) in ACN (1 mL) was added HCl (2 M, 1 mL). The mixture was stirred at 70° C. for 4 hr. The reaction mixture was concentrated under reduced pressure to give a residue. The residue was purified by prep-HPLC (column: Phenomenex Synergi C18 150×25 mm×10 μm; mobile phase: [water (0.225% FA)-ACN]; B %: 23%-53%, 10 min) to give Compound 16 (23 mg, 14% yield, FA salt) as a white solid. LCMS: tR=0.863 min, (ES⁺) m/z (M+H)⁺=549.3. ¹H-NMR (CDCl₃; 400 MHz): δ 8.54 (d, J=7.2 Hz, 1H), 8.25 (d, J=5.2 Hz, 1H), 7.37 (t, J=8.0 Hz, 1H), 7.18-7.17 (m, 2H), 7.14-7.12 (m, 1H), 7.01 (d, J=10.4 Hz, 1H), 6.83 (dd, J₁=5.2 Hz, J₂=1.2 Hz, 1H), 6.69 (s, 1H), 4.01 (s, 3H), 3.62 (s, 2H), 3.06-2.99 (m, 2H), 2.86-2.81 (m, 2H), 2.46-2.44 (m, 1H), 1.15-1.05 (m, 1H), 0.97 (d, J=6.4 Hz, 12H), 0.68-0.59 (m, 1H), 0.54-0.44 (m, 1H), 0.39-0.31 (m, 1H), 0.26-0.17 (m, 1H).

### Example 9: (3S)-3-(3-((4-(1-(2,5-bis(trifluoromethyl)phenyl)ethyl)piperazine-1-carbonyl)oxy)phenyl)-3-cyclopropylpropanoic acid (Compound 20)

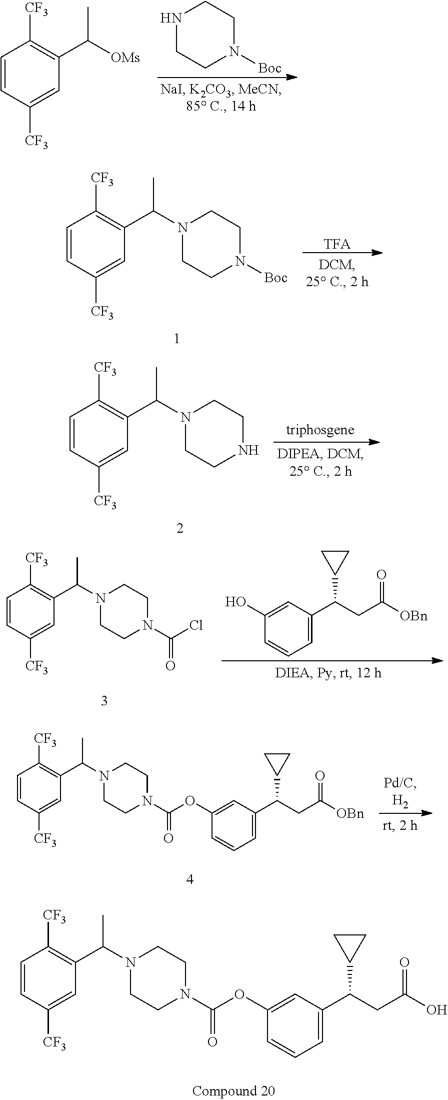

Step 1: tert-butyl 4-(1-(2,5-bis(trifluoromethyl)phenyl)ethyl)piperazine-1-carboxylate (1): To a solution of 1-(2,5-bis(trifluoromethyl)phenyl)ethyl methanesulfonate (0.65 g, 1.9 mmol) in MeCN (4 mL) was added NaI (0.29 g, 1.9 mmol), K₂CO₃ (1.3 g, 10 mmol) and tert-butyl piperazine-1-carboxylate (1.1 g, 5.8 mmol). The mixture was stirred at 85° C. for 14 hr. The reaction mixture was concentrated under reduced pressure to give a residue. The residue was purified by prep-TLC (SiO₂, PE:EA=10:1) to give 1 (0.63 g, 44% yield) as a yellow oil. LCMS: tR=0.896 min., (ES⁺) m/z (M+H)⁺=427.1. ¹H NMR (400 MHz, CDCl₃): δ 8.20 (s, 1H), 7.76 (d, J=8.0 Hz, 1H), 7.61 (d, J=8.0 Hz, 1H), 3.81-3.68 (m, 1H), 3.42 (s, 4H), 2.56 (s, 2H), 2.30-2.20 (m, 2H), 1.47 (s, 9H), 1.32 (d, J=6.4 Hz, 3H).

Step 2: 1-(1-(2,5-bis(trifluoromethyl)phenyl)ethyl)piperazine (2): A solution of 1 (0.63 g, 1.5 mmol) in DCM (5 mL) and TFA (1 mL) was stirred at 25° C. for 2 hr. The reaction mixture was concentrated under reduced pressure to give 2 (0.40 g, crude, TFA) as a yellow oil. LCMS: tR=0.735 min., (ES⁺) m/z (M+H)⁺=327.1.

Step 3: 4-(1-(2,5-bis(trifluoromethyl)phenyl)ethyl)piperazine-1-carbonyl chloride (3): To a mixture of 2 (0.40 g, 1.1 mmol) and DIPEA (0.55 g, 4.2 mmol) in DCM (10 mL) was added a solution of triphosgene (0.94 g, 3.2 mmol) in DCM (10 mL) slowly. The mixture was stirred at 25° C. for 2 hr. The reaction mixture was quenched by addition water (20 mL), and then diluted with ethyl acetate (20 mL), extracted with ethyl acetate (20 mL×3). The combined organic layers were washed with saturated brine (10 mL), dried over Na₂SO₄, filtered and concentrated under reduced pressure to give a residue. The residue was purified by column chromatography (SiO₂, Petroleum ether:Ethyl acetate=200:1 to 40:1) to give 3 (0.31 g, 54% yield, 72% purity) as a yellow oil. LCMS: tR=0.893 min., (ES⁺) m/z (M+H)⁺=389.0.

Step 4: 3-((S)-3-(benzyloxy)-1-cyclopropyl-3-oxopropyl)phenyl 4-(1-(2,5-bis(trifluoromethyl)phenyl)ethyl)piperazine-1-carboxylate (4): To a solution of 3 (0.10 mg, 0.26 mmol) in pyridine (3 mL) was added (S)-benzyl 3-cyclopropyl-3-(3-hydroxyphenyl)propanoate (91 mg, 0.31 mmol) and DIPEA (0.10 g, 0.77 mmol). The mixture was stirred at room temperature for 10 hr. The reaction mixture was quenched by water (10 mL), then diluted with ethyl acetate (20 mL), extracted with ethyl acetate (10 mL×3). The combined organic layers were washed with saturated brine (20 mL), dried over Na₂SO₄, filtered and concentrated under reduced pressure to give 4 (0.13 g, 69% yield) as a yellow oil. LCMS: tR=1.047 min., (ES⁺) m/z (M+H)⁺=649.2.

Step 5: (3S)-3-(3-((4-(1-(2,5-bis(trifluoromethyl)phenyl)ethyl)piperazine-1-carbonyl)oxy)phenyl)-3-cyclopropylpropanoic acid (Compound 20): To a solution of 4 (0.11 g, 0.16 mmol) in THE (1 mL) was added 10% Pd/C (2.0 mg). The mixture was stirred at 25° C. for 2 hr under H₂ (15 psi). The reaction mixture was concentrated under reduced pressure to give a residue. The residue was purified by prep-HPLC (column: Phenomenex Synergi C18 150×25 mm×10 μm; mobile phase: [water (0.225% FA)-ACN]; B %: 60%-90%, 10 min) to give Compound 20 (66 mg, 65% yield, FA salt) as a yellow solid. LCMS: tR=0.939 min., (ES⁺) m/z (M+H)⁺=559.3. ¹H NMR (400 MHz, CDCl₃): δ 8.23 (s, 1H), 7.78 (d, J=8.4 Hz, 1H), 7.64 (d, J=8.0 Hz, 1H), 7.29 (t, J=7.6 Hz, 1H), 7.09 (d, J=8.0 Hz, 1H), 6.99-6.95 (m, 2H), 3.94-3.77 (m, 1H), 3.76-3.45 (m, 4H), 2.86-2.65 (m, 4H), 2.40 (q, J=9.2 Hz, 3H), 1.40 (d, J=6 Hz, 3H), 1.16-0.96 (m, 1H), 0.66-0.56 (m, 1H), 0.51-0.41 (m, 1H), 0.36-0.28 (m, 1H), 0.23-0.13 (m, 1H).

### Example 10: (2S,3R)-3-cyclopropyl-3-(3-((5-((diisopropylamino)methyl)-4-(5-fluoro-2-methoxypyridin-4-yl)-2-methylbenzoyl)oxy)phenyl)-2-methylpropanoic acid (Compound 21)

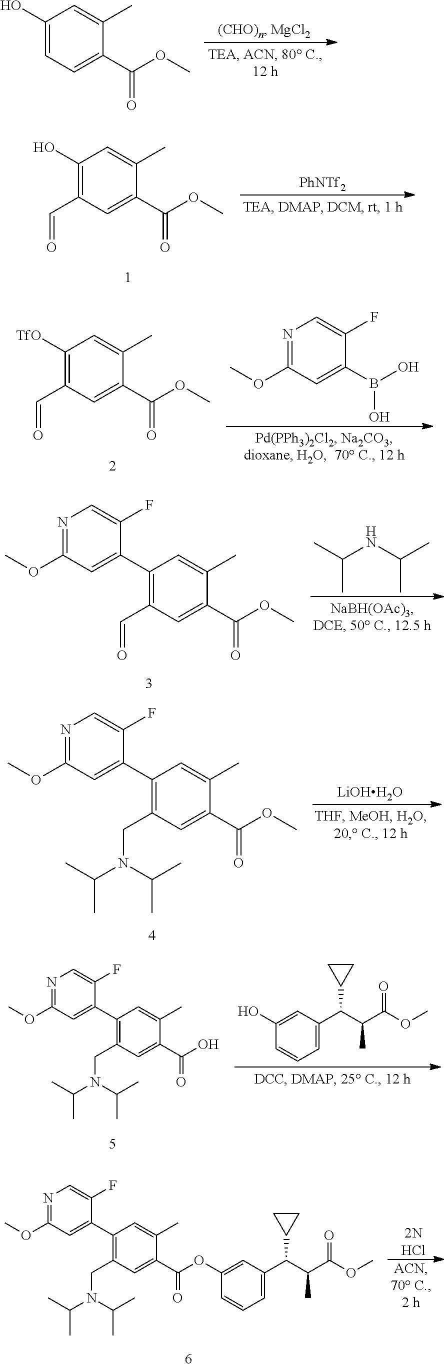

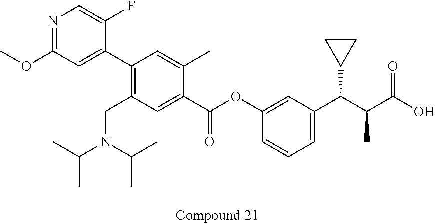 [SMILES: COc1cc(-c2cc(C)c(C(=O)Oc3cccc([C@H](C4CC4)[C@H](C)C(=O)O)c3)cc2CN(C(C)C)C(C)C)c(F)cn1]

Step 1: methyl 5-formyl-4-hydroxy-2-methylbenzoate (1): To a mixture of methyl 4-hydroxy-2-methyl-benzoate (5.0 g, 30 mmol, 1 eq), paraformaldehyde (4.5 g, 0.15 mol, 5 eq), MgCl₂ (4.3 g, 45 mmol, 1.5 eq) in ACN (500 mL) was added TEA (12 g, 0.11 mol, 3.8 eq). The mixture was stirred at 80° C. for 12 hr under N₂ atmosphere. The yellow solution was poured into 5% HCl (100 mL), and then extracted with ethyl acetate (200 mL). The organic phase was concentrated in vacuo to give a residue. The residue was purified by column chromatography (SiO₂, Petroleum ether:Ethyl acetate=1:0 to 10:1) to give 1 (2.0 g, 34% yield) as a white solid. LCMS: tR=0.763 min., (ES⁺) m/z (M+H)⁺=195.1.

Step 2: methyl 5-formyl-2-methyl-4-(((trifluoromethyl)sulfonyl)oxy)benzoate (2): To a solution of 1 (1.6 g, 8.2 mmol, 1 eq) in DCM (22 mL) was added TEA (1.7 g, 16 mmol, 2 eq) and DMAP (0.10 g, 0.82 mmol, 0.1 eq) at 20° C. for 0.5 hr. Then 1,1,1-trifluoro-N-phenyl-N-(trifluoromethylsulfonyl)methanesulfonamide (3.5 g, 9.9 mmol, 1.2 eq) was added to the mixture. The mixture was stirred at 20° C. for another 0.5 hr. The reaction mixture was partitioned between DCM (50 mL) and H₂O (50 mL). The organic phase was separated, washed with saturated brine (20 mL×2), dried over Na₂SO₄, filtered and concentrated under reduced pressure to give 2 (3.7 g, crude) as a yellow oil. LCMS: tR=0.876 min., (ES⁺) m/z (M+H)⁺=326.9.

Step 3: methyl 4-(5-fluoro-2-methoxypyridin-4-yl)-5-formyl-2-methylbenzoate (3): To a solution of 2 (3.5 g, 11 mmol, 1 eq) and (5-fluoro-2-methoxypyridin-4-yl)boronic acid (2.7 g, 16 mmol, 1.5 eq) in dioxane (34 mL) and H₂O (7 mL) was added Pd(PPh₃)₂Cl₂ (0.37 g, 0.53 mmol, 0.05 eq) and Na₂CO₃ (2.3 g, 21 mmol, 2 eq). The mixture was stirred at 70° C. for 12 hr. The reaction mixture was partitioned between ethyl acetate (50 mL) and H₂O (50 mL). The organic phase was separated, washed with saturated brine (20 mL×2), dried over Na₂SO₄, filtered and concentrated under reduced pressure to give a residue. The residue was purified by column chromatography (SiO₂, Petroleum ether:Ethyl acetate=1:0 to 20:1) to give 3 (3 g, 91% yield) as a white solid. LCMS: tR=0.909 min., (ES⁺) m/z (M+H)⁺=304.0. ¹H NMR (CD₃OD, 400 MHz): δ 9.89 (d, J=2.4 Hz, 1H), 8.42 (s, 1H), 8.25 (s, 1H), 7.55 (s, 1H), 6.99 (d, J=5.2 Hz, 1H), 3.92 (s, 3H), 3.90 (s, 3H), 2.65 (s, 3H).

Step 4: methyl 5-((diisopropylamino)methyl)-4-(5-fluoro-2-methoxypyridin-4-yl)-2-methylbenzoate (4): To a solution of 3 (3.0 g, 9.9 mmol, 1 eq) in DCE (20 mL) and AcOH (5.9 mg, 99 μmol, 0.01 eq) was added N-isopropylpropan-2-amine (10 g, 99 mmol, 10 eq). The mixture was stirred at 50° C. for 0.5 h. Then NaBH(OAc)₃ (4.2 g, 20 mmol, 2 eq) was added. The mixture was stirred at 50° C. for 12 h. The reaction mixture was partitioned between ethyl acetate (50 mL) and H₂O (50 mL). The organic phase was separated, washed with saturated brine (25 mL×2), dried over Na₂SO₄, filtered and concentrated under reduced pressure to give a residue. The residue was purified by column chromatography (SiO₂, Petroleum ether:Ethyl acetate=1:0 to 50:1) to give 4 (1.5 g, 38% yield) as a yellow oil. LCMS: tR=0.711 min., (ES⁺) m/z (M+H)+=389.1.

Step 5: 5-((diisopropylamino)methyl)-4-(5-fluoro-2-methoxypyridin-4-yl)-2-methylbenzoic acid (5): To a solution of 4 (0.5 g, 1.3 mmol, 1 eq) in H₂O (5 mL), MeOH (5 mL) and THE (5 mL) was added LiOH.H₂O (0.11 g, 2.6 mmol, 2 eq). The mixture was stirred at 20° C. for 12 hr. The mixture was adjusted pH to 7 with 3 N HCl, ethyl acetate (20 mL) was added. The mixture was partitioned. The organic phase was dried over Na₂SO₄, filtered and concentrated under reduced pressure to give 5 (0.40 g, crude) as a colorless oil. LCMS: tR=0.946 min., (ES⁻) m/z (M−H)⁻=373.2.

Step 6: 3-((1R,2S)-1-cyclopropyl-3-methoxy-2-methyl-3-oxopropyl)phenyl 5-((diisopropylamino)methyl)-4-(5-fluoro-2-methoxypyridin-4-yl)-2-methylbenzoate (6): To a solution of 5 (0.25 g, 0.67 mmol, 1 eq) and methyl (2S,3R)-3-cyclopropyl-3-(3-hydroxyphenyl)-2-methyl-propanoate (0.16 g, 0.67 mmol, 1 eq) in DCM (2.5 mL) was added DCC (0.21 g, 1.0 mmol, 1.5 eq) and DMAP (82 mg, 0.67 mmol, 1 eq). The mixture was stirred at 25° C. for 12 hr. The reaction mixture was partitioned between ethyl acetate (10 mL) and H₂O (10 mL). The organic phase was separated, washed with saturated brine (10 mL×2), dried over Na₂SO₄, filtered and concentrated under reduced pressure to give a residue. The residue was purified by column chromatography (SiO₂, Petroleum ether:Ethyl acetate=1:0 to 30:1) to give 6 (0.35 g, 83% yield) as a light yellow oil. LCMS: tR=0.939 min., (ES⁺) m/z (M+H)⁺=591.4.

Step 7: (2S,3R)-3-cyclopropyl-3-(3-((5-((diisopropylamino)methyl)-4-(5-fluoro-2-methoxypyridin-4-yl)-2-methylbenzoyl)oxy)phenyl)-2-methylpropanoic acid (Compound 21): To a solution of 6 (0.35 g, 0.59 mmol, 1 eq) in ACN (3.5 mL) was added HCl (2 M, 3.5 mL, 12 eq). The mixture was stirred at 70° C. for 2 hr. The reaction mixture was purified by prep-HPLC (column: Phenomenex Synergi C18 150×25 mm×10 μm; mobile phase: [water (0.225% FA)-ACN]; B %: 23%-53%, 10 min) to give Compound 21 (72 mg, 21% yield, FA salt) as a white solid. LCMS: tR=0.891 min., (ES⁺) m/z (M+H)⁺=577.4. ¹H-NMR (CD₃OD, 400 MHz): δ 8.46 (s, 1H), 8.11 (s, 1H), 7.43-7.39 (m, 1H), 7.24 (s, 1H), 7.16 (d, J=8.0 Hz, 1H), 7.12-7.11 (m, 2H), 6.78 (d, J=4.8 Hz, 1H), 3.95 (s, 3H), 3.74 (s, 2H), 3.13-3.06 (m, 2H), 2.86-2.80 (m, 1H), 2.68 (s, 3H), 2.08 (t, J=9.6 Hz, 1H), 1.18-1.14 (m, 1H), 1.00-0.96 (m, 15H), 0.64-0.61 (m, 1H), 0.42-0.35 (m, 2H), 0.06-0.01 (m, 1H).

### Example 11: (2S,3R)-3-(3-((1-(1-(2,5-bis(trifluoromethyl)phenyl)ethyl)piperidine-4-carbonyl)oxy)phenyl)-3-cyclopropyl-2-methylpropanoic acid (Compound 22)

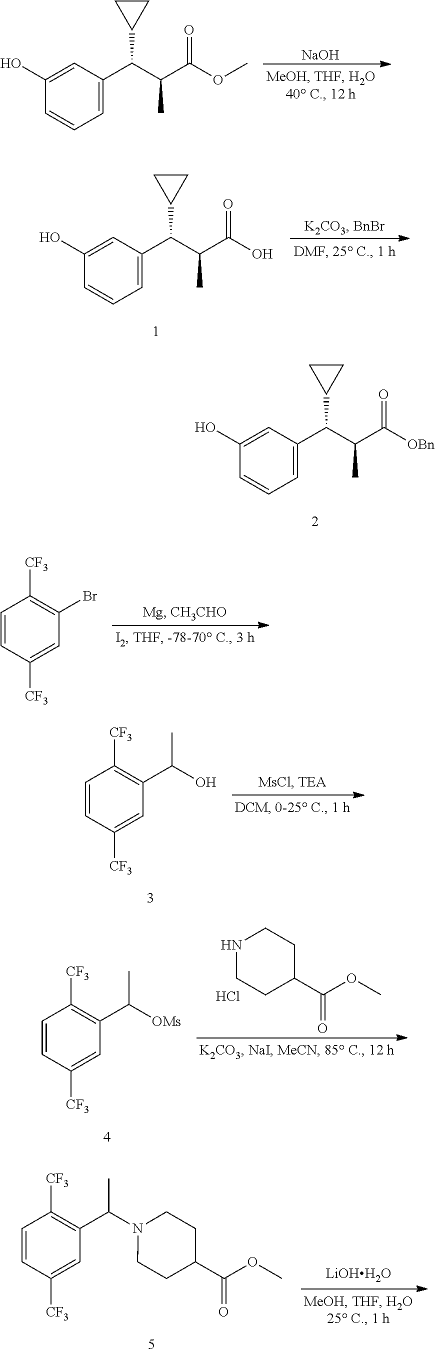

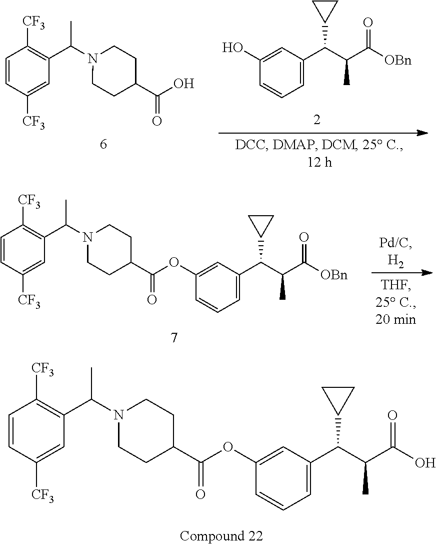 [SMILES: CC(c1cc(C(F)(F)F)ccc1C(F)(F)F)N1CCC(C(=O)O)CC1 ; C[C@H](C(=O)OCc1ccccc1)[C@H](c1cccc(O)c1)C1CC1 ; CC(c1cc(C(F)(F)F)ccc1C(F)(F)F)N1CCC(C(=O)Oc2cccc([C@H](C3CC3)[C@H](C)C(=O)OCc3ccccc3)c2)CC1 ; CC(c1cc(C(F)(F)F)ccc1C(F)(F)F)N1CCC(C(=O)Oc2cccc([C@H](C3CC3)[C@H](C)C(=O)O)c2)CC1]

Step 1: (2S,3R)-3-cyclopropyl-3-(3-hydroxyphenyl)-2-methylpropanoic acid (1): To a solution of (2S,3R)-methyl 3-cyclopropyl-3-(3-hydroxyphenyl)-2-methylpropanoate (0.20 g, 0.85 mmol) in MeOH (2 mL), THF (2 mL) and H₂O (2 mL) was added NaOH (34 mg, 0.85 mmol). The mixture was stirred at 40° C. for 12 h. The reaction mixture was concentrated under reduced pressure to give a residue. The residue was diluted by H₂O (10 mL) and adjusted pH to 7 with 1 N HCl, then extracted with ethyl acetate (20 mL×3). The combined organic layers were dried over anhydrous Na₂SO₄, filtered and concentrated under reduced pressure to give 1 (0.20 g, crude) as a yellow oil. ¹H NMR (400 MHz, MeOH) δ=7.13-7.07 (m, 1H), 6.67-6.60 (m, 3H), 2.76-2.70 (m, 1H), 1.88 (t, J=10.0 Hz, 1H), 1.08 (m, 1H), 0.91 (d, J=7.2 Hz, 3H), 0.63-0.53 (m, 1H), 0.37-0.26 (m, 2H), 0.03-0.06 (m, 1H).

Step 2: (2S,3R)-benzyl 3-cyclopropyl-3-(3-hydroxyphenyl)-2-methylpropanoate (2): To a solution of 1 (0.20 g, 0.91 mmol) in DMF (5 mL) was added K₂CO₃ (0.25 g, 1.8 mmol) and BnBr (0.17 g, 1.0 mmol). The mixture was stirred at 25° C. for 1 h. The residue was quenched by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by column chromatography (SiO₂, Petroleum ether:Ethyl acetate=20:1 to 5:1) to give 2 (0.20 g, 66% yield) as a yellow oil.

¹H NMR (400 MHz, CD₃OD) δ=7.42-7.30 (m, 5H), 7.12-7.05 (m, 1H), 6.66-6.59 (m, 3H), 5.21-5.10 (m, 2H), 2.85-2.83 (m, 1H), 1.85 (t, J=10.0 Hz, 1H), 1.09-0.98 (m, 1H), 0.93 (d, J=7.2 Hz, 3H), 0.46-0.36 (m, 1H), 0.31-0.22 (m, 1H), 0.15-0.13 (m, 1H), 0.08-0.01 (m, 1H).

Step 3: 1-(2,5-bis(trifluoromethyl)phenyl)ethanol (3): To a mixture of Mg (2.1 g, 85 mmol) and iodine (63 mg, 0.25 mmol) in THE (80 mL) at 25° C. was added 2-bromo-1,4-bis(trifluoromethyl)benzene (25 g, 85 mmol). The mixture was warmed to 70° C. for 1 h to generate the Grignard reagent and then the solution was cooled to −78° C. A solution of acetaldehyde (3.8 g, 85 mmol) in THE (80 mL) was added to the above solution and the reaction was warmed to 25° C. and stirred for 2 h. The residue was quenched by saturated NH₄Cl solution (100 mL) and water (300 mL), then extracted with ethyl acetate (200 mL×2). The combined organic layers were washed with saturated brine (400 mL), dried over anhydrous Na₂SO₄, filtered and concentrated under reduced pressure to give a residue. The residue was purified by column chromatography (SiO₂, Petroleum ether:Ethyl acetate=1:0 to 40:1) to give 3 (17 g, 75% yield) as a yellow oil. ¹H NMR (400 MHz, CDCl₃) δ=8.15 (s, 1H), 7.75 (d, J=8.4 Hz, 1H), 7.64 (d, J=8.4 Hz, 1H), 5.39-5.38 (m, 1H), 1.52 (d, J=6.4 Hz, 3H).

Step 4: 1-(2,5-bis(trifluoromethyl)phenyl)ethyl methanesulfonate (4): To a solution of 3 (3.0 g, 12 mmol) in DCM (30 mL) was added TEA (1.8 g, 17 mmol). Then MsCl (1.7 g, 15 mmol) was added dropwise at 0° C. The mixture was stirred at 25° C. for 1 h. The residue was quenched by water (100 mL), then extracted with DCM (80 mL×2). The combined organic layers were washed with saturated brine (100 mL), dried over anhydrous Na₂SO₄, filtered and concentrated under reduced pressure to give a residue. The residue was purified by column chromatography (SiO₂, Petroleum ether:Ethyl acetate=40:1 to 20:1) to give 4 (3.5 g, 88% yield) as a colorless oil. ¹H NMR (400 MHz, CDCl₃) δ=8.01 (s, 1H), 7.85-7.82 (m, 1H), 7.77-7.73 (m, 1H), 6.15-6.14 (m, 1H), 2.93 (s, 3H), 1.74 (d, J=6.4 Hz, 3H).

Step 5: methyl 1-(1-(2,5-bis(trifluoromethyl)phenyl)ethyl)piperidine-4-carboxylate (5): To a solution of 4 (0.30 g, 0.89 mmol) in MeCN (3 mL) was added NaI (0.13 g, 0.89 mmol), K₂CO₃ (0.62 g, 4.5 mmol) and methyl piperidine-4-carboxylate hydrochloride (0.48 g, 2.7 mmol). The mixture was stirred at 85° C. for 12 h. The residue was diluted by saturated NaHCO₃ solution (30 mL) and extracted with ethyl acetate (20 mL×3). The combined organic layers were washed with saturated brine (30 mL), dried over anhydrous Na₂SO₄, filtered and concentrated under reduced pressure to give a residue. The residue was purified by column chromatography (SiO₂, Petroleum ether:Ethyl acetate=100:1 to 40:1) to give 5 (0.28 g, 71% yield) as a colorless oil. ¹H NMR (400 MHz, CDCl₃) δ=8.19 (s, 1H), 7.73 (d, J=8.4 Hz, 1H), 7.58 (d, J=8.4 Hz, 1H), 3.68 (s, 3H), 3.22-3.21 (m, 1H), 2.51-2.50 (m, 1H), 2.31 (m, 1H), 2.10-1.92 (m, 3H), 1.88-1.71 (m, 2H), 1.69-1.58 (m, 2H), 1.29 (d, J=6.6 Hz, 3H).

Step 6: 1-(1-(2,5-bis(trifluoromethyl)phenyl)ethyl)piperidine-4-carboxylic acid (6): To a solution of 5 (0.28 g, 0.64 mmol) in MeOH (2 mL), THE (2 mL) and H₂O (2 mL) was added LiOH.H₂O (53 mg, 1.3 mmol). The mixture was stirred at 25° C. for 1 h. The reaction mixture was concentrated under reduced pressure to give a residue. The residue was diluted by H₂O (10 mL) and adjusted pH to 7 by 1 N HCl, then extracted with ethyl acetate (20 mL×3). The combined organic layers were dried over anhydrous Na₂SO₄, filtered and concentrated under reduced pressure to give 6 (0.27 g, crude) as a white solid. LCMS: tR=0.746 min., (ES⁺) m/z (M+H)⁺=370.1.

Step 7: 3-((1R,2S)-3-(benzyloxy)-1-cyclopropyl-2-methyl-3-oxopropyl)phenyl 1-(1-(2,5-bis(trifluoromethyl)phenyl)ethyl)piperidine-4-carboxylate (7): To a solution of 2 (0.10 g, 0.32 mmol) in DCM (2 mL) was added DCC (0.10 g, 0.48 mmol), DMAP (20 mg, 0.16 mmol) and 6 (0.12 g, 0.32 mmol). The mixture was stirred at 25° C. for 12 h. The residue was quenched by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by prep-TLC (SiO₂, PE:EA=4:1) to give 7 (0.14 g, 64% yield) as a yellow oil. LCMS: tR=0.949 min., (ES⁺) m/z (M+H)+=662.3.

Step 8: (2S,3R)-3-(3-((1-(1-(2,5-bis(trifluoromethyl)phenyl)ethyl)piperidine-4-carbonyl)oxy)phenyl)-3-cyclopropyl-2-methylpropanoic acid (Compound 22): To a solution of 7 (0.13 g, 0.20 mmol) in THE (2 mL) was added 5% Pd/C (13 mg). The mixture was stirred at 25° C. for 20 min under H₂ (15 psi). The reaction mixture was filtered and concentrated under reduced pressure to give a residue. The residue was purified by prep-HPLC (neutral condition; column: Waters Xbridge 150×25 mm×5 μm; mobile phase: [water (10 mM NH₄HCO₃)-ACN]; B %: 40%-70%, 10 min) to give Compound 22 (74 mg, 66% yield) as a white solid. LCMS: tR=0.859 min., (ES⁺) m/z (M+H)⁺=572.2. ¹H NMR (400 MHz, CD₃OD) δ=8.25 (s, 1H), 7.88 (d, J=8.4 Hz, 1H), 7.74 (d, J=8.4 Hz, 1H), 7.35-7.28 (m, 1H), 7.08 (d, J=7.6 Hz, 1H), 6.95-6.90 (m, 2H), 3.81-3.72 (m, 1H), 3.38-3.32 (m, 1H), 2.79-2.74 (m, 1H), 2.70-2.52 (m, 2H), 2.22-2.07 (m, 3H), 2.05-1.87 (m, 3H), 1.78-1.65 (m, 1H), 1.34 (d, J=6.4 Hz, 3H), 1.16-1.06 (m, 1H), 0.90 (d, J=7.2 Hz, 3H), 0.65-0.56 (m, 1H), 0.41-0.27 (m, 2H), 0.01-−0.03 (m, 1H).

### Example 12: (2S,3R)-3-cyclopropyl-3-(3-((3-((diisopropylamino)methyl)-4-(5-fluoro-2-methoxypyridin-4-yl)benzoyl)oxy)phenyl)-2-methylpropanoic acid (Compound 26)

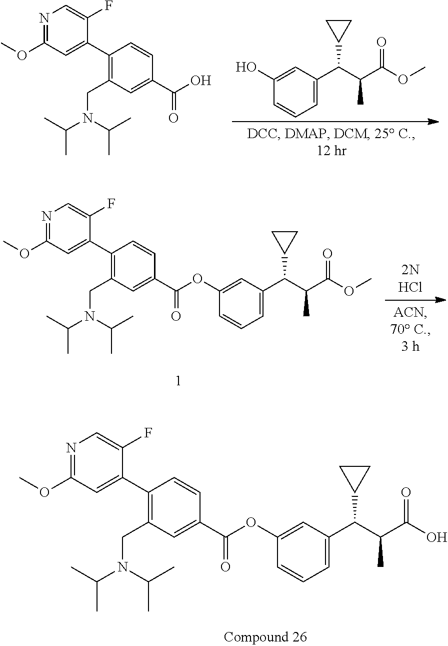

Step 1: 3-((1R,2S)-1-cyclopropyl-3-methoxy-2-methyl-3-oxopropyl)phenyl 3-((diisopropylamino)methyl)-4-(5-fluoro-2-methoxypyridin-4-yl)benzoate (1): To a solution of 3-((diisopropylamino)methyl)-4-(5-fluoro-2-methoxypyridin-4-yl)benzoic acid (Example 7, Step 4) (0.20 g, 0.46 mmol) and methyl (2S,3R)-methyl 3-cyclopropyl-3-(3-hydroxyphenyl)-2-methylpropanoate (0.11 g, 0.46 mmol) in DCM (3 mL) was added DMAP (29 mg, 0.23 mmol) and DCC (0.14 g, 0.70 mmol). The mixture was stirred at 25° C. for 12 hr. The reaction mixture was concentrated under reduced pressure to give a residue. The residue was purified by prep-TLC (SiO₂, Petroleum ether:Ethyl acetate=5:1) to give a 1 (0.22 g, 82% yield) as a yellow oil. LCMS: tR=0.775 min, (ES⁺) m/z (M+H)⁺=577.2.

Step 2: (2S,3R)-3-cyclopropyl-3-(3-((3-((diisopropylamino)methyl)-4-(5-fluoro-2-methoxypyridin-4-yl)benzoyl)oxy)phenyl)-2-methylpropanoic acid (Compound 26): To a solution of 1 (0.20 g, 0.35 mmol) in ACN (1 mL) was added HCl (2 M, 1 mL). The mixture was stirred at 70° C. for 3 hr. The reaction mixture was filtered to give a solution. The solution was purified by prep-HPLC (column: Phenomenex Synergi C18 150×30 mm×4 μm; mobile phase: [water (0.225% FA)-ACN]; B %: 25%-55%, 10 min) to give Compound 26 (18 mg, 8% yield, FA salt) as a white solid. LCMS: tR=0.864 min, (ES⁺) m/z (M+H)⁺=563.3. ¹H-NMR (CD₃Cl, 400 MHz): δ 8.59 (s, 1H), 8.12-8.08 (m, 2H), 7.41-7.37 (m, 1H), 7.29-7.27 (m, 1H), 7.15-7.09 (m, 3H), 6.65 (d, J=4.8 Hz, 1H), 3.98 (s, 3H), 3.57 (s, 2H), 2.98-2.87 (m, 3H), 2.10-2.09 (m, 1H), 1.18-1.11 (m, 1H), 1.06 (d, J=6.8 Hz, 3H), 0.93 (d, J=6.4 Hz, 12H), 0.69-0.64 (m, 1H), 0.45-0.39 (m, 2H), 0.11-0.04 (m, 1H).

### Example 13: (2S,3R)-3-cyclopropyl-3-(3-((5′-methoxy-3-methyl-2′-((6-methylpyridin-2-yl)(neopentyl)carbamoyl)-[1,1′-biphenyl]-4-carbonyl)oxy)phenyl)-2-methylpropanoic acid (Compound 31)

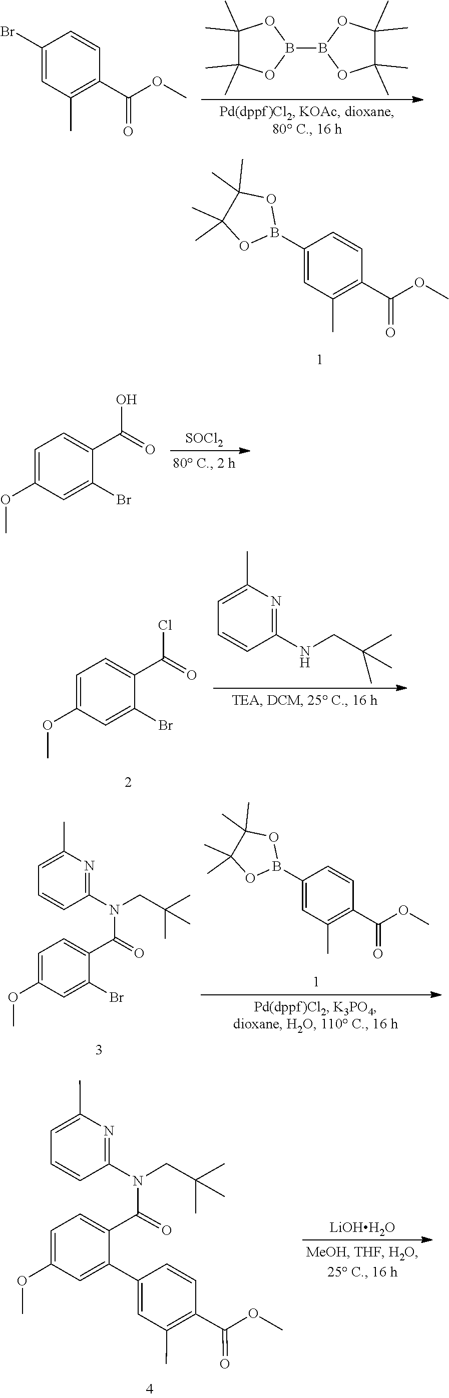

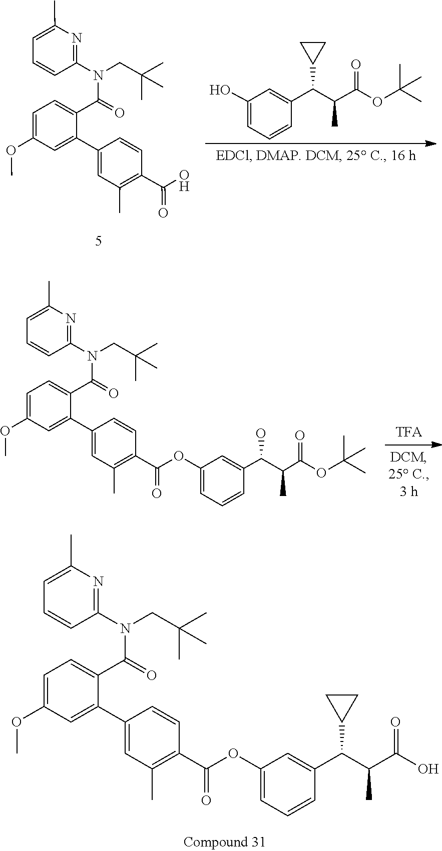

Step 1: methyl 2-methyl-4-(4,4,5,5-tetramethyl-1,3,2-dioxaborolan-2-yl)benzoate (1): To a solution of methyl 4-bromo-2-methyl-benzoate (10 g, 44 mmol, 1 eq), KOAc (13 g, 0.13 mol, 3 eq), 4,4,5,5-tetramethyl-2-(4,4,5,5-tetramethyl-1,3,2-dioxaborolan-2-yl)-1,3,2-dioxaborolane (12 g, 48 mmol, 1.1 eq) in dioxane (100 mL) was added Pd(dppf)Cl₂ (1.6 g, 2.2 mmol, 0.05 eq). The reaction mixture was stirred at 80° C. for 16 h under N₂ atmosphere. The reaction mixture was filtrated. The filtrate was concentrated in vacuo to give a residue. The residue was purified by column chromatography (SiO₂, PE:EA=1:0 to 20:1) to give 1 (12 g, 99% yield) as a yellow solid. LCMS: tR=1.034 min, (ES+) m/z (M+H)⁺=277.2.

Step 2: 2-bromo-4-methoxybenzoyl chloride (2): A solution of 2-bromo-4-methoxybenzoic acid (1.2 g, 5.2 mmol, 1 eq) in SOCl₂ (12 mL) was stirred at 80° C. for 2 h under N₂ atmosphere. The reaction mixture was concentrated under reduced pressure to give 2 (1.3 g, crude) as a white solid which was used for next step directly.

Step 3: 2-bromo-4-methoxy-N-(6-methylpyridin-2-yl)-N-neopentylbenzamide (3): To a solution of 6-methyl-N-neopentylpyridin-2-amine (1.1 g, 6.2 mmol, 1.18 eq) and TEA (2.1 g, 21 mmol, 4 eq) in DCM (10 mL) was added dropwise a solution of 2 (1.3 g, 5.2 mmol, 1 eq) in DCM (10 mL). The mixture was stirred at 25° C. for 16 hr under N₂ atmosphere. The reaction mixture was concentrated in vacuo to give a residue. The residue was purified by silica gel chromatography to give 3 (1.7 g, 70% yield) as a yellow oil. LCMS: tR=1.015 min, (ES+) m/z (M+H)⁺=391.1. ¹H NMR (400 MHz, CDCl₃) δ ppm 7.02 (d, J=2.4 Hz, 1H), 6.90-6.81 (m, 2H), 6.74 (d, J=7.6 Hz, 1H), 6.58 (dd, J₁=8.8 Hz, J₂=2.4 Hz, 1H), 6.46-6.22 (m, 1H), 3.74 (s, 3H), 2.48 (s, 3H), 2.39 (s, 2H), 0.94-0.87 (m, 9H).

Step 4: methyl 5′-methoxy-3-methyl-2′-((6-methylpyridin-2-yl)(neopentyl)carbamoyl)-[1,1′-biphenyl]-4-carboxylate (4): To a solution of 3 (1.6 g, 4.1 mmol, 1 eq), 1 (1.4 g, 4.9 mmol, 1.2 eq) and K₂CO₃ (1.1 g, 8.2 mmol, 2 eq) in dioxane (20 mL) and H₂O (4 mL) was added Pd(dppf)Cl₂ (0.15 g, 0.20 mmol, 0.05 eq). The mixture was stirred at 110° C. for 16 hr. The reaction mixture was concentrated under reduced pressure to give a residue. The residue was diluted with H₂O (100 mL) and extracted with ethyl acetate (50 mL×3). The combined organic layers were washed with saturated brine (50 mL×2), dried over Na₂SO₄, filtered and concentrated under reduced pressure to give a residue. The residue was purified by column chromatography (SiO₂, PE:EA=1:0 to 10:1) to give 4 (2.1 g, crude) as a yellow solid. LCMS: tR=1.083 min, (ES+) m/z (M+H)⁺=461.4.

Step 5: 5′-methoxy-3-methyl-2′-((6-methylpyridin-2-yl)(neopentyl)carbamoyl)-[1,1′-biphenyl]-4-carboxylic acid (5): To a solution of 4 (1.8 g, 3.9 mmol, 1 eq) in MeOH (6 mL), THF (12 mL) and H₂O (6 mL) was added LiOH.H₂O (0.49 g, 12 mmol, 3 eq). The reaction was stirred at 25° C. for 16 h under N₂ atmosphere. The mixture was adjusted pH to 6 by adding 2N HCl, and then concentrated under reduced pressure to give a residue. The residue was diluted with H₂O (100 mL) and extracted with ethyl acetate (50 mL×4). The combined organic layers were washed with saturated brine (100 mL), dried over Na₂SO₄, filtered and concentrated under reduced pressure to give 5 (1.8 g, 92% yield, 89% purity) was obtained as a yellow solid. LCMS: tR=0.892 min, (ES+) m/z (M+H)⁺=447.1.

Step 6: 3-((1R,2S)-3-(tert-butoxy)-1-cyclopropyl-2-methyl-3-oxopropyl)phenyl 5′-methoxy-3-methyl-2′-((6-methylpyridin-2-yl)(neopentyl)carbamoyl)-[1,1′-biphenyl]-4-carboxylate (6): A mixture of 5 (0.20 g, 0.45 mmol, 1 eq), (2S,3R)-tert-butyl 3-cyclopropyl-3-(3-hydroxyphenyl)-2-methylpropanoate (0.25 g, 0.90 mmol, 2 eq), EDCI (0.17 g, 0.90 mmol, 2 eq), DMAP (0.11 g, 0.90 mmol, 2 eq) in DCM (2 mL) was stirred at 25° C. for 16 hr under N₂ atmosphere. The reaction mixture was purified by silica gel chromatography to give 6 (0.30 g, 78% yield) as a colorless oil. LCMS: tR=1.287 min, (ES+) m/z (M+H)⁺=705.5.

Step 7: (2S,3R)-3-cyclopropyl-3-(3-((5′-methoxy-3-methyl-2′-((6-methylpyridin-2-yl)(neopentyl)carbamoyl)-[1,1′-biphenyl]-4-carbonyl)oxy)phenyl)-2-methylpropanoic acid (Compound 31): To a solution of 6 (0.20 g, 0.28 mmol, 1 eq) in DCM (2 mL) was added TFA (0.4 mL). The mixture was stirred at 25° C. for 3 hr. The reaction mixture was concentrated under reduced pressure to give a residue. The residue was purified by prep-HPLC (column: Phenomenex luna C18 150×25 mm×10 μm; mobile phase: [water (0.1% TFA)-ACN]; B %: 62%-92%, 10 min) to give Compound 31 (45 mg, 24% yield) as a white solid. LCMS: tR=1.116 min, (ES+) m/z (M+H)⁺=649.4. ¹H NMR (400 MHz, DMSO-d₆) δ ppm 8.04 (d, J=8.0 Hz, 1H), 7.41 (t, J=7.2 Hz, 2H), 7.28-7.24 (m, 3H), 7.18-7.14 (m, 3H), 6.91-6.87 (m, 2H), 6.76 (d, J=2.4 Hz, 1H), 6.58 (d, J=8.4 Hz, 1H), 3.83 (s, 2H), 3.78 (s, 3H), 2.79-2.75 (m, 1H), 2.58 (s, 3H), 2.16 (s, 3H), 2.06 (t, J=10 Hz, 1H), 1.17-1.05 (m, 1H), 0.87 (d, J=6.8 Hz, 3H), 0.72 (s, 9H), 0.56-0.55 (m, 1H), 0.32-0.29 (m, 2H), 0.03-0.01 (m, 1H).

### Example 14: (2S,3R)-3-cyclopropyl-3-[3-[3-[(3,3-dimethylpyrrolidin-1-yl)methyl]-4-(5-fluoro-2-methoxy-4-pyridyl)benzoyl]oxyphenyl]-2-methyl-propanoic acid (Compound 33)

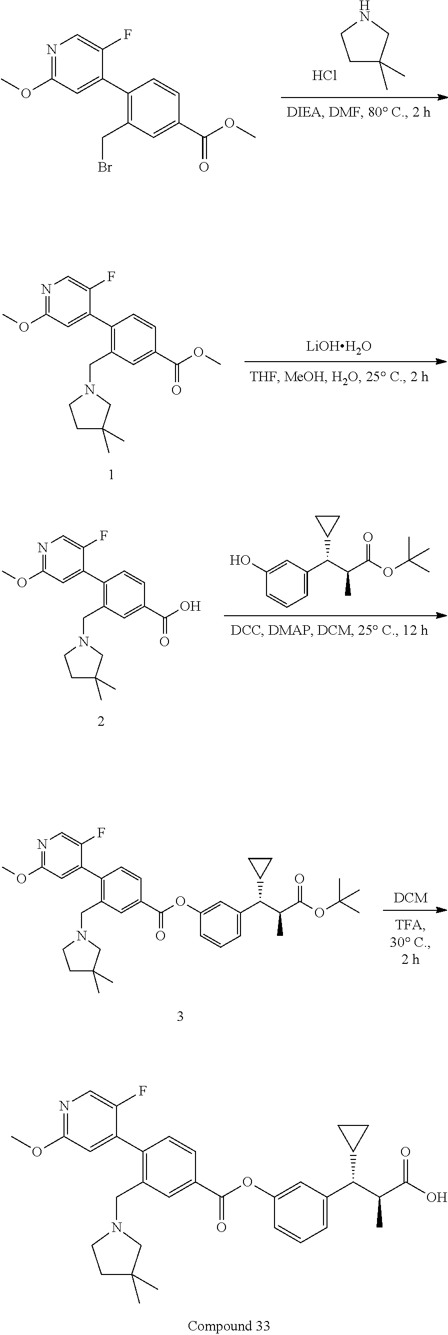

Step 1: methyl 3-[(3,3-dimethylpyrrolidin-1-yl)methyl]-4-(5-fluoro-2-methoxy-4-pyridyl)benzoate (1): To a solution of methyl 3-(bromomethyl)-4-(5-fluoro-2-methoxy-4-pyridyl)benzoate (0.47 g, 1.3 mmol, 1 eq) in DMF (5 mL) was added DIEA (0.17 g, 1.3 mmol, 1 eq) and 3,3-dimethylpyrrolidine hydrochloride (0.18 g, 1.3 mmol, 1 eq). The mixture was stirred at 80° C. for 2 hours. The mixture was concentrated in vacuo to give crude. The crude was purified by reversed-phase HPLC (0.1% FA condition) to give 1 (0.27 g, 55% yield) as a colorless oil. LCMS: (ES⁺) m/z (M+H)⁺=373.0.

Step 2: 3-[(3,3-dimethylpyrrolidin-1-yl)methyl]-4-(5-fluoro-2-methoxy-4-pyridyl)benzoic acid (2): To a solution of 1 (0.25 g, 0.67 mmol, 1 eq) in THE (1.5 mL), MeOH (1.5 mL) H₂O (1.5 mL) was added LiOH.H₂O (80 mg, 3.4 mmol, 5 eq). The mixture was stirred at 25° C. for 2 hours. The reaction solution was acidified to pH 3-5 with 1 M HCl solution. Then mixture was concentrated under reduced pressure to give 2 (0.37 g, crude, HCl salt) as a yellow solid. LCMS: (ES⁺) m/z (M+H)⁺=359.0.

Step 3: [3-[(1R,2S)-3-tert-butoxy-1-cyclopropyl-2-methyl-3-oxo-propyl]phenyl] 3-[(3,3-dimethylpyrrolidin-1-yl)methyl]-4-(5-fluoro-2-methoxy-4-pyridyl)benzoate (3): To a solution of 2 (0.37 g, 1.0 mmol, 1 eq), tert-butyl (2S,3R)-3-cyclopropyl-3-(3-hydroxyphenyl)-2-methyl-propanoate (0.29 g, 1.0 mmol, 1 eq) in DCM (10 mL) was added DCC (0.32 g, 1.6 mmol, 1.5 eq) and DMAP (0.14 g, 1.1 mmol, 1.1 eq). The mixture was stirred at 25° C. for 12 hours. The mixture was filtered and concentrated under reduced pressure to give a residue. The residue was purified by column chromatography (SiO₂, Petroleum ether:Ethyl acetate=20:1 to 10:1) to give 3 (0.37 g, 54% yield) as a colorless oil. LCMS: (ES⁺) m/z (M+H)⁺=617.3.

Step 4: (2S,3R)-3-cyclopropyl-3-[3-[3-[(3,3-dimethylpyrrolidin-1-yl)methyl]-4-(5-fluoro-2-methoxy-4-pyridyl)benzoyl]oxyphenyl]-2-methyl-propanoic acid (Compound 33): To a solution of 3 (0.37 g, 0.6 mmol, 1 eq) in DCM (5 mL) was added TFA (1.5 g, 14 mmol, 1 mL, 22.5 eq). The mixture was stirred at 30° C. for 2 hours. The mixture was concentrated under reduced pressure to remove solvent. The residue was diluted with DMF (2 mL). The residue was purified by prep-HPLC (column: Phenomenex Synergi C18 150×30 mm×4 μm; mobile phase: [water (0.225% FA)-ACN]; B %: 20%-50%, 12 min) to give Compound 33 (0.17 g, 50% yield, FA salt) as a white solid. LCMS: (ES⁺) m/z (M+H)⁺=561.2. ¹HNMR (400 MHz, CD₃OD) δ 8.50 (d, J=4.8 Hz, 1H), 8.29 (dd, J₁=8.0 Hz, J₂=1.6 Hz, 1H), 8.16 (s, 1H), 7.56 (d, J=8.0 Hz, 1H), 7.45-7.35 (m, 1H), 7.22-7.09 (m, 3H), 6.89 (d, J=4.8 Hz, 1H), 4.09 (s, 2H), 3.96 (s, 3H), 2.99 (s, 2H), 2.84-2.79 (m, 1H), 2.67 (s, 2H), 2.07 (t, J=9.6 Hz, 1H), 1.70 (t, J=6.8 Hz, 2H), 1.21-1.11 (m, 1H), 1.06 (s, 6H), 0.96 (d, J=6.8 Hz, 3H), 0.67-0.59 (m, 1H), 0.42-0.31 (m, 2H), 0.07-0.01 (m, 1H)

### Example 15: (2S,3R)-3-cyclopropyl-3-(3-((3-((ethyl(isopropyl)amino)methyl)-4-(5-fluoro-2-methoxypyridin-4-yl)benzoyl)oxy)phenyl)-2-methylpropanoic acid (Compound 34)

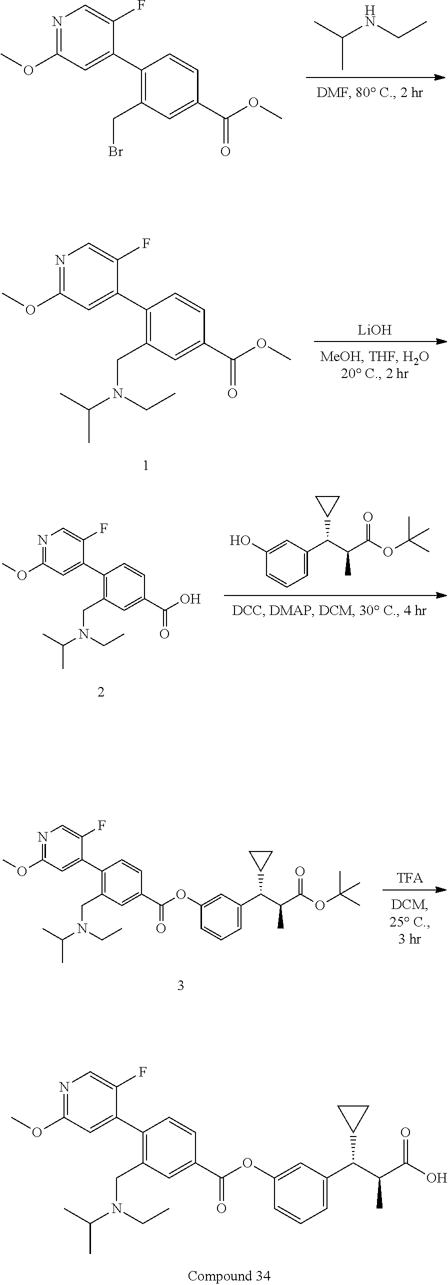

Step 1: methyl 3-((ethyl(isopropyl)amino)methyl)-4-(5-fluoro-2-methoxypyridin-4-yl)benzoate (1): To a solution of methyl 3-(bromomethyl)-4-(5-fluoro-2-methoxypyridin-4-yl)benzoate (0.50 g, 1.4 mmol, 1 eq) in DMF (5 mL) was added N-ethylpropan-2-amine (0.25 g, 2.8 mmol, 0.34 mL, 2 eq). The mixture was stirred at 80° C. for 2 hr. The mixture was purified by reversed-phase flash (0.1% FA condition) to give 1 (0.33 g, 65% yield) as a colorless oil. LCMS: tR=0.595 min., (ES⁺) m/z (M+H)⁺=361.0. ¹H-NMR (CDCl₃, 400 MHz): δ 8.44 (s, 1H), 8.06 (s, 1H), 8.01 (dd, J₁=8.0 Hz, J₂=2.0 Hz, 1H), 7.26 (d, J=8.0 Hz, 1H), 6.65 (d, J=5.2 Hz, 1H), 3.97 (s, 3H), 3.96 (s, 3H), 3.62 (s, 2H), 3.01-2.95 (m, 1H), 2.45 (q, J=7.2 Hz, 2H), 0.97-0.89 (m, 9H).

Step 2: 3-((ethyl(isopropyl)amino)methyl)-4-(5-fluoro-2-methoxypyridin-4-yl) benzoic acid (2): To a solution of 1 (0.33 g, 0.91 mmol, 1 eq) in MeOH (2 mL), THF (2 mL) and H₂O (2 mL) was added LiOH H₂O (0.19 g, 4.6 mmol, 5 eq). The mixture was stirred at 20° C. for 2 hr. The mixture was added 1 N HCl to pH=5-6. The suspension was filtered to collect filter cake. The filtrate was concentrated under reduced pressure to give a residue. The filter cake and the residue were combined to give 2 (0.30 mg, crude) as a white solid. LCMS: tR=0.558 min., (ES+) m/z (M+H)⁺=346.9.

Step 3: 3-((1R,2S)-3-(tert-butoxy)-1-cyclopropyl-2-methyl-3-oxopropyl)phenyl 3-((ethyl(isopropyl)amino)methyl)-4-(5-fluoro-2-methoxypyridin-4-yl)benzoate (3): To a solution of 2 (0.30 g, 0.86 mmol, 1 eq) and tert-butyl (2S,3R)-tert-butyl 3-cyclopropyl-3-(3-hydroxyphenyl)-2-methylpropanoate (0.40 g, 1.4 mmol, 1.67 eq) in DCM (5 mL) was added DCC (0.27 g, 1.3 mmol, 1.5 eq) and DMAP (53 mg, 0.43 mmol, 0.5 eq). The mixture was stirred at 30° C. for 4 hr. The mixture was concentrated under reduced pressure to give a residue. The residue was purified by column chromatography (SiO₂, Petroleum ether:Ethyl acetate=20:1 to 10:1) to give 3 (0.54 g, crude) as a colorless oil. LCMS: tR=0.772 min., (ES+) m/z (M+H)⁺=605.3.

Step 4: (2S,3R)-3-cyclopropyl-3-(3-((3-((ethyl(isopropyl)amino)methyl)-4-(5-fluoro-2-methoxypyridin-4-yl)benzoyl)oxy)phenyl)-2-methylpropanoic acid (Compound 34): To a solution of 3 (0.25 g, 0.41 mmol, 1 eq) in DCM (2 mL) was added TFA (0.4 mL). The mixture was stirred at 25° C. for 3 hr. The mixture was concentrated under reduced pressure to give a residue. The residue was purified by prep-HPLC [column: Phenomenex Gemini 150×25 mm×10 μm; mobile phase: [water (0.225% FA)-ACN]; B %: 20%-50%, 10 min] to give Compound 34 (57 mg, 25% yield, FA salt) as a white solid. LCMS: tR=0.712 min., (ES+) m/z (M+H)⁺=549.2. ¹H-NMR (MeOD, 400 MHz): δ 8.50 (s, 1H), 8.26 (d, J=8.0 Hz, 1H), 8.15 (s, 1H), 7.52 (d, J=8.0 Hz, 1H), 7.41 (t, J=8.0 Hz, 1H), 7.17 (d, J=8.0 Hz, 1H), 7.15-7.10 (m, 2H), 6.85 (d, J=4.8 Hz, 1H), 3.96 (s, 5H), 3.21-3.14 (m, 1H), 2.87-2.76 (m, 1H), 2.71 (q, J=7.2 Hz, 2H), 2.08 (t, J=10.0 Hz, 1H), 1.21-1.10 (m, 1H), 1.07-0.99 (m, 9H), 0.96 (d, J=6.8 Hz, 3H), 0.68-0.57 (m, 1H), 0.44-0.30 (m, 2H), 0.09-0.01 (m, 1H).

### Example 16: (2S,3R)-3-cyclopropyl-3-(3-((4-(5-fluoro-2-methoxypyridin-4-yl)-3-((isopropyl((2S,3R,4R,5R)-2,3,4,5,6-pentahydroxyhexyl)amino)methyl)benzoyl)oxy)phenyl)-2-methylpropanoic acid (Compound 35)

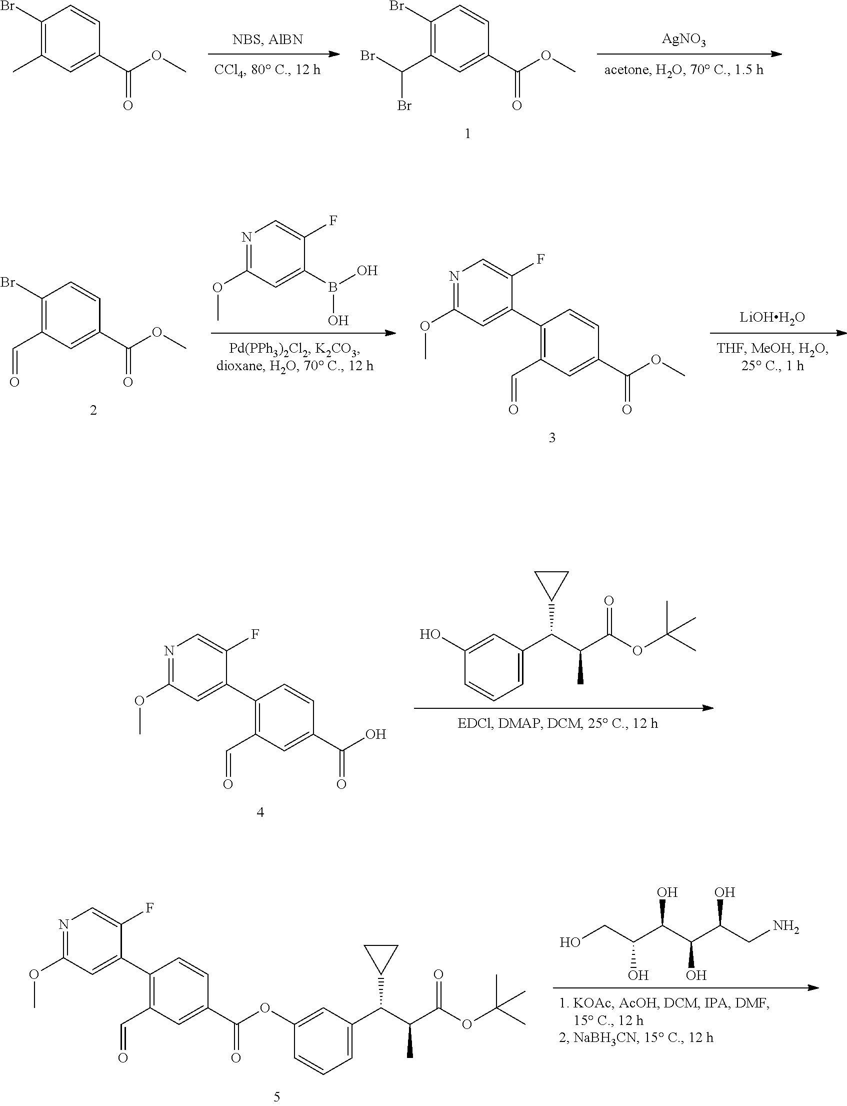

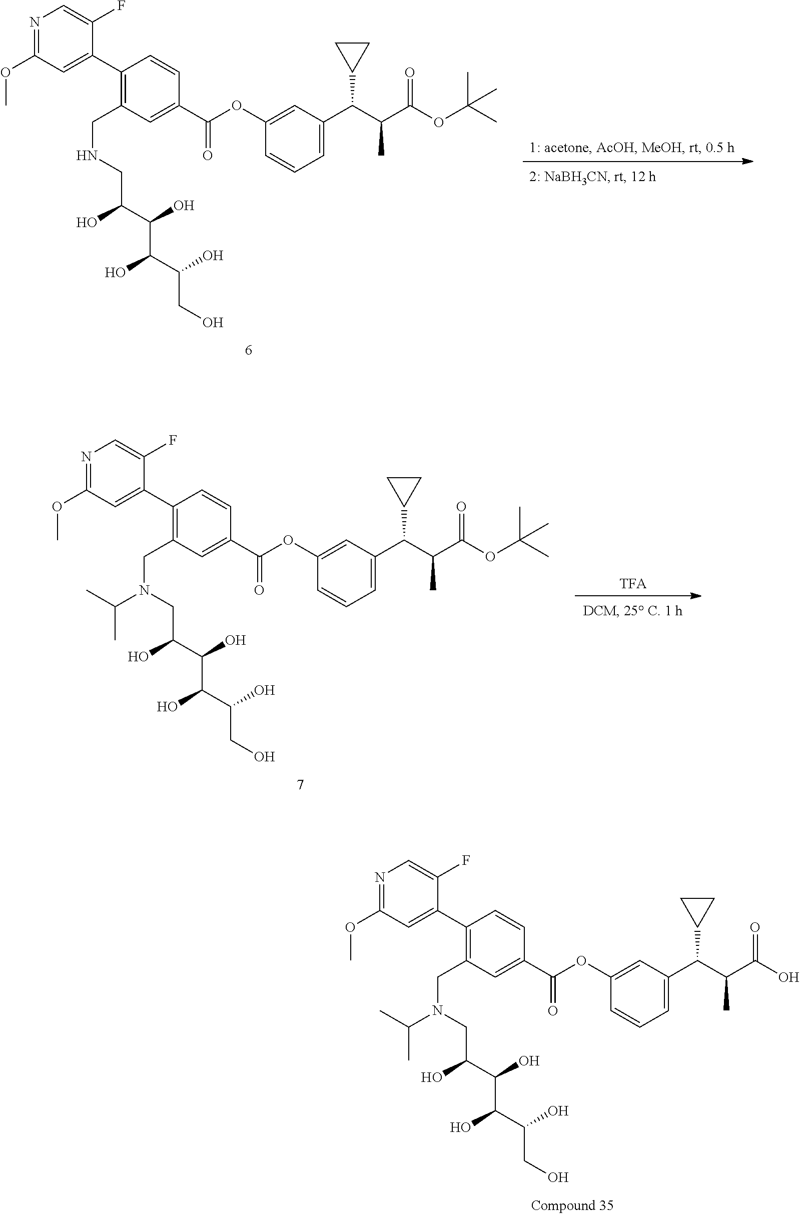

Step 1: methyl 4-bromo-3-(dibromomethyl)benzoate (1): To a solution of methyl 4-bromo-3-methyl-benzoate (5.0 g, 22 mmol, 1 eq) in CCl₄ (200 mL) was added NBS (12 g, 65 mmol, 3 eq) and AIBN (0.72 g, 4.4 mmol, 0.2 eq). The mixture was stirred at 80° C. for 12 hours. The mixture was filtered. The filtrate was concentrated in vacuo to give crude product. The residue was added n-hexane (80 mL) and stirred at 20° C. for 10 min, and then filtered. The filter residue was dried in vacuo to give 1 (8.0 g, crude) as a yellow solid.

Step 2: methyl 4-bromo-3-formylbenzoate (2): To a solution of 2 (8.0 g, 21 mmol, 1 eq) in acetone (240 mL) and H₂O (48 mL), then AgNO₃ (7.0 g, 41 mmol, 2 eq) was added. The reaction was stirred at 70° C. for 1.5 hours. The mixture was filtered. The filtrate was concentrated in vacuo to remove acetone. The residue was added ethyl acetate (200 mL), washed with 2 N HCl solution (50 mL×2), saturated brine (100 mL×2), and then concentrated in vacuo to give 2 (6.0 g, crude) as a yellow solid.

Step 3: methyl 4-(5-fluoro-2-methoxypyridin-4-yl)-3-formylbenzoate (3): To a solution of 2 (0.5 g, 2.1 mmol, 1 eq) and (5-fluoro-2-methoxy-4-pyridyl)boronic acid (0.35 g, 2.1 mmol, 1 eq) in dioxane (5 mL) and H₂O (1 mL), then Pd(PPh₃)₂Cl₂ (72 mg, 0.1 mmol, 0.05 eq) and K₂CO₃ (0.85 g, 6.2 mmol, 3 eq) was added. The mixture was stirred at 70° C. for 12 hours. The mixture dissolved with ethyl acetate (100 mL), washed with saturated brine (30 mL×3), dried over Na₂SO₄, filtered and concentrated under reduced pressure to give a residue. The residue was purified by column chromatography (SiO₂, Petroleum ether:Ethyl acetate=20:1 to 10:1) to give 3 (0.42 g, 71% yield) as a colorless oil. LCMS: tR=0.922 min., (ES⁺) m/z (M+H)⁺=290.0. ¹H-NMR (CDCl₃, 400 MHz): δ 9.97 (d, J=2.4 Hz, 1H), 8.67 (d, J=1.6 Hz, 1H), 8.34 (dd, J₁=2 Hz, J₂=2 Hz, 1H), 8.10 (d, J=0.8 Hz, 1H), 7.49 (d, J=8.0 Hz, 1H), 6.72 (d, J=4.8 Hz, 1H), 3.99 (s, 3H), 3.97 (s, 3H).

Step 4: 4-(5-fluoro-2-methoxypyridin-4-yl)-3-formylbenzoic acid (4): To a solution of 3 (0.37 g, 1.3 mmol, 1 eq) in H₂O (2 mL), THF (4 mL), MeOH (2 mL) was added LiOH.H₂O (0.11 g, 2.6 mmol, 2 eq). The mixture was stirred at 25° C. for 1 hour. The pH was adjusted to around 6 by progressively adding 1 N HCl. The mixture was dissolved with ethyl acetate (50 mL), washed with saturated brine (30 mL×3), dried over Na₂SO₄, filtered and concentrated under reduced pressure to give crude 4 (0.27 g, crude) as a white solid. LCMS: tR=0.825 min., (ES⁺) m/z (M+H)⁺=276.0.

Step 5: 3-((1R,2S)-3-(tert-butoxy)-1-cyclopropyl-2-methyl-3-oxopropyl)phenyl 4-(5-fluoro-2-methoxypyridin-4-yl)-3-formylbenzoate (5): To a solution of 4 (0.22 g, 0.8 mmol, 1 eq), tert-butyl (2S,3R)-3-cyclopropyl-3-(3-hydroxyphenyl)-2-methyl-propanoate (0.27 g, 0.96 mmol, 1.2 eq) in DCM (2 mL) was added EDCI (0.23 g, 1.2 mmol, 1.5 eq) and DMAP (98 mg, 0.8 mmol, 1 eq). The mixture was stirred at 25° C. for 12 hours. The mixture was dissolved with ethyl acetate (100 mL), washed with saturated brine (30 mL×3), dried over Na₂SO₄, filtered and concentrated under reduced pressure to give a residue. The residue was purified by column chromatography (SiO₂, Petroleum ether:Ethyl acetate=20:1 to 5:1) to give 5 (0.42 g, 94% yield) as a yellow oil. LCMS: tR=1.201 min., (ES⁺) m/z (M+H)⁺=534.4.

Step 6: 3-((1R,2S)-3-(tert-butoxy)-1-cyclopropyl-2-methyl-3-oxopropyl)phenyl 4-(5-fluoro-2-methoxypyridin-4-yl)-3-((((2S,3R,4R,5R)-2,3,4,5,6-pentahydroxyhexyl)amino)methyl)benzoate (6): To a solution of 5 (0.10 g, 0.19 mmol, 1 eq), (2R,3R,4R,5S)-6-aminohexane-1,2,3,4,5-pentol (0.10 g, 0.56 mmol, 3 eq) in DCM (1 mL) and IPA (5 mL) was added KOAc (55 mg, 0.56 mmol, 3 eq) and AcOH (34 mg, 0.56 mmol, 3 eq). The mixture was stirred at 15° C. for 12 hours, then NaBH₃CN (12 mg, 0.19 mmol, 1 eq) was added. The mixture was stirred at 15° C. for 12 hours. The reaction was concentrated under reduced pressure to give a residue. The residue was purified by prep-HPLC (FA condition: column: Boston Green ODS 150×30 mm×5 μm; mobile phase: [water (0.225% FA)-ACN]; B %: 30%-60%, 10 min) to give 6 (45 mg, 32% yield, FA salt) as a yellow solid. LCMS: tR=0.894 min., (ES⁺) m/z (M+H)⁺=699.5. ¹H-NMR (CD₃OD, 400 MHz): δ 8.54-8.46 (m, 2H), 8.28 (dd, J₁=8.0 Hz, J₂=1.6 Hz, 1H), 8.17 (s, 1H), 7.56 (d, J=8.0 Hz, 1H), 7.41 (t, J=8.0 Hz, 1H), 7.18-7.10 (m, 3H), 6.88 (d, J=4.8 Hz, 1H), 4.15-4.01 (m, 2H), 3.97-3.89 (m, 4H), 3.79-3.72 (m, 2H), 3.69-3.56 (m, 3H), 3.00-2.87 (m, 2H), 2.80-2.70 (m, 1H), 2.05 (t, J=9.6 Hz, 1H), 1.49 (s, 9H), 1.20-1.06 (m, 1H), 0.93 (d, J=7.2 Hz, 3H), 0.70-0.57 (m, 1H), 0.41-0.29 (m, 2H), 0.09-0.03 (m, 1H).

Step 7: 3-((1R,2S)-3-(tert-butoxy)-1-cyclopropyl-2-methyl-3-oxopropyl)phenyl 4-(5-fluoro-2-methoxypyridin-4-yl)-3-((isopropyl((2S,3R,4R,5R)-2,3,4,5,6-pentahydroxyhexyl)amino)methyl)benzoate (7): To a solution of 6 (70 mg, 0.10 mmol, 1 eq), acetone (29 mg, 0.50 mmol, 5 eq) in MeOH (1 mL) was added AcOH (3.0 mg, 50 μmol, 0.5 eq). The reaction mixture was stirred at 25° C. for 0.5 hr. Then NaBH₃CN (19 mg, 0.30 mmol, 3 eq) was added. The mixture was stirred at 25° C. for 12 hr. The reaction mixture was concentrated under reduced pressure to give 7 (0.20 g, crude) as a yellow solid. LCMS: tR=0.920 min., (ES⁺) m/z (M+H)⁺=741.4.

Step 8: (2S,3R)-3-cyclopropyl-3-(3-((4-(5-fluoro-2-methoxypyridin-4-yl)-3-((isopropyl((2S,3R,4R,5R)-2,3,4,5,6-pentahydroxyhexyl)amino)methyl)benzoyl)oxy)phenyl)-2-methylpropanoic acid (Compound 35): To a solution of 7 (25 mg, 34 μmol, 1 eq) in DCM (0.2 mL) was added HCl/dioxane (4 M, 0.1 mL). The mixture was stirred at 25° C. for 1 hr. The reaction mixture was concentrated under reduced pressure to give a residue. The residue was purified by prep-HPLC (FA condition; column: Phenomenex Synergi C18 150×25 mm×10 μm; mobile phase: [water (0.225% FA)-ACN]; B %: 28%-58%, 9 min) to give Compound 35 (23 mg, 90% yield, 98% purity, FA salt) as a white solid. LCMS: tR=0.813 min., (ES⁺) m/z (M+H)⁺=685.5. ¹H-NMR (CD₃OD, 400 MHz): δ 8.48 (s, 1H), 8.18 (dd, J₁=8.0 Hz, J₂=1.6 Hz, 1H), 8.13 (s, 1H), 7.45-7.40 (m, 2H), 7.17-7.13 (m, 3H), 6.84 (d, J=4.8 Hz, 1H), 3.95 (s, 3H), 3.82-3.51 (m, 8H), 2.92-2.89 (m, 1H), 2.83-2.81 (m, 1H), 2.64-2.63 (m, 1H), 2.59-2.56 (m, 1H), 2.08 (t, J=10.0 Hz, 1H), 1.16-1.15 (m, 1H), 0.97-0.91 (m, 9H), 0.64-0.61 (m, 1H), 0.40-0.36 (m, 2H), 0.05-0.03 (m, 1H).

### Example 17: (2R,3S)-3-cyclopropyl-3-(3-((3-((diisopropylamino)methyl)-4-(5-fluoro-2-methoxypyridin-4-yl)benzoyl)oxy)phenyl)-2-fluoro-2-methylpropanoic acid (Compound 36)

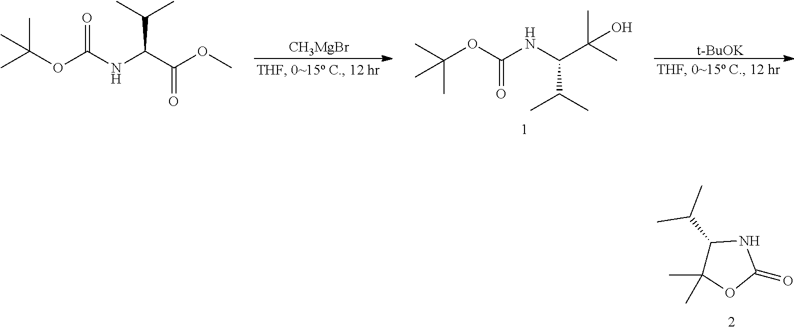

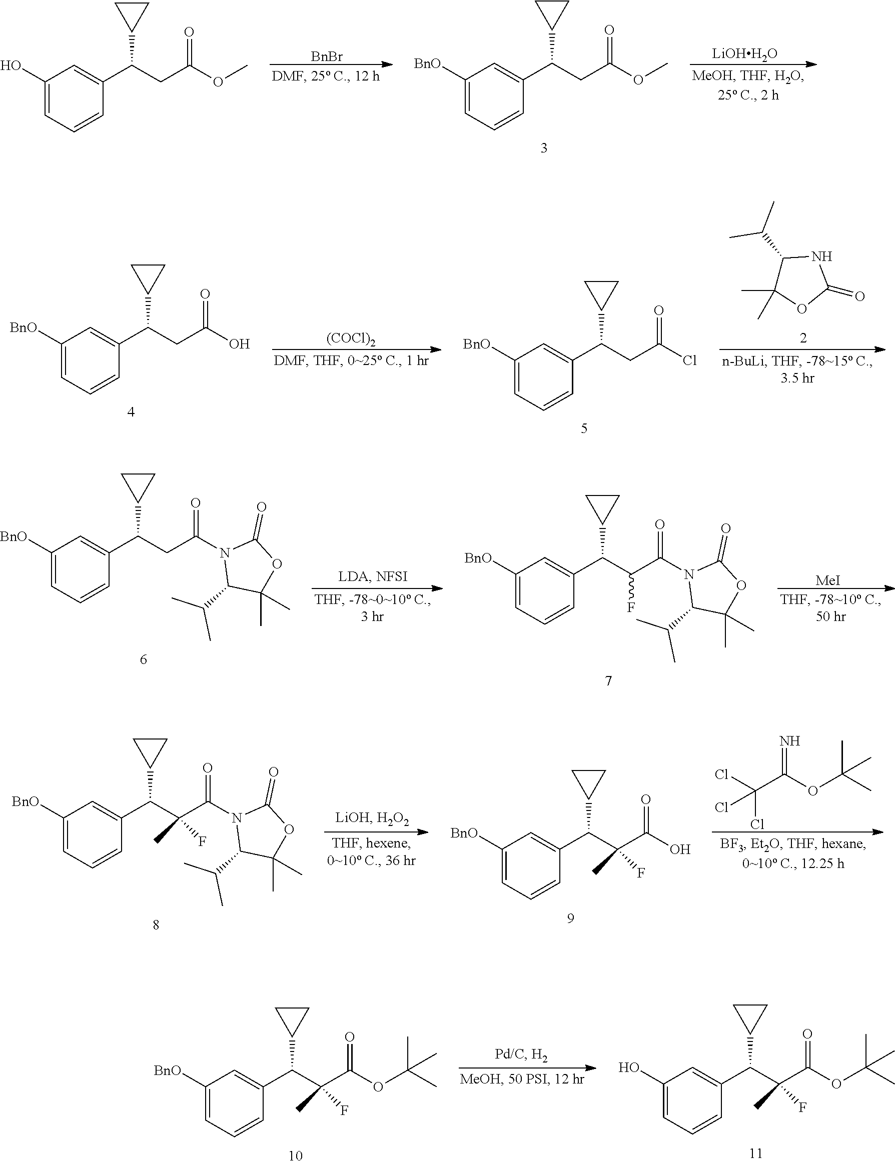

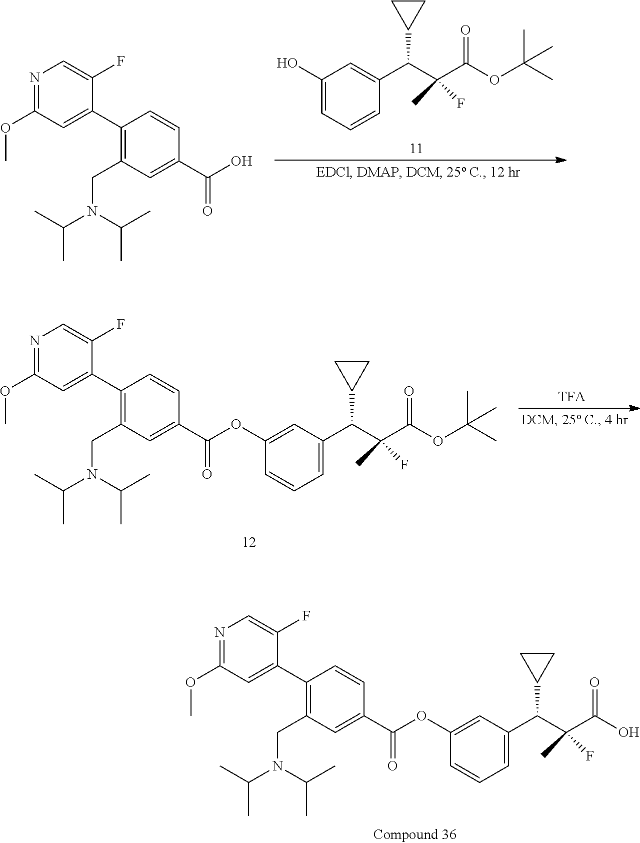 [SMILES: COc1cc(-c2ccc(C(=O)O)cc2CN(C(C)C)C(C)C)c(F)cn1 ; CC(C)(C)OC(=O)[C@](C)(F)[C@H](c1cccc(O)c1)C1CC1 ; COc1cc(-c2ccc(C(=O)Oc3cccc([C@H](C4CC4)[C@@](C)(F)C(=O)OC(C)(C)C)c3)cc2CN(C(C)C)C(C)C)c(F)cn1 ; COc1cc(-c2ccc(C(=O)Oc3cccc([C@H](C4CC4)[C@@](C)(F)C(=O)O)c3)cc2CN(C(C)C)C(C)C)c(F)cn1]

Step 1: (S)-tert-butyl (2-hydroxy-2,4-dimethylpentan-3-yl)carbamate (1): To a solution of (S)-methyl 2-((tert-butoxycarbonyl)amino)-3-methylbutanoate (5 g, 22 mmol) in THF (80 mL) was added methylmagnesium bromide (3 M, 30 mL) at 0° C. The mixture was stirred at 15° C. for 12 hr. The reaction mixture was quenched by addition MeOH 5 mL and saturated NH₄Cl solution 50 mL at 0° C., and then diluted with petroleum ether (30 mL) and extracted with petroleum ether (30 mL×3). The combined organic layers were washed with saturated brine (20 mL×2), dried over Na₂SO₄, filtered and concentrated under reduced pressure to give a residue to give 1 (4.6 g, 91.98% yield) as a colorless oil. ¹H-NMR (CDCl₃, 400 MHz): δ 5.98 (d, J=10.4 Hz, 1H), 4.15 (s, 1H), 3.22 (dd, J₁=3.2 Hz, J₂=10.4 Hz, 1H), 2.03˜1.97 (m, 1H), 1.39 (s, 9H), 1.1 (s, 3H), 1.03 (s, 3H), 0.84˜0.80 (m, 6H).

Step 2: (S)-4-isopropyl-5,5-dimethyloxazolidin-2-one (2): To a solution of 1 (4.6 g, 20 mmol) in THE (0.1 L) was added t-BuOK (2.5 g, 22 mmol) at 0° C. The mixture was stirred at 15° C. for 12 hr. The reaction mixture was concentrated under reduced pressure to give a residue. The residue was dissolved in ethyl acetate, washed with 0.5 M HCl (30 mL) and saturated brine (30 mL). The solution was concentrated under reduced pressure to give a residue. The residue was purified by column chromatography (SiO₂, Petroleum ether:Ethyl acetate=20:1 to 4:1) to give 2 (2.6 g, 83.17% yield) as a white solid. ¹H-NMR (CDCl₃, 400 MHz): δ 6.92 (s, 1H), 3.18 (d, J=8.4 Hz, 1H), 1.85˜1.78 (m, 1H), 1.47 (s, 3H), 1.36 (s, 3H), 0.99 (d, J=6.8 Hz, 3H), 0.91 (d, J=6.4 Hz, 3H).

Step 3: (S)-methyl 3-(3-(benzyloxy)phenyl)-3-cyclopropylpropanoate (3): To a solution of (S)-methyl 3-cyclopropyl-3-(3-hydroxyphenyl)propanoate (5.0 g, 23 mmol) in DMF (20 mL) was added BnBr (5.8 g, 34 mmol, 4 mL) and K₂CO₃ (6.3 g, 45 mmol). The mixture was stirred at 25° C. for 12 hr. The reaction mixture was quenched by addition water (30 mL), and extracted with ethyl acetate (40 mL×3). The combined organic layers were washed with saturated brine (10 mL×3), dried over Na₂SO₄, filtered and concentrated under reduced pressure to give 3 (7.0 g, 99% yield) as a yellow oil. LCMS: tR=1.060 min., (ES⁺) m/z (M+H)⁺=311.5.

Step 4: (S)-3-(3-(benzyloxy)phenyl)-3-cyclopropylpropanoic acid (4): To a solution of 3 (7.0 g, 23 mmol) in MeOH (20 mL), H₂O (20 mL) and THE (20 mL) was added LiOH H₂O (1.9 g, 45 mmol). The mixture was stirred at 25° C. for 2 hr. The reaction mixture was quenched by addition water (20 mL) and HCl (20 mL), and then diluted with ethyl acetate (20 mL) and extracted with ethyl acetate (40 mL×3). The combined organic layers were washed with saturated brine (15 mL×2), dried over Na₂SO₄, filtered and concentrated under reduced pressure to give a residue. The residue was dissolved in petroleum ether and filtered to give 4 (5.2 g, 74% yield, 94% purity) as a white solid. LCMS: tR=0.963 min., (ES⁺) m/z (M+H)⁺=297.5. ¹H-NMR (CDCl₃, 400 MHz): δ 7.43˜7.42 (m, 2H), 7.37-7.33 (m, 2H), 7.31˜7.27 (m, 1H), 7.18 (t, J=8 Hz, 1H), 6.89-6.88 (m, 1H), 6.84-6.80 (m, 2H), 5.06 (s, 2H), 2.76-2.66 (m, 2H), 2.34-2.27 (m, 1H), 1.04-1.00 (m, 1H), 0.58-0.54 (m, 1H), 0.39-0.34 (m, 1H), 0.30-0.24 (m, 1H), 0.14-0.08 (m, 1H).

Step 5: (S)-3-(3-(benzyloxy)phenyl)-3-cyclopropylpropanoyl chloride (5): To a solution of 4 (4.0 g, 13 mmol) in THF (20 mL) was added (COCl)₂ (5.1 g, 40 mmol, 3.5 mL) and DMF (99 mg, 1.3 mmol, 0.1 mL) at 0° C. The mixture was stirred at 25° C. for 1 hr. The reaction mixture was concentrated under reduced pressure to give 5 (4.0 g, crude) as a yellow oil.

Step 6: (S)-3-((S)-3-(3-(benzyloxy)phenyl)-3-cyclopropylpropanoyl)-4-isopropyl-5,5-dimethyloxazolidin-2-one (6): To a solution of 2 (2.3 g, 14.67 mmol) in THE (53 mL) was added n-BuLi (2.5 M, 6.4 mL) at −78° C. The mixture was stirred at −78° C. for 0.5 hr. A solution of 5 (4.2 g, 13.34 mmol) in THE (16 mL) was added dropwise. The mixture was stirred at −78° C. for 2 hr and stirred at 15° C. for 1 hr. The reaction mixture was quenched by addition saturated NH₄Cl solution (30 mL) and water (30 mL) at 0° C., and then diluted with ethyl acetate (40 mL) and extracted with ethyl acetate (30 mL×3). The combined organic layers were washed with saturated brine (30 mL×2), dried over Na₂SO₄, filtered and concentrated under reduced pressure to give a residue. The residue was purified by column chromatography (SiO₂, Petroleum ether:Ethyl acetate=100:1 to 30:1) to give 6 (5.7 g, 96% yield, 96% purity) as a colorless oil. LCMS: tR=1.018 min., (ES⁺) m/z (M+H)⁺=436.2.

Step 7: (4S)-3-((3S)-3-(3-(benzyloxy)phenyl)-3-cyclopropyl-2-fluoropropanoyl)-4-isopropyl-5,5-dimethyloxazolidin-2-one (7): To a solution of 6 (5.2 g, 12 mmol) in THE (0.13 L) was added LDA (2 M, 12 mL) at −78° C. The mixture was stirred at 0° C. for 1 hr. Then N-(benzenesulfonyl)-N-fluoro-benzenesulfonamide (7.5 g, 24 mmol) was added to the mixture at -78° C. The mixture was stirred at 10° C. for 2 hr. The reaction mixture was quenched by addition NH₄Cl (10 mL) and water (10 mL) at 0° C., and then diluted with ethyl acetate (20 mL) and extracted with ethyl acetate (20 mL×3). The combined organic layers were washed with saturated brine (15 mL×2), dried over Na₂SO₄, filtered and concentrated under reduced pressure to give a residue. The residue was purified by prep-HPLC (column: Phenomenex Synergi Max-RP 150×50 mm×10 μm; mobile phase: [water (0.225% FA)-ACN]; B %: 59%-89%, 10 min) to give 7 (4.1 g, 76% yield, 100% purity) as a yellow oil. LCMS: tR=1.138 min., (ES⁺) m/z (M+H)+=454.2. ¹H-NMR (CDCl₃, 400 MHz): δ 7.28-7.27 (m, 2H), 7.24-7.20 (m, 2H), 7.18-7.14 (m, 1H), 7.07 (t, J=4 Hz, 1H), 6.81 (s, 1H), 6.73˜6.70 (m, 2H), 6.29-6.15 (m, 1H), 4.89 (s, 2H), 2.88 (d, J=3.2 Hz, 1H), 2.23˜2.12 (m, 1H), 2.04˜1.98 (m, 1H), 1.36 (s, 3H), 1.23˜1.16 (m, 4H), 0.88 (d, J=7.2 Hz, 3H), 0.81 (d, J=7.2 Hz, 3H), 0.49-0.44 (m, 1H), 0.37-0.31 (m, 2H), 0.06˜−0.05 (m, 1H).

Step 8: (S)-3-((2R,3S)-3-(3-(benzyloxy)phenyl)-3-cyclopropyl-2-fluoro-2-methylpropanoyl)-4-isopropyl-5,5-dimethyloxazolidin-2-one (8): To a solution of 7 (3.5 g, 7.7 mmol) in THE (70 mL) was added LiHMDS (1 M, 23 mL) at −78° C. The mixture was stirred at -78° C. for 0.5 hr and 0° C. for 90 min. Then Mel (6.6 g, 46 mmol, 2.9 mL) was added to the mixture at −78° C. The mixture was stirred at 10° C. for 48 hr. The reaction mixture was quenched by addition saturated brine (20 mL) and water (20 mL) at 0° C., and then diluted with ethyl acetate (30 mL) and extracted with ethyl acetate (30 mL×3). The combined organic layers were washed with saturated brine (15 mL×2), dried over Na₂SO₄, filtered and concentrated under reduced pressure to give a residue. The residue was purified by column chromatography (SiO₂, Petroleum ether:Ethyl acetate=80:1 to 50:1) to give 8 (2.2 g, 59% yield, 97% purity) as a yellow oil. LCMS: tR=1.159 min., (ES⁺) m/z (M+H)⁺=468.3. ¹H-NMR (CDCl₃, 400 MHz): δ 7.46-7.44 (m, 2H), 7.40-7.37 (m, 2H), 7.34-7.30 (m, 1H), 7.23 (t, J=4 Hz, 1H), 7.01 (s, 1H), 6.95˜6.88 (m, 2H), 6.29˜6.15 (m, 1H), 5.07 (s, 2H), 3.21˜3.15 (m, 1H), 2.33˜2.27 (m, 1H), 1.55 (s, 3H), 1.47 (s, 3H), 1.41 (s, 5H), 1.17 (d, J=7.2 Hz, 3H), 1.08 (d, J=7.2 Hz, 3H), 0.61˜0.55 (m, 1H), 0.46-0.38 (m, 2H), 0.08-0.01 (m, 1H).

Step 9: (2R,3S)-3-(3-(benzyloxy)phenyl)-3-cyclopropyl-2-fluoro-2-methylpropanoic acid (9): To a solution of 8 (2.1 g, 4.5 mmol) in THE (30 mL) was added LiOH H₂O (0.75 g, 18 mmol) and H₂O₂ (4.1 g, 36 mmol, 3.5 mL, 30% purity) at 0° C. The mixture was stirred at 10° C. for 36 hr. The reaction mixture was quenched by addition saturated NaHCO₃ (20 mL) and water (20 mL), and then diluted with ethyl acetate (20 mL) and extracted with ethyl acetate (20 mL×3). The combined organic layers were washed with saturated brine (15 mL×2), dried over Na₂SO₄, filtered and concentrated under reduced pressure to give a residue. The residue was purified by prep-HPLC (column: Phenomenex Synergi Max-RP 250×50 mm×10 μm; mobile phase: [water (0.225% F A)-ACN]; B %: 30%-60%, 20 min) to give 9 (0.9 g, 61% yield) as a white solid. LCMS: tR=0.823 min., (ES⁺) m/z (M−H)⁻=327.1. ¹H-NMR (CDCl₃, 400 MHz): δ 7.46-7.45 (m, 2H), 7.41˜7.38 (m, 2H), 7.35-7.31 (m, 1H), 7.28-7.24 (m, 1H), 6.94-6.92 (m, 2H), 6.89˜6.87 (m, 1H), 5.08 (s, 2H), 2.28˜2.18 (m, 1H), 1.48˜1.35 (m, 4H), 0.70˜0.66 (m, 1H), 0.49-0.42 (m, 2H), 0.08-0.02 (m, 1H).

Step 10: (2R,3S)-tert-butyl 3-(3-(benzyloxy)phenyl)-3-cyclopropyl-2-fluoro-2-methylpropanoate (10): To a solution of 9 (0.85 g, 2.6 mmol) in THE (10 mL) and Hexane (10 mL) was added tert-butyl 2,2,2-trichloroacetimidate (1.1 g, 5.2 mmol, 0.93 mL). The mixture was stirred at 0° C. for 15 min. Then BF₃.Et₂O (37 mg, 0.26 mmol, 32 μL) was added to the mixture at 0° C. The mixture was stirred at 10° C. for 12 hr. The reaction mixture was quenched by addition saturated NaHCO₃ solution (20 mL) and water (20 mL) at 0° C., and then diluted with ethyl acetate (20 mL) and extracted with ethyl acetate (20 mL×3). The combined organic layers were washed with saturated brine (15 mL×2), dried over Na₂SO₄, filtered and concentrated under reduced pressure to give a residue. The residue was purified by column chromatography (SiO₂, Petroleum ether:Ethyl acetate=30:1 to 20:1) to give 10 (0.85 g, 85% yield, 100% purity) as a colorless oil. LCMS: tR=1.038 min., (ES⁺) m/z (M+H₂O)=402.4. ¹H-NMR (CDCl₃, 400 MHz): δ 7.46-7.44 (m, 2H), 7.41˜7.37 (m, 2H), 7.35-7.31 (m, 1H), 7.25-7.21 (m, 1H), 6.93˜6.86 (m, 3H), 5.07 (s, 2H), 2.27˜2.15 (m, 1H), 1.55 (s, 9H), 1.47˜1.38 (m, 1H), 1.29˜1.24 (m, 3h), 0.69-0.62 (m, 1H), 0.44-0.37 (m, 2H), 0.01˜-0.05 (m, 1H).

Step 11: (2R,3S)-tert-butyl 3-cyclopropyl-2-fluoro-3-(3-hydroxyphenyl)-2-methylpropanoate (11): To a solution of 10 (0.85 g, 2.2 mmol) in MeOH (10 mL) was added 5% Pd/C (85 mg). The mixture was stirred at 25° C. for 12 hr under H₂. The reaction mixture was filtered and the solution was concentrated under reduced pressure to give 11 (0.65 g, 94.89% yield, 95% purity) as a white solid. LCMS: tR=0.870 min., (ES⁺) m/z (M+H₂O)=312.4. ¹H-NMR (CDCl₃, 400 MHz): δ7.18 (t, J=8 Hz, 1H), 6.82˜6.80 (m, 2H), 6.76-6.74 (m, 1H), 2.23˜2.13 (m, 1H), 1.55 (s, 9H), 1.44˜1.39 (m, 1H), 1.31˜1.26 (m, 3H), 0.69˜0.62 (m, 1H), 0.44˜0.37 (m, 2H), 0.01˜-0.05 (m, 1H).

Step 12: 3-((1S,2R)-3-(tert-butoxy)-1-cyclopropyl-2-fluoro-2-methyl-3-oxopropyl) phenyl 3-((diisopropylamino)methyl)-4-(5-fluoro-2-methoxypyridin-4-yl)benzoate (12): To a solution of 3-((diisopropylamino)methyl)-4-(5-fluoro-2-methoxypyridin-4-yl)benzoic acid (Example 7, Step 4) (0.12 g, 0.31 mmol, FA salt) and 11 (0.1 g, 0.34 mmol) in DCM (3 mL) was added EDCI (0.12 g, 0.62 mmol) and DMAP (75 mg, 0.62 mmol). The mixture was stirred at 25° C. for 12 hr. The reaction mixture was used for next step without work-up and purification. LCMS: tR=0.849 min., (ES⁺) m/z (M+H)⁺=637.5.

Step 13: (2R,3S)-3-cyclopropyl-3-(3-((3-((diisopropylamino)methyl)-4-(5-fluoro-2-methoxypyridin-4-yl)benzoyl)oxy)phenyl)-2-fluoro-2-methylpropanoic acid (Compound 36): To the DCM solution of 12 was added TFA (3.1 g, 27 mmol, 2 mL). The mixture was stirred at 25° C. for 4 hr. The reaction mixture was concentrated under reduced pressure to give a residue. The residue was purified by prep-HPLC (column: Phenomenex Synergi C18 150×25 mm×10 μm; mobile phase: [water (0.225% FA)-ACN]; B %: 22%-52%, 10 min]; B %: 22%-52%, 10 min) to give a Compound 36 (52 mg, 28% yield, FA salt) as a white solid. LCMS: tR=0.855 min., (ES⁺) m/z (M+H)⁺=581.5. ¹H-NMR (CDCl₃, 400 MHz): δ 8.67 (s, 1H), 8.14˜8.09 (m, 2H), 7.39˜7.35 (m, 1H), 7.31 (d, J=8 Hz, 1H), 7.18˜7.15 (m, 3H), 6.67 (d, J=4.8 Hz, 1H), 3.98 (s, 3H), 3.71 (s, 2H), 3.1 (s, 2H), 2.40˜2.30 (m, 1H), 1.4˜1.38 (m, 3H), 0.99˜0.98 (m, 12H), 0.88˜0.84 (m, 1H), 0.65 (s, 2H), 0.48˜0.43 (m, 1H), 0.02 (s, 1H).

### Example 18: (2S,3R)-3-cyclopropyl-3-(3-(((1r,4R)-4-(2-fluoro-5-methoxyphenyl)cyclohexanecarbonyl)oxy)phenyl)-2-methylpropanoic acid (Compound 40)

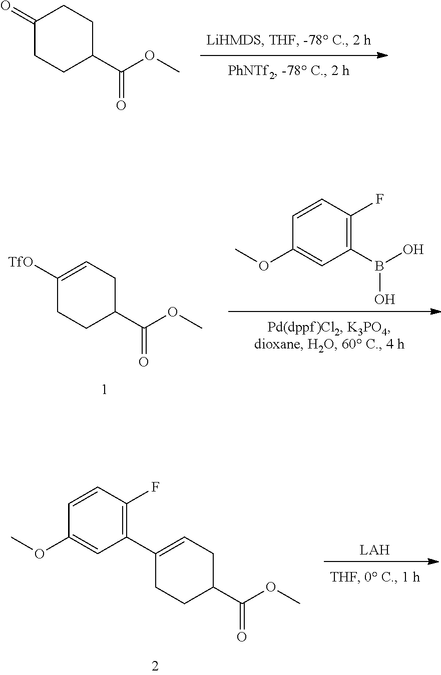

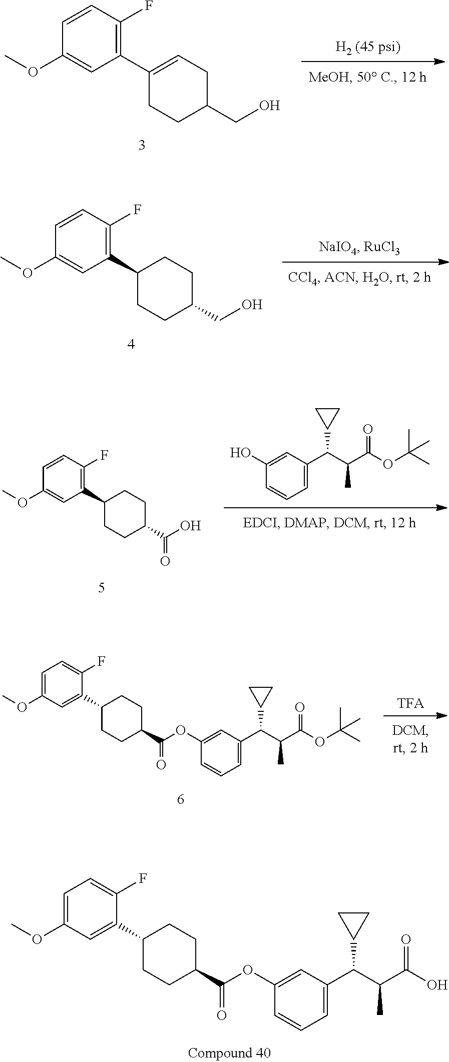

Step 1: methyl 4-(((trifluoromethyl)sulfonyl)oxy)cyclohex-3-enecarboxylate (1): To a −78° C. solution of KHMDS (1 M, 48 mL, 1.50 eq) was added a solution of methyl 4-oxocyclohexanecarboxylate (5.0 g, 32 mmol, 1 eq) in THF (50 mL). The resulting solution was stirred for 2 h at −78° C., then a solution of 1,1,1-trifluoro-N-phenyl-N-((trifluoromethyl)sulfonyl)methanesulfonamide (14 g, 38 mmol, 1.2 eq) in THF (50 mL) was added drop-wise with stirring at −78° C. The resulting solution was stirred for 2 hr at −78° C. The reaction mixture was quenched by addition water (100 mL), and then diluted with Ethyl acetate (100 mL), extracted with ethyl acetate (100 mL×3). The combined organic layers were washed with saturated brine (200 mL×2), dried over Na₂SO₄, filtered and concentrated under reduced pressure to give a residue. The residue was purified by column chromatography (SiO₂, Petroleum ether:Ethyl acetate=100:1 to 20:1) to give 1 (3.0 g, 33% yield) as a white solid.

Step 2: methyl 2′-fluoro-5′-methoxy-2,3,4,5-tetrahydro-[1,1′-biphenyl]-4-carboxylate (2): To a mixture of (2-fluoro-5-methoxyphenyl)boronic acid (1.7 g, 9.7 mmol, 1 eq) in dioxane (90 mL) was added K₃PO₄ (3.2 g, 36 mmol, 3.7 eq) in H₂O (1.93 g, 0.11 mol, 11 eq), 1 (2.8 g, 9.7 mmol, 1 eq) and Pd(dppf)Cl₂ (0.57 g, 0.78 mmol, 0.08 eq. The flask was evacuated and backfilled with nitrogen gas. The reaction was heated to 60° C. for 4 h under an inert atmosphere of nitrogen. The reaction mixture was concentrated under reduced pressure. The residue was diluted with water (150 mL) and extracted with ethyl acetate (150 mL×2). The combined organic layers were washed with saturated brine (90 mL×2), dried over Na₂SO₄, filtered and concentrated under reduced pressure. The residue was purified by column chromatography (SiO₂, Petroleum ether:Ethyl acetate=100:1 to 95:5) to give 2 (2.2 g, 86% yield) as a yellow oil. ¹H-NMR (CD₃Cl, 400 MHz): δ 6.96-6.91 (m, 1H), 6.75-6.71 (m, 2H), 5.94 (s, 1H), 3.82 (s, 3H), 3.73 (s, 3H), 2.68-2.66 (m, 1H), 2.48-2.46 (m, 4H), 2.17-2.15 (m, 1H), 1.86-1.84 (m, 1H).

Step 3: (2′-fluoro-5′-methoxy-2,3,4,5-tetrahydro-[1,1′-biphenyl]-4-yl)methanol (3): To a solution of LAH (0.43 g, 11 mmol, 1.5 eq) in THE (40 mL) was added 2 (2.0 g, 7.6 mmol, 1 eq) at 0° C. The mixture was stirred at 0° C. for 1 hr. The reaction mixture was concentrated under reduced pressure. The residue was diluted with water (50 mL) and extracted with ethyl acetate (50 mL×2). The combined organic layers were washed with saturated brine (40 mL×2), dried over Na₂SO₄, filtered and concentrated under reduced pressure. The residue was purified by column chromatography (SiO₂, Petroleum ether:Ethyl acetate=15:1 to 5:1) to give 3 (1.6 g, 91% yield) as a white solid.

Step 4: ((1r,4r)-4-(2-fluoro-5-methoxyphenyl)cyclohexyl)methanol (4): To a solution of 3 (1.3 g, 5.5 mmol, 1 eq) in MeOH (13 mL) was added 10% Pd/C (0.13 g) under N₂. The suspension was degassed under vacuum and purged with H₂ several times. The mixture was stirred under H₂ (45 psi) at 50° C. for 12 hours. The mixture was filtered and concentrated in vacuo to give a residue. The residue was purified by prep-HPLC (column: Phenomenex luna C18 250×50 mm×10 μm; mobile phase: [water (0.225% FA)-ACN]; B %: 35%-65%, 25 min) and separated by SFC (column: DAICEL CHIRALPAK AD-H (250×30 mm×5 μm); mobile phase: [0.1% NH₃—H₂O in EtOH]; B %: 25%-25%, 2.6 min; 70 min) to give 4 (0.25 g, 19% yield) as a red solid. ¹H-NMR (CD₃Cl, 400 MHz): δ 6.95-6.90 (m, 1H), 6.77-6.75 (m, 1H), 6.68-6.64 (m, 1H), 3.78 (s, 3H), 3.52 (d, J=6.4 Hz, 2H), 2.82 (t, J=12.0 Hz, 1H), 1.94 (d, J=10.8 Hz, 4H), 1.58-1.45 (m, 3H), 1.39-1.37 (m, 1H), 1.21-1.12 (m, 2H).

Step 5: (1r,4r)-4-(2-fluoro-5-methoxyphenyl)cyclohexanecarboxylic acid (5): To a solution of 4 (0.20 g, 84 μmol, 1 eq) in CCl₄ (2 mL), ACN (2 mL), H₂O (2.4 mL) was added RuCl₃ (4.4 mg, 21 μmol, 0.025 eq) and NaIO₄ (0.36 g, 1.7 mmol, 2 eq). The mixture was stirred at 20° C. for 2 hr. The reaction mixture was adjusted pH between 3 to 4 with 1 N HCl. The residue was diluted with H₂O (10 mL) and extracted with ethyl acetate (50 mL×3). The combined organic layers were washed with saturated. brine (10 mL×2), dried over Na₂SO₄, filtered and concentrated under reduced pressure to give a residue. The residue was purified by prep-TLC (SiO₂, PE:EA=1:1) to give 5 (90 mg, 42% yield, 98% purity) as a white solid. LCMS: tR=1.125 min., (ES⁻) m/z (M−H)⁻=251.1. ¹H-NMR (CDCl₃, 400 MHz): δ 6.93 (t, J=9.6 Hz, 1H), 6.74 (dd, J₁=6.0 Hz, J₂=3.2 Hz, 1H), 6.70-6.65 (m, 1H), 3.79 (s, 3H), 2.91-2.80 (m, 1H), 2.47-2.35 (m, 1H), 2.21-2.12 (m, 2H), 2.03-1.94 (m, 2H), 1.71-1.58 (m, 2H), 1.58-1.46 (m, 2H).

Step 6: (1r,4R)-3-((1R,2S)-3-(tert-butoxy)-1-cyclopropyl-2-methyl-3-oxopropyl)phenyl 4-(2-fluoro-5-methoxyphenyl)cyclohexanecarboxylate (6): To a solution of 5 (90 mg, 36 μmol, 1 eq) in DCM (1 mL) was added EDCI (0.1. g, 54 μmol, 1.5 eq) and DMAP (44 mg, 36 μmol, 1 eq) and tert-butyl (2S,3R)-3-cyclopropyl-3-(3-hydroxyphenyl)-2-methyl-propanoate (99 mg, 36 μmol, 1 eq). The mixture was stirred at 25° C. for 12 hr. The reaction mixture was diluted with H₂O (10 mL) and extracted with EtOAc (25 mL×2), dried over Na₂SO₄, filtered and concentrated under reduced pressure to give a residue. The residue was purified by prep-TLC (SiO₂, PE:EA=5:1) to give 6 (0.12 g, 66% yield, 100% purity) as a colorless oil. ¹H-NMR (CDCl₃, 400 MHz): δ 7.33-7.27 (m, 1H), 7.02 (d, J=7.8 Hz, 1H), 6.98-6.92 (m, 2H), 6.90 (t, J=1.6 Hz, 1H), 6.76 (dd, J=5.6 Hz, J=2.8 Hz, 1H), 6.71-6.65 (m, 1H), 3.79 (s, 3H), 2.89-2.97 (m, 1H), 2.74-2.60 (m, 2H), 2.31-2.67 (m, 2H), 2.04-1.98 (m, 3H), 1.78-1.75 (m, 2H), 1.60-1.53 (m, 2H), 1.49 (s, 9H), 1.12-1.02 (m, 1H), 0.92 (d, J=6.8 Hz, 3H), 0.61-0.60 (m, 1H), 0.41-0.28 (m, 2H), 0.05-−0.05 (m, 1H).

Step 7: (2S,3R)-3-cyclopropyl-3-(3-(((1r,4R)-4-(2-fluoro-5-methoxyphenyl)cyclohexanecarbonyl)oxy)phenyl)-2-methylpropanoic acid (Compound 40): To a solution of 6 (0.12 g, 24 μmol, 1 eq) in DCM (1.2 mL) was added TFA (0.4 mL). The mixture was stirred at 25° C. for 2 hr. The reaction was concentrated under reduced pressure to give a residue. The residue was purified by prep-HPLC (FA condition; column: Phenomenex Synergi C18 150×30 mm×4 μm; mobile phase: [water (0.225% FA)-ACN]; B %: 53%-83%, 10 min) to give Compound 40 (65 mg, 58% yield, 95% purity) as an off-white solid. LCMS: tR=1.072 min., (ES⁺) m/z (M+H)⁺=455.4. ¹H-NMR (CDCl₃, 400 MHz): δ 7.37-7.31 (m, 1H), 7.05 (d, J=7.8 Hz, 1H), 7.01-6.97 (m, 1H), 6.96-6.92 (m, 2H), 6.77 (dd, J₁=5.6 Hz, J₂=2.8 Hz 1H), 6.72-6.66 (m, 1H), 3.80 (s, 3H), 2.95-2.80 (m, 2H), 2.68-2.57 (m, 1H), 2.34-2.24 (m, 2H), 2.10-1.99 (m, 3H), 1.70-1.85 (m, 2H), 1.67-1.50 (m, 2H), 1.19-1.07 (m, 1H), 1.02 (d, J=7.2 Hz, 3H), 0.72-0.59 (m, 1H), 0.45-0.34 (m, 2H), 0.12-−0.02 (m, 1H).

The following compounds were prepared according to the procedures described in Examples 1 to 18 using the appropriate intermediates.

| Cpd | Structure | Characterization Data |
|---|---|---|
| 2 | 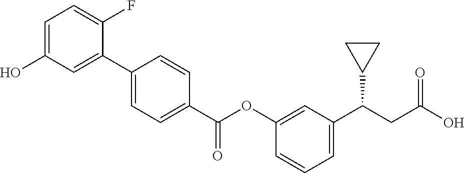 [SMILES: O=C(O)C[C@H](c1cccc(OC(=O)c2ccc(-c3cc(O)ccc3F)cc2)c1)C1CC1] | LCMS: tR = 10.878 min, (ES+) m/z (M + Na)⁺ = 443.1. ¹H HMR (CDCl₃, 400 MHz,) δ = 8.26 (d, J = 8.4 Hz, 2H), 7.68 (m, 2H), 7.42-7.35 (m, 1H), 7.18 (m, 1H), 7.15-7.03 (m, 3H), 6.94 (m, 1H), 6.83 (m, 1H), 2.82 (m, 2H), 2.48-2.42 (m, 1H), 1.12-1.01 (m, 1H), 0.67-0.59 (m, 1H), 0.53-0.44 (m, 1H), 0.34 (m, 1H), 0.21 (m, 1H). |
| 3 | 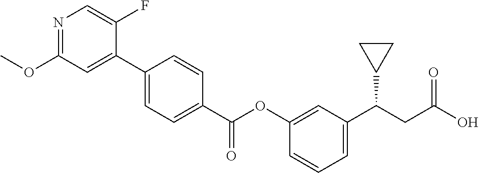 [SMILES: COc1cc(-c2ccc(C(=O)Oc3cccc([C@@H](CC(=O)O)C4CC4)c3)cc2)c(F)cn1] | LCMS: tR = 0.955 min, (ES⁺) m/z (M + H)⁺ = 436.2, ¹H- NMR (CDCl₃, 400 MHz): δ 8.31 (d, J = 7.6 Hz, 2H), 8.16 (s, 1H), 7.74 (d, J = 7.6 Hz, 2H), 6.83 (t, J = 7.6 Hz, 1H), 7.19-7.11 (m, 3H), 6.92 (s, 1H), 4.03 (s, 3H), 2.87-2.76 (m, 2H), 2.48-2.41 (m, 1H), 1.09-1.03 (m, 1H), 0.65-0.59 (m, 1H), 0.51-0.44 (m, 1H), 0.36-0.30 (m, 1H), 0.23-0.17 (m, 1H). |
| 4 | 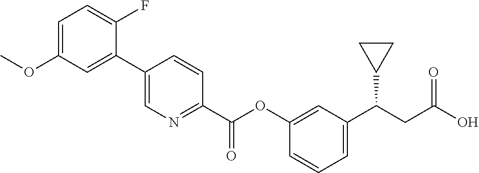 [SMILES: COc1ccc(F)c(-c2ccc(C(=O)Oc3cccc([C@@H](CC(=O)O)C4CC4)c3)nc2)c1] | LCMS: tR = 0.873 min, (ES⁺) m/z (M + H)⁺ = 436.2. ¹H- NMR (CDCl₃, 400 MHz): δ 9.05 (s, 2H), 8.36 (s, 1H), 8.14 (s, 2H), 7.39 (t, J = 7.2 Hz, 1H), 7.20-7.14 (m, 4H), 7.00-6.95 (m, 2H), 3.85 (s, 3H), 2.83-2.80 (m, 2H), 2.49-2.43 (m, 1H), 1.08-1.01 (m, 1H), 0.65-0.58 (m, 1H), 0.50-0.43 (m, 1H), 0.35-0.30 (m, 1H), 0.2-0.17 (m, 1H). |
| 6 | 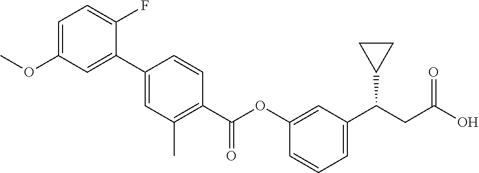 [SMILES: COc1ccc(F)c(-c2ccc(C(=O)Oc3cccc([C@@H](CC(=O)O)C4CC4)c3)c(C)c2)c1] | LCMS: tR = 1.012, (ES+) m/z (M + H)⁺ = 449.2. ¹H NMR (CDCl₃, 400 MHz) δ = 8.24 (d, J = 7.8 Hz, 1H), 7.54-7.48 (m, 2H), 7.42-7.36 (m, 1H), 7.17 (d, J = 7.8 Hz, 1H), 7.14-7.08 (m, 3H), 6.98 (m, 1H), 6.92-6.86 (m, 1H), 3.88-3.83 (m, 3H), 2.91-2.81 (m, 2H), 2.81-2.65 (m, 4H), 1.14-1.00 (m, 1H), 0.68-0.58 (m, 1H), 0.53-0.45 (m, 1H), 0.34 (m, 1H), 0.22 (m, 1H). |
| 9 | 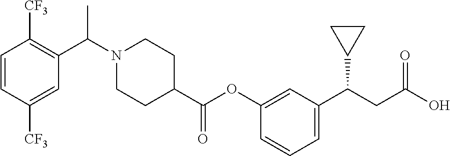 [SMILES: CC(c1cc(C(F)(F)F)ccc1C(F)(F)F)N1CCC(C(=O)Oc2cccc([C@@H](CC(=O)O)C3CC3)c2)CC1] | LCMS: tR = 0.880 min., (ES⁺) m/z (M + H)⁺ = 558.2. ¹H NMR (DMSO-d₆, 400 MHz,) δ = 8.16 (s, 1H), 7.97-7.81 (m, 2H), 7.33-7.27 (m, 1H), 7.14 (d, J = 7.6 Hz, 1H), 7.01-6.98 (m, 1H), 6.95-6.89 (m, 1H), 3.67 (d, J = 5.2 Hz, 1H), 3.19 (d, J = 10.4 Hz, 1H), 2.69-2.56 (m, 3H), 2.45 (d, J = 11.6 Hz, 1H), 2.34-2.25 (m, 1H), 2.16-1.99 (m, 3H), 1.91-1.67 (m, 2H), 1.65-1.51 (m, 1H), 1.28 (d, J = 6.4 Hz, 3H), 1.04-0.92 (m, 1H), 0.48 (m, 1H), 0.35-0.20 (m, 2H), 0.15-0.07 (m, 1H). |
| 12 | 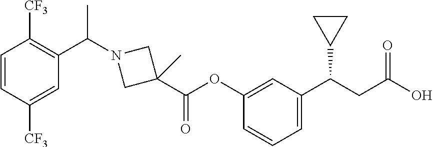 [SMILES: CC(c1cc(C(F)(F)F)ccc1C(F)(F)F)N1CC(C)(C(=O)Oc2cccc([C@@H](CC(=O)O)C3CC3)c2)C1] | LCMS: tR = 0.971 min, (ES+) m/z (M + H)⁺ = 544.2. ¹H NMR (400 MHz, DMSO-d₆) δ = 8.15 (s, 1H), 7.95 (d, J = 8.4 Hz, 1H), 7.85 (d, J = 8.4 Hz, 1H), 7.33 (t, J = 8.0 Hz, 1H), 7.17 (d, J = 7.6 Hz, 1H), 7.04 (s, 1H), 6.96 (d, J = 8.0 Hz, 1H), 3.85 (d, J = 5.2 Hz, 1H), 3.56 (d, J = 7.2 Hz, 1H), 3.43 (d, J = 6.8 Hz, 1H), 3.17 (d, J = 7.2 Hz, 1H), 3.03 (d, J = 7.2 Hz, 1H), 2.66 (s, 2H), 2.35-2.26 (m, 1H), 1.62 (s, 3H), 1.18 (d, J = 6.4 Hz, 3H), 1.06-0.94 (m, 1H), 0.54-0.45 (m, 1H), 0.36-0.21 (m, 2H), 0.13 (d, J = 4.8 Hz, 1H). |
| 13 | 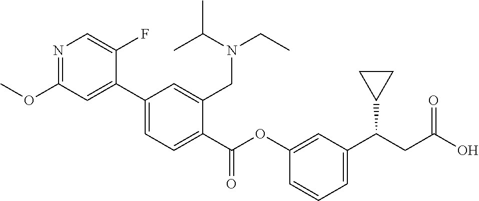 [SMILES: CCN(Cc1cc(-c2cc(OC)ncc2F)ccc1C(=O)Oc1cccc([C@@H](CC(=O)O)C2CC2)c1)C(C)C] | LCMS: tR = 0.873 min, (ES⁺) m/z (M + H)⁺ = 535.2. ¹H NMR (400 MHz, CDCl₃) δ = 8.23-8.15 (m, 2H), 8.14-8.11 (d, J = 2.4 Hz, 1H), 7.66-7.60 (d, J = 8.0 Hz, 1H), 7.40-7.33 (t, J = 8.0 Hz, 1H), 7.22-7.17 (d, J = 8.0 Hz, 1H), 7.17-7.13 (m, 1H), 7.11-7.04 (m, 1H), 6.97-6.91 (d, J = 5.2 Hz, 1H), 4.33-4.10 (m, 2H), 3.98 (s, 3H), 3.25-3.16 (m, 1H), 2.84-2.79 (q, J = 3.6 Hz, 2H), 2.71-2.62 (q, J = 7.2 Hz, 2H), 2.49-2.38 (m, 1H), 1.15-1.07 (d, J = 6.4 Hz, 6H), 1.08-1.00 (t, J = 7.2 Hz, 4H), 0.64-0.57 (m, 1H), 0.51-0.44 (m, 1H), 0.37-0.28 (m, 1H), 0.23-0.17 (m, 1H). |
| 14 | 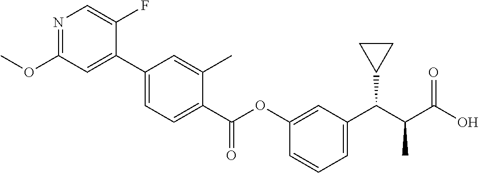 [SMILES: COc1cc(-c2ccc(C(=O)Oc3cccc([C@H](C4CC4)[C@H](C)C(=O)O)c3)c(C)c2)c(F)cn1] | LCMS: tR = 1.090 min., (ES+) m/z (M + H)⁺ = 464.2. ¹H NMR (400 MHz, CDCl₃) δ ppm 8.27 (d, J = 8.8 Hz, 1H), 8.11 (d, J = 2.0 Hz, 1H), 7.52-7.57 (m, 2H), 7.37-7.43 (m, 1H), 7.09-7.15 (m, 2H), 7.05-7.08 (m, 1H), 6.85 (d, J = 6.0 Hz, 1H), 3.97 (s, 3H), 2.91-2.87 (m, 1H), 2.75 (s, 3H), 2.12-2.17 (m, 1H), 1.17-1.14 (m, 1H), 1.06 (d, J = 6.8 Hz, 3H), 0.68-0.65 (m, 1H), 0.44-0.40 (m, 2H), 0.11-0.08 (m, 1H). |
| 17 | 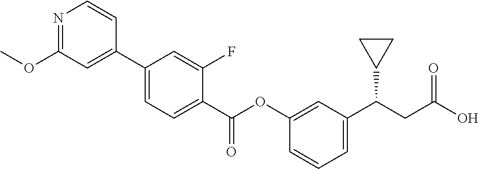 [SMILES: COc1cc(-c2ccc(C(=O)Oc3cccc([C@@H](CC(=O)O)C4CC4)c3)c(F)c2)ccn1] | LCMS: tR = 1.014 min, (ES⁺) m/z (M + H)⁺ = 436.1. ¹H- NMR (CDCl₃, 400 MHz): δ 8.43-8.42 (m, 1H), 8.30 (t, J = 8.0 Hz, 1H), 7.94-7.89 (m, 2H), 7.80-7.79 (m, 2H), 7.42-7.38 (m, 1H), 7.25 (d, J = 8 Hz, 1H), 7.19 (s, 1H), 7.13-7.10 (m, 1H), 4.29 (s, 3H), 2.83-2.73 (m, 2H), 2.44-2.38 (m, 1H), 1.14-1.06 (m, 1H), 0.65-0.58 (m, 1H), 0.46-0.42 (m, 1H), 0.36-0.32 (m, 1H), 0.21-0.18 (m, 1H). |
| 18 | 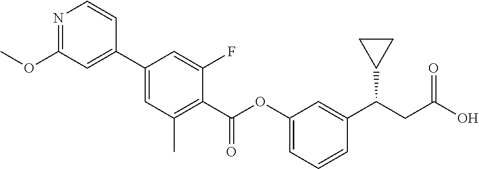 [SMILES: COc1cc(-c2cc(C)c(C(=O)Oc3cccc([C@@H](CC(=O)O)C4CC4)c3)c(F)c2)ccn1] | LCMS: tR = 1.001 min., (ES⁺) m/z (M + H)⁺ = 450.2, ¹H- NMR (DMSO-d₆, 400 MHz): δ 8.11 (d, J = 5.6 Hz, 1H), 7.57-7.54 (m, 2H), 7.26-7.22 (m, 2H), 7.09-7.07 (m, 2H), 7.03 (s, 1H), 6.97 (dd, J₁ = 8.0 Hz, J₂ = 2.0 Hz, 1H), 3.74 (s, 3H), 2.54-2.51 (m, 2H), 2.40 (s, 3H), 2.19-2.17 (m, 1H), 0.89-0.84 (m, 1H), 0.39-0.36 (m, 1H), 0.20-0.07 (m, 2H), 0.03--0.01 (m, 1H). |
| 19 | 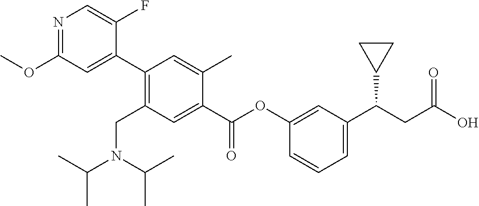 [SMILES: COc1cc(-c2cc(C)c(C(=O)Oc3cccc([C@@H](CC(=O)O)C4CC4)c3)cc2CN(C(C)C)C(C)C)c(F)cn1] | LCMS: tR = 0.807 min, (ES+) m/z (M + H)⁺ = 563.2. ¹H NMR (400 MHz, CDCl₃) δ 8.46 (s, 1H), 8.07 (s, 1H), 7.42-7.35 (m, 1H), 7.19-7.10 (m, 4H), 6.64 (d, J = 4.8 Hz, 1H), 3.98 (s, 3H), 3.56 (s, 2H), 2.99-2.96 (m, 2H), 2.84-2.82 (m, 2H), 2.67 (s, 3H), 2.50-2.44 (m, 1H), 1.10-1.07 (m, 1H), 0.94 (d, J = 6.4 Hz, 12 H), 0.64-0.61 (m, 1H), 0.50-0.48 (m, 1H), 0.36-0.33 (m, 1H), 0.24-0.22 (m, 1H). |
| 23 | 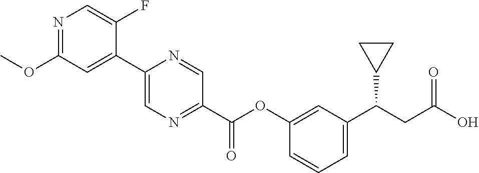 [SMILES: COc1cc(-c2cnc(C(=O)Oc3cccc([C@@H](CC(=O)O)C4CC4)c3)cn2)c(F)cn1] | LCMS: tR = 0.923 min, (ES⁺) m/z (M + H)⁺ = 438.2. ¹H- NMR (CDCl₃, 400 MHz): δ = 9.57 (s, 1H), 9.35 (s, 1H), 8.24 (d, J = 2.4 Hz, 1H), 7.51 (d, J = 4.8 Hz, 1H), 7.43 (d, J = 7.6 Hz, 1H), 7.24-7.19 (m, 3H), 4.01 (s, 3H), 2.86-2.82 (m, 2H), 2.49-2.46 (m, 1H), 1.09-1.06 (m, 1H), 0.66-0.63 (m, 1H), 0.52-0.49 (m, 1H), 0.36-0.34 (m, 1H), 0.24-0.20 (m, 1H). |
| 24 | 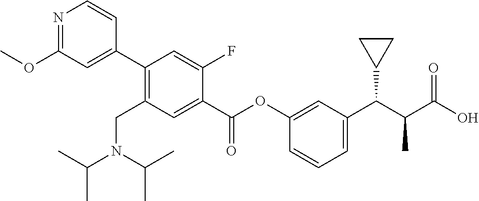 [SMILES: COc1cc(-c2cc(F)c(C(=O)Oc3cccc([C@H](C4CC4)[C@H](C)C(=O)O)c3)cc2CN(C(C)C)C(C)C)ccn1] | LCMS: tR = 0.830 min, (ES+) m/z (M + H)⁺ = 563.3. ¹H NMR (400 MHz, DMSO-d₆) δ 9.20 (s, 1H), 8.61 (d, J = 7.2, 1H), 7.59 (d, J = 7.2, 1H), 7.54 (d, J = 15.6, 1H), 7.45-7.41 (m, 1H), 7.26-7.21 (m, 2H), 6.47 (s, 1H), 6.30 (d, J = 6.4, 1H), 4.43 (d, J = 6.4, 1H), 3.63 (s, 6H), 2.91-2.86 (m, 1H), 2.04-1.91 (m, 1H), 1.29-1.18 (m, 12H), 1.13-1.10 (m, 1H), 0.88-0.84 (m, 3H), 0.54-0.53 (m, 1H), 0.30-0.28 (m, 1H), 0.18-0.16 (m, 1H), 0.01--0.04 (m, 1H). |
| 25 | 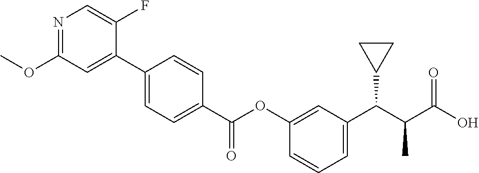 [SMILES: COc1cc(-c2ccc(C(=O)Oc3cccc([C@H](C4CC4)[C@H](C)C(=O)O)c3)cc2)c(F)cn1] | LCMS: tR = 1.053 min, (ES⁺) m/z (M + H)⁺ = 450.3. ¹H- NMR (CD₃Cl, 400 MHz): δ 8.31 (d, J = 8.4 Hz, 2H), 8.13 (d, J = 2 Hz, 1H), 7.73 (dd, J₁ = 8.4 Hz, J₂ = 1.2 Hz, 2H), 7.41-7.37 (m, 1H), 7.15-7.08 (m, 3H), 6.87 (d, J = 5.2 Hz, 1H), 3.97 (s, 3H), 2.92-2.84 (m, 1H), 2.10-2.09 (m, 1H), 1.20-1.11 (m, 1H), 1.05 (d, J = 6.8 Hz, 1H), 0.70-0.62 (m, 1H), 0.45-0.38 (m, 2H), 0.10-0.04 (m, 1H). |
| 27 | 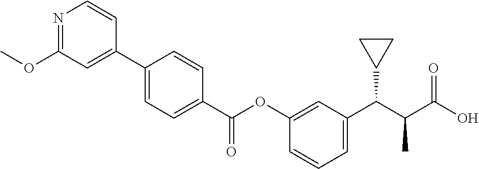 [SMILES: COc1cc(-c2ccc(C(=O)Oc3cccc([C@H](C4CC4)[C@H](C)C(=O)O)c3)cc2)ccn1] | LCMS: tR = 1.023 min., (ES⁺) m/z (M + H)⁺ = 432.2. ¹H- NMR (CDCl₃, 400 MHz): δ = 8.33-8.26 (m, 3H), 7.79-7.74 (m, 2H), 7.43-7.38 (m, 1H), 7.18-7.07 (m, 4H), 7.02 (s, 1H), 4.02 (s, 3H), 2.95-2.83 (m, 1H), 2.10 (t, J = 9.6 Hz, 1H), 1.15 (m, 1H), 1.06 (d, J = 7.2 Hz, 3H), 0.70-0.63 (m, 1H), 0.47-0.36 (m, 2H), 0.13-0.04 (m, 1H). |
| 28 | 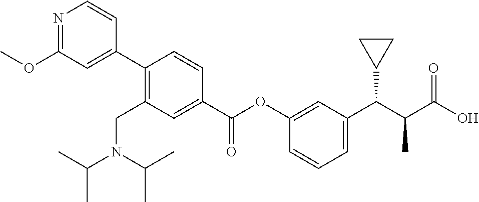 [SMILES: COc1cc(-c2ccc(C(=O)Oc3cccc([C@H](C4CC4)[C@H](C)C(=O)O)c3)cc2CN(C(C)C)C(C)C)ccn1] | LCMS: tR = 0.707 min., (ES⁺) m/z (M + H)⁺ = 545.2. ¹H- NMR (CDCl₃, 400 MHz): δ 8.61 (s, 1H), 8.24 (d, J = 5.2 Hz, 1H), 8.09 (dd, J₁ = 8.0 Hz, J₂ = 1.6 Hz, 1H), 7.39 (t, J = 8.4 Hz, 1H), 7.27-7.25 (m, 1H), 7.15-7.10 (m, 3H), 6.85 (dd, J = 1.2, 5.2 Hz, 1H), 6.71 (s, 1H), 4.02 (s, 3H), 3.60 (s, 2H), 3.00-2.86 (m, 3H), 2.09 (t, J = 10.0 Hz, 1H), 1.19-1.13 (m, 1H), 1.06 (d, J = 6.8 Hz, 3H), 0.95 (d, J = 6.4 Hz, 12H), 0.69-0.64 (m, 1H), 0.45-0.40 (m, 2H), 0.15-0.05 (m, 1H). |
| 29 | 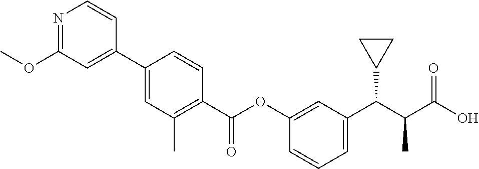 [SMILES: COc1cc(-c2ccc(C(=O)Oc3cccc([C@H](C4CC4)[C@H](C)C(=O)O)c3)c(C)c2)ccn1] | LCMS: tR = 0.876 min., (ES⁺) m/z (M + H)⁺ = 446.0. ¹H NMR (DMSO-d₆, 400 MHz,) δ = 12.31-12.12 (m, 1H), 8.29 (d, J = 5.6 Hz, 1H), 8.19 (d, J = 8.0 Hz, 1H), 7.89-7.78 (m, 2H), 7.47-7.33 (m, 2H), 7.26-7.21 (m, 1H), 7.21-7.13 (m, 3H), 3.92 (s, 3H), 2.79-2.73 (m, 1H), 2.68 (s, 3H), 2.12-1.98 (m, 1H), 1.18-1.09 (m, 1H), 0.85 (d, J = 6.8 Hz, 3H), 0.61-0.48 (m, 1H), 0.34-0.23 (m, 2H), 0.04--0.08 (m, 1H). |
| 30 | 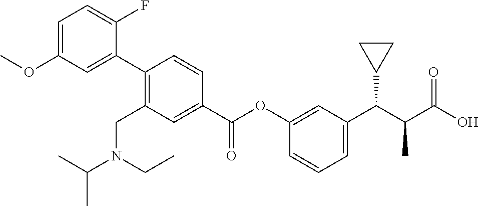 [SMILES: CCN(Cc1cc(C(=O)Oc2cccc([C@H](C3CC3)[C@H](C)C(=O)O)c2)ccc1-c1cc(OC)ccc1F)C(C)C] | LCMS: tR = 0.858 min, (ES+) m/z (M + H)⁺ = 548.3. ¹H NMR (400 MHz, CDCl₃) δ: 9.61-9.52 (m, 1H), 8.66 (s, 1H), 8.28 (d, J = 7.6 Hz, 1H), 7.64 (d, J = 8.0 Hz, 1H), 7.43 (t, J = 8.0 Hz, 1H), 7.3-7.25 (m, 1H), 7.255-7.0 (m, 5H), 4.49-4.46 (m, 1H), 4.16-4.13 (m, 1H), 3.82 (s, 3H), 3.15-2.98 (m, 2H), 2.90-2.76 (m, 1H), 2.68-2.67 (m, 1H), 2.15-2.05 (m, 1H), 1.24-1.15 (m, 6H), 1.07-1.06 (m, 3H), 0.88-0.81 (m, 5H), 0.57-0.56 (m, 1H), 0.33-0.29 (m, 2H), 0.02-0.01 (m, 1H). |
| 32 | 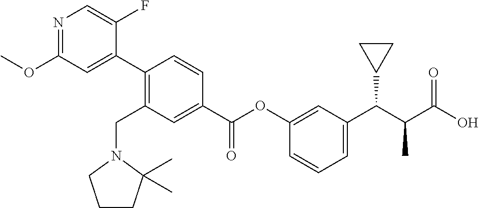 [SMILES: COc1cc(-c2ccc(C(=O)Oc3cccc([C@H](C4CC4)[C@H](C)C(=O)O)c3)cc2CN2CCCC2(C)C)c(F)cn1] | LCMS: tR = 0.698 min., (ES⁺) m/z (M + H)⁺ = 561.3. ¹H NMR (400 MHz, CD₃OD) δ = 8.48 (s, 1H), 8.34 (d, J₁= 8.0 Hz, J₂ = 1.6 Hz, 1H), 8.20 (s, 1H), 7.60 (d, J = 8.0 Hz, 1H), 7.44-7.40 (m, 1H), 7.19-7.13 (m, 3H), 6.91 (d, J = 4.8 Hz, 1H), 4.15-4.04 (m, 2H), 3.96 (s, 3H), 3.14-3.03 (m, 2H), 2.84-2.80 (m, 1H), 2.15-2.01 (m, 1H), 1.96-1.82 (m, 4H), 1.16 (s, 7H), 0.96 (d, J = 6.8 Hz, 3H), 0.68-0.59 (m, 1H), 0.44-0.31 (m, 2H), 0.08-0.01 (m, 1H). |
| 37 | 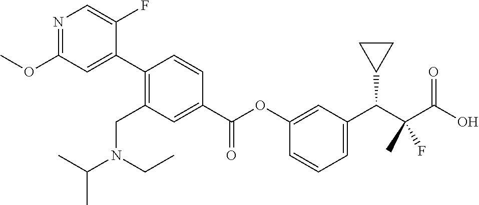 [SMILES: CCN(Cc1cc(C(=O)Oc2cccc([C@H](C3CC3)[C@@](C)(F)C(=O)O)c2)ccc1-c1cc(OC)ncc1F)C(C)C] | LCMS: tR = 0.866 min, (ES⁺) m/z (M + H)⁺ = 567.3. ¹H NMR (400 MHz, CDCl3) δ = 8.73 (s, 1H), 8.21 (dd, J₁ = 8.0 Hz, J₂ = 1.6 Hz, 2H), 8.13 (s, 1H), 7.38 (d, J = 8.0 Hz, 1H), 7.32 (t, J = 8.0 Hz, 1H), 7.22 (d, J = 8.0 Hz, 1H), 7.10~7.07 (m, 2H), 6.68 (d, J = 4.8 Hz, 1H), 4.03 (s, 2H), 3.99 (s, 3H), 3.36~3.33 (m, 1H), 2.70~2.68 (m, 2H), 2.46~2.36 (m, 1H), 1.45~1.42 (m, 1H), 1.20 (d, J = 21.2 Hz, 3H), 1.11~1.04 (m, 9H), 0.57~0.48 (m, 2H), 0.33~0.31 (m, 1H), 0.04~-0.08 (m, 1H). |
| 38 | 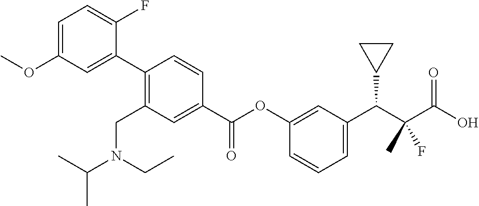 [SMILES: CCN(Cc1cc(C(=O)Oc2cccc([C@H](C3CC3)[C@@](C)(F)C(=O)O)c2)ccc1-c1cc(OC)ccc1F)C(C)C] | LCMS: tR = 0.865 min, (ES⁺) m/z (M + H)⁺ = 566.3. ¹H NMR (400 MHz, CDCl3) δ = 8.77 (s, 1H), 8.21 (d, J = 8.0 Hz, 1H), 7.43 (d, J = 8.0 Hz, 1H), 7.31 (t, J = 8.0 Hz, 1H), 7.26~7.22 (m, 1H), 7.16 (t, J = 8.8 Hz, 1H), 7.07~6.98 (m, 3H), 6.83-6.82 (m, 1H), 4.31~4.12 (m, 2H), 3.87 (s, 3H), 3.55~3.53 (m, 1H), 2.95~2.70 (m, 2H), 2.49~2.39 (m, 1H), 1.44~1.43 (m, 1H), 1.19~1.14 (m, 12H), 0.55~0.52 (m, 2H), 0.31~0.29 (m, 1H), −0.06~-0.08 (m, 1H). |
| 39 | 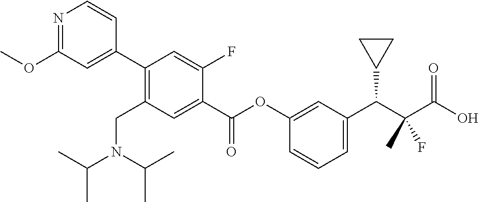 [SMILES: COc1cc(-c2cc(F)c(C(=O)Oc3cccc([C@H](C4CC4)[C@@](C)(F)C(=O)O)c3)cc2CN(C(C)C)C(C)C)ccn1] | LCMS: tR = 0.833 min, (ES⁺) m/z (M + H)⁺ = 581.3. ¹H NMR (400 MHz, DMSO-d₆) δ = 8.63~8.04 (m, 2H), 7.73~6.63 (m, 7H), 3.92 (s, 3H), 3.58~3.57 (m, 2H), 2.90~2.87 (m, 1H), 2.39~2.27 (m, 2H), 1.29~1.14 (m, 4H), 0.89-0.86 (m, 12H), 0.52-0.50 (m, 1H), 0.42-0.39 (m, 2H), 0.10~-0.10 (m, 1H). |
| 50 | 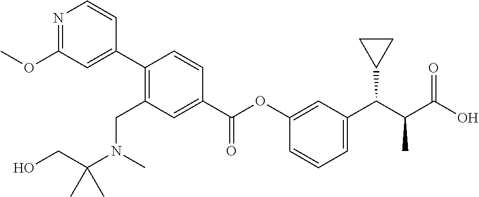 | LCMS: tR = 0.678 min, (ES+) m/z (M + H)+ = 565.2. ¹H NMR (400 MHz, CDCl₃-d6) δ = 8.42 (s, 1H), 8.19 (dd, J₁ = 4.0 Hz, J₂ = 1.6 Hz, 1H), 8.11 (s, 1H), 7.42-7.34 (m, 2H), 7.17 (dd, J₁ = 8.4 Hz, J₂ = 1.6 Hz 1H), 7.12 (d, J = 7.6 Hz, 1H), 7.08 (s, 1H), 6.67 (d, J = 4.8 Hz, 1H), 3.99 (s, 3H), 3.69 (br s, 2H), 3.41 (s, 2H), 2.89 (m, 1H), 2.13 (t, J = 10 Hz, 1H), 2.06 (s, 3H), 1.23-1.14 (m, 1H), 1.06 (s, 3H), 1.04 (s, 6H), 0.66 (m, 1H), 0.47-0.36 (m, 2H), 0.13-0.04 (m, 1H). |
| 51 | 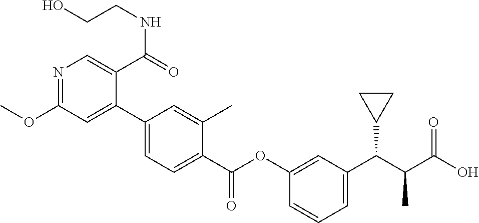 [SMILES: COc1cc(-c2ccc(C(=O)Oc3cccc([C@H](C4CC4)[C@H](C)C(=O)O)c3)c(C)c2)c(C(=O)NCCO)cn1] | LCMS: tR = 0.834 min., (ES⁺) m/z (M + H)⁺ = 533.3. ¹H- NMR (CD₃OD, 400 MHz): δ 8.48 (s, 1H), 8.23 (d, J = 7.6 Hz, 1H), 7.52-7.50 (m, 2H), 7.44-7.38 (m, 1H), 7.24 (s, 1H), 7.16-7.10 (m, 3H), 4.16 (s, 3H), 3.60-3.57 (m,, 2H), 3.39-3.36 (m, 2H), 2.86-2.80 (m, 1H), 2.71 (s, 3H), 2.09-2.03 (m, 1H), 1.17-1.11 (m, 1H), 0.97 (d, J = 6.8 Hz, 3H), 0.65-0.61 (m, 1H), 0.41-0.33 (m, 2H), 0.07-0.01 (m, 1H). |
| 52 | 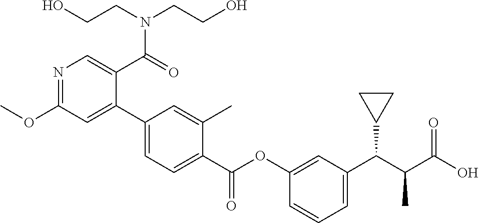 [SMILES: COc1cc(-c2ccc(C(=O)Oc3cccc([C@H](C4CC4)[C@H](C)C(=O)O)c3)c(C)c2)c(C(=O)N(CCO)CCO)cn1] | LCMS: tR = 0.805 min., (ES⁺) m/z (M + H)⁺ = 577.4. ¹H- NMR (CD₃OD, 400 MHz): δ 8.25-8.21 (m, 2H), 7.50-7.49 (m, 2H), 7.42-7.38 (m, 1H), 7.16-7.10 (m, 3H), 6.92 (s, 1H), 4.00 (s, 3H), 3.81-3.41 (m, 8H), 2.86-2.78 (m, 1H), 2.70 (s, 3H), 2.09-2.03 (m, 1H), 1.19-1.11 (m, 1H), 0.97 (d, J = 7.2 Hz, 3H), 0.65-0.61 (m, 1H), 0.40-0.35 (m, 2H), 0.05-0.02 (m, 1H). |
| 53 | 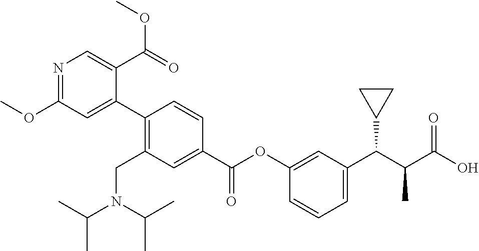 [SMILES: COC(=O)c1cnc(OC)cc1-c1ccc(C(=O)Oc2cccc([C@H](C3CC3)[C@H](C)C(=O)O)c2)cc1CN(C(C)C)C(C)C] | LCMS: tR = 0.795 min., (ES⁺) m/z (M + H)⁺ = 603.4. ¹H- NMR (CD₃OD, 400 MHz): δ 8.86 (s, 1H), 8.5 (s, 1H), 8.12 (d, J = 7.6 Hz, 1H), 7.43-7.38 (m, 1H), 7.26-7.24 (m, 1H), 7.17-7.11 (m, 3H), 6.72 (s, 1H), 4.05 (s, 3H), 3.68 (s, 5H), 3.17 (s, 2H), 2.85-2.78 (m, 1H), 2.10-2.05 (m, 1H), 1.18-1.14 (m, 1H), 1.12-0.96 (m, 15H), 0.65-0.61 (m, 1H), 0.40-0.35 (m, 2H), 0.05-0.01 (m, 1H). |
| 62 | 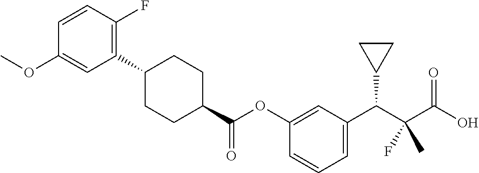 [SMILES: COc1ccc(F)c([C@H]2CC[C@H](C(=O)Oc3cccc([C@H](C4CC4)[C@@](C)(F)C(=O)O)c3)CC2)c1] | LCMS: tR = 0.873 min, (ES⁺) m/z (M + H₂O) = 490.0. ¹H- NMR (CD₃OD, 400 MHz): δ = 7.35 (t, J = 8 Hz, 1H), 7.18 (d, J = 7.2 Hz, 1H), 7.07-6.98 (m, 2H), 6.94 (t, J = 9.2 Hz, 1H), 6.82 (dd, J₁ = 3.2 Hz, J₂ = 6 Hz, 1H), 6.75-6.70 (m, 1H), 3.76 (s, 3H), 2.94-2.80 (m, 1H), 2.73-2.60 (m, 1H), 2.40-2.23 (m, 3H), 1.96 (d, J = 11.2 Hz, 2H), 1.79-1.57 (m, 4H), 1.47-1.36 (m, 1H), 1.35-1.23 (m, 3H), 0.70-0.58 (m, 1H), 0.50-0.35 (m, 2H), 0.05--0.08 (m, 1H). |

### Example 19: (S)-3-cyclopropyl-3-(3-(((2′-fluoro-5′-methoxy-3-methyl-[1,1′-biphenyl]-4-yl) oxy)carbonyl)phenyl)propanoic acid (Compound 45)

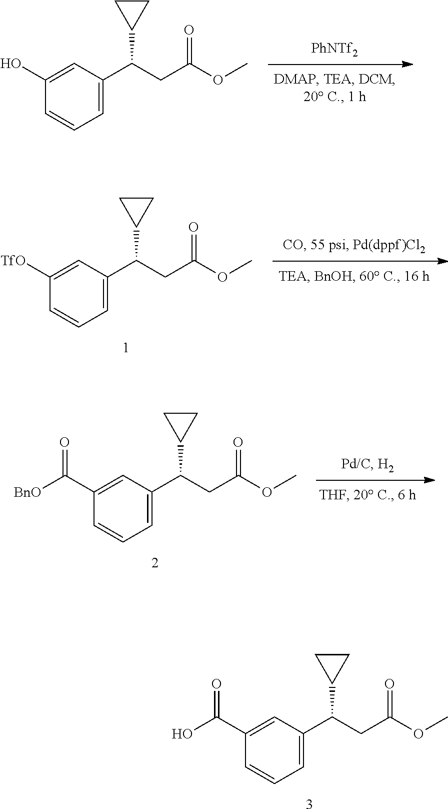

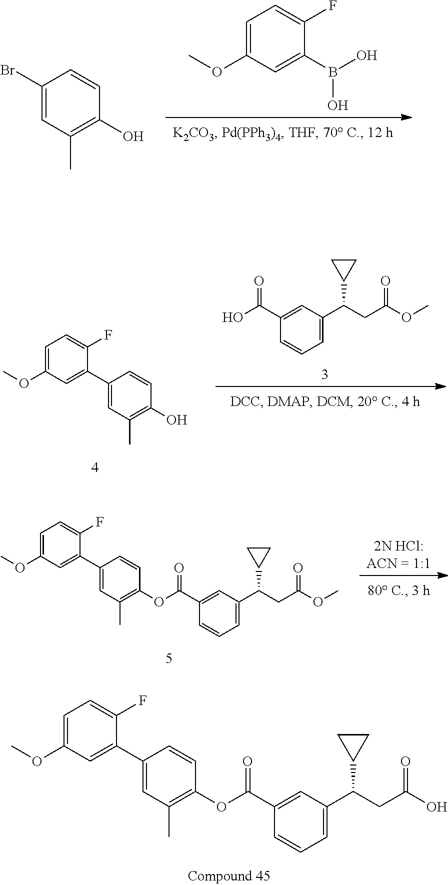

Step 1: (S)-methyl 3-cyclopropyl-3-(3-(((trifluoromethyl)sulfonyl)oxy)phenyl)propanoate (1): To a solution of methyl (S)-methyl 3-cyclopropyl-3-(3-hydroxyphenyl)propanoate (0.30 g, 1.4 mmol, 1 eq) in DCM (4 mL) was added DMAP (17 mg, 0.14 mmol, 0.1 eq) and TEA (0.28 mg, 2.7 mmol, 2 eq). The mixture was stirred at 20° C. for 0.5 hr, then 1,1,1-trifluoro-N-phenyl-N-(trifluoromethylsulfonyl) methanesulfonamide (0.58 g, 1.6 mmol, 1.2 eq) was added to the mixture. The mixture was stirred at 20° C. for 0.5 hr, then concentrated to give a residue. The residue was purified by column chromatography (SiO₂, Petroleum ether:Ethyl acetate=100:1 to 20:1), to give 1 (0.48 g, 91.% yield) as a colorless oil. LCMS: tR=0.875, (ES⁺) m/z (M+H)⁺=353.2. ¹H NMR (400 MHz, CDCl₃) δ=7.41-7.34 (m, 1H), 7.25 (s, 1H), 7.16-7.09 (m, 2H), 3.59 (s, 3H), 2.83-2.65 (m, 2H), 2.47-2.34 (m, 1H), 1.04-0.93 (m, 1H), 0.67-0.56 (m, 1H), 0.52-0.40 (m, 1H), 0.28 (m, 1H), 0.13 (m, 1H).

Step 2: (S)-benzyl 3-(1-cyclopropyl-3-methoxy-3-oxopropyl)benzoate (2): To a solution of 1 (0.30 g, 0.85 mmol, 1 eq) in phenylmethanol (2 mL) was added TEA (0.34 g, 3.4 mmol, 4 eq), Pd(dppf)Cl₂ (62 mg, 85 μmol, 0.1 eq). The mixture was stirred at 60° C. for 16 hrs under CO with 55 psi. The mixture was concentrated to give a residue. The residue was purified by prep-TLC (SiO₂, PE:EA=10:1). to give 2 (0.12 g, 32% yield) as a colorless oil. LCMS: tR=0.979, (ES+) m/z (M+H)⁺=339.1.

Step 3: (S)-3-(1-cyclopropyl-3-methoxy-3-oxopropyl)benzoic acid (3): To a solution of 2 (0.10 g, 0.30 mmol, 1 eq) in THF (1 mL) was added 10% Pd/C (5 mg). The mixture was stirred at 20° C. for 6 hr under H₂. The mixture was filtered and concentrated under reduced pressure to give 3 (80 mg, crude) as a colorless oil.

Step 4: 2′-fluoro-5′-methoxy-3-methyl-[1,1′-biphenyl]-4-ol (4): To a solution of 4-bromo-2-methyl-phenol (0.50 g, 2.7 mmol) in THE (10 mL) as added (2-fluoro-5-methoxy-phenyl)boronic acid (0.50 g, 2.9 mmol) and K₂CO₃ (2 M, 7 mL). The reaction mixture was purged with nitrogen and then Pd(PPh₃)₄ (0.15 g, 0.13 mmol) was added. The reaction mixture was heated at 70° C. for 12 hours. The reaction mixture was diluted with H₂O (30 mL) and extracted with ethyl acetate (50 mL×3). The combined organic layers were washed with brine (50 mL×3), dried over Na₂SO₄, filtered and concentrated under reduced pressure to give a residue. The residue was purified by column chromatography (SiO₂, Petroleum ether:Ethyl acetate=20:1 to 3:1) to give 4 (0.58 g, 90% yield) as a brown oil. ¹H NMR (400 MHz, CDCl₃) δ 7.35 (s, 1H), 7.28-7.32 (m, 1H), 7.04-7.09 (m, 1H), 6.93 (dd, J₁=3.2 Hz, J₂=6.4 Hz, 1H), 6.87 (d, J=8.4 Hz, 1H), 6.81 (m, 1H), 5.09 (s, 1H), 3.85 (s, 3H), 2.33 (s, 3H).

Step 5: (S)-2′-fluoro-5′-methoxy-3-methyl-[1,1′-biphenyl]-4-yl3-(1-cyclopropyl-3-methoxy-3-oxopropyl)benzoate (5): To a solution of (S)-3-(1-cyclopropyl-3-methoxy-3-oxopropyl)benzoic acid (0.11 g, 0.43 mmol) in DCM (2 mL) was slowly added 4 (0.10 g, 0.43 mmol), DCC (0.13 mg, 0.65 mmol) and DMAP (26 mg, 0.22 mmol) under N₂ atmosphere. The mixture was stirred at 20° C. for 4 hours. The reaction mixture was concentrated under reduced pressure to remove DCM. The residue was diluted with H₂O (20 mL) and extracted with ethyl acetate (20 mL×3). The combined organic layers were washed with saturated brine (20 mL×3), dried over Na₂SO₄, filtered and concentrated under reduced pressure to give a residue. The residue was purified by prep-TLC (Petroleum ether:Ethyl acetate=3:1) to give 5 (0.12 g, 60% yield) as a white oil. ¹H NMR (400 MHz, CDCl₃) δ 8.04 (m, 2H), 7.45-7.49 (m, 1H), 7.34-7.43 (m, 3H), 7.15 (d, J=8.4 Hz, 1H), 6.97-7.03 (m, 1H), 6.88 (dd, J₁=3.2 Hz, J₂=6.4 Hz, 1H), 6.76 (m, 1H), 3.76 (s, 3H), 3.55 (s, 3H), 2.68-2.82 (m, 2H), 2.38-2.45 (m, 1H), 2.23 (s, 3H), 0.99-1.07 (m, 1H), 0.52-0.63 (m, 1H), 0.36-0.45 (m, 1H), 0.25 (m, 1H), 0.08-0.18 (m, 1H).

Step 6: (S)-3-cyclopropyl-3-(3-(((2′-fluoro-5′-methoxy-3-methyl-[1,1′-biphenyl]-4-yl) oxy)carbonyl)phenyl)propanoic acid (Compound 45): A solution of 5 (60 mg, 0.13 mmol) in ACN (0.6 mL) and HCl (2 M, 0.6 mL) was stirred at 80° C. for 3 hours. The reaction mixture was concentrated under reduced pressure to remove ACN and HCl. The residue was purified by prep-HPLC (column: Phenomenex Synergi C18 150×25 mm×10 μm; mobile phase: [water (0.05% HCl)-ACN]; B %: 55%-75%, 9 min) to give Compound 45 (14 mg, 23% yield) as a yellow gum. LCMS: tR=1.026, (ES+) m/z (M−H)⁺=447.1. ¹H NMR (400 MHz, CDCl₃) δ 7.87-7.94 (m, 2H), 7.31-7.36 (m, 1H), 7.19-7.29 (m, 3H), 7.01 (d, J=8.4 Hz, 1H), 6.86 (t, J=9.2 Hz, 1H), 6.75 (dd, J₁=3.2 Hz, J₂=6.0 Hz, 1H), 6.59-6.65 (m, 1H), 3.62 (s, 3H), 2.58-2.74 (m, 2H), 2.24-2.32 (m, 1H), 2.09 (s, 3H), 0.85-0.96 (m, 1H), 0.40-0.49 (m, 1H), 0.24-0.32 (m, 1H), 0.15 (m, 1H), −0.05-0.05 (m, 1H).

The following compounds were prepared according to the procedures described in Example 19 using the appropriate intermediates.

| Cpd | Structure | Characterization Data |
|---|---|---|
| 41 | 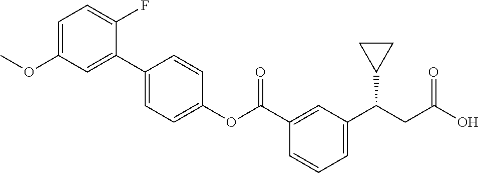 [SMILES: COc1ccc(F)c(-c2ccc(OC(=O)c3cccc([C@@H](CC(=O)O)C4CC4)c3)cc2)c1] | LCMS: tR = 1.022, (ES−) m/z (M − H)⁻ = 433.1. ¹H NMR (400 MHz, CDCl₃) δ = 8.14-8.08 (m, 2H), 7.62 (d, J = 7.8 Hz, 2H), 7.56-7.44 (m, 2H), 7.31 (d, J = 8.6 Hz, 2H), 7.09 (t, J = 9.6 Hz, 1H), 6.97 (m, 1H), 6.85 (m, 1H), 3.84 (s, 3H), 2.94-2.78 (m, 2H), 2.55-2.41 (m, 1H), 1.11 (m, 1H), 0.70-0.59 (m, 1H), 0.52-0.43 (m, 1H), 0.35 (m, 1H), 0.20 (m, 1H). |
| 43 | 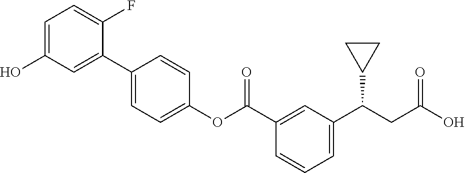 [SMILES: O=C(O)C[C@H](c1cccc(C(=O)Oc2ccc(-c3cc(O)ccc3F)cc2)c1)C1CC1] | LCMS: tR = 4.739, (ES−) m/z (M − H)⁻ = 419.1. ¹H NMR (400 MHz, CDCl₃) δ = 8.16-8.00 (m, 2H), 7.61-7.49 (m, 3H), 7.48-7.40 (m, 1H), 7.25 (s, 2H), 7.00 (t, J = 9.6 Hz, 1H), 6.87 (m, 1H), 6.74 (m, 1H), 2.94-2.75 (m, 2H), 2.52-2.39 (m, 1H), 1.16-1.02 (m, 1H), 0.70-0.56 (m, 1H), 0.51-0.41 (m, 1H), 0.33 (m, 1H), 0.18 (m, 1H). |
| 44 | 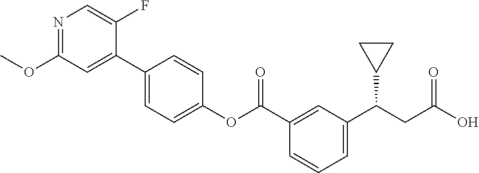 [SMILES: COc1cc(-c2ccc(OC(=O)c3cccc([C@@H](CC(=O)O)C4CC4)c3)cc2)c(F)cn1] | LCMS: tR = 0.953 min, (ES⁺) m/z (M + H)⁺ = 436.0, ¹H- NMR (CDCl₃; 400 MHz): δ 8.10-8.08 (m, 3H), 7.67-7.65 (m, 2H), 7.55-7.45 (m, 2H), 7.36-7.32 (m, 2H), 6.84 (d, J = 5.4 Hz, 1H), 3.96 (s, 3H), 2.93-2.80 (m, 2H), 2.51-2.45 (m, 1H), 1.14-1.01 (m, 1H), 0.69-0.62 (m, 1H), 0.52-0.45 (m, 1H), 0.37-0.32 (m, 1H), 0.23-0.18 (m, 1H). |
| 54 | 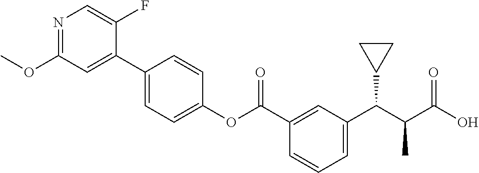 [SMILES: COc1cc(-c2ccc(OC(=O)c3cccc([C@H](C4CC4)[C@H](C)C(=O)O)c3)cc2)c(F)cn1] | LCMS: tR = 0.857 min., (ES⁺) m/z (M + H)⁺ = 450.0. ¹H- NMR (CDCl₃, 400 MHz): δ = 8.13-8.10 (m, 2H), 8.05 (s, 1H), 7.67 (dd, J₁ = 8.4 Hz, J₂ = 1.6 Hz, 2H), 7.53-7.47 (m, 2H), 7.38-7.33 (m, 2H), 6.85 (d, J = 5.6 Hz, 1H), 3.97 (s, 3H), 3.01-2.88 (m, 1H), 2.17 (t, J = 9.6 Hz, 1H), 1.30-1.17 (m, 1H), 1.05 (d, J = 7.2 Hz, 3H), 0.76-0.66 (m, 1H), 0.51-0.37 (m, 2H), 0.13-0.01 (m, 1H). |
| 55 | 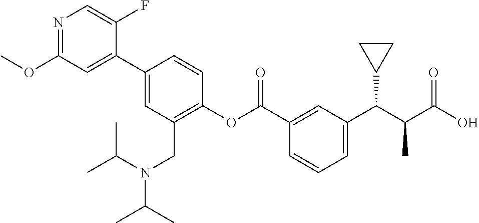 [SMILES: COc1cc(-c2ccc(OC(=O)c3cccc([C@H](C4CC4)[C@H](C)C(=O)O)c3)c(CN(C(C)C)C(C)C)c2)c(F)cn1] | LCMS: tR = 0.699 min., (ES⁺) m/z (M + H)⁺ = 563.2. ¹H- NMR (CD₃OD, 400 MHz): δ 8.16-8.09 (m, 3H), 7.95 (s, 1H), 7.75 (d, J = 8.4 Hz, 1H), 7.64-7.57 (m, 2H), 7.46 (d, J = 8.4 Hz, 1H), 6.99 (d, J = 5.2 Hz, 1H), 4.17 (s, 2H), 3.95 (s, 3H), 3.57-3.54 (m, 2H), 2.90-2.87 (m, 1H), 2.17 (t, J = 10 Hz, 1H), 1.43-1.42 (m, 1H), 1.25 (d, J = 6.4 Hz, 12H), 0.96 (d, J = 6.8 Hz, 3H), 0.73-0.63 (m, 1H), 0.47-0.32 (m, 2H), 0.08--0.05 (m, 1H). |
| 56 | 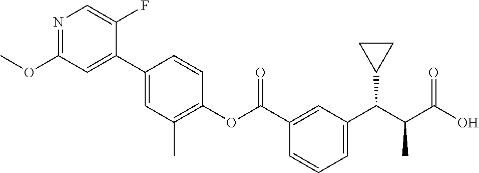 [SMILES: COc1cc(-c2ccc(OC(=O)c3cccc([C@H](C4CC4)[C@H](C)C(=O)O)c3)c(C)c2)c(F)cn1] | LCMS: tR = 1.079 min., (ES⁺) m/z (M + H)⁺ = 464.2. ¹H- NMR (CD₃OD, 400 MHz): δ 8.10-8.00 (m, 3H), 7.60-7.46 (m, 4H), 7.30-7.22 (m, 1H), 6.93-6.83 (m, 1H), 3.92-3.88 (m, 3H), 2.89-2.80 (m, 1H), 2.26 (s, 3H), 2.13 (t, J = 9.6 Hz, 1H), 1.27-1.08 (m, 1H), 0.94 (d, J = 6.8 Hz, 3H), 0.68-0.60 (m, 1H), 0.43-0.31 (m, 2H), 0.03--0.04 (m, 1H). |
| 57 | 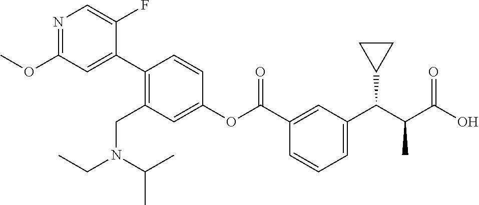 [SMILES: CCN(Cc1cc(OC(=O)c2cccc([C@H](C3CC3)[C@H](C)C(=O)O)c2)ccc1-c1cc(OC)ncc1F)C(C)C] | LCMS: tR = 0.863 min., (ES⁺) m/z (M + H)⁺ = 549.3. ¹H- NMR (CD₃OD, 400 MHz): δ 8.14 (d, J = 0.8 Hz, 1H), 8.09-8.05 (m, 2H), 7.61-7.51 (m, 3H), 7.45-7.39 (m, 2H), 6.84 (d, J = 4.8 Hz, 1H), 3.95 (s, 5H), 3.27-3.20 (m, 1H), 2.87-2.73 (m, 3H), 2.16 (t, J = 10 Hz, 1H), 1.24-1.17 (m, 1H), 1.06-1.03 (m, 9H), 0.95 (d, J = 6.8 Hz, 3H), 0.69-0.63 (m, 1H), 0.46-0.32 (m, 2H), 0.03--0.02 (m, 1H). |
| 58 | 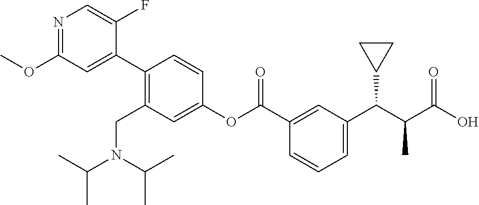 [SMILES: COc1cc(-c2ccc(OC(=O)c3cccc([C@H](C4CC4)[C@H](C)C(=O)O)c3)cc2CN(C(C)C)C(C)C)c(F)cn1] | LCMS: tR = 0.887 min., (ES⁺) m/z (M + H)⁺ = 563.4. ¹H- NMR (CD₃OD, 400 MHz): δ 8.10-8.01 (m, 3H), 7.61-7.48 (m, 3H), 7.29-7.19 (m, 2H), 6.74 (d, J = 4.8 Hz, 1H), 3.94 (s, 3H), 3.66 (s, 2H), 3.06-3.03 (m, 2H), 2.88-2.83 (m, 1H), 2.16 (t, J = 9.6 Hz, 1H), 1.30-1.15 (m, 1H), 0.97-0.95 (m, 15H), 0.68-0.60 (m, 1H), 0.45-0.31 (m, 2H), 0.03--0.04 (m, 1H). |

### Example 20: (3S)-3-(3-(((1-(1-(2,5-bis(trifluoromethyl)phenyl)ethyl)piperidin-4-yl)oxy)carbonyl)phenyl)-3-cyclopropylpropanoic acid (Compound 46)

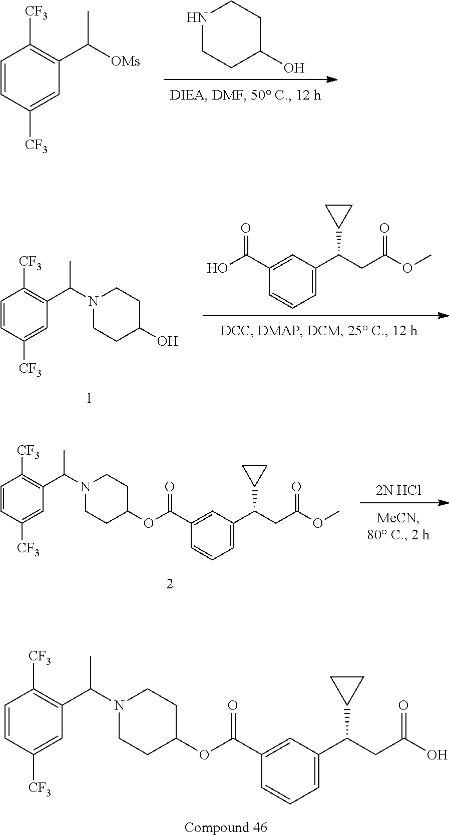

Step 1: 1-(1-(2,5-bis(trifluoromethyl)phenyl)ethyl)piperidin-4-ol (1): To a solution of 1-(2,5-bis(trifluoromethyl)phenyl)ethyl methanesulfonate (0.15 g, 0.45 mmol) in DMF (3 mL) was added DIEA (0.29 g, 2.2 mmol) and piperidin-4-ol (0.14 g, 1.3 mmol). The mixture was stirred at 50° C. for 12 h. The residue was quenched by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by column chromatography (SiO2, Petroleum ether:Ethyl acetate=30:1 to 10:1) to give 1 (0.70 g, 46% yield) as a yellow oil. ¹H NMR (400 MHz, CDCl₃) δ=8.20 (s, 1H), 7.73 (d, J=8.4 Hz, 1H), 7.58 (d, J=8.4 Hz, 1H), 3.78-3.66 (m, 2H), 3.03 (s, 1H), 2.53-2.43 (m, 1H), 2.24-2.07 (m, 2H), 1.94 (dd, J₁=3.2 Hz, J₂=3.2 Hz, 1H), 1.80 (dd, J₁=3.2 Hz, J₂=3.2 Hz, 1H), 1.67-1.58 (m, 2H), 1.57-1.44 (m, 2H), 1.29 (d, J=6.4 Hz, 3H).

Step 2: 1-(1-(2,5-bis(trifluoromethyl)phenyl)ethyl)piperidin-4-yl 3-((S)-1-cyclopropyl-3-methoxy-3-oxopropyl)benzoate (2): To a solution of 1 (70 mg, 0.21 mmol) in DCM (1 mL) was added DCC (64 mg, 0.31 μmol), DMAP (16 mg, 0.10 mmol) and (S)-3-(1-cyclopropyl-3-methoxy-3-oxopropyl)benzoic acid (51 mg, 0.21 mmol). The mixture was stirred at 25° C. for 12 h. The residue was quenched by H₂O (40 mL) and extracted with ethyl acetate (50 mL×3). The combined organic layers were washed with saturated brine (40 mL×2), dried over anhydrous Na₂SO₄, filtered and concentrated under reduced pressure to give a residue. The residue was purified by prep-TLC (SiO₂, PE:EA=5:1) to give 2 (70 mg, 59% yield) as a yellow oil. ¹H NMR (400 MHz, CDCl₃) δ=8.23 (s, 1H), 7.94-7.89 (m, 2H), 7.74 (d, J=8.4 Hz, 1H), 7.60 (d, J=8.0 Hz, 1H), 7.47-7.36 (m, 2H), 5.06 (d, J=3.6 Hz, 1H), 3.78 (d, J=6.0 Hz, 1H), 3.61 (s, 3H), 2.96 (s, 1H), 2.86-2.71 (m, 2H), 2.58-2.39 (m, 3H), 2.31-2.22 (m, 1H), 2.04-1.91 (m, 2H), 1.90-1.75 (m, 2H), 1.32 (d, J=6.4 Hz, 3H), 1.11-1.01 (m, 1H), 0.66-0.57 (m, 1H), 0.49-0.40 (m, 1H), 0.30 (d, J=4.8, 9.6 Hz, 1H), 0.15 (d, J=5.2 Hz, 1H).

Step 3: (3S)-3-(3-(((1-(1-(2,5-bis(trifluoromethyl)phenyl)ethyl)piperidin-4-yl)oxy)carbonyl)phenyl)-3-cyclopropylpropanoic acid (Compound 46): To a solution of 2 (60 mg, 0.11 mmol) in MeCN (1.2 mL) was added HCl (2 M, 1.2 mL). The mixture was stirred at 80° C. for 2 h. The reaction mixture was filtered to give a residue. The residue was purified by prep-HPLC (HCl condition; column: Phenomenex Synergi C18 150×25 mm×10 μm; mobile phase: [water (0.05% HCl)-ACN]; B %: 27%-47%, 10 min) to give Compound 46 (51 mg, 80% yield, HCl salt) as a white solid. LCMS: tR=0.838, (ES+) m/z (M+H)⁺=558.2. ¹H NMR (400 MHz, DMSO-d₆) δ=12.21-12.00 (m, 1H), 9.34-9.19 (m, 1H), 8.13-8.01 (m, 2H), 7.95-7.76 (m, 2H), 7.60 (d, J=6.0 Hz, 1H), 7.52-7.42 (m, 1H), 5.30-5.09 (m, 1H), 4.78-4.46 (m, 1H), 4.07-3.86 (m, 1H), 3.07-2.56 (m, 5H), 2.48-1.92 (m, 5H), 1.78 (s, 3H), 1.05 (s, 1H), 0.53 (s, 1H), 0.31 (d, J=6.0 Hz, 2H), 0.21-0.06 (m, 1H).

The following compounds were prepared according to the procedures described in Example 20 using the appropriate intermediates.

| Cpd | Structure | Characterization Data |
|---|---|---|
| 47 | 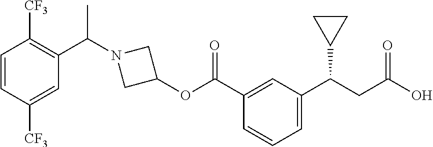 [SMILES: CC(c1cc(C(F)(F)F)ccc1C(F)(F)F)N1CC(OC(=O)c2cccc([C@@H](CC(=O)O)C3CC3)c2)C1] | LCMS: tR = 0.860, (ES+) m/z (M + H)⁺ = 530.2. ¹H NMR (400 MHz, DMSO-d₆) δ = 13.17-12.68 (m, 1H), 12.06 (s, 1H), 8.93-8.69 (m, 1H), 8.15-7.80 (m, 4H), 7.63 (d, J = 7.2 Hz, 1H), 7.54-7.45 (m, 1H), 5.52-3.40 (m, 6H), 2.79-2.63 (m, 2H), 2.41-2.30 (m, 1H), 1.54 (s, 3H), 1.05 (s, 1H), 0.59-0.48 (m, 1H), 0.37-0.22 (m, 2H), 0.13 (d, J = 4.4 Hz, 1H). |
| 48 | 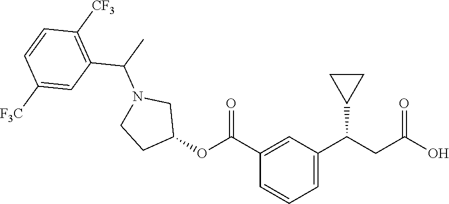 [SMILES: CC(c1cc(C(F)(F)F)ccc1C(F)(F)F)N1CC[C@@H](OC(=O)c2cccc([C@@H](CC(=O)O)C3CC3)c2)C1] | LCMS: tR = 0.873 min, (ES⁺) m/z (M + H)⁺ = 544.1. ¹H NMR (400 MHz, DMSO-d₆) δ = 12.88-11.72 (m, 1H), 9.04 (s, 1H), 8.16-8.00 (m, 2H), 7.87 (bd, J = 19.2 Hz, 2H), 7.69-7.37 (m, 2H), 5.71-5.37 (m, 1H), 5.00-4.64 (m, 1H), 4.21-3.97 (m, 1H), 2.97-2.57 (m, 4H), 2.44-1.99 (m, 4H), 1.73 (d, J = 17.0 Hz, 3H), 1.07 (bs, 1H), 0.53 (s, 1H), 0.32-0.11 (m, 3H). |
| 49 | 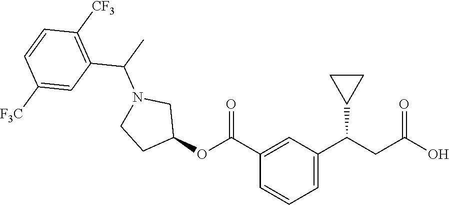 [SMILES: CC(c1cc(C(F)(F)F)ccc1C(F)(F)F)N1CC[C@H](OC(=O)c2cccc([C@@H](CC(=O)O)C3CC3)c2)C1] | LCMS (C): tR = 0.850, (ES⁺) m/z (M + H)⁺ = 544.2. ¹H NMR (400 MHz, DMSO-d₆) δ 11.92 (s, 1H), 9.00-8.90 (m, 1H), 7.92-7.90 (m, 2H), 7.71-7.68 (m, 2H), 7.48-7.38 (m, 1H), 7.35-7.33 (m, 1H), 5.48-5.39 (m, 1H), 4.73-4.62 (m, 1H), 3.92-3.90 (m, 1H), 2.60-2.58 (m, 4H), 2.34-2.19 (m, 4H), 1.62-1.58 (m, 3H), 0.94-0.93 (m, 1H), 0.40-0.01 (m, 4H). |

### Example 21: (S)-2′-fluoro-5′-methoxy-[1,1′-biphenyl]-4-yl 3-(1-cyclopropyl-3-methoxy-3-oxopropyl)benzoate (Compound 42)

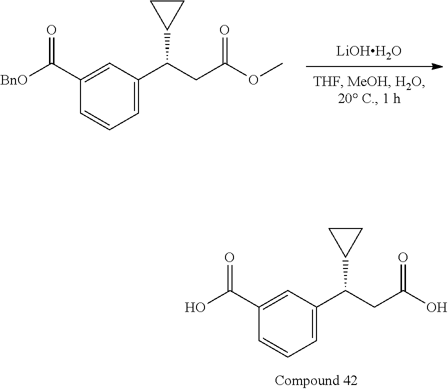 [SMILES: COC(=O)C[C@H](c1cccc(C(=O)OCc2ccccc2)c1)C1CC1 ; "LiOH·H2O" ; O=C(O)C[C@H](c1cccc(C(=O)O)c1)C1CC1]

To a solution of (S)-benzyl 3-(1-cyclopropyl-3-methoxy-3-oxopropyl)benzoate (50 mg, 0.15 mmol, 1 eq) in THF (0.5 mL), H₂O (0.5 mL) and MeOH (0.5 mL) was added LiOH.H₂O (12 mg, 0.30 mmol, 2 eq). The mixture was stirred at 20° C. for 1 hr. The mixture was added 2 N HCl to adjusted pH to 6 to give a residue. The residue was purified by prep-HPLC (column: Phenomenex Synergi C18 150×25 mm×10 μm; mobile phase: [water (0.1% TFA) -ACN]; B %: 30%-60%, 10 min) to give Compound 42 (20 mg, 57% yield) as a white solid. LCMS: tR=7.32, (ES+) m/z (M+Na)⁺=257.0. ¹H NMR (400 MHz, CDCl₃) δ=7.92 (m, 2H), 7.55 (d, J=7.8 Hz, 1H), 7.46-7.38 (m, 1H), 2.96-2.85 (m, 1H), 2.82-2.69 (m, 1H), 2.43 (m, 1H), 1.17-1.03 (m, 1H), 0.70-0.59 (m, 1H), 0.52-0.42 (m, 1H), 0.34 (m, 1H), 0.19 (m, 1H).

### Example 22: (2S,3R)-3-cyclopropyl-3-(3-((5-((diisopropylamino)methyl)-2-fluoro-4-(2-methoxypyridin-4-yl)phenoxy)carbonyl)phenyl)-2-methylpropanoic acid (Compound 59)

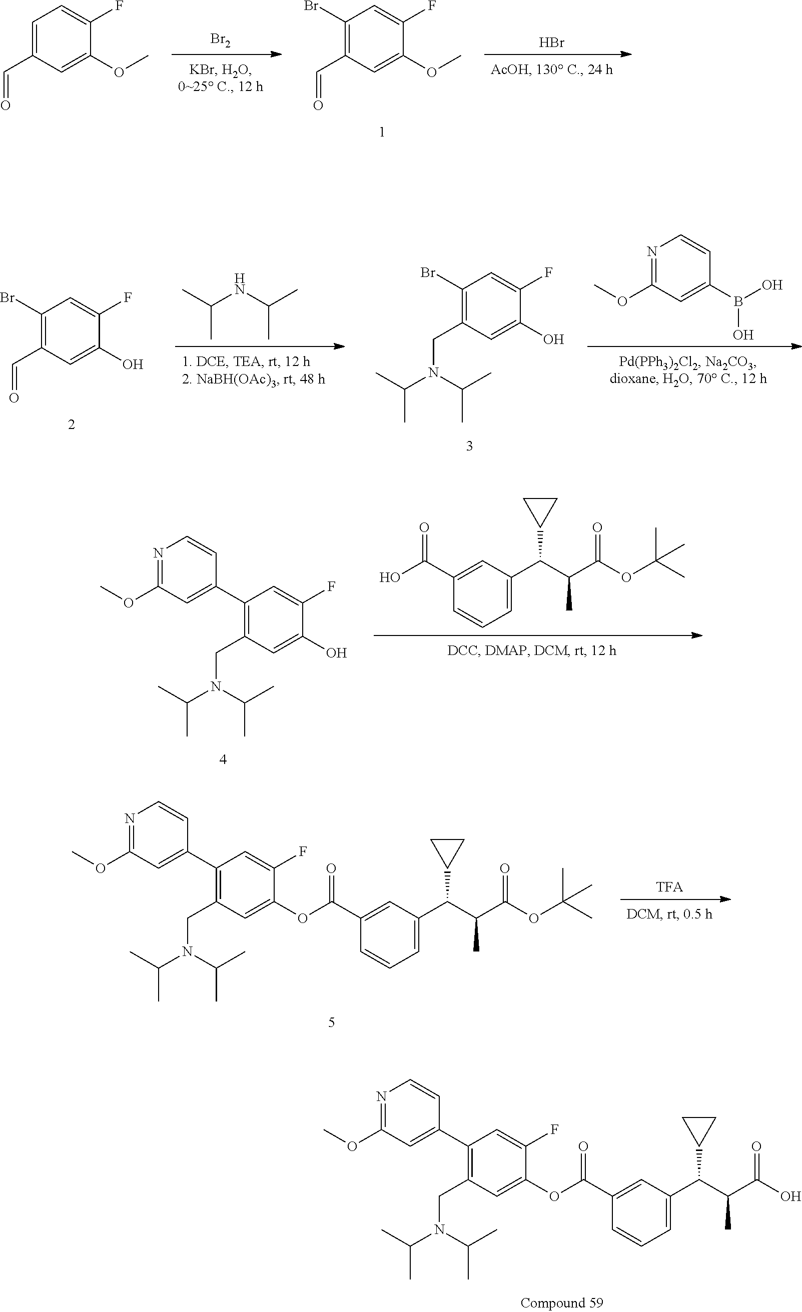

Step 1: 2-bromo-4-fluoro-5-methoxybenzaldehyde (1): To a solution of 4-fluoro-3-methoxybenzaldehyde (2.0 g, 13 mmol) in H₂O (20 mL) was added Br₂ (5.2 g, 32 mmol, 1.7 mL) and KBr (7.7 g, 65 mmol) at 0° C. The mixture was stirred at 25° C. for 12 hours. The product precipitated out of solution and was collected via filtration and dried under reduced pressure to give 1 (5.0 g) as a white solid. ¹H NMR (400 MHz, CDCl₃): δ 10.25 (s, 1H), 7.55 (d, J=8.8 Hz, 1H), 7.39 (d, J=10.0 Hz, 1H), 3.95 (s, 3H).

Step 2: 2-bromo-4-fluoro-5-hydroxybenzaldehyde (2): A solution of 1 (5.0 g, 21 mmol) in HBr (50 mL) and AcOH (50 mL) was stirred at 130° C. for 24 hours. The reaction mixture was concentrated under reduced pressure to give a residue. The residue was quenched by addition sat. aqueous NaHCO₃ to pH=7 and water (100 mL), then diluted with ethyl acetate (300 mL), and extracted with ethyl acetate (200 mL×3). The combined organic layers were washed with sat. brine (100 mL), dried over Na₂SO₄, filtered and concentrated under reduced pressure to give a residue. The residue was purified by column chromatography (SiO₂, petroleum ether/ethyl acetate, 20:1 to 1:1) to give 2 (4.5 g, 94% yield) as a yellow solid. LCMS: tR=0.375 min, (ES⁻) m/z (M−H)⁻=216.9.

Step 3: 4-bromo-5-((diisopropylamino)methyl)-2-fluorophenol (3): To a solution of 2 (4.5 g, 21 mmol) and diisopropylamine (4.2 g, 41 mmol) in DCE (30 mL) was added TEA (6.2 g, 62 mmol). The resulting solution was stirred for 12 hours at 25° C. NaBH(OAc)₃ (8.7 g, 41 mmol) was added. The mixture was stirred for another 48 hours at 25° C. The reaction mixture was quenched by addition water (10 mL), then diluted with DCM (30 mL), and extracted with DCM (20 mL×3). The combined organic layers were washed with sat. brine (20 mL), dried over Na₂SO₄, filtered and concentrated under reduced pressure to give a residue. The residue was purified by column chromatography (SiO₂, petroleum ether/ethyl acetate, 20:1 to 1:1) to give 3 (2.0 g, 30% yield) as a colorless oil. LCMS: tR=1.096 min, (ES⁻) m/z (M−H)⁻=303.9.

Step 4: 5-((diisopropylamino)methyl)-2-fluoro-4-(2-methoxypyridin-4-yl)phenol (4): To a solution of 3 (0.50 g, 1.6 mmol) and (2-methoxypyridin-4-yl)boronic acid (0.38 g, 2.5 mmol) in dioxane (6 mL) and H₂O (2 mL) was added Pd(PPh₃)₂Cl₂ (58 mg, 82 μmol) and Na₂CO₃ (0.52 g, 4.9 mmol) under N₂. The mixture was stirred at 70° C. for 12 hours. The reaction mixture was quenched by addition water (10 mL), then diluted with ethyl acetate (30 mL) and extracted with ethyl acetate (20 mL×3). The combined organic layers were washed with sat. brine (20 mL), dried over Na₂SO₄, filtered and concentrated under reduced pressure to give a residue. The residue was purified by column chromatography (SiO₂, petroleum ether/ethyl acetate, 20:1 to 1:1) to give 4 (0.36 g, 0.98 mmol, 59% yield) as a yellow oil. LCMS: tR=0.740 min, (ES⁺) m/z (M+H)⁺=333.2.

Step 5: 5-((diisopropylamino)methyl)-2-fluoro-4-(2-methoxypyridin-4-yl)phenyl 3-((1R,2S)-3-(tert-butoxy)-1-cyclopropyl-2-methyl-3-oxopropyl)benzoate (5): To a solution of 4 (0.11 g, 0.33 mmol) and 3-((1R,2S)-3-(tert-butoxy)-1-cyclopropyl-2-methyl-3-oxopropyl)benzoic acid (0.10 g, 0.33 mmol) in DCM (2 mL) was added DCC (0.10 g, 0.49 mmol) and DMAP (60 mg, 0.49 mmol). The mixture was stirred at 25° C. for 12 hours. The reaction mixture was quenched by addition water (10 mL), then diluted with DCM (30 mL) and extracted with DCM (20 mL×3). The combined organic layers were washed with sat. brine (20 mL), dried over Na₂SO₄, filtered and concentrated under reduced pressure to give 5 (0.20 g) as a yellow oil. LCMS: tR=0.997 min, (ES⁺) m/z (M+H)⁺=619.4.

Step 6: (2S,3R)-3-cyclopropyl-3-(3-((5-((diisopropylamino)methyl)-2-fluoro-4-(2-methoxypyridin-4-yl)phenoxy)carbonyl)phenyl)-2-methylpropanoic acid (Compound 59): A solution of 5 (0.20 g, 0.32 mmol) in TFA (2 mL) and DCM (5 mL) was stirred at 25° C. for 0.5 hour. The reaction mixture was concentrated under reduced pressure to give a residue. The residue was purified by prep-HPLC (column: Shim-pack C18 150×25 mm×10 μm; mobile phase: [A; water (0.225% FA); B:ACN]; B %: 28%˜48%, 10 min) to give Compound 59 (38 mg, 19% yield, FA salt) as a white solid. LCMS: tR=0.997 min, (ES⁺) m/z (M+H)⁺=563.3. ¹H NMR (400 MHz, CD₃OD): δ 8.26 (d, J=5.2 Hz, 1H), 8.14-8.08 (m, 1H), 8.07 (s, 1H), 7.71 (d, J=7.6 Hz, 1H), 7.65-7.54 (m, 2H), 7.26 (d, J=10.4 Hz, 1H), 7.03˜6.99 (m, 1H), 6.87 (s, 1H), 4.00 (s, 3H), 3.94 (s, 2H), 3.29-3.18 (m, 2H), 2.94-2.84 (m, 1H), 2.18 (t, J=9.6 Hz, 1H), 1.29-1.19 (m, 1H), 1.06 (d, J=6.8 Hz, 12H), 0.99 (d, J=6.8 Hz, 3H), 0.73˜0.64 (m, 1H), 0.48-0.35 (m, 2H), 0.08˜-0.01 (m, 1H).

The following compounds were prepared according to the procedures described in Example 22 using the appropriate intermediates.

| Cpd | Structure | Characterization Data |
|---|---|---|
| 60 | 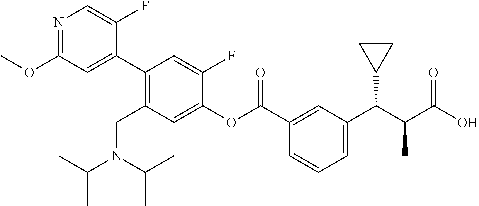 [SMILES: COc1cc(-c2cc(F)c(OC(=O)c3cccc([C@H](C4CC4)[C@H](C)C(=O)O)c3)cc2CN(C(C)C)C(C)C)c(F)cn1] | LCMS: tR = 0.890 min, (ES⁺) m/z (M + H)⁺ = 581.4. ¹H NMR (400 MHz, CD₃OD): δ 8.18-8.01 (m, 3H), 7.71 (d, J = 8 Hz, 1H), 7.66-7.49 (m, 2H), 7.22 (d, J = 10.4 Hz, 1H), 6.81 (d, J = 4.8 Hz, 1H), 3.97 (s, 3H), 3.66 (s, 2H), 3.18-2.99 (m, 2H), 2.97-2.80 (m, 1H), 2.19 (t, J = 10 Hz, 1H), 1.36-1.15 (m, 1H), 1.02-0.92 (m, 15H), 0.74-0.62 (m, 1H), 0.51-0.33 (m, 2H), 0.10-0 (m, 1H). |
| 61 | 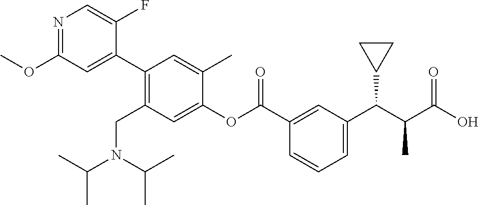 [SMILES: COc1cc(-c2cc(C)c(OC(=O)c3cccc([C@H](C4CC4)[C@H](C)C(=O)O)c3)cc2CN(C(C)C)C(C)C)c(F)cn1] | ¹H HMR (400 MHz, CD₃OD) δ = 8.11 (t, J = 0.8 Hz, 2H), 8.07 (s, 1H), 7.60 (m, 1H), 7.56 (d, J = 7.6 Hz, 1H), 7.51 (s, 1H), 7.23 (s, 1H), 6.79 (d, J = 5.2 Hz, 1H), 3.95 (s, 3H), 3.79 (s, 2H), 3.20 (m, 2H), 2.87 (m, 1H), 2.26 (s, 3H), 2.17 (t, J = 19.2 Hz, 1H), 1.23 (m, 1H), 1.02 (d, J = 6.8 Hz, 12H), 0.97 (d, J = 6.8 Hz, 3H), 0.66 (m, 1H), 0.41 (m, 2H), 0.028 (m, 1H). |
| 62 | 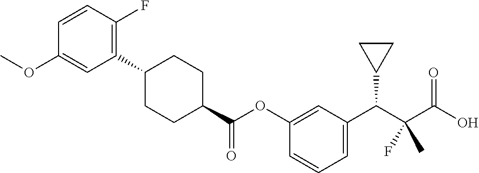 [SMILES: COc1ccc(F)c([C@H]2CC[C@H](C(=O)Oc3cccc([C@H](C4CC4)[C@@](C)(F)C(=O)O)c3)CC2)c1] | ¹H-NMR (CD₃OD, 400 MHz): δ = 7.35 (t, J = 8 Hz, 1H), 7.18 (d, J = 7.2 Hz, 1H), 7.07-6.98 (m, 2H), 6.94 (t, J = 9.2 Hz, 1H), 6.82 (dd, J₁ = 3.2 Hz, J₂ = 6 Hz, 1H), 6.75-6.70 (m, 1H), 3.76 (s, 3H), 2.94-2.80 (m, 1H), 2.73-2.60 (m, 1H), 2.40-2.23 (m, 3H), 1.96 (d, J = 11.2 Hz, 2H), 1.79-1.57 (m, 4H), 1.47-1.36 (m, 1H), 1.35-1.23 (m, 3H), 0.70-0.58 (m, 1H), 0.50-0.35 (m, 2H), 0.05--0.08 (m, 1H). |

### Example 23: (2R, 3S)-3-cyclopropyl-3-(3-((3-((diisopropylamino)methyl)-4-(5-fluoro -2-methoxypyridin-4-yl)phenoxy)carbonyl)phenyl)-2-fluoro-2-methylpropanoic acid (Compound 63)

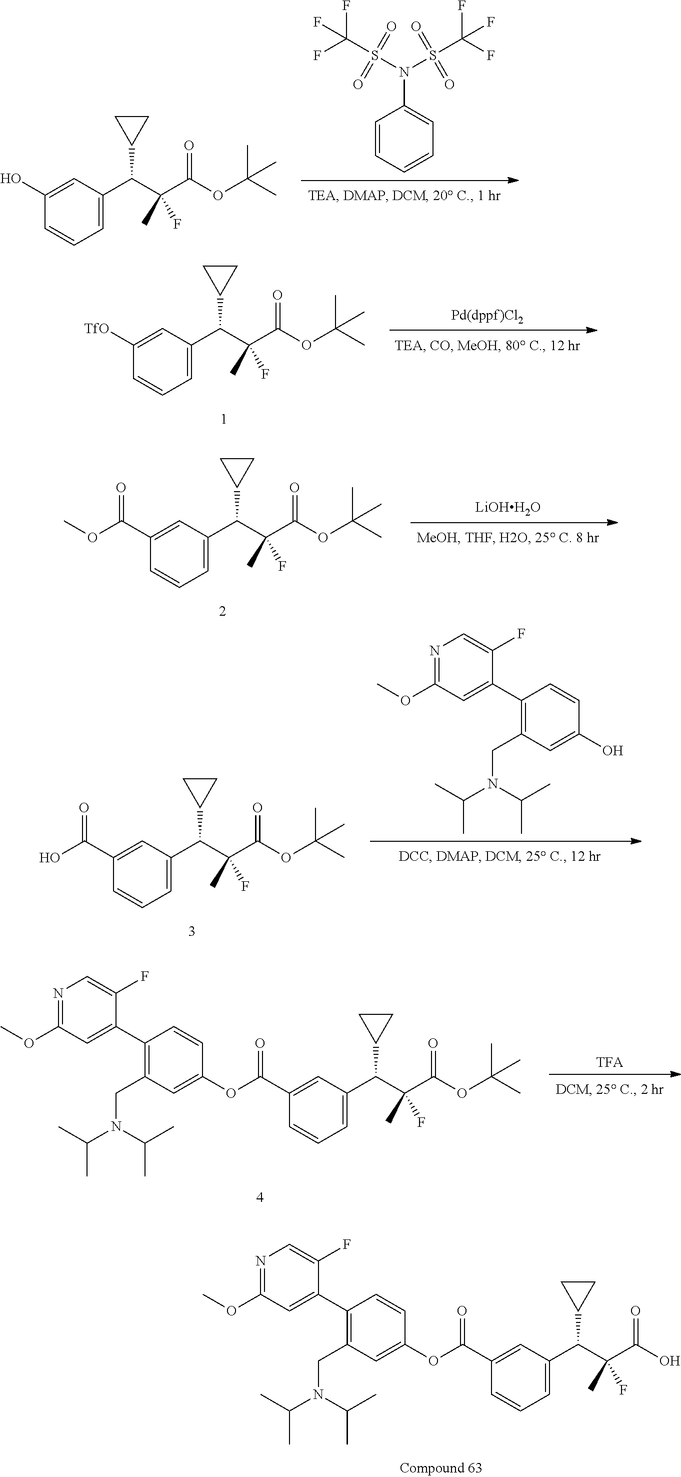

Step 1: tert-butyl (2R, 3S)-3-cyclopropyl-2-fluoro-2-methyl-3-(3-(((trifluoromethyl) sulfonyl)oxy)phenyl)propanoate (1): To a solution of tert-butyl (2R, 3S)-3-cyclopropyl-2-fluoro-3-(3-hydroxyphenyl)-2-methylpropanoate (0.25 g, 0.85 mmol) in DCM (3 mL) was added TEA (0.17 g, 1.7 mmol, 0.24 mL) and DMAP (10 mg, 85 μmol). The mixture was stirred at 20° C. for 0.5 hr. Then 1,1,1-trifluoro-N-phenyl-N-((trifluoromethyl)sulfonyl)methanesulfonamide (0.36 g, 1.0 mmol) was added to the mixture. The mixture was stirred at 20° C. for 30 min. The reaction mixture was concentrated under reduced pressure to give a residue. The residue was purified by prep-TLC (SiO₂, petroleum ether:ethyl acetate, 10:1) to give 1 (0.30 g, 81% yield) as a colorless oil. LCMS: tR=1.033 min, (ES+) m/z (M+H₂O)=444.1.

Step 2: methyl 3-((1S, 2R)-3-(tert-butoxy)-1-cyclopropyl-2-fluoro-2-methyl-3-oxopropyl)benzoate (2): To a solution of 1 (0.30 g, 0.70 mmol) in MeOH (5 mL) was added Pd(dppf)Cl₂ (51 mg, 70 μmol) and TEA (0.28 g, 2.8 mmol, 0.39 mL). The mixture was stirred at 80° C. for 12 hr under CO (15 psi). The reaction mixture was concentrated under reduced pressure to give a residue. The residue was purified by prep-TLC (SiO₂, petroleum ether:ethyl acetate, 10:1) to give 2 (0.23 g, 92% yield) as a white solid. LCMS: tR=0.974 min, (ES+) m/z (M+H₂O)=354.1. ¹H-NMR (CD₃OD, 400 MHz): δ 7.95-7.92 (m, 2H), 7.54 (dd, J₁=7.6 Hz, J₂=1.2 Hz, 1H), 7.46-7.42 (m, 1H), 3.91 (s, 3H), 2.42-2.33 (m, 1H), 1.56-1.45 (m, 10H), 1.31-1.24 (m, 3H), 0.72-0.67 (m, 1H), 0.49-1.40 (m, 2H), −0.04-−0.1 (m, 1H).

Step 3: 3-((1S, 2R)-3-(tert-butoxy)-1-cyclopropyl-2-fluoro-2-methyl-3-oxopropyl)benzoic acid (3): To a solution of 2 (0.23 g, 0.68 mmol) in THF (3.0 mL), MeOH (1.5 mL) and H₂O (1.5 mL) was added LiOH.H₂O (0.11 g, 2.7 mmol). The mixture was stirred at 25° C. for 8 hr. The reaction mixture was quenched by addition 1N aqueous HCl (5 mL) and water (10 mL), then diluted with ethyl acetate (20 mL) and extracted with ethyl acetate (15 mL×2). The combined organic layers were washed with saturated brine (15 mL×2), dried over Na₂SO₄, filtered and concentrated under reduced pressure to give a 3 (0.19 g, 84% yield) as a white solid. LCMS: tR=0.818 min, (ES+) m/z (M−H)⁻=321.1.

Step 4: 3-((diisopropylamino)methyl)-4-(5-fluoro-2-methoxypyridin-4-yl)phenyl 3-((1S,2R)-3-(tert-butoxy)-1-cyclopropyl-2-fluoro-2-methyl-3-oxopropyl)benzoate (4): 4 (0.15 g, 74% yield) was prepared from 3 (0.10 g, 0.31 mmol) and 3-((diisopropylamino)methyl)-4-(5-fluoro-2-methoxypyridin-4-yl)phenol (0.12 g, 0.37 mmol) in a similar manner to that of Example 22, Step 5. It was purified by prep-TLC (SiO₂, petroleum ether:ethyl acetate=10:1). LCMS: tR=0.921 min, (ES+) m/z (M+H)⁺=637.3.

Step 5: (2R, 3S)-3-cyclopropyl-3-(3-((3-((diisopropylamino)methyl)-4-(5-fluoro -2-methoxypyridin-4-yl)phenoxy)carbonyl)phenyl)-2-fluoro-2-methylpropanoic acid (Compound 63: Compound 63 (75 mg, 48% yield, FA salt) was prepared from 4 (0.15 g, 0.23 mmol) in a similar manner to that of Compound 59 (Example 22). It was purified by purified by prep-HPLC (column: UniSil 3-100 C18 Ultra 150×25 mm×3 μm; mobile phase: [A: water (0.225% FA), B: ACN]; B %: 28%-58%, 10 min). LCMS: tR=0.795 min, (ES+) m/z (M+H)+=581.2. ¹H-NMR (CD₃OD, 400 MHz): δ 8.18-8.17 (m, 2H), 8.11 (dd, J₁=7.6 Hz, J₂=1.2 Hz, 1H), 7.67-7.65 (m, 2H), 7.55-7.51 (m, 1H), 7.47-7.42 (m, 2H), 4.14-3.96 (m, 5H), 3.48 (s, 2H), 2.52-2.42 (m, 1H), 1.52-1.47 (m, 1H), 1.25 (d, J=21.2 Hz, 3H), 1.15 (d, J=6.8 Hz, 12H), 0.70-0.63 (m, 1H), 0.60-0.56 (m, 1H), 0.41-0.37 (m, 1H), −0.04-−0.1 (m, 1H).

### Example 24: (2R,3S)-3-cyclopropyl-3-(3-((5-((diisopropylamino)methyl)-4-(5-fluoro-2-hydroxypyridin-4-yl)-2-methylbenzoyl)oxy)phenyl)-2-fluoro-2-methylpropanoic acid (Compound 64)

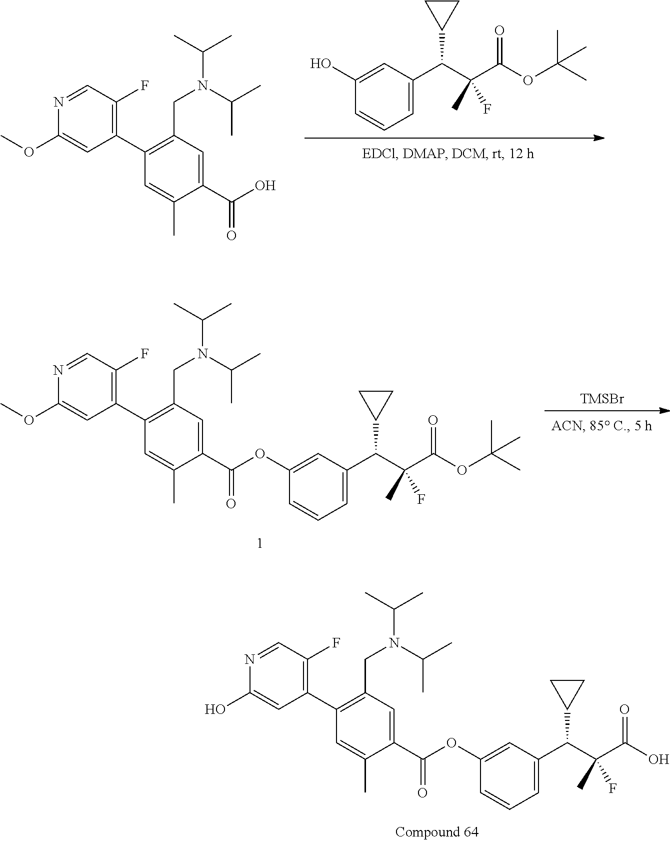 [SMILES: COc1cc(-c2cc(C)c(C(=O)O)cc2CN(C(C)C)C(C)C)c(F)cn1 ; "TMSBr" ; CC(C)(C)OC(=O)[C@](C)(F)[C@H](c1cccc(O)c1)C1CC1 ; COc1cc(-c2cc(C)c(C(=O)Oc3cccc([C@H](C4CC4)[C@@](C)(F)C(=O)OC(C)(C)C)c3)cc2CN(C(C)C)C(C)C)c(F)cn1 ; Cc1cc(-c2cc(O)ncc2F)c(CN(C(C)C)C(C)C)cc1C(=O)Oc1cccc([C@H](C2CC2)[C@@](C)(F)C(=O)O)c1]

Step 1: 3-((1S,2R)-3-(tert-butoxy)-1-cyclopropyl-2-fluoro-2-methyl-3-oxopropyl)phenyl 5-((diisopropylamino)methyl)-4-(5-fluoro-2-methoxypyridin-4-yl)-2-methylbenzoate (1): To a solution of 5-[(diisopropylamino)methyl]-4-(5-fluoro-2-methoxy-4-pyridyl)-2-methyl-benzoic acid (50 mg, 0.13 mmol, 1 eq) and tert-butyl (2R,3S)-3-cyclopropyl-2-fluoro-3-(3-hydroxyphenyl)-2-methylpropanoate (44 mg, 0.13 mmol, 1 eq) in DCM (2 mL) was added EDCI (51 mg, 0.27 mmol, 2 eq) and DMAP (33 mg, 0.27 mmol, 2 eq). The mixture was stirred at 25° C. for 12 hr. The reaction mixture was diluted with water (20 mL) and extracted with ethyl acetate (20 mL×2). The combined organic layers were washed with sat brine (40 mL), dried over Na₂SO₄, filtered and concentrated under reduced pressure to give a residue. The residue was purified by column chromatography (SiO₂, petroleum ether:ethyl acetate, 10:1 to 3:1) to give 1 (75 mg, 78% yield) as a colorless oil. ¹H NMR (400 MHz, CDCl₃) δ=8.49 (s, 1H), 8.06 (s, 1H), 7.43-7.35 (m, 1H), 7.24-7.14 (m, 3H), 7.09 (s, 1H), 6.64 (d, J=4.8 Hz, 1H), 3.97 (s, 3H), 3.54 (br s, 2H), 2.99-2.87 (m, 2H), 2.67 (s, 3H), 2.37-2.24 (m, 1H), 1.59-1.52 (m, 10H), 1.38-1.31 (m, 3H), 1.28-1.23 (m, 1H), 0.92 (d, J=6.8 Hz, 12H), 0.72-0.64 (m, 1H), 0.45 (br s, 2H), 0.07-−0.01 (m, 1H).

Step 2: (2R,3S)-3-cyclopropyl-3-(3-((5-((diisopropylamino)methyl)-4-(5-fluoro-2-hydroxypyridin-4-yl)-2-methylbenzoyl)oxy)phenyl)-2-fluoro-2-methylpropanoic acid (Compound 64): To a solution of 1 (65 mg, 0.10 mmol, 1 eq) in MeCN (1 mL) was added TMSBr (46 mg, 0.30 mmol, 3 eq). The reaction was stirred at 85° C. for 5 hrs. The reaction mixture was concentrated under reduced pressure to give a residue. The residue was purified by prep-HPLC (column: Phenomenex Synergi C18 150×25 mm×10 μm; mobile phase: [A: water (0.225% FA), B: ACN]; B %: 16%-46%, 10 min) to give Compound 64 (31 mg, 46% yield, FA salt) as white solid. LCMS: tR=0.813, (ES+) m/z (M+H)⁺=581.3. ¹H NMR (400 MHz, CD₃OD) δ=8.45 (s, 1H), 7.68 (br d, J=3.6 Hz, 1H), 7.40 (d, J=7.6 Hz, 1H), 7.36 (s, 1H), 7.30-7.20 (m, 2H), 7.20-7.09 (m, 1H), 6.55 (d, J=6.0 Hz, 1H), 4.40-3.74 (m, 2H), 3.52-3.34 (m, 2H), 2.71 (s, 3H), 2.48-2.31 (m, 1H), 1.51-1.36 (m, 1H), 1.35-1.23 (m, 3H), 1.15 (br d, J=6 Hz, 12H), 0.70-0.60 (m, 1H), 0.59-0.49 (m, 1H), 0.45-0.30 (m, 1H), 0.04-−0.08 (m, 1H).

### Example 25: 3-((1R, 2S)-1-cyclopropyl-2-methyl-3-((5-methyl-2-oxo-1,3-dioxol-4-yl) methoxy)-3-oxopropyl)phenyl 3-((diisopropylamino)methyl)-4-(5-fluoro-2-methoxypyridin-4-yl)benzoate (Compound 65)

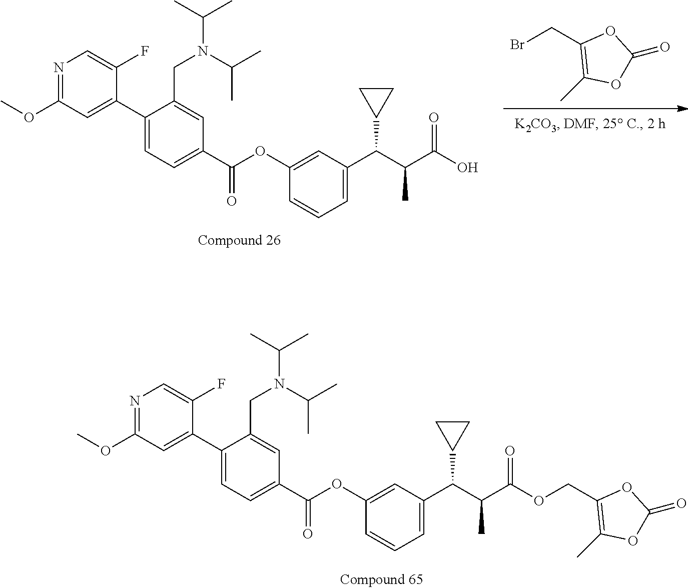

To a solution of Compound 26 (0.10 g, 0.18 mmol) and 4-(bromomethyl)-5-methyl-1,3-dioxol-2-one (34 mg, 0.18 mmol) in DMF (1.5 mL) was added K₂CO₃ (50 mg, 0.35 mmol). The mixture was stirred at 25° C. for 2 hr. The reaction mixture was filtered to give a solution. The solution was purified by prep-HPLC (column: Phenomenex Synergi C18 150×25 mm×10 μm; mobile phase: [A: water (0.225% FA), B: ACN]; B %: 42%-72%, 10 min) and lyophilized to give Compound 65 (63.87 mg, 49% yield, FA salt) as a white solid. LCMS: tR=0.834 min., (ES⁺) m/z (M+H)⁺=675.4. ¹H-NMR (DMSO-d₆, 400 MHz): δ 8.41 (s, 1H), 8.26 (s, 1H), 8.07 (dd, J₁=8 Hz, J₂=1.2 Hz, 1H), 7.43-7.37 (m, 2H), 7.21-7.14 (m, 3H), 6.87 (d, J=5.2 Hz, 1H), 5.05-4.96 (m, 2H), 3.89 (s, 3H), 3.56 (s, 2H), 2.95-2.83 (m, 3H), 2.18 (s, 3H), 2.03 (t, J=10 Hz, 1H), 1.17-1.10 (m, 1H), 0.91-0.84 (m, 15H), 0.49-0.43 (m, 1H), 0.32-0.26 (m, 1H), 0.20-0.014 (m, 1H), 0.02-−0.03 (m, 1H).

### Example 26: (2-cyclopropyl-2-(3-((3-((diisopropylamino)methyl)-4-(5-fluoro-2-methoxypyridin-4-yl)benzoyl)oxy)phenyl)ethyl)(methyl)phosphinic acid (Compound 66)

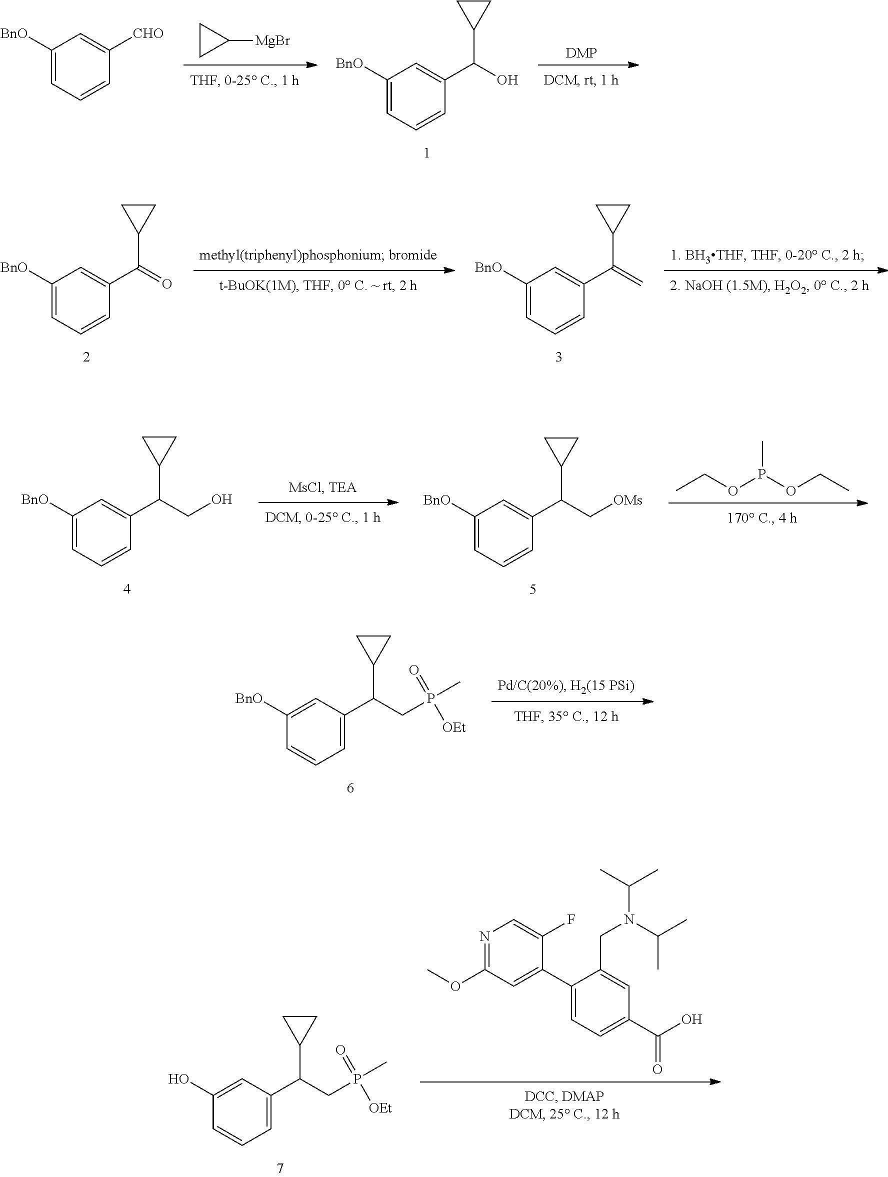

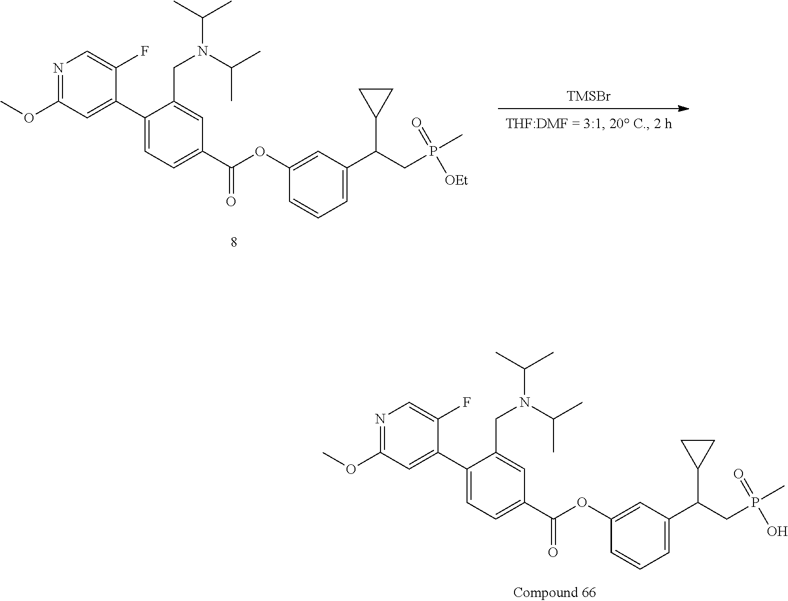

Step 1: (3-(benzyloxy)phenyl)(cyclopropyl)methanol (1): To a mixture of 3-benzyloxybenzaldehyde (10 g, 47 mmol, 1.0 eq) in THF (30 mL) was added bromo(cyclopropyl)magnesium (1.0 M in THF, 57 mL, 1.2 eq) at 0° C. under N₂. The mixture was stirred at 25° C. for 1 h. The mixture was quenched by water and extracted with ethyl acetate (100 mL×2), the combined organic phase was washed with brine (150 mL×2), dried with anhydrous Na₂SO₄, filtered and concentrated in vacuo. The residue was purified by column chromatography (SiO₂, petroleum ether:ethyl acetate, 100:1 to 3:1) to give 1 (6.5 g, 54% yield) as a colorless oil.

Step 2: (3-(benzyloxy)phenyl)(cyclopropyl)methanone (2): To a solution of 1 (6.0 g, 24 mmol, 1.0 eq) in DCM (50 mL) was added DMP (10 g, 24 mmol, 7.3 mL, 1.0 eq) at 0° C. under N₂, and the mixture was stirred at 25° C. for 20 min. The mixture was filtered and extracted with DCM (50 mL×2), and the combined organic phase was washed with brine (100 mL×2), dried with anhydrous Na₂SO₄, filtered and concentrated in vacuo. The residue was purified by column chromatography (SiO₂, petroleum ether:ethyl acetate, 100:1 to 5:1) to give 2 (5.0 g, 84% yield) as a colorless oil.

Step 3: 1-(benzyloxy)-3-(1-cyclopropylvinyl)benzene (3): To a mixture of methyl(triphenyl)phosphonium bromide (13 g, 36 mmol, 2.0 eq) in THE (50 mL) was added t-BuOK (1.0 M, 36 mL, 2.0 eq) at 0° C. under N₂. The mixture was stirred at 25° C. for 30 min. Then 2 (4.6 g, 18 mmol, 1.0 eq) was added, and the mixture stirred for 1.5 h. The mixture was quenched by water and extracted with ethyl acetate (100 mL×2). The combined organic phase was washed with brine (150 mL×2), dried with anhydrous Na₂SO₄, filtered and concentrated in vacuo. The residue was purified by column chromatography (SiO₂, petroleum ether:ethyl acetate, 100:1 to 3:1) to give 3 (6.5 g, 26 mmol, 54% yield) as a colorless oil.

Step 4: 2-(3-(benzyloxy)phenyl)-2-cyclopropylethanol (4): To a mixture of 3 (3.5 g, 14 mmol, 1.0 eq) in THE (20 mL) was added BH₃.THF (1.0 M in THF, 42 mL, 3.0 eq) at 0° C. under N₂, and 30 min later, aqueous NaOH (6.0 M, 14 mL, 6.0 eq) and H₂O₂ (32 g, 0.28 mol, 27 mL, 30% purity, 20 eq) was added by dropwise at 0° C. The mixture was stirred at 20° C. for 2 hours. The mixture was quenched by water and extracted with ethyl acetate (50 mL×2). The combined organic phase was washed with brine (150 mL×2), dried with anhydrous Na₂SO₄, filtered and concentrated in vacuo. The residue was purified by column chromatography (SiO₂, petroleum ether:ethyl acetate, 100:1 to 10:1) to give 4 (3.5 g, 13 mmol, 93% yield) as a colorless oil.

Step 5: 2-(3-(benzyloxy)phenyl)-2-cyclopropylethyl methanesulfonate (5): To a solution of 4 (1.0 g, 3.7 mmol, 1.0 eq) and TEA (1.9 g, 19 mmol, 2.6 mL, 5.0 eq) in DCM (10 mL) was added MsCl (0.85 g, 7.5 mmol, 0.58 mL, 2.0 eq) at 0° C. under N₂, and the mixture was stirred at 20° C. for 1 hr. The mixture was quenched by water and extracted with ethyl acetate (30 mL×2), the combined organic phase was washed with brine (100 mL×2), dried with anhydrous Na₂SO₄, filtered and concentrated in vacuo. The residue was purified by column chromatography (SiO₂, petroleum ether:ethyl acetate, 100:1 to 3:1) to give 5 (1.1 g, 3.2 mmol, 85% yield) as an off-white solid.

Step 6: ethyl (2-(3-(benzyloxy)phenyl)-2-cyclopropylethyl)(methyl)phosphinate (6): 5 (0.33 g, 0.95 mmol, 1.0 eq) and diethoxy(methyl)phosphane (0.39 g, 2.9 mmol, 3.0 eq) were taken up into a microwave tube, and the sealed tube was heated at 170° C. for 4 h under microwave. The mixture was concentrated and purified by column chromatography (SiO₂, petroleum ether:ethyl acetate, 100:1 to 0:1) to give 6 (0.28 g, 0.78 mmol, 82% yield) as a colorless oil.

Step 7: ethyl (2-cyclopropyl-2-(3-hydroxyphenyl)ethyl) (methyl)phosphinate (7): To a solution of 6 (0.27 g, 0.75 mmol, 1.0 eq) in THE (5.0 mL) was added 10% Pd—C (38 mg, 75 μmol, 0.10 eq) under N₂. The suspension was degassed under vacuum and purged with H₂ several times, and the mixture was stirred under H₂ (15 psi) at 35° C. for 12 hours. The mixture was filtered and concentrated. The residue was purified by prep-TLC (SiO₂, ethyl acetate) to give 7 as a colorless oil.

Step 8: 3-(1-cyclopropyl-2-(ethoxy(methyl)phosphoryl)ethyl)phenyl 3-((diisopropylamino)methyl)-4-(5-fluoro-2-methoxypyridin-4-yl)benzoate (8): To a solution of 7 (30 mg, 0.11 mmol, 0.90 eq) and 3-((diisopropylamino)methyl)-4-(5-fluoro-2-methoxypyridin-4-yl)benzoic acid (Example 7, Step 4) (45 mg, 0.12 mmol, 1.0 eq) in DCM (5.0 mL) was added DMAP (3.1 mg, 25 μmol, 0.20 eq) and DCC (52 mg, 0.25 mmol, 51 μL, 2.0 eq). The mixture was stirred at 25° C. for 12 hr. The mixture was filtered and extracted with ethyl acetate (10 mL×3). The combined organic phase was washed with brine (15 mL×2), dried with anhydrous Na₂SO₄, filtered and concentrated in vacuo. The residue was purified by prep-TLC (SiO₂, ethyl acetate) to give 8 (50 mg, 82 μmol, 66% yield) as a yellow oil.

Step 9: (2-cyclopropyl-2-(3-((3-((diisopropylamino)methyl)-4-(5-fluoro-2-methoxypyridin-4-yl)benzoyl)oxy)phenyl)ethyl)(methyl)phosphinic acid (Compound 66): To a solution of 8 (20 mg, 33 μmol, 1.0 eq) in THE (1.5 mL) and DMF (0.50 mL) was added TMSBr (0.25 g, 1.6 mmol, 0.21 mL, 49 eq) at 0° C. under N₂, and the mixture was stirred at 20° C. for 2 h. The mixture was concentrated and purified by prep-HPLC (column: Phenomenex Luna C18 200×40 mm×10 μm; mobile phase: [A: water (0.2% FA), B: ACN]; B %: 30%-70%, 10 min) to give Compound 66 (10 mg, 17 μmol, 52% yield, FA salt, 99% purity) as a white solid. LCMS: tR=1.091 min., (ES⁺) m/z (M+H)⁺=583.3. ¹HNMR (400 MHz, MeOD-d₄) δ 8.55 (d, J=1.6 Hz, 1H), 8.29 (br d, J=7.6 Hz, 1H), 8.20 (s, 1H), 7.57 (br d, J=8.0 Hz, 1H), 7.41 (t, J=8.0 Hz, 1H), 7.27 (d, J=7.6 Hz, 1H), 7.21 (s, 1H), 7.11 (dd, J=1.6, 8.0 Hz, 1H), 6.89 (d, J=4.8 Hz, 1H), 3.97 (s, 3H), 3.57-3.38 (m, 2H), 2.36-2.25 (m, 1H), 2.24-2.09 (m, 2H), 1.43-1.23 (m, 1H), 1.23-1.09 (m, 12H), 0.93-0.81 (m, 3H), 0.65-0.57 (m, 1H), 0.44-0.35 (m, 2H), 0.22-0.15 (m, 1H).

### Example 27: 2-cyclopropyl-2-(3-((3-((diisopropylamino)methyl)-4-(5-fluoro-2-methoxypyridin-4-yl)benzoyl)oxy)phenyl)ethanesulfonic acid (Compound 67)

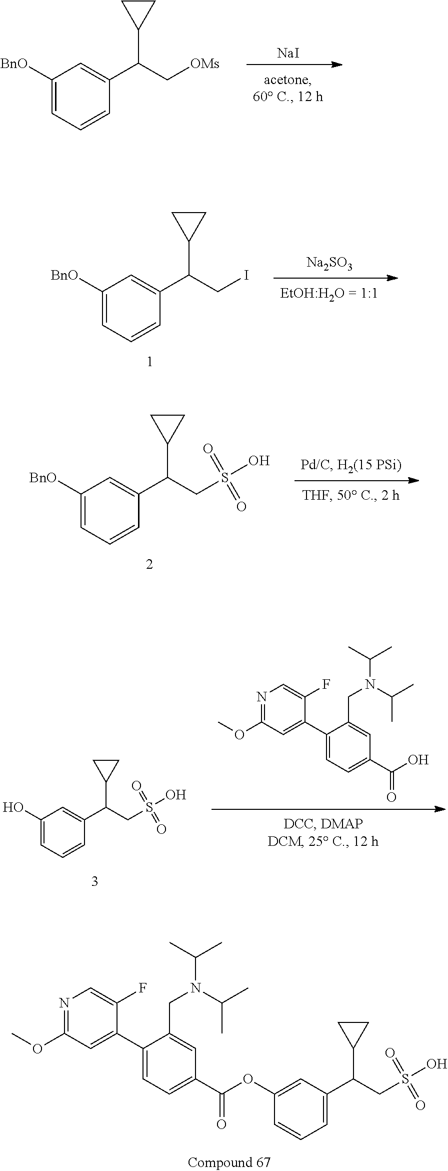

Step 1: 1-(benzyloxy)-3-(1-cyclopropyl-2-iodoethyl)benzene (1): To a solution of 2-(3-(benzyloxy)phenyl)-2-cyclopropylethyl methanesulfonate (1.1 g, 3.2 mmol, 1 eq) in acetone (10 mL) was added NaI (2.4 g, 16 mmol, 5.0 eq), and the mixture was stirred at 60° C. for 12 hr. The mixture was poured into water and extracted with ethyl acetate (5 mL×2). The combined organic phase was washed with brine (10 mL×2), dried with anhydrous Na₂SO₄, filtered and concentrated in vacuo. The residue was purified by prep-TLC (SiO₂, petroleum ether:ethyl acetate, 10:1) to give 1 (1.2 g, 100% yield) as a white solid.

Step 2: 2-(3-(benzyloxy)phenyl)-2-cyclopropylethanesulfonic acid (2): To a solution of 1 (0.6 g, 1.6 mmol, 1.0 eq) in H₂O (10 mL) and EtOH (10 mL) was added Na₂SO₃ (0.60 g, 4.8 mmol, 3.0 eq), and the mixture was stirred at 90° C. for 36 hr. The mixture was concentrated, poured into aqueous HCl (1.0 M) to adjust the pH to 5-6, and extracted with ethyl acetate (20 mL×2). The combined organic phase was washed with brine (50 mL), dried with anhydrous Na₂SO₄, filtered and concentrated in vacuo. The residue was purified by prep-TLC (SiO₂, ethyl acetate:methanol, 10:1) to give 2 (0.12 g, 23% yield) as a white solid.

Step 3: 2-cyclopropyl-2-(3-hydroxyphenyl)ethanesulfonic acid (3): To a solution of 2 (70 mg, 0.21 mmol, 1.0 eq) in THE (3.0 mL) was added 10% Pd—C (11 mg, 21 μmol, 0.1 eq) under N₂. The suspension was degassed under vacuum and purged with H₂ several times. The mixture was stirred under H₂ (15 psi) at 50° C. for 2 h. The mixture was filtered and concentrated to give 3 (50 mg, 98% yield) as a white solid.

Step 4: 2-cyclopropyl-2-(3-((3-((diisopropylamino)methyl)-4-(5-fluoro-2-methoxypyridin-4-yl)benzoyl)oxy)phenyl)ethanesulfonic acid (Compound 67): To a solution of 3 (50 mg, 0.21 mmol, 1.0 eq) and 3-((diisopropylamino)methyl)-4-(5-fluoro-2-methoxypyridin-4-yl)benzoic acid (Example 7, Step 4) (82 mg, 0.23 mmol, 1.0 eq) in DCM (2.0 mL) was added DCC (94 mg, 0.45 mmol, 92 μL, 2.0 eq) and DMAP (5.6 mg, 45 μmol, 0.20 eq). The mixture was stirred at 25° C. for 12 hr. The mixture was filtered and concentrated. The residue was purified prep-TLC (SiO₂, ethyl acetate:methanol, 8:1) to give Compound 67 (40 mg, 63 μmol, 28% yield, 92% purity) as a white solid. LCMS: tR=2.130 min., (ES⁺) m/z (M+H)⁺=585.3. ¹HNMR (400 MHz, MeOD-d₄): δ 8.54 (s, 1H), 8.17-8.06 (m, 2H), 7.41-7.33 (m, 2H), 7.28-7.17 (m, 2H), 7.09 (d, J=7.6 Hz, 1H), 6.77 (d, J=4.8 Hz, 1H), 3.95 (s, 3H), 3.63 (br s, 2H), 3.37-3.33 (m, 2H), 3.09-2.87 (m, 2H), 2.55 (td, J=6.4, 9.6 Hz, 1H), 1.24-1.14 (m, 1H), 0.94 (br d, J=6.4 Hz, 12H), 0.66-0.58 (m, 1H), 0.49-0.40 (m, 2H), 0.25-0.14 (m, 1H).

### Example 28: (2-(((2S, 3R)-3-cyclopropyl-3-(3-((3-((diisopropylamino)methyl)-4-(5-fluoro-2-methoxypyridin-4-yl)benzoyl)oxy)phenyl)-2-methylpropanoyl)oxy)ethyl)phosphonic acid (Compound 68)

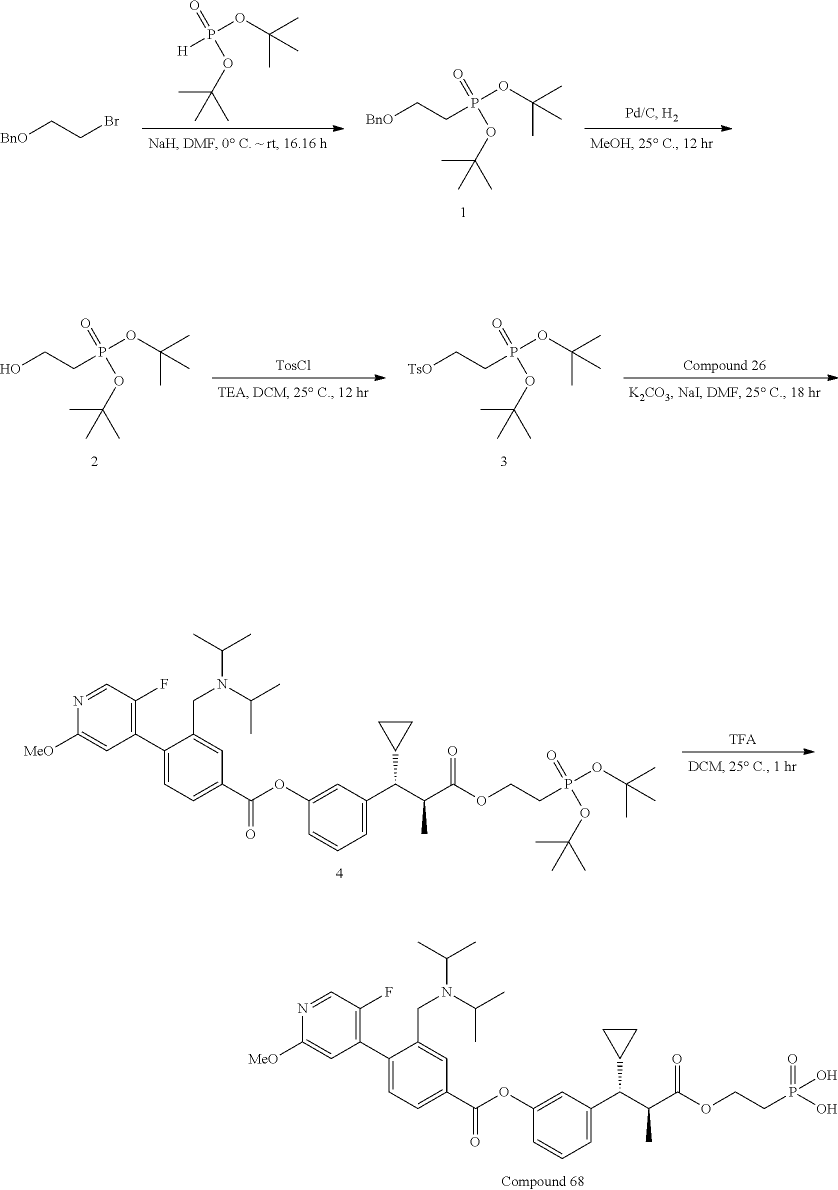

Step 1: di-tert-butyl (2-(benzyloxy)ethyl)phosphonate (1): To a solution of di-tert-butyl phosphonate (1.0 g, 5.1 mmol) in DMF (12 mL) was added NaH (0.62 g, 15 mmol, 60% purity) at 0° C. The mixture was stirred at 0° C. for 10 min. Then ((2-bromoethoxy)methyl)benzene (1.7 g, 7.7 mmol, 1.2 mL) in DMF (12 mL) was added to the mixture. The mixture was stirred at 25° C. for 16 hr. The reaction mixture was quenched by addition water 30 (mL) and extracted with ethyl acetate (30 mL×3). The combined organic layers were washed with saturated brine (10 mL×3), dried over Na₂SO₄, filtered and concentrated under reduced pressure to give a residue. The residue was purified by column chromatography (SiO₂, petroleum ether:ethyl acetate, 100:1 to 40:1) to give 1 (0.98 g, 52.16% yield, 90% purity) as a white solid. LCMS: tR=0.965 min., (ES⁺) m/z (M+H)⁺=329.2. ¹H-NMR (DMSO-d₆, 400 MHz): δ 7.34-7.27 (m, 5H), 4.47 (s, 2H), 3.62-3.53 (m, 2H), 1.99-1.90 (m, 2H), 1.41-1.39 (m, 18H).

Step 2: di-tert-butyl (2-hydroxyethyl)phosphonate (2): To a solution of 1 (0.88 g, 2.7 mmol) in MeOH (10 mL) was added 10% Pd/C (0.1 g). The mixture was stirred at 25° C. for 12 hr under H₂ (15 psi). The reaction mixture was filtered, and the solution was concentrated under reduced pressure to give 2 (0.58 g, 91% yield) as a colorless oil. ¹H-NMR (DMSO-d₆, 400 MHz): δ 4.63 (s, 1H), 3.55 (d, J=6.4 Hz, 2H), 1.86˜1.78 (m, 2H), 1.41 (s, 18H).

Step 3: 2-(di-tert-butoxyphosphoryl)ethyl 4-methylbenzenesulfonate (3): To a solution of 2 (0.58 g, 2.4 mmol) in DCM (10 mL) was added TEA (0.49 g, 4.9 mmol, 0.68 mL) and TsCl (0.70 g, 3.6 mmol). The mixture was stirred at 25° C. for 12 hr. The reaction mixture was concentrated under reduced pressure to give a residue. The residue was purified by column chromatography (SiO₂, petroleum ether:ethyl acetate, 10:1 to 5:1) to give 3 (0.44 g, 46% yield) as a colorless oil. LCMS: tR=0.964 min., (ES⁺) m/z (M−2×t-Bu)⁺=281.1.

Step 4: 3-((1R, 2S)-1-cyclopropyl-3-(2-(di-tert-butoxyphosphoryl)ethoxy)-2-methyl-3-oxopropyl)phenyl 3-((diisopropylamino)methyl)-4-(5-fluoro-2-methoxypyridin-4-yl) benzoate (4): To a solution of Compound 26 (60 mg, 0.11 mmol) and 3 (63 mg, 0.16 mmol) in DMF (0.6 mL) was added K₂CO₃ (30 mg, 0.20 mmol) and NaI (8.0 mg, 53 μmol). The mixture was stirred at 25° C. for 18 hr. The reaction mixture was quenched by addition of water (20 mL) and extracted with ethyl acetate (10 mL×3). The combined organic layers were washed with saturated brine (10 mL×3), dried over Na₂SO₄, filtered and concentrated under reduced pressure to give a residue. The residue was purified by prep-HPLC (column: Phenomenex Synergi C18 150×25 mm×10 μm; mobile phase: [A: water (0.225% FA), B: ACN]; B %: 44%-74%, 10 min) and lyophilized to give 4 (55 mg, 62% yield, FA salt) as a brown oil. LCMS: tR=0.981 min., (ES⁺) m/z (M+H)⁺=783.2.

Step 5: (2-(((2S, 3R)-3-cyclopropyl-3-(3-((3-((diisopropylamino)methyl)-4-(5-fluoro-2-methoxypyridin-4-yl)benzoyl)oxy)phenyl)-2-methylpropanoyl)oxy)ethyl)phosphonic acid (Compound 68): To a solution of 4 (55 mg, 70 μmol) in DCM (2 mL) was added TFA (0.77 g, 6.7 mmol, 0.5 mL). The mixture was stirred at 25° C. for 1 hr. The reaction mixture was concentrated under reduced pressure to give a residue. The residue was purified by prep-HPLC (column: Phenomenex Synergi C18 150×25 mm×10 μm; mobile phase: [A: water (0.225% FA), B: ACN]; B %: 28%-58%, 10 min) and lyophilized to give Compound 68 (26 mg, 50% yield, FA salt) as a white solid. LCMS: tR=0.857, (ES⁺) m/z (M+H)⁺=671.5. ¹H-NMR (DMSO-d₆, 400 MHz): δ 8.77 (d, J=1.2 Hz, 1H), 8.3 (dd, J₁=8 Hz, J₂=1.6 Hz, 1H), 8.23 (d, J=0.8 Hz, 1H), 7.59 (d, J=8 Hz, 1H), 7.44-7.40 (m, 1H), 7.23 (d, J=7.6 Hz, 1H), 7.12 (dd, J₁=8 Hz, J₂=1.6 Hz, 1H), 7.06 (s, 1H), 6.92 (d, J=4.8 Hz, 1H), 4.38-4.15 (m, 4H), 3.97 (s, 3H), 3.63-3.56 (m, 2H), 2.95-2.88 (m, 2H), 2.21-2.17 (m, 2H), 2.00-2.92 (m, 2H), 1.33-1.27 (m, 13H), 1.08 (d, J=6.8 Hz, 3H), 0.71-0.64 (m, 1H), 0.48-0.42 (m, 1H), 0.37-0.31 (m, 1H), 0.07-0.01 (m, 1H).

### Example 29: (2S,3R)-3-cyclopropyl-3-[3-[3-[(diisopropylamino)methyl]-4-(5-fluoro-2-methoxy-4-pyridyl)benzoyl]oxy-2-fluoro-phenyl]-2-methyl-propanoic acid (Compound 69)

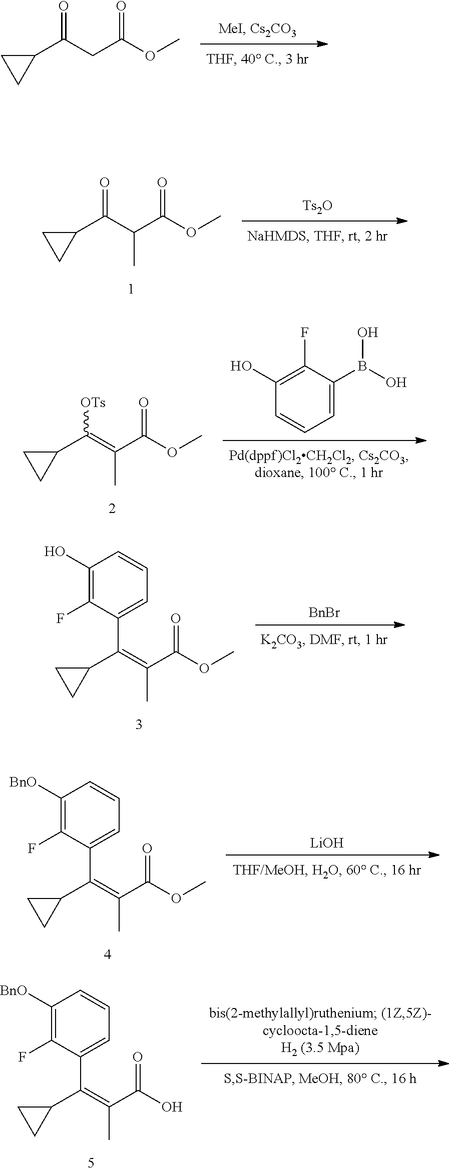

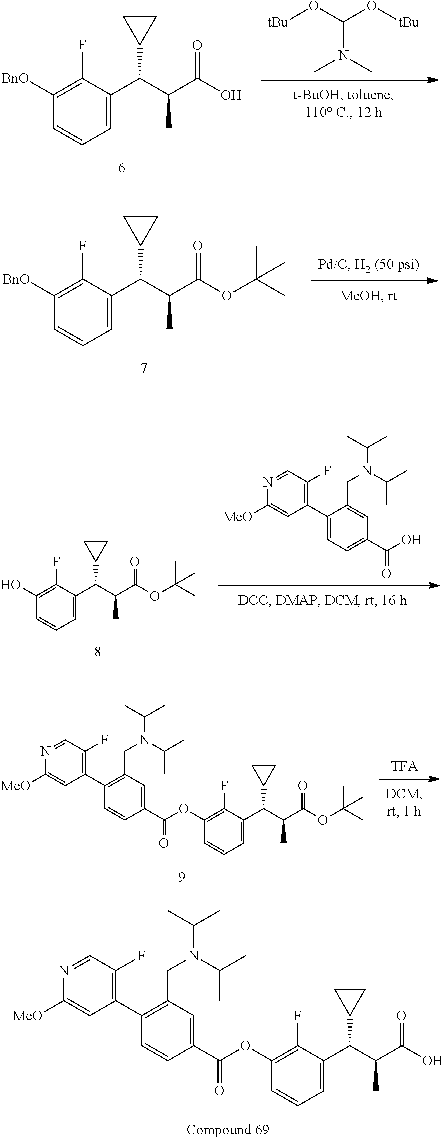

Step 1: methyl 3-cyclopropyl-2-methyl-3-oxo-propanoate (1): To a mixture of methyl 3-cyclopropyl-3-oxopropanoate (15 g, 0.11 mol, 1.0 eq) in THF (0.10 L) was added Cs₂CO₃ (52 g, 0.16 mol, 1.5 eq). The mixture was stirred at 20° C. for 0.5 hours and then was added CH₃I (15 g, 0.11 mol, 6.6 mL, 1.0 eq) at 20° C. under N₂. The mixture was stirred at 40° C. for 2.5 hours. The reaction mixture was filtered, and the filtrate cake was washed with EtOAc (150 mL×3). The combined organic phase was concentrated to give 1 (15 g, 91% yield) as a yellow oil. ¹H NMR (400 MHz, CDCl₃-d) 6 ppm 3.75-3.73 (m, 3H), 3.71-3.64 (m, 1H), 2.05 (t, J=7.6, 4.4 Hz, 1H), 1.42-1.38 (m, 3H), 1.12-1.01 (m, 2H), 0.97-0.86 (m, 2H).

Step 2: methyl (Z)-3-cyclopropyl-2-methyl-3-(p-tolylsulfonyloxy)prop-2-enoate (2): To a mixture of 1 (15 g, 96 mmol, 1.0 eq) in THF (0.10 L) was added NaHMDS (1 M in THF, 0.12 L, 1.3 eq) at 0° C. under N₂. The mixture was stirred at 0° C. for 30 mins, then p-tolylsulfonyl 4-methylbenzenesulfonate (38 g, 0.12 mol, 1.2 eq) was added at 0° C. The mixture was stirred at 20° C. for 1.5 hours. The reaction mixture was poured into water (200 mL), then extracted with ethyl acetate (500 mL×3). The combined organic phase was washed with brine (300 mL), dried with anhydrous Na₂SO₄, filtered and concentrated in vacuo. The residue was purified by silica gel chromatography (SiO₂, petroleum ether:ethyl acetate, 20:1, 10:1) to give 2 (13 g, 42% yield) as a white solid. ¹H NMR (400 MHz, CDCl₃-d) 6 ppm 7.85-7.80 (m, 2H), 7.34 (d, J=8.0 Hz, 2H), 3.59 (s, 3H), 2.46 (s, 3H), 2.02 (d, J=1.2 Hz, 3H), 1.61 (s, 1H), 0.75-0.68 (m, 4H).

Step 3: methyl (Z)-3-cyclopropyl-3-(2-fluoro-3-hydroxyphenyl)-2-methylprop-2-enoate (3): To a mixture of 2 (11 g, 34 mmol, 1.0 eq) and (2-fluoro-3-hydroxyphenyl)boronic acid (5.8 g, 37 mmol, 1.1 eq) in dioxane (50 mL) and H₂O (5 mL) was added Cs₂CO₃ (12 g, 37 mmol, 1.1 eq) and Pd(dppf)Cl₂.CH₂Cl₂ (1.4 g, 1.7 mmol, 0.05 eq) at 20° C. under N₂. The mixture was stirred at 100° C. for 1 hour. The reaction mixture was filtrated, and the filtrate was poured into water (80 mL). Then the mixture was extracted with ethyl acetate (200 mL×3). The combined organic phase was washed with brine (100 mL), dried with anhydrous Na₂SO₄, filtered and concentrated in vacuo. The residue was purified by column chromatography (SiO₂, petroleum ether:ethyl acetate, 20:1 to 5:1) to give 3 (7.0 g, 83% yield) as a yellow oil.

Step 4: methyl (Z)-3-(3-benzyloxy-2-fluorophenyl)-3-cyclopropyl-2-methylprop-2-enoate (4): To a mixture of 3 (8.0 g, 32 mmol, 1.0 eq) and bromomethylbenzene (8.2 g, 48 mmol, 5.7 mL, 1.5 eq) in DMF (40 mL) was added K₂CO₃ (6.6 g, 48 mmol, 1.5 eq) at 20° C. under N₂. The mixture was stirred at 20° C. for 1 hour. The reaction mixture was poured into water (50 mL), and the mixture was extracted with ethyl acetate (250 mL×3). The combined organic phase was washed with brine (100 mL), dried with anhydrous Na₂SO₄, filtered and concentrated in vacuo. The residue was purified by column chromatography (SiO₂, petroleum ether:ethyl acetate, 40:1 to 10:1) to give 4 (7.5 g, 69% yield) as a white solid. ¹H NMR (400 MHz, CDCl₃-d) 6 ppm 7.49-7.30 (m, 5H), 6.95-6.91 (m, 2H), 6.48 (td, J=6.0, 3.6 Hz, 1H), 5.14 (s, 2H), 3.40 (s, 3H), 2.19 (s, 3H), 1.96-1.87 (m, 1H), 0.81-0.73 (m, 2H), 0.33 (q, J=5.2 Hz, 2H).

Step 5: (Z)-3-(3-benzyloxy-2-fluorophenyl)-3-cyclopropyl-2-methylprop-2-enoic acid (5): To a mixture of 4 (7.5 g, 22 mmol, 1.0 eq) in THE (15 mL) and MeOH (45 mL) was added LiOH.H₂O (4.4 g, 0.10 mol, 4.8 eq) in H₂O (15 mL) at 0° C. under N₂. The mixture was stirred at 60° C. for 16 hours. The reaction mixture was concentrated under reduced pressure. The residue was diluted with H₂O (30 mL) and extracted with EtOAc (30 mL×3). The combined aqueous layers was acidified with 1M aqueous HCl to pH 7 and extracted with EtOAc (50 mL×3). Then the combined organic layer was washed with sat. aqueous NaCl (100 mL), dried over Na₂SO₄, filtered and concentrated to give 5 (6.0 g, 83% yield) as a white solid.

Step 6: (2S,3R)-3-(3-benzyloxy-2-fluorophenyl)-3-cyclopropyl-2-methylpropanoic acid (6): To a solution of 5 (3.0 g, 9.2 mmol, 1.0 eq) in MeOH (30 mL) was added bis(2-methylallyl)ruthenium, (1Z,5Z)-cycloocta-1,5-diene (59 mg, 0.18 mmol, 0.020 eq) and [1-(2-diphenylphosphanyl-1-naphthyl)-2-naphthyl]diphenylphosphane (0.14 g, 0.23 mmol, 0.025 eq) under N₂ protection. The suspension was degassed and purged with H₂ 3 times. The mixture was stirred under H₂ (3.5 MPa) at 80° C. for 16 hours. The reaction mixture was filtrated, and the filtrate was concentrated. The residue was diluted with sat. aqueous NaHCO₃ to pH 8-10 and extracted with MTBE (50 mL×3). The combined aqueous layer was acidified with 1 M aqueous HCl to pH 4-6 and extracted with EtOAc (100 mL×2). Then the combined organic layer was washed with sat. aqueous NaCl (50 mL), dried over Na₂SO₄, filtered and concentrated to give 6 (2.4 g, 78% yield) as a gray solid. LCMS: tR=1.323 min., (ES⁺) m/z (M+H)⁺=329.1. ¹H NMR (400 MHz, CDCl₃-d) 6 ppm 7.45-7.24 (m, 5H), 6.99-6.91 (m, 1H), 6.90-6.81 (m, 1H), 6.81-6.71 (m, 1H), 5.10-5.03 (m, 2H), 2.95-2.82 (m, 1H), 2.40-2.26 (m, 1H), 1.22-1.09 (m, 1H), 1.00-0.91 (m, 3H), 0.64-0.54 (m, 1H), 0.43-0.23 (m, 2H), 0.08-0.05 (m, 1H).

Step 7: tert-butyl (2S,3R)-3-(3-benzyloxy-2-fluorophenyl)-3-cyclopropyl-2-methyl-propanoate (7): To a solution of 6 (1.5 g, 4.6 mmol, 1.0 eq) in toluene (15 mL) was added 2-methylpropan-2-ol (1.7 g, 23 mmol, 2.2 mL, 5.0 eq) and N,N-dimethylformamide di-neopentyl acetal (5.3 g, 23 mmol, 5.0 eq) under N₂. The mixture was stirred at 110° C. for 12 hours. The mixture was concentrated in vacuo to give crude product. The residue was purified by prep-TLC (SiO₂, petroleum ether:ethyl acetate, 10:1) to give 7 (0.60 g, 34% yield) as a colorless oil. LCMS: tR=1.617 min., (ES⁺) m/z (M-tBu+H)⁺=329.1. ¹H NMR (400 MHz, CDCl₃-d) 6 ppm 7.48-7.31 (m, 5H), 7.05-6.95 (m, 1H), 6.89 (dt, J=1.2, 8.0 Hz, 1H), 6.85-6.78 (m, 1H), 5.13 (s, 2H), 2.87-2.71 (m, 1H), 2.39 (t, J=10.0 Hz, 1H), 1.50 (s, 9H), 1.22-1.11 (m, 1H), 0.94 (d, J=6.8 Hz, 3H), 0.68-0.56 (m, 1H), 0.46-0.27 (m, 2H), 0.06-0.02 (m, 1H).

Step 8: tert-butyl (2S, 3R)-3-cyclopropyl-3-(2-fluoro-3-hydroxyphenyl)-2-methyl-propanoate (8): To a solution of 7 (0.50 g, 1.3 mmol, 1.0 eq) in THE (5 mL) was added Pd—C (10%, 0.10 g) under N₂ atmosphere. The suspension was degassed and purged with H₂ 3 times. The mixture was stirred under H₂ (15 Psi) at 35° C. for 16 hours. The reaction mixture was filtrated, and the filtrate was concentrated to give 8 (0.38 g, 98% yield) as a white solid. LCMS: tR=1.358 min., (ES⁺) m/z (M-tBu+H)⁺=239.0. ¹H NMR (400 MHz, MeOD-d₄) δ ppm 6.96-6.88 (m, 1H), 6.77 (dt, J=1.6, 8.4 Hz, 1H), 6.69 (ddd, J=1.6, 6.0, 7.6 Hz, 1H), 3.35 (s, 1H), 2.87-2.71 (m, 1H), 2.29 (t, J=10.0 Hz, 1H), 1.49 (s, 9H), 1.20-1.08 (m, 1H), 0.89 (d, J=6.8 Hz, 3H), 0.65-0.55 (m, 1H), 0.40-0.24 (m, 2H), 0.11-0.04 (m, 1H).

Step 9: [3-[(1R,2S)-3-tert-butoxy-1-cyclopropyl-2-methyl-3-oxopropyl]-2-fluoro-phenyl] 3-[(diisopropylamino)methyl]-4-(5-fluoro-2-methoxy-4-pyridyl)benzoate (9): To a solution of 3-((diisopropylamino)methyl)-4-(5-fluoro-2-methoxypyridin-4-yl)benzoic acid (Example 7, Step 4) (0.12 g, 0.34 mmol, 1.0 eq) and 8 (0.1 g, 0.34 mmol, 1.0 eq) in DCM (3 mL) was added DMAP (21 mg, 0.17 mmol, 0.50 eq) and DCC (0.11 g, 0.51 mmol, 0.10 mL, 1.5 eq). The mixture was stirred at 25° C. for 16 hours. The reaction mixture was partitioned between water (10 mL) and DCM (30 mL). The combined organic phase was washed with brine (10 mL), dried with anhydrous Na₂SO₄, filtered and concentrated in vacuo. The residue was purified by prep-TLC (SiO₂, petroleum ether:ethyl acetate, 5:1) to give 9 (0.15 g, 69% yield) as a white solid. LCMS: tR=1.404 min., (ES⁺) m/z (M+H)⁺=637.4. ¹H NMR (400 MHz, CDCl₃-d) 6 ppm 8.60 (s, 1H), 8.15-8.05 (m, 2H), 7.31-7.28 (m, 1H), 7.16 (br d, J=2.8 Hz, 3H), 6.65 (d, J=4.8 Hz, 1H), 3.99-3.97 (m, 3H), 3.57 (br s, 2H), 2.94 (td, J=6.4, 13.2 Hz, 2H), 2.89-2.74 (m, 1H), 2.42 (t, J=10.0 Hz, 1H), 1.49 (s, 9H), 1.23-1.12 (m, 1H), 1.01-0.98 (m, 3H), 0.93 (d, J=6.4 Hz, 12H), 0.68-0.59 (m, 1H), 0.47-0.32 (m, 2H), 0.09-0.00 (m, 1H).

Step 10: (2S,3R)-3-cyclopropyl-3-[3-[3-[(diisopropylamino)methyl]-4-(5-fluoro-2-methoxy-4-pyridyl)benzoyl]oxy-2-fluorophenyl]-2-methylpropanoic acid (Compound 69): The solution of 9 (0.15 g, 0.24 mmol, 1.0 eq) in DCM (3 mL) and TFA (1 mL) was stirred at 25° C. for 1 hour. The reaction mixture was concentrated under reduced pressure to give a residue. To the residue was added aq. NaHCO₃ to bring the pH to 6. The residue was extracted with EtOAc (25 mL×2). The combined organic phase was washed with saturated brine (10 mL×2), dried over Na₂SO₄, filtered and concentrated under reduced pressure to give a residue. The residue was purified by reversed-phase HPLC (column: Xtimate C18 100×30 mm×3 μm; mobile phase: [A: water (0.2% FA), B: ACN]; B %: 20%-50%, 10 min) to give Compound 69 (90 mg, 63% yield, FA salt) as a white solid. LCMS: tR=2.323 min., (ES⁺) m/z (M+H)⁺=581.3. ¹H NMR (400 MHz, MeOD-d₄) δ ppm 8.55 (s, 1H), 8.45-8.38 (m, 1H), 8.27 (s, 1H), 7.70 (d, J=8.0 Hz, 1H), 7.36-7.21 (m, 3H), 6.97 (d, J=4.8 Hz, 1H), 4.56 (br s, 1H), 4.31 (br s, 1H), 3.98 (s, 3H), 3.71 (br s, 2H), 2.94-2.85 (m, 1H), 2.42 (t, J=10.4 Hz, 1H), 1.40-1.13 (m, 13H), 0.98 (d, J=7.2 Hz, 3H), 0.71-0.59 (m, 1H), 0.46-0.31 (m, 2H), 0.06-0.03 (m, 1H).

### Example 30: (2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid (Compound 70)

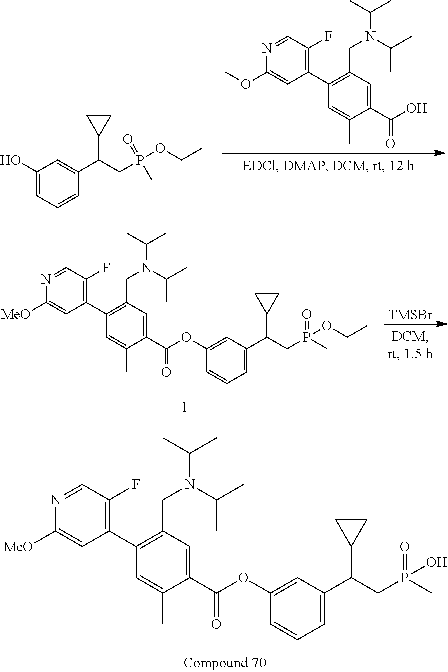 [SMILES: "TMSBr" ; CCOP(C)(=O)CC(c1cccc(O)c1)C1CC1 ; COc1cc(-c2cc(C)c(C(=O)O)cc2CN(C(C)C)C(C)C)c(F)cn1 ; CCOP(C)(=O)CC(c1cccc(OC(=O)c2cc(CN(C(C)C)C(C)C)c(-c3cc(OC)ncc3F)cc2C)c1)C1CC1 ; COc1cc(-c2cc(C)c(C(=O)Oc3cccc(C(CP(C)(=O)O)C4CC4)c3)cc2CN(C(C)C)C(C)C)c(F)cn1]

Step 1: 3-(1-cyclopropyl-2-(ethoxy(methyl)phosphoryl)ethyl)phenyl 5-((diisopropylamino)methyl)-4-(5-fluoro-2-methoxypyridin-4-yl)-2-methylbenzoate (1): To a solution of 5-((diisopropylamino)methyl)-4-(5-fluoro-2-methoxypyridin-4-yl)-2-methylbenzoic acid (Example 10, Step 5) (0.35 g, 0.93 mmol) in DCM (5.0 mL) was added ethyl (2-cyclopropyl-2-(3-hydroxyphenyl)ethyl) (methyl)phosphinate (Example 26, Step 7) (0.25 g, 0.93 mol), EDCI (0.27 g, 1.4 mmol) and DMAP (0.17 g, 1.4 mmol). The mixture was stirred at 25° C. for 12 hours. The reaction mixture was concentrated under reduced pressure to give a residue. The residue was purified by column chromatography (SiO₂, petroleum ether:ethyl acetate, 10:1 to 1:1) to give 1 (0.55 g, 89% yield) as a white solid. ¹H NMR (400 MHz, CD₃OD) δ 8.46 (s, 1H), 8.09-8.03 (m, 4H), 7.18 (s, 1H), 7.15-7.07 (m, 1H), 6.73 (d, J=5.2 Hz, 1H), 3.94 (s, 3H), 3.58 (s, 2H), 2.99-2.87 (m, 2H), 2.66 (s, 3H), 2.49-2.20 (m, 3H), 1.28-1.12 (m, 8H), 0.92 (d, J=6.8 Hz, 12H), 0.74-0.55 (m, 1H), 0.53-0.41 (m, 1H), 0.40-0.30 (m, 1H), 0.29-0.13 (m, 1H).

Step 2: (2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid (Compound 70): To a solution of 1 (0.20 g, 0.32 mmol) in DCM (1.0 mL) was added TMSBr (98 mg, 0.64 mmol, 83 μL) at 0° C. The mixture was stirred at 25° C. for 1.5 hours. The reaction mixture was concentrated under reduced pressure to give a residue. The residue was purified by prep-HPLC (column: Waters Xbridge 150×25 mm×5 μm; mobile phase: [A: water (10M NH₄HCO₃), B: ACN]; B %: 33%-63%, 9 min) to give Compound 70 (31 mg, 15% yield) as a white solid. LCMS: tR=0.814 min, (ES+) m/z (M+H)⁺=597.4. ¹H NMR (400 MHz, CD₃OD) δ 8.48 (s, 1H), 8.24 (d, J=0.8, 1H), 7.45 (m, 2H), 7.29 (d, J=7.6 Hz, 1H), 7.23 (t, J=3.2 Hz, 1H), 7.14 (d, J=1.2 Hz, 1H), 6.94 (d, J=4.8 Hz, 1H), 4.32 (m, 2H), 3.98 (s, 3H), 3.66 (m, 2H), 2.74 (s, 3H), 2.26 (m, 3H), 1.25 (d, J=0.8 Hz, 12H), 1.15 (m, 1H), 0.95 (d, J=14.0 Hz, 3H), 0.63 (m, 1H), 0.40 (m, 2H), 0.20 (m, 1H).

### Example 31: ((R)-2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid & ((S)-2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid (Compounds 71a and 71b)*

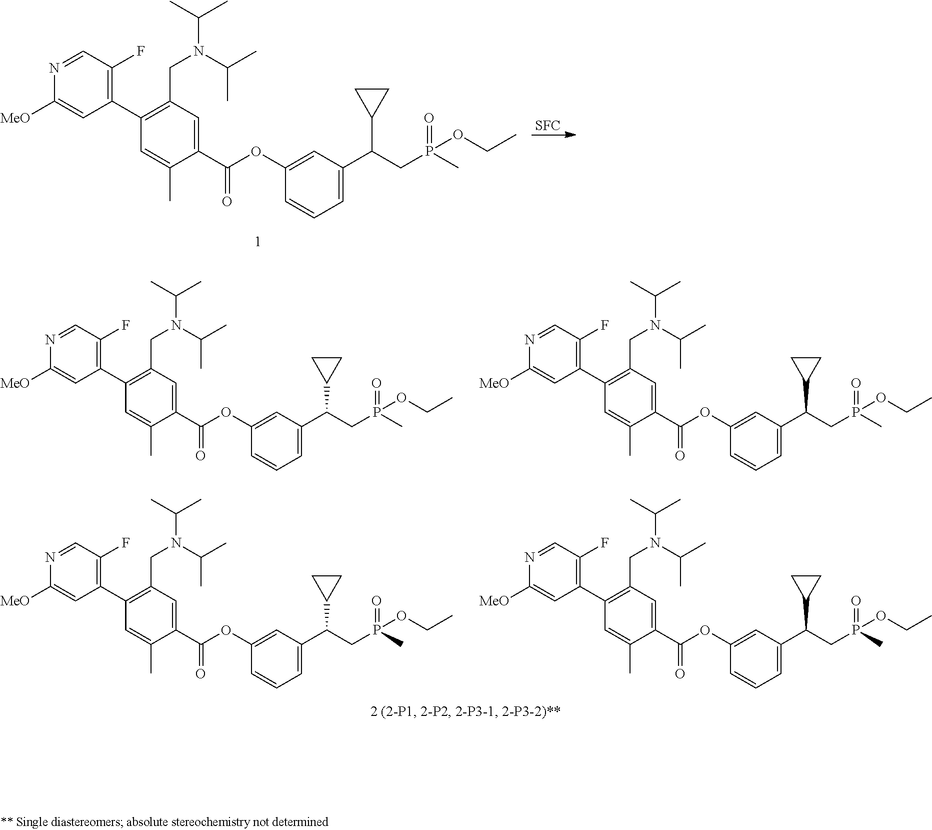

Step 1: 3-((1S & 1R)-1-cyclopropyl-2-(ethoxy(methyl)phosphoryl)ethyl)phenyl 5-((diisopropylamino)methyl)-4-(5-fluoro-2-methoxypyridin-4-yl)-2-methylbenzoate (2): Starting Material 1 (Example 30, Step 1) (350 mg, 0.56 mmol) was purified by prep-HPLC (column: Phenomenex Gemini NX-C18 (75×30 mm×3 μm); mobile phase: [A: water (10 mM NH₄HCO₃), B: ACN]; B %: 70%-100%, 8 min), followed by a second purification by prep-HPLC (column: Waters Xbridge 150×25 mm×5 μm; mobile phase: [A: water (0.05% ammonia hydroxide v/v), B: ACN]; B %: 75%-100%, 10 min) to give pure Starting Material 1.

Starting Material 1 was then separated by SFC (column: REGIS (s,s) WHELK-01 (250 mm×50 mm×10 μm); mobile phase: [A: 0.1% NH₃ in H₂O, B: IPA]; B %: 30%; 140 min) to give three isomers: 2-P1 (25 mg, 7% yield), 2-P2 (15 mg, 4% yield) and 2-P3 (55 mg, 15% yield), each as a white solid.

2-P3 (55 mg, 88 μmol) was separated by prep-SFC (column: DAICEL CHIRALPAK AS-H (250 mm×30 mm×5 μm); mobile phase: [A: 0.1% NH₃ in H₂O, B: MeOH]; B %: 20%; 75 min) to give two isomers: 2-P3-1 (14 mg, 25% yield) and 2-P3-2 (10 mg, 17% yield), each as a white solid.

Each of 2-P1, 2-P2, 2-P3-1, and 2-P3-2 is a single diastereomer; absolute stereochemistry not determined.

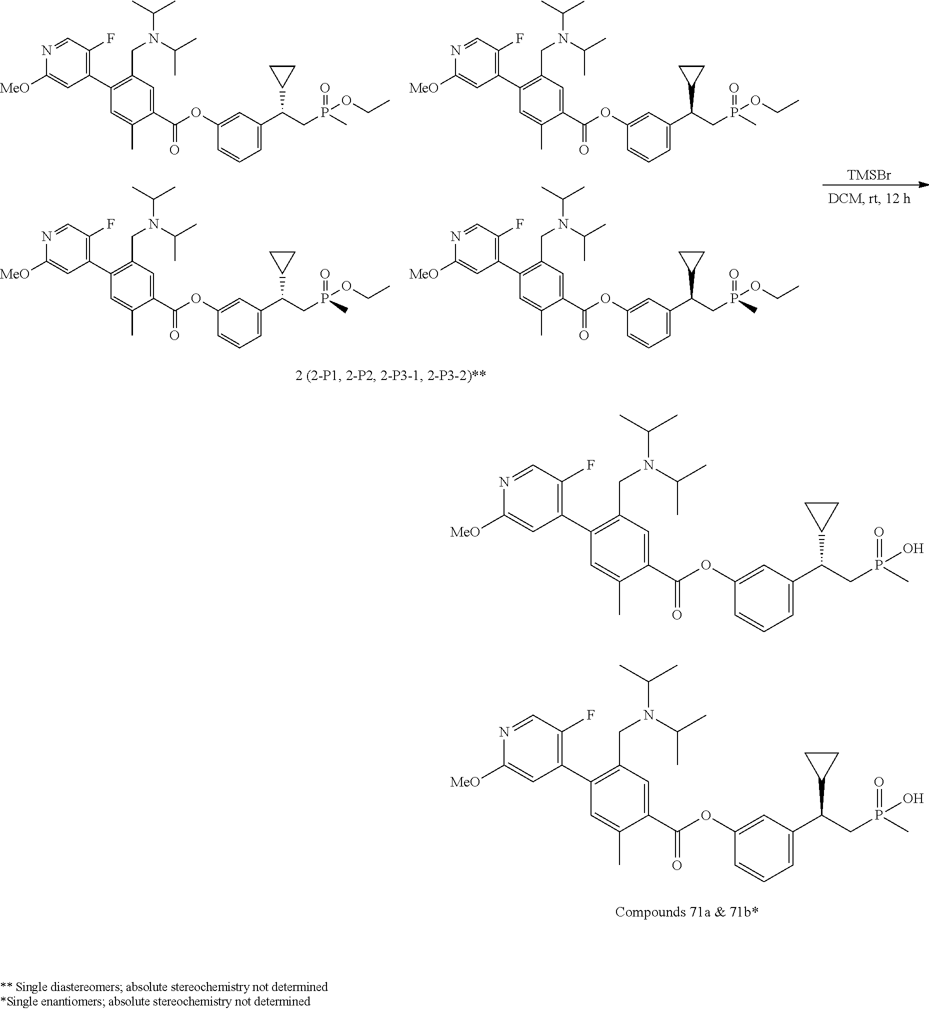

Step 2: ((R)-2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid & ((S)-2-cyclopropyl-2-(3-((5-((diisopropylamino)methyl)-4-(5-fluoro-2-methoxypyridin-4-yl)-2-methylbenzoyl)oxy)phenyl)ethyl)(methyl)phosphinic acid (Compounds 71a and 71b)*: Each of 2-P1, 2-P2, 2-P3-1, and 2-P3-2 was deprotected using a similar procedure to that of Example 30, Step 2. The four reactions each yielded a single enantiomer (71-1, 71-2, 71-3-1, and 71-3-2, respectively), where each corresponds to either Compound 71a or Compound 71b; absolute stereochemistry for each product was not determined.

71-1 (3.1 mg, 13% yield) was prepared from 2-P1 (25 mg, 40 μmol). It was purified by prep-HPLC (column: Waters Xbridge 150×25 mm×5 μm; mobile phase: [A: water (10 mM NH₄HCO₃); B: ACN]; B %: 38%-68%, 10 min). LCMS: tR=0.812 min, (ES+) m/z (M+H)⁺=597.4. ¹H NMR (400 MHz, CD₃OD) δ 8.48 (s, 1H), 8.23 (s, 1H), 7.44 (m, 2H), 7.29 (d, J=7.6 Hz, 1H), 7.21 (s, 1H), 7.12 (dd, J₁=1.6 Hz, J₂=1.6 Hz, 1H), 6.93 (d, J=4.8 Hz, 1H), 4.27 (m, 2H), 3.97 (s, 3H), 3.63 (m, 2H), 2.73 (s, 3H), 2.23 (m, 3H), 1.24 (d, J=0.8 Hz, 12H), 1.15 (m, 1H), 0.89 (d, J=14.0 Hz, 3H), 0.62 (m, 1H), 0.40 (m, 2H), 0.19 (m, 1H).

71-2 (3.6 mg, 25% yield) was prepared from 2-P2 (15 mg, 24 μmol). It was purified by prep-HPLC (column: Waters Xbridge 150×25 mm×5 μm; mobile phase: [A: water (10 mM NH₄HCO₃), B: ACN]; B %: 38%-68%, 10 min). LCMS: tR=0.815 min, (ES+) m/z (M+H)⁺=597.4. ¹H NMR (400 MHz, CD₃OD) δ 8.48 (s, 1H), 8.23 (s, 1H), 7.44 (m, 2H), 7.29 (d, J=7.6 Hz, 1H), 7.22 (s, 1H), 7.12 (dd, J₁=1.6 Hz, J₂=1.6 Hz, 1H), 6.93 (d, J=4.8 Hz, 1H), 4.29 (m, 2H), 3.97 (s, 3H), 3.62 (m, 2H), 2.73 (s, 3H), 2.23 (m, 3H), 1.23 (d, J=0.8 Hz, 12H), 1.15 (m, 1H), 0.89 (d, J=14.0 Hz, 3H), 0.62 (m, 1H), 0.40 (m, 2H), 0.19 (m, 1H).

71-3-1 (4.0 mg, 29% yield) was prepared from 2-P3-1 (14 mg, 22 μmol). It was purified by prep-HPLC (column: Waters Xbridge 150×25 mm×5 μm; mobile phase: [A: water (0.05% ammonia hydroxide v/v); B: ACN]; B %: 20%-50%, 10 min). LCMS: tR=0.808 min, (ES+) m/z (M+H)⁺=597.4. ¹H NMR (400 MHz, CD₃OD) δ 8.47 (s, 1H), 8.17 (s, 1H), 7.39 (m, 1H), 7.34 (s, 1H), 7.26 (d, J=7.6 Hz, 1H), 7.20 (s, 1H), 7.09 (dd, J₁=1.6 Hz, J₂=1.6 Hz, 1H), 6.85 (d, J=4.8 Hz, 1H), 3.96 (m, 5H), 3.34 (m, 2H), 2.70 (s, 3H), 2.32 (m, 1H), 2.12 (m, 2H), 1.11 (d, J=6.0 Hz, 13H), 0.81 (d, J=14.0 Hz, 3H), 0.60 (m, 1H), 0.39 (m, 2H), 0.19 (m, 1H).

71-3-2 (3.0 mg, 30% yield) was prepared from 2-P3-2 (10 mg, 16 μmol). It was purified by prep-HPLC (column: Waters Xbridge 150×25 mm×5 μm; mobile phase: [A: water (0.05% ammonia hydroxide v/v), B: ACN]; B %: 20%-50%, 10 min). LCMS: tR=0.819 min, (ES+) m/z (M+H)⁺=597.4. ¹H NMR (400 MHz, CD₃OD) δ 8.47 (s, 1H), 8.18 (s, 1H), 7.39 (m, 2H), 7.27 (d, J=7.6 Hz, 1H), 7.20 (s, 1H), 7.09 (dd, J₁=1.6 Hz, J₂=1.6 Hz, 1H), 6.87 (d, J=4.8 Hz, 1H), 4.08 (m, 2H), 3.96 (s, 3H), 3.43 (m, 2H), 2.71 (s, 3H), 2.32 (m, 1H), 2.12 (m, 2H), 1.30 (m, 1H), 1.14 (d, J=6.0 Hz, 12H), 0.82 (d, J=14.0 Hz, 3H), 0.60 (m, 1H), 0.40 (m, 2H), 0.18 (m, 1H).

### II. Biological Evaluation

### Example A-1: In Vitro Activity Assay

### Cell Lines Expressing GPR40/FFAR1

CHO-KI cells expressing human GPR40 were purchased from DiscoverX (95-1005C2). HEK293 cells expressing mouse FFAR1 were prepared using a mouse FFAR1 carrying plasmid purchased from OriGene Technologies (MR222997). The cells were transfected using Lipofectamine 2000 using manufacturer instructions and stable cell line was established from a single cell using geneticine selection. Assay ready frozen (ARF) cells were prepared and used throughout the study.

### Inositol Phosphate Accumulation Assay

The assay was performed in a 384-well plate format using IP1 assay kit from Cis-Bio. ARF cells expressing FFAR1 (mouse and human) were thawed, washed and then plated in the appropriate medium (F12 based medium for CHO hFFAR1 and DMEM based medium for HEK293 mFFAR1—both were supplemented with 10% FBS and penicillin/streptomycin). 20 μL of 3.5×10⁵ cells/mL were plated on a Poly D-Lysine coated 384-well white plate. The cells were then incubated for 16 hr at 37° C./5% CO₂. After 16 hr the medium was removed and 15 μL of stimulation buffer containing the test compounds was added to the cells. The plates were then incubated for 90 min at 37° C./5% CO₂. 5 μL of detection buffer (prepared as described in the IP-one kit) was added to each well and the plates were incubated at RT for 1 hr.

RT-FRET was measured using ClarioSTAR plate reader, calculating the ratio between emissions at 665 nm and 620 nm (HTRF ratio). HTRF ratio for positive (Max) and negative (Min) controls were used to normalize HTRF data and generate values for % activity. EC₅₀ and Max activity values were determined using a standard 4-parameter fit.

Results for exemplary compounds in the human GPR40 assay are shown in Table 1.

TABLE 1

| Compound | EC₅₀ |
|---|---|
| 1 | B |
| 2 | B |
| 3 | B |
| 4 | D |
| 5 | D |
| 6 | B |
| 7 | D |
| 8 | B |
| 9 | B |
| 10 | C |
| 11 | C |
| 12 | B |
| 13 | D |
| 14 | A |
| 15 | A |
| 16 | A |
| 17 | C |
| 18 | C |
| 19 | B |
| 20 | B |
| 21 | A |
| 22 | A |
| 23 | D |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | D |
| 36 | A |
| 37 | A |
| 38 | A |
| 39 | A |
| 40 | A |
| 41 | C |
| 42 | D |
| 43 | C |
| 44 | C |
| 45 | C |
| 46 | D |
| 47 | D |
| 48 | D |
| 49 | D |
| 50 | B |
| 51 | D |
| 52 | D |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | D |
| 65 | A |
| 66 | D |
| 67 | D |
| 68 | C |
| 69 | A |
| 70 | D |
| 71a | D |
| 71b | D |

A ≤ 10 nM;
10 nM < B ≤ 100 nM;
100 nM < C ≤ 500 nM;
D > 500 nM.

### Example A-2: In Vivo Plasma Levels in Mice

Male C57BL/6J mice 10-12 weeks old were acclimated to dosing (e.g., oral gavage) 2-3 times prior to the study. On the day of the study, food was removed for 5-6 hours, then the mice were dosed with test article or vehicle (e.g., by oral gavage at a volume of 10 mL/kg). Animals were euthanized with carbon dioxide typically 30 min post dose. Blood was collected via cardiac puncture for measurement of plasma concentrations of test article (parent) or metabolite resulting from ester cleavage.

Results for exemplary compounds are shown in Table 2.

TABLE 2

| | | 30 min | 30 min |
|---|---|---|---|
| | | Exposure: | Exposure: |
| | Dose | Parent | Metabolite |
| Compound | (mpk) | (nM) | (nM) |
| 15 | 30 | <2 | 6930 |
| 26 | 30 | <2 | 27700 |
| 36 | 30 | <5 | Not determined |

We claim:

1. A compound of Formula (I): 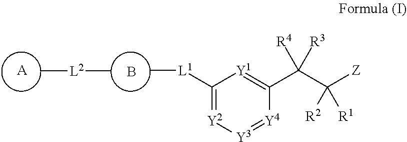
or a pharmaceutically acceptable salt, solvate, stereoisomer, or prodrug thereof, wherein:
Z is —C(O)OH, —C(O)OR⁵, —C(O)NHR⁶, —C(O)NHS(O)₂R⁵, —S(O)₂NHC(O)R⁵, —P(O)(R⁵)OR⁶, —P(O)(OR⁶)₂, —S(O)₂OR⁶;
or Z is a 4- or 5-membered heterocycle which is unsubstituted or substituted with 1, 2, 3, or 4 substituents selected from C₁-C₆ alkyl, —O—(C₁-C₆ alkyl), —OH, and =0;
R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —P(O)(OH)₂, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 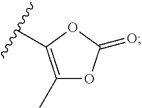 [SMILES: *c1oc(=O)oc1C]
R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
R¹, R², R³, and R⁴ are each independently hydrogen, halogen, —OH, —O—(C₁-C₆ alkyl), C₁-C₆ alkyl, C₃-C₆ cycloalkyl, or 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
Y¹, Y², Y³, and Y⁴ are each independently N, CH, or C—R^{Y};
each R^{Y} is independently halogen, —CN, —OH, —O—(C₁-C₆ alkyl), —NH₂, —NH—(C₁-C₆ alkyl), —N(C₁-C₆ alkyl)₂, C₁-C₆ alkyl, C₃-C₆ cycloalkyl, and 3- to 6-membered heterocycloalkyl; wherein each alkyl, cycloalkyl, and heterocycloalkyl is unsubstituted or substituted with 1, 2, or 3 substituents selected from halogen, —CN, —OH, —O—(C₁-C₆ alkyl), and C₁-C₆ alkyl;
L¹ is *—O—C(O)—, or *—C(O)—O—; wherein * represents the connection to Ring B;
Ring B is arylene, heteroarylene, C₃-C₁₀ cycloalkylene, or 3- to 10-membered heterocycloalkylene; wherein the arylene, heteroarylene, cycloalkylene, or heterocycloalkylene is unsubstituted or substituted with 1, 2, 3, or 4 R^{B} substituents;
Ring A is aryl, heteroaryl, C₃-C₁₀ cycloalkyl, or 3- to 10-membered heterocycloalkyl;
wherein the aryl, heteroaryl, cycloalkyl, or heterocycloalkyl is unsubstituted or substituted with 1, 2, 3, 4, or 5 R^{A} substituents;
L² is a bond, C₁-C₆ alkylene, or —(C₁-C₆ alkylene)-O—; wherein the alkylene is unsubstituted or substituted with 1, 2, or 3 substituents selected from the group consisting of halogen, —CN, —OH, C₁-C₆ alkyl, and —O—(C₁-C₆ alkyl);
each R^{A} is independently halogen, C₁-C₁₀ alkyl, C₂-C₁₀ alkenyl, C₂-C₁₀ alkynyl, C₁-C₁₀ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-OC(═O)R¹¹, -L^{A}-C(═O)NR¹¹R¹¹, -L^{A}-NR¹¹C(═O)R¹¹, -L^{A}-NR¹C(═O)NR¹¹R¹¹, -L^{A}-OC(═O)NR¹¹R¹¹, -L^{A}-NR¹¹C(═O)OR¹⁰, -L^{A}-OC(═O)OR¹⁰, -L^{A}-aryl, -L^{A}-heteroaryl, -L^{A}-(C₃-C₁₀ cycloalkyl), or -L^{A}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each R^{B} is independently halogen, C₁-C₁₀ alkyl, C₂-C₁₀ alkenyl, C₂-C₁₀ alkynyl, C₁-C₁₀ fluoroalkyl, -L^{B}-CN, -L^{B}-OH, -L^{B}-OR¹⁰, -L^{B}-NR¹¹R¹¹, -L^{B}-C(═O)R¹⁰, -L^{B}-C(═O)OR¹¹, -L^{B}-OC(═O)R¹¹, -L^{B}-C(═O)NR¹¹R¹¹, -L^{B}-NR¹¹C(═O)R¹¹, -L^{B}-NR¹C(═O)NR¹¹R¹¹, -L^{B}-OC(═O)NR¹¹R¹¹, -L^{B}-NR¹¹C(═O)OR¹⁰, -L^{B}-OC(═O)OR¹⁰, -L^{B}-aryl, -L^{B}-heteroaryl, -L^{B}-(C₃-C₁₀ cycloalkyl), or -L^{B}-(3- to 10-membered heterocycloalkyl); wherein each alkyl, alkenyl, alkynyl, aryl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
each L^{A} and L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl;
each R¹⁰ is independently C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl); and
each R¹¹ is independently hydrogen, C₁-C₁₀ alkyl, C₂-C₁₀ alkenyl, C₂-C₁₀ alkynyl, C₃-C₁₀ cycloalkyl, 3- to 10-membered heterocycloalkyl, phenyl, or monocyclic heteroaryl; wherein each alkyl, alkenyl, alkynyl, phenyl, heteroaryl, cycloalkyl, and heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl);
or two R¹¹ on the same nitrogen atom are taken together with the nitrogen to which they are attached to form a 3- to 10-membered N-heterocycloalkyl; wherein the heterocycloalkyl is unsubstituted or substituted with 1, 2, 3, 4, or 5 substituents selected from the group consisting of halogen, —CN, —OH, C₁-C₆ alkyl, C₁-C₆ fluoroalkyl, C₁-C₆ hydroxyalkyl, —O—(C₁-C₆ alkyl), and —O—(C₁-C₆ fluoroalkyl).

2. The compound of claim 1, or a pharmaceutically acceptable salt, solvate, stereoisomer, or prodrug thereof, wherein:
Y¹, Y², Y³, and Y⁴ are each independently N, CH, or C—R^{Y}; and
each R^{Y} is independently F, Cl, Br, —CN, —OH, —O—(C₁-C₆ alkyl), C₁-C₆ alkyl.

3. The compound of claim 1 or claim 2, or a pharmaceutically acceptable salt, solvate, stereoisomer, or prodrug thereof, wherein:
Y¹, Y², Y³, and Y⁴ are each independently N or CH.

4. The compound of any one of claims 1-3, or a pharmaceutically acceptable salt, solvate, stereoisomer, or prodrug thereof, wherein:
Z is —C(O)OH, —C(O)OR⁵, —C(O)NHR⁶, —C(O)NHS(O)₂R⁵, —S(O)₂NHC(O)R⁵, —P(O)(R⁵)OR⁶, —P(O)(OR⁶)₂, or —S(O)₂OR⁶
R⁵ is C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —P(O)(OH)₂, —O—(C₁-C₆ alkyl), C₁-C₆ alkyl, C₁-C₆ hydroxyalkyl, and 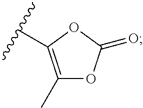 [SMILES: *c1oc(=O)oc1C]
and
R⁶ is hydrogen, C₁-C₆ alkyl, C₃-C₆ cycloalkyl, phenyl, or —(C₁-C₆ alkyl)-phenyl; wherein each alkyl, cycloalkyl, and phenyl is unsubstituted or substituted with one, two, or three substituents selected from —F, —Cl, —OH, —O—(C₁-C₆ alkyl), C₁-C₆ alkyl, and C₁-C₆ hydroxyalkyl.

5. The compound of any one of claims 1-4, or a pharmaceutically acceptable salt, solvate, stereoisomer, or prodrug thereof, wherein:
Z is —C(O)OH.

6. The compound of any one of claims 1-5, having the structure of Formula (II): 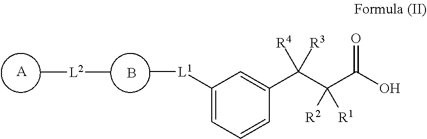
or a pharmaceutically acceptable salt, solvate, stereoisomer, or prodrug thereof.

7. The compound of any one of claims 1-6, or a pharmaceutically acceptable salt, solvate, stereoisomer, or prodrug thereof, wherein:
R¹, R², R³, and R⁴ are each independently hydrogen, halogen, C₁-C₆ alkyl, C₃-C₆ cycloalkyl.

8. The compound of any one of claims 1-7, or a pharmaceutically acceptable salt, solvate, stereoisomer, or prodrug thereof, wherein:
R¹, R², and R³ are each independently hydrogen, halogen, or C₁-C₆ alkyl; and
R⁴ is C₃-C₆ cycloalkyl.

9. The compound of any one of claims 1-8, having the structure of Formula (III): 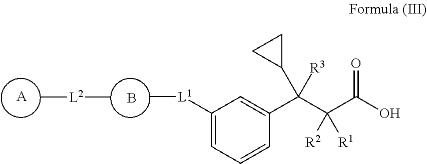
or a pharmaceutically acceptable salt, solvate, stereoisomer, or prodrug thereof, wherein:
R¹, R², and R³ are each independently hydrogen, —F, or methyl.

10. The compound of any one of claims 1-9, having the structure of Formula (IV): 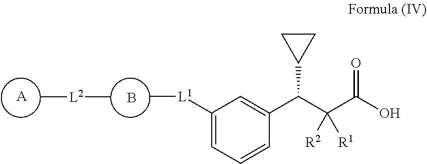
or a pharmaceutically acceptable salt, solvate, stereoisomer, or prodrug thereof, wherein:
R¹ and R² are each independently hydrogen, —F, or methyl.

11. The compound of any one of claims 1-10, having the structure of Formula (Va) or Formula (Vb): 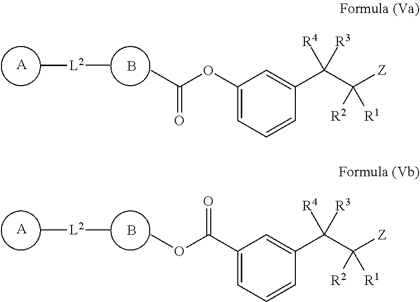
or a pharmaceutically acceptable salt, solvate, stereoisomer, or prodrug thereof.

12. The compound of any one of claims 1-11, or a pharmaceutically acceptable salt, solvate, stereoisomer, or prodrug thereof, wherein:
Ring B is 3- to 6-membered heterocycloalkylene; wherein the heterocycloalkylene is unsubstituted or substituted with 1, 2, 3, or 4 R^{B} substituents;
each R^{B} is independently unsubstituted C₁-C₁₀ alkyl;
L² is C₁-C₆ alkylene; wherein the alkylene is unsubstituted or substituted with 1, 2, or 3 substituents selected from the group consisting of —OH, C₁-C₆ alkyl, and —O—(C₁-C₆ alkyl); and
Ring A is aryl or heteroaryl; wherein the aryl or heteroaryl is unsubstituted or substituted with 1, 2, or 3 R^{A} substituents.

13. The compound of claim 12, having the structure of Formula (VIa) or Formula (VIb): 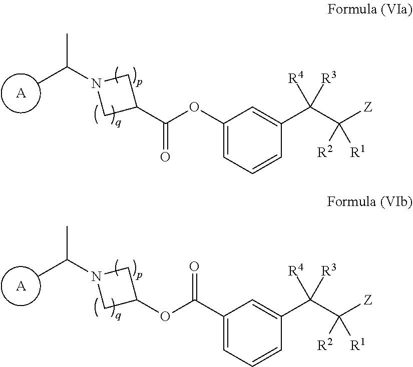
or a pharmaceutically acceptable salt, solvate, stereoisomer, or prodrug thereof, wherein:
p and q are each independently 1 or 2.

14. The compound of claim 12 or claim 13, or a pharmaceutically acceptable salt, solvate, stereoisomer, or prodrug thereof, wherein:
Ring A is phenyl or 5- or 6-membered monocyclic heteroaryl; wherein the phenyl or heteroaryl is unsubstituted or is substituted with 1, 2, or 3 R^{A} substituents;
each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹;
wherein the alkyl is unsubstituted or substituted with 1, 2, or 3 substituents selected from the group consisting of halogen, —OH, C₁-C₆ fluoroalkyl, —O—(C₁-C₆ alkyl), and —O—(C₁-C₆ fluoroalkyl); and
each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl.

15. The compound of claim 14, or a pharmaceutically acceptable salt, solvate, stereoisomer, or prodrug thereof, wherein:
each R^{A} is independently halogen, C₁-C₆ alkyl, C₁-C₆ fluoroalkyl, -L^{A}-OH, -L^{A}-OR¹⁰;
wherein the alkyl is unsubstituted or substituted with 1, 2, or 3 substituents selected from the group consisting of halogen, —OH, and C₁-C₆ fluoroalkyl; and
each L^{A} is independently a bond or unsubstituted C₁-C₆ alkylene.

16. The compound of any one of claims 1-11 having the structure of Formula (VIIa) or Formula (VIIb): 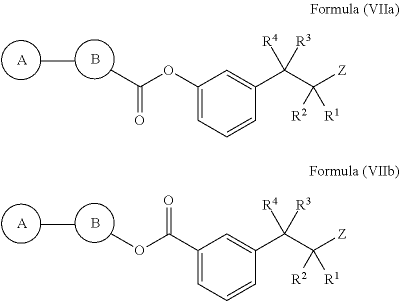
or a pharmaceutically acceptable salt, solvate, stereoisomer, or prodrug thereof; wherein:
Ring B is arylene or heteroarylene; wherein the arylene or heteroarylene is unsubstituted or substituted with 1, 2, 3, or 4 R^{B} substituents; and
Ring A is aryl or heteroaryl; wherein the aryl or heteroaryl is unsubstituted or substituted with 1, 2, 3, 4, or 5 R^{A} substituents.

17. The compound of claim 16, or a pharmaceutically acceptable salt, solvate, stereoisomer, or prodrug thereof, wherein:
Ring B is phenylene or 5- or 6-membered monocyclic heteroarylene; wherein the phenylene or heteroarylene is unsubstituted or is substituted with 1, 2, or 3 R^{B} substituents;
each R^{B} is independently halogen, C₁-C₆ alkyl, C₁-C₆ fluoroalkyl, -L^{B}-CN, -L^{B}-OH, -L^{B}-OR¹⁰, -L^{B}-NR¹¹R¹¹, -L^{B}-C(═O)OR¹¹, -L^{B}-C(═O)NR¹¹R¹¹, or -L^{B}-(3- to 10-membered heterocycloalkyl); wherein each alkyl and heterocycloalkyl is unsubstituted or substituted with 1, 2, or 3 substituents selected from the group consisting of halogen, —CN, —OH, C₁-C₆ alkyl, C₁-C₆ fluoroalkyl, —O—(C₁-C₆ alkyl), and —O—(C₁-C₆ fluoroalkyl);
each L^{B} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl;
Ring A is phenyl or 5- or 6-membered monocyclic heteroaryl; wherein the phenyl or heteroaryl is unsubstituted or is substituted with 1, 2, or 3 R^{A} substituents;
each R^{A} is independently halogen, C₁-C₆ alkyl, C₁-C₆ fluoroalkyl, -L^{A}-CN, -L^{A}-OH, -L^{A}-OR¹⁰, -L^{A}-NR¹¹R¹¹, -L^{A}-C(═O)R¹⁰, -L^{A}-C(═O)OR¹¹, -L^{A}-C(═O)NR¹¹R¹¹; wherein the alkyl is unsubstituted or substituted with 1, 2, or 3 substituents selected from the group consisting of —OH, C₁-C₆ alkyl, and —O—(C₁-C₆ alkyl); and
each L^{A} is independently a bond or C₁-C₆ alkylene; wherein the alkylene is unsubstituted or substituted with 1, 2, or 3 substituents selected from the group consisting of halogen, —CN, —OH, —O—(C₁-C₆ alkyl), and C₁-C₆ alkyl.

18. The compound of claim 16 or claim 17, or a pharmaceutically acceptable salt, solvate, stereoisomer, or prodrug thereof, wherein:
Ring B is phenylene or 5- or 6-membered monocyclic heteroarylene; wherein the phenylene or heteroarylene is unsubstituted or is substituted with 1, 2, or 3 R^{B} substituents;
each R^{B} is independently fluoro, chloro, C₁-C₆ alkyl, C₁-C₆ fluoroalkyl, -L^{B}-NR¹¹R¹¹, or -L^{B}-(3- to 10-membered heterocycloalkyl); wherein heterocycloalkyl is unsubstituted or substituted with 1, 2, or 3 substituents selected from the group consisting of C₁-C₆ alkyl;
each L^{B} is independently a bond or unsubstituted C₁-C₆ alkylene;
Ring A is phenyl or 6-membered monocyclic heteroaryl; wherein the phenyl or heteroaryl is unsubstituted or is substituted with 1, 2, or 3 R^{A} substituents;
each R^{A} is independently fluoro, chloro, C₁-C₆ alkyl, C₁-C₆ fluoroalkyl, -L^{A}-OH, -L^{A}-OR¹⁰, -L^{A}-NR¹¹R¹¹, or -L^{A}-C(═O)NR¹¹R¹¹; and
each L^{A} is independently a bond or unsubstituted C₁-C₆ alkylene.

19. The compound of any one of claims 16-18, having the structure of Formula (VIIIa) or Formula (VIIIb): 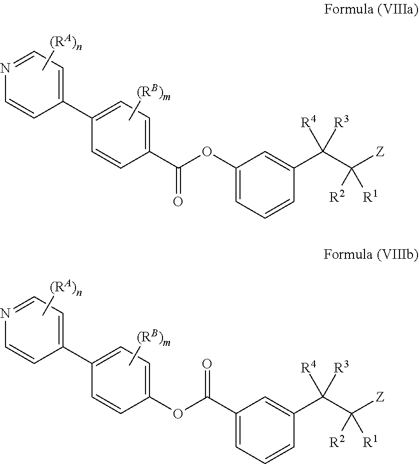
or a pharmaceutically acceptable salt, solvate, stereoisomer, or prodrug thereof; wherein:
n and m are each independently 0, 1, 2, or 3.

20. The compound of any one of claims 1-19, or a pharmaceutically acceptable salt, solvate, stereoisomer, or prodrug thereof, wherein:
each R¹⁰ is independently C₁-C₆ alkyl; wherein each alkyl is unsubstituted or substituted with 1, 2, 3, 4, or 5 substituents selected from the group consisting of halogen, —OH, C₁-C₆ alkyl and C₁-C₆ hydroxyalkyl; and
each R¹¹ is independently hydrogen, C₁-C₆ alkyl, or monocyclic heteroaryl; wherein each alkyl and heteroaryl is unsubstituted or substituted with 1, 2, 3, 4, or 5 substituents selected from the group consisting of halogen, —OH, C₁-C₆ alkyl and C₁-C₆ hydroxyalkyl;
or two R¹¹ on the same nitrogen atom are taken together with the nitrogen to which they are attached to form a 3- to 6-membered N-heterocycloalkyl; wherein the heterocycloalkyl is unsubstituted or substituted with 1, 2, 3, 4, or 5 substituents selected from the group consisting of halogen, —OH, C₁-C₆ alkyl, and C₁-C₆ hydroxyalkyl.

21. The compound of claim 1, or a pharmaceutically acceptable salt, solvate, stereoisomer, or prodrug thereof, selected from: 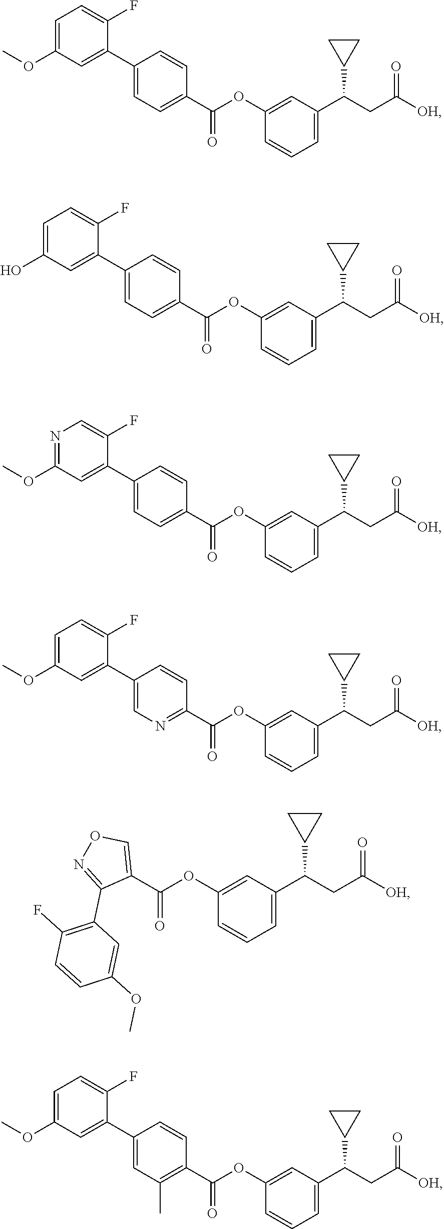 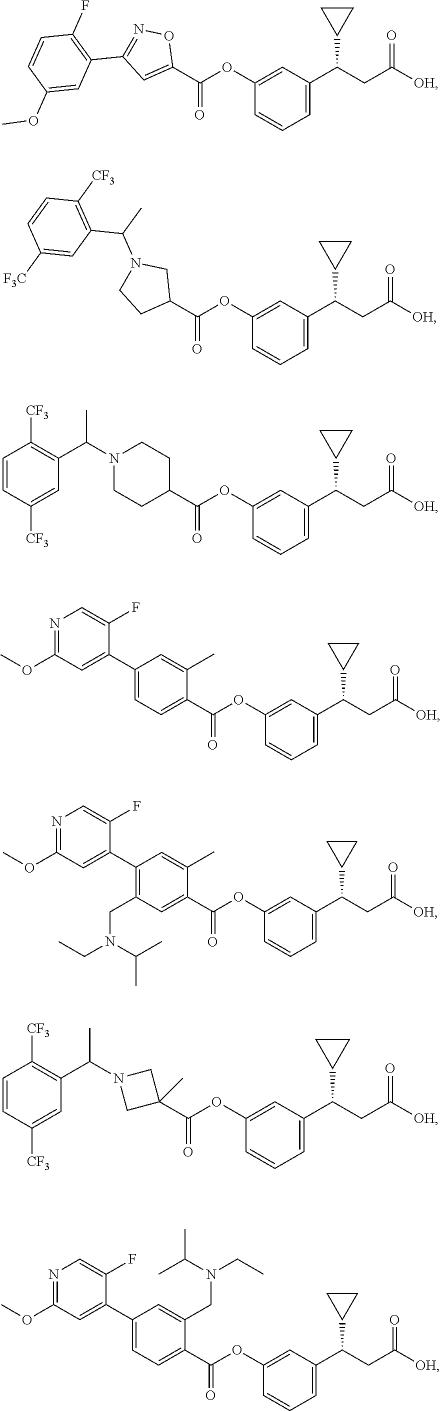 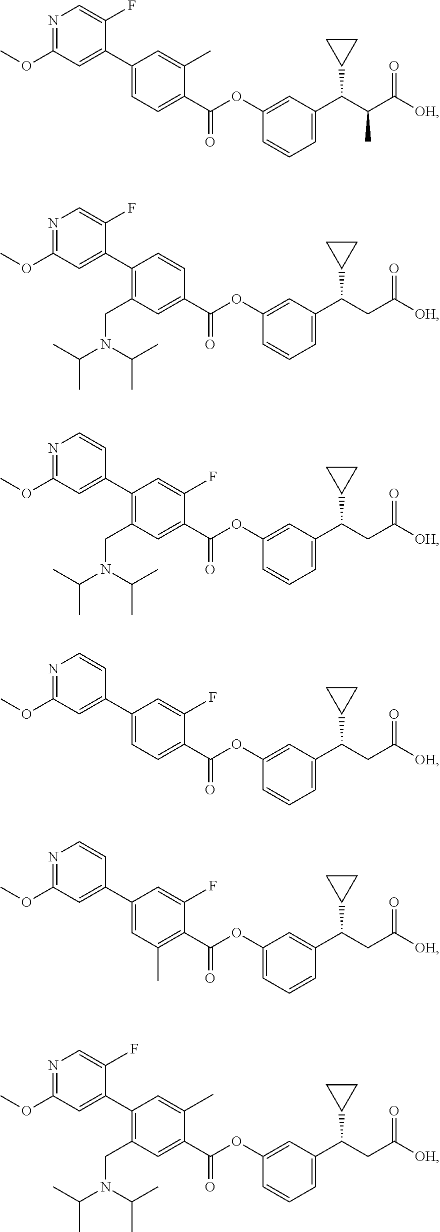 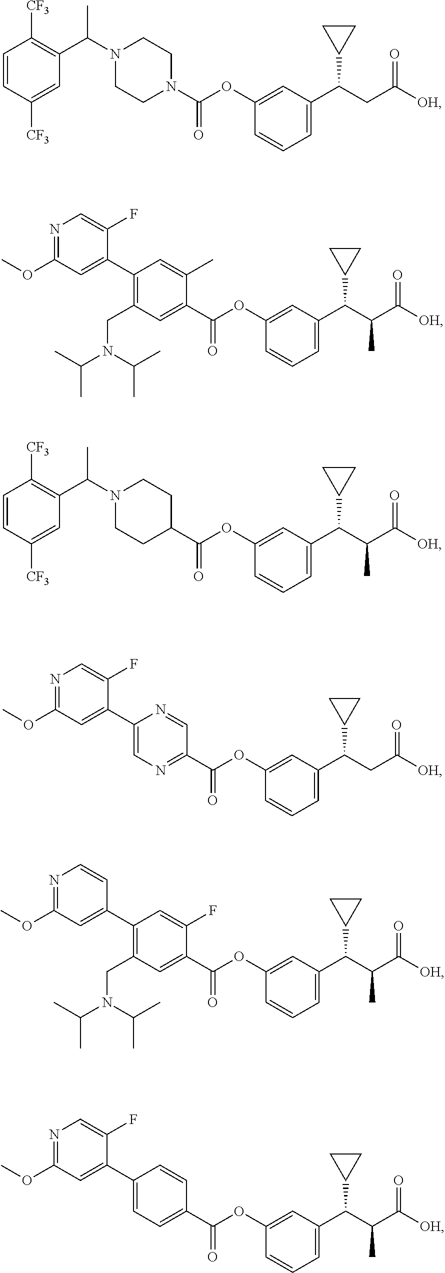 [SMILES: CC(c1cc(C(F)(F)F)ccc1C(F)(F)F)N1CCN(C(=O)Oc2cccc([C@@H](CC(=O)O)C3CC3)c2)CC1 ; COc1cc(-c2cc(C)c(C(=O)Oc3cccc([C@H](C4CC4)[C@H](C)C(=O)O)c3)cc2CN(C(C)C)C(C)C)c(F)cn1 ; CC(c1cc(C(F)(F)F)ccc1C(F)(F)F)N1CCC(C(=O)Oc2cccc([C@H](C3CC3)[C@H](C)C(=O)O)c2)CC1 ; COc1cc(-c2cnc(C(=O)Oc3cccc([C@@H](CC(=O)O)C4CC4)c3)cn2)c(F)cn1 ; COc1cc(-c2cc(F)c(C(=O)Oc3cccc([C@H](C4CC4)[C@H](C)C(=O)O)c3)cc2CN(C(C)C)C(C)C)ccn1 ; COc1cc(-c2ccc(C(=O)Oc3cccc([C@H](C4CC4)[C@H](C)C(=O)O)c3)cc2)c(F)cn1] 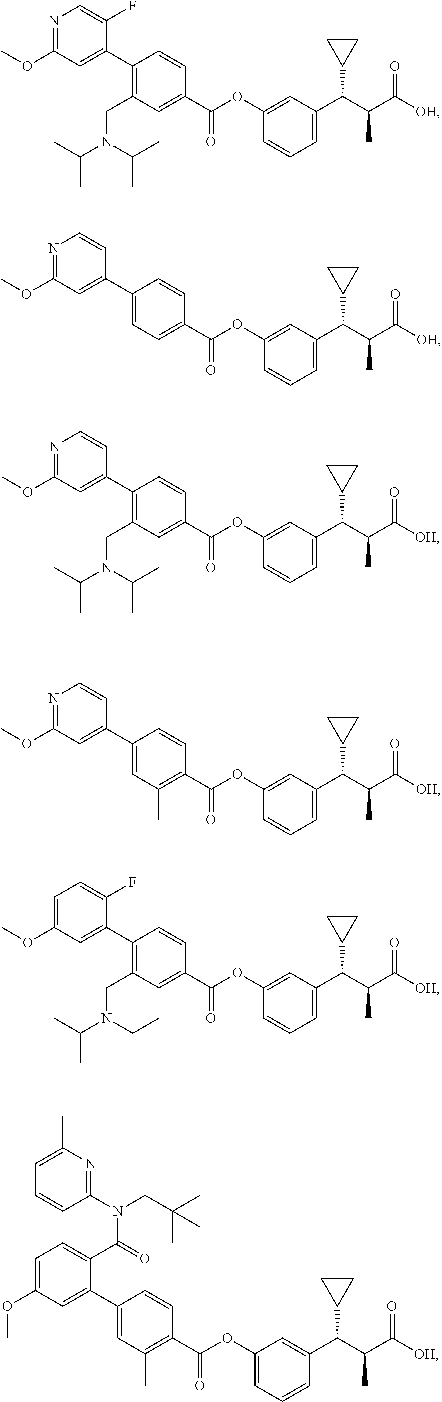 [SMILES: COc1cc(-c2ccc(C(=O)Oc3cccc([C@H](C4CC4)[C@H](C)C(=O)O)c3)cc2CN(C(C)C)C(C)C)c(F)cn1 ; COc1cc(-c2ccc(C(=O)Oc3cccc([C@H](C4CC4)[C@H](C)C(=O)O)c3)cc2)ccn1 ; COc1cc(-c2ccc(C(=O)Oc3cccc([C@H](C4CC4)[C@H](C)C(=O)O)c3)cc2CN(C(C)C)C(C)C)ccn1 ; COc1cc(-c2ccc(C(=O)Oc3cccc([C@H](C4CC4)[C@H](C)C(=O)O)c3)c(C)c2)ccn1 ; CCN(Cc1cc(C(=O)Oc2cccc([C@H](C3CC3)[C@H](C)C(=O)O)c2)ccc1-c1cc(OC)ccc1F)C(C)C ; COc1ccc(C(=O)N(CC(C)(C)C)c2cccc(C)n2)c(-c2ccc(C(=O)Oc3cccc([C@H](C4CC4)[C@H](C)C(=O)O)c3)c(C)c2)c1] 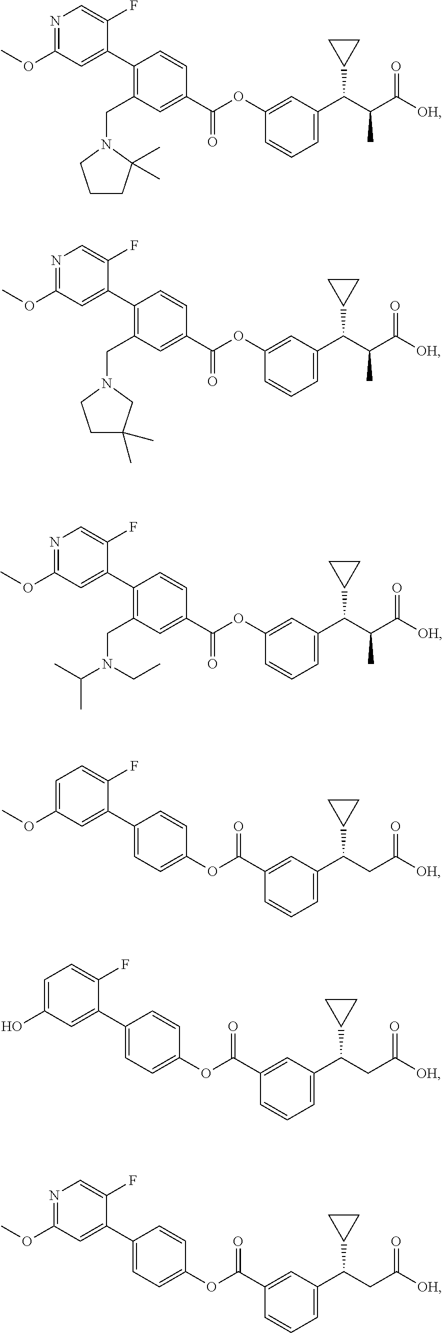 [SMILES: COc1cc(-c2ccc(C(=O)Oc3cccc([C@H](C4CC4)[C@H](C)C(=O)O)c3)cc2CN2CCCC2(C)C)c(F)cn1 ; COc1cc(-c2ccc(C(=O)Oc3cccc([C@H](C4CC4)[C@H](C)C(=O)O)c3)cc2CN2CCC(C)(C)C2)c(F)cn1 ; CCN(Cc1cc(C(=O)Oc2cccc([C@H](C3CC3)[C@H](C)C(=O)O)c2)ccc1-c1cc(OC)ncc1F)C(C)C ; COc1ccc(F)c(-c2ccc(OC(=O)c3cccc([C@@H](CC(=O)O)C4CC4)c3)cc2)c1 ; O=C(O)C[C@H](c1cccc(C(=O)Oc2ccc(-c3cc(O)ccc3F)cc2)c1)C1CC1 ; COc1cc(-c2ccc(OC(=O)c3cccc([C@@H](CC(=O)O)C4CC4)c3)cc2)c(F)cn1] 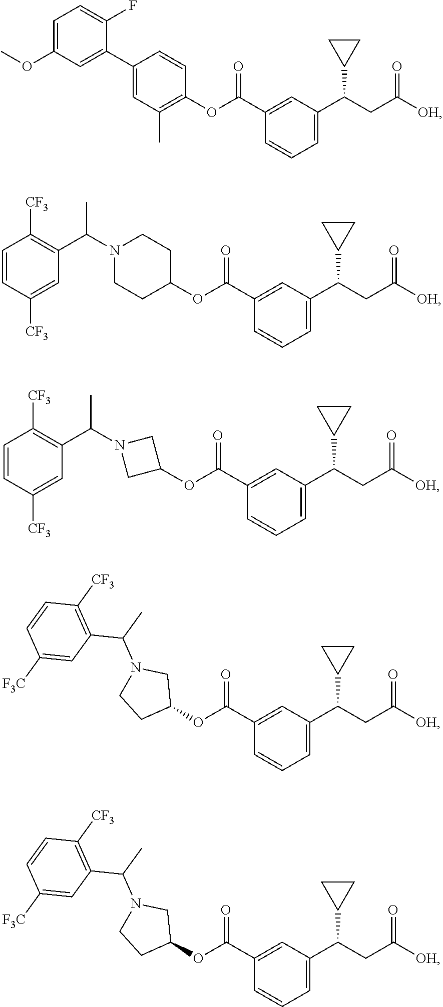 [SMILES: COc1ccc(F)c(-c2ccc(OC(=O)c3cccc([C@@H](CC(=O)O)C4CC4)c3)c(C)c2)c1 ; CC(c1cc(C(F)(F)F)ccc1C(F)(F)F)N1CCC(OC(=O)c2cccc([C@@H](CC(=O)O)C3CC3)c2)CC1 ; CC(c1cc(C(F)(F)F)ccc1C(F)(F)F)N1CC(OC(=O)c2cccc([C@@H](CC(=O)O)C3CC3)c2)C1 ; CC(c1cc(C(F)(F)F)ccc1C(F)(F)F)N1CC[C@@H](OC(=O)c2cccc([C@@H](CC(=O)O)C3CC3)c2)C1 ; CC(c1cc(C(F)(F)F)ccc1C(F)(F)F)N1CC[C@H](OC(=O)c2cccc([C@@H](CC(=O)O)C3CC3)c2)C1] 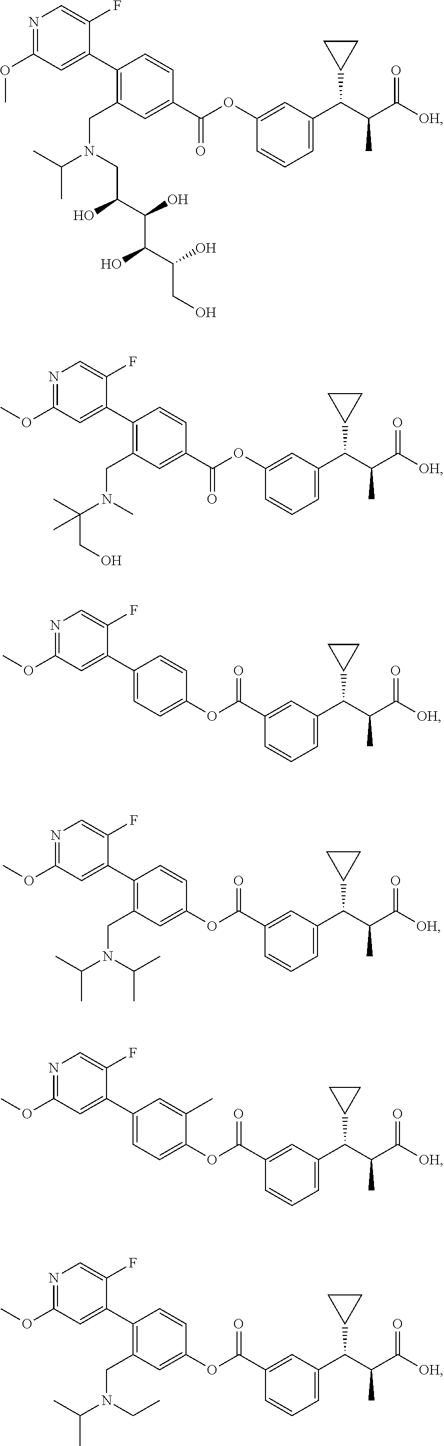 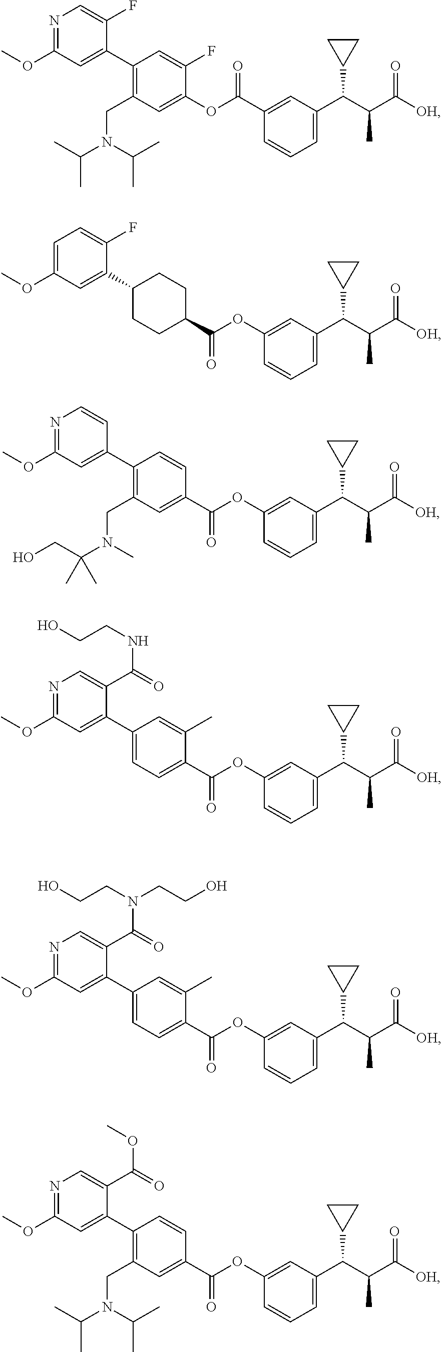 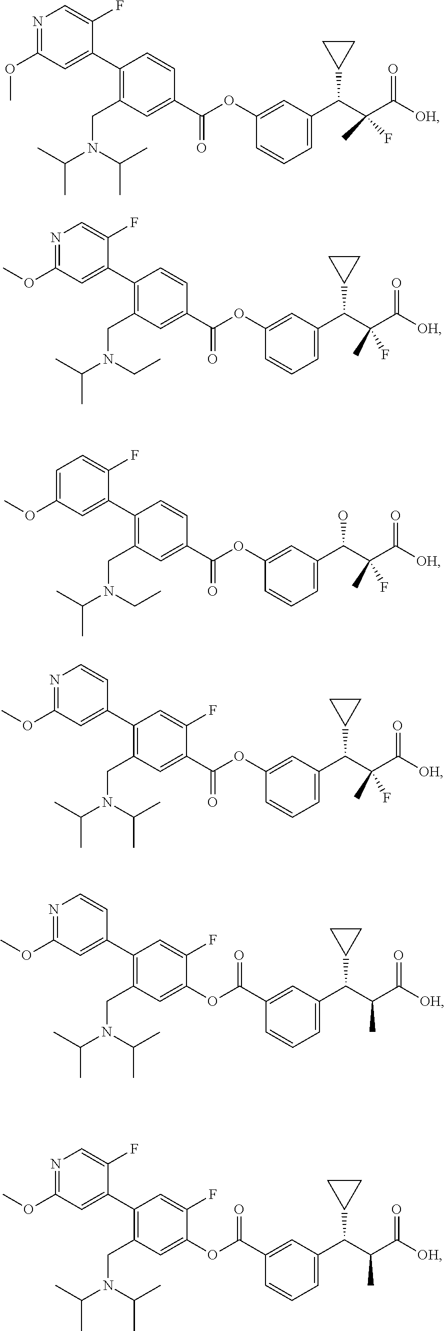 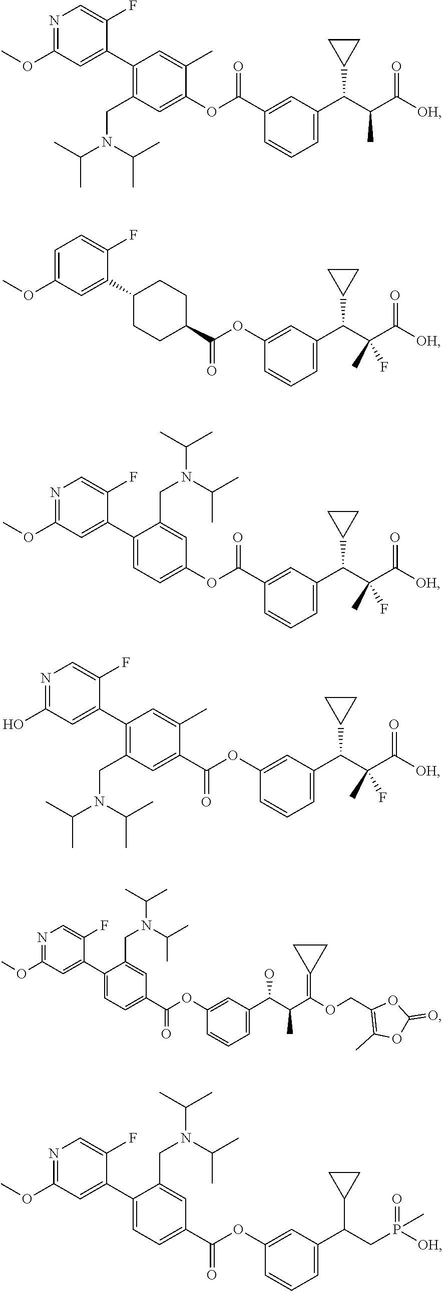 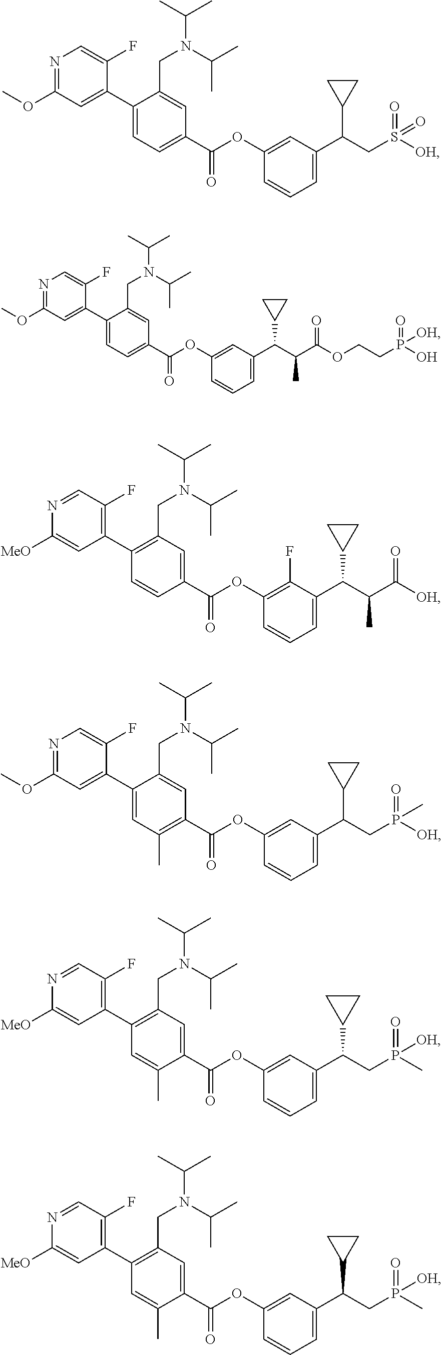
or a pharmaceutically acceptable salt, solvate, stereoisomer, or prodrug thereof.

22. The compound of claim 1, or a pharmaceutically acceptable salt, solvate, stereoisomer, or prodrug thereof, selected from: 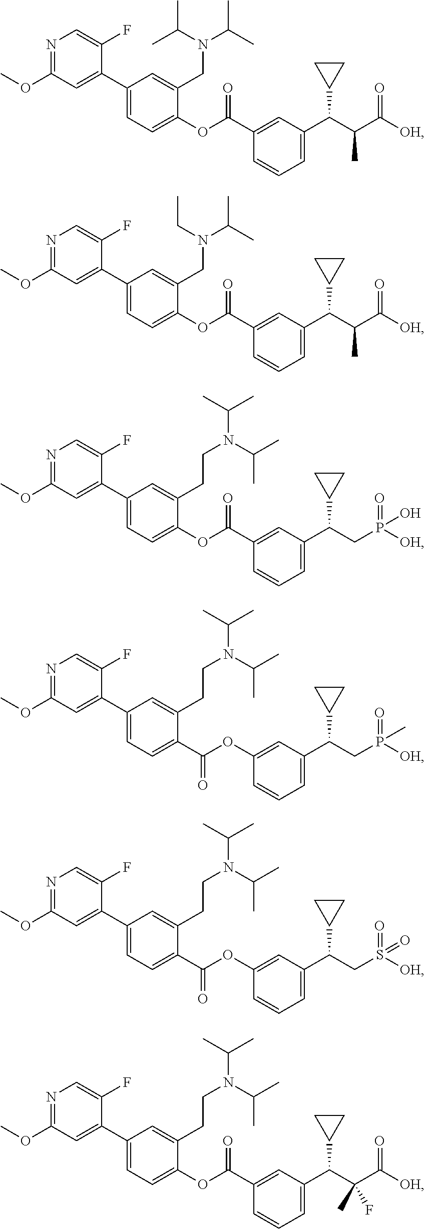 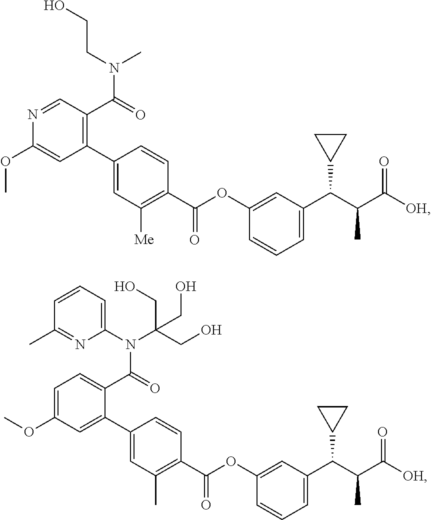 [SMILES: COc1cc(-c2ccc(C(=O)Oc3cccc([C@H](C4CC4)[C@H](C)C(=O)O)c3)c(C)c2)c(C(=O)N(C)CCO)cn1 ; COc1ccc(C(=O)N(c2cccc(C)n2)C(CO)(CO)CO)c(-c2ccc(C(=O)Oc3cccc([C@H](C4CC4)[C@H](C)C(=O)O)c3)c(C)c2)c1]
or a pharmaceutically acceptable salt,
solvate, stereoisomer, or prodrug thereof.

23. A pharmaceutical composition comprising a compound of any one of claims 1-22, or a pharmaceutically acceptable salt, solvate, stereoisomer, or prodrug thereof, and at least one pharmaceutically acceptable excipient.

24. A method of treating a condition or disorder involving the gut-brain axis in a subject in need thereof, the method comprising administering to the subject a therapeutically effective amount of a compound of any one of claims 1-22, or a pharmaceutically acceptable salt, solvate, stereoisomer, or prodrug thereof.

25. The method of claim 24, wherein the condition or disorder is associated with GPR40 activity.

26. The method of claim 24 or claim 25, wherein the condition or disorder is a metabolic disorder.

27. The method of claim 26, wherein the condition or disorder is type 2 diabetes, hyperglycemia, metabolic syndrome, obesity, hypercholesterolemia, nonalcoholic steatohepatitis, or hypertension.

28. The method of claim 24 or claim 25, wherein the condition or disorder is a nutritional disorder.

29. The method of claim 28, wherein the condition or disorder is short bowel syndrome, intestinal failure, or intestinal insufficiency.

30. The method of any one of claims 24-29, wherein the compound is gut-restricted.

31. The method of claim 30, wherein the compound is a soft drug.

32. The method of claim 30, wherein the compound has low systemic exposure.

33. The method of any one of claims 24-32, further comprising administering one or more additional therapeutic agents to the subject.

34. The method of claim 33, wherein the one or more additional therapeutic agents are selected from a TGR5 agonist, a GPR119 agonist, an SSTR5 antagonist, an SSTR5 inverse agonist, a CCK1 agonist, a PDE4 inhibitor, a DPP-4 inhibitor, or a combination thereof.

35. The method of claim 34, wherein the TGR5 agonist, GPR119 agonist, SSTR5 antagonist, SSTR5 inverse agonist or CCK1 agonist is gut-restricted.

36. Use of a compound of any one of claims 1-22, or a pharmaceutically acceptable salt, solvate, stereoisomer, or prodrug thereof, for the preparation of a medicament for the treatment of a condition or disorder involving the gut-brain axis in a subject in need thereof.

37. The use of claim 36, wherein the condition or disorder is associated with GPR40 activity.

38. The use of claim 36 or claim 37, wherein the condition or disorder is a metabolic disorder.

39. The use of claim 38, wherein the condition or disorder is type 2 diabetes, hyperglycemia, metabolic syndrome, obesity, hypercholesterolemia, nonalcoholic steatohepatitis, or hypertension.

40. The use of claim 36 or claim 37, wherein the condition or disorder is a nutritional disorder.

41. The use of claim 40, wherein the condition or disorder is short bowel syndrome, intestinal failure, or intestinal insufficiency.

42. The use of any one of claims 36-41, wherein the compound is gut-restricted.

43. The use of claim 42, wherein the compound is a soft drug.

44. The use of claim 42, wherein the compound has low systemic exposure.

45. A method of treating a condition or disorder involving the gut-brain axis in a subject in need thereof, the method comprising administering to the subject a therapeutically effective amount of a gut-restricted GPR40 modulator.

46. The method of claim 45, wherein the condition or disorder is associated with GPR40 activity.

47. The method of claim 45 or claim 46, wherein the modulator is an agonist, full agonist, or partial agonist of GPR40.

48. The method of any one of claims 45-47, further comprising administering one or more additional therapeutic agents to the subject.

49. The method of claim 48, wherein the one or more additional therapeutic agents are selected from a TGR5 agonist, a GPR119 agonist, an SSTR5 antagonist, an SSTR5 inverse agonist, a CCK1 agonist, a PDE4 inhibitor, a DPP-4 inhibitor, a GLP-1 receptor agonist, a GOAT inhibitor, metformin, or a combination thereof.

50. The method of claim 49, wherein the TGR5 agonist, GPR119 agonist, SSTR5 antagonist, SSTR5 inverse agonist or CCK1 agonist is gut-restricted.

51. The method of any one of claims 45-50, wherein the condition or disorder is a metabolic disorder.

52. The method of claim 51, wherein the condition or disorder is type 2 diabetes, hyperglycemia, metabolic syndrome, obesity, hypercholesterolemia, nonalcoholic steatohepatitis, or hypertension.

53. The method of any one of claims 45-50, wherein the condition or disorder is a nutritional disorder.

54. The method of claim 53, wherein the condition or disorder is short bowel syndrome, intestinal failure, or intestinal insufficiency.

55. The method of any one of claims 45-54, wherein the modulator is a soft drug.

56. The method of any one of claims 45-55, wherein the modulator is a compound of any one of claims 1-22.

57. Use of a gut-restricted GPR40 modulator for the preparation of a medicament for the treatment of a condition or disorder involving the gut-brain axis in a subject in need thereof.

58. The use of claim 57, wherein the condition or disorder is associated with GPR40 activity.

59. The use of claim 57 or claim 58, wherein the modulator is an agonist, full agonist, or partial agonist of GPR40.

60. The use of any one of claims 57-59, wherein the condition or disorder is a metabolic disorder.

61. The method of claim 60, wherein the condition or disorder is type 2 diabetes, hyperglycemia, metabolic syndrome, obesity, hypercholesterolemia, nonalcoholic steatohepatitis, or hypertension.

62. The method of any one of claims 57-59, wherein the condition or disorder is a nutritional disorder.

63. The method of claim 62, wherein the condition or disorder is short bowel syndrome, intestinal failure, or intestinal insufficiency.

64. The method of any one of claims 57-63, wherein the modulator is a soft drug.

65. The method of any one of claims 57-64, wherein the modulator is a compound of any one of claims 1-22.
